(19) United States
(12) Patent Application Publication
(10) Pub. No.: US 2023/0124851 A1
(43) Pub. Date: Apr. 20, 2023

(54) B7H3 SINGLE DOMAIN ANTIBODIES AND THERAPEUTIC COMPOSITIONS THEREOF

(71) Applicant: Inhibrx, lnc., La Jolla, CA (US)

(72) Inventors: Brendan P. ECKELMAN, La Jolla, CA (US); Michael D. KAPLAN, La joiia, CA (US); Katelyn M. WILLIS, La Jolla, CA (US); Kyle S. JONES, La Jolla, CA (US); Angelica N. SANABRIA, La Jolla, CA (US); Sydney A. BARNES, La Jolla, CA (US); Margaret E. HAERR, La Joiia, CA (US); John C. TIMMER, La Jolla, CA (US)

(73) Assignee: Inhibrx, lnc., La Jolla, CA (US)

(21) Appl. No.: 17/283,902

(22) PCT Filed: Oct. 9, 2019
(86) PCT No.: PCT/US2019/055427

§ 371 Date: Apr. 8, 2021

Related U.S. Application Data
(60) Provisional application No. 62/877,812, filed on Jul. 23, 2019.
(60) Provisional application No. 62/832,274, filed on Apr. 10, 2019.
(60) Provisional application No. 62/744,640, filed on Oct. 11, 2018.

(51) Int. Cl. C07K 16/28; A61P 35/00

(52) U.S. Cl. CPC C07K 16/2827; C07K 16/2809; A61P 35/00; C07K 2317/565; C07K 2317/24; C07K 2317/52; C07K 2317/31

(57) ABSTRACT

Provided herein are binding polypeptides that specifically bind B7H3. More specifically, provided herein are fusion proteins, including multivalent and/or multispecific constructs and chimeric antigen receptors, that bind B7H3. Also provided are pharmaceutical compositions containing the polypeptides, nucleic acid molecules encoding the polypeptides and vectors and cells thereof, and methods of use and uses of the provided B7H3 binding polypeptides for treating diseases and conditions, such as cancer.

### CROSS-REFERENCE TO RELATED APPLICATIONS

This application is a national stage application under 35 U.S.C. § 371 of International Application No. PCT/US2019/055427, filed on Oct. 9, 2019, which claims priority to U.S. provisional applications: 62/744,640, filed Oct. 11, 2018, entitled “B7H3 SINGLE DOMAIN ANTIBODIES AND THERAPEUTIC COMPOSITIONS THEREOF”; 62/832,274, filed Apr. 10, 2019, entitled “B7H3 SINGLE DOMAIN ANTIBODIES AND THERAPEUTIC COMPOSITIONS THEREOF”; and 62/877,812 filed Jul. 23, 2019, entitled “B7H3 SINGLE DOMAIN ANTIBODIES AND THERAPEUTIC COMPOSITIONS THEREOF” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 744952000400SubSeqList.TXT, created Jan. 10, 2022, which is 676,515 bytes in size. The information in the electronic format of the Sequence Listing is incorporated by reference in its entirety.

### FIELD

This disclosure generally provides binding polypeptides that specifically bind B7H3. More specifically, the disclosure relates to fusion proteins, including multivalent and/or multispecific constructs and chimeric antigen receptors, that bind at least B7H3. The disclosure also provides nucleic acid molecules encoding the polypeptides and vectors and cells thereof, and methods of use and uses of the provided B7H3 binding polypeptides for treating diseases and conditions, such as cancer.

### BACKGROUND

B7H3 is a member of the B7 family of immune cell modulating molecules. It is expressed on the surface of a wide variety of tumor cells and tumor vasculature, and its expression has been associated with poor prognosis in a variety of cancers. The expression of B7H3 on a variety of cancers in humans, including solid tumors, makes B7H3 a desirable therapeutic target. Improved therapeutic molecules and agents targeting B7H3 are needed. Provided herein are embodiments that meet such needs.

### SUMMARY

Provided herein is a B7H3-binding polypeptide construct, comprising at least one heavy chain only variable domain (B7H3 VHH domain) that specifically binds B7H3. In some embodiments, the B7H3-binding polypeptide construct further comprises one or more additional binding domain that binds to a target other than B7H3.

Provided herein is a B7H3-binding construct, comprising at least one heavy chain only variable domain (B7H3 VHH domain) comprising a complementarity determining region 1 (CDR1) comprising an amino acid sequence selected from the group consisting of SEQ ID NO: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and 145; a complementarity determining region 2 (CDR2) comprising an amino acid sequence selected from the group consisting of SEQ ID NO: 146, 147, 148, 149, 150, 151, 152, 153, 154, 155, 156, 157, 158, 159, 160, 161, 162, 163, 164, 165, 166 and 167; and a complementarity determining region 3 (CDR3) comprising an amino acid sequence selected from the group consisting of SEQ ID NO: 168, 169, 170, 171, 172, 173, 174, 175, 176, 177, 178, 179, 180, 181, 182, 183, 184, 185, 186, 187, 188, 189, and 483-488. In some embodiments, the B7H3-binding polypeptide construct further comprises one or more additional binding domain that binds to a target other than B7H3.

In some embodiments, the B7H3-binding polypeptide construct is a dimer.

In some embodiments, the B7H3 has the sequence set forth in SEQ ID NO:190 or a mature form thereof lacking the signal sequence. In some embodiments, the B7H3 is a human B7H3.

In some embodiments, the at least one B7H3 VHH domain is humanized. In some embodiments, the B7H3 VHH is a camelid VHH. In some embodiments, the B7H3 VHH is a humanized form of a camelid VHH.

In some embodiments, the one or more additional binding domains binds to an activating receptor on an immune cell. In some embodiments, the immune cell is a T cell. In some embodiments, the activating receptor is CD3 (CD3ε ). In some embodiments, the binding polypeptide construct is bispecific for B7H3 and CD3. In some embodiments, the immune cell is a Natural Killer (NK) cell. In some embodiments, the activating receptor is CD16 (CD16a). In some embodiments, the B7H3-bnding polypeptide construct is bispecific for B7H3 and CD16a.

In some embodiments, the one or more additional binding domain binds to a cytokine receptor. In some embodiments, the one or more additional binding domain is a cytokine or is a truncated fragment or variant thereof capable of binding to the cytokine receptor. In some embodiments, the cytokine is an interferon, or is a truncated fragment or variant of an interferon. In some embodiments, the interferon is a type I interferon, a type II interferon, a truncated fragment or variant of a type I interferon, or a truncated fragment of variant of a type II interferon. In some embodiments, the interferon is selected from a type I interferon that is an IFN-alpha or an IFN-beta, or is a truncated fragment or variant thereof or a type II interferon that is an or IFN-gamma or a truncated fragment or variant thereof.

In some embodiments, the one or more additional binding domain comprises an antibody or antigen-binding fragment thereof. In some embodiments, the one or more additional binding domain is monovalent. In some embodiments, the antibody or antigen-binding fragment thereof is an Fv, a disulfide-stabilized Fv (dsFv), scFv, a Fab, a single domain antibody (sdAb), a VNAR, or a VHH.

In some of any of the provided embodiments, the polypeptide comprises an immunoglobulin Fc region. In some embodiments, the polypeptide comprises an immunoglobulin Fc region that links the at least one VHH domain and the one or more additional binding domain. In some embodiemnts, the B7H3-binding polypeptide construct is a dimer. In some embodiments, the Fc region is a homodimeric Fc region.

In some of any of the provided embodiments, the Fc region comprises the sequence of amino acids set forth in any of SEQ ID NOS: 198, 200, 201, 202 or 203, or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 198, 200, 201, 202 or 203. In some embodiments, the Fc region is a human IgG1.

In some of any of the provided embodiments, the B7H3-binding polypeptide construct is a dimer. In some embodiments, the Fc region is a homodimeric Fc region.

In some of any of the provided embodiments, the Fc region is a human IgG1.

In some of any of the provided embodiments, the Fc region comprises the sequence of amino acids set forth in SEQ ID NO: 198 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 198.

In some embodiments, the Fc region is a heterodimeric Fc region. In some embodiments, the Fc region exhibits effector function. In some embodiments, the Fc region comprises a polypeptide comprising one or more amino acid modification that reduces effector function and/or reduces binding to an effector molecule selected from an Fc gamma receptor or C1q. In some embodiments, the one or more amino acid modification is deletion of one or more of Glu233, Leu234 or Leu235.

In some of any of the provided embodiments, the Fc region comprises the sequence of amino acids set forth in SEQ ID NO: 199 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 199.

In some embodiments, the at least one B7H3 VHH domain comprises the VHH domain sequence set forth in any of SEQ ID NOS: 1-114, 466, 467, 489, 490, or 492-518 or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 1-114, 466, 467, 489, 490, or 492-518 and binds B7H3.

In some embodiments, the at least one B7H3 VHH domain comprises the VHH domain sequence set forth in any of SEQ ID NOS: 1, 8-35, 40, 41, 44, 56-110, 466, 467, 489, 490, or 492-518 or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 1, 8-35, 40, 41, 44, 56-110, 466, 467, 489, 490, or 492-518 and binds B7H3.

In some of any of the provided embodiments, the at least one B7H3 domain comprises the sequence set forth in (i) SEQ ID NO:1, (ii) a humanized variant of SEQ ID NO:1, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:1, and binds B7H3. In some embodiments, the at least one B7H3 VHH domain comprises a CDR1 comprising an amino acid sequence selected from the group consisting of SEQ ID NO: 115, 116, 117, 118, 119, 120 and 121; a CDR2 comprising an amino acid sequence selected from the group consisting of SEQ ID NO: 146, 147, 148, 149, 150 and 151; and a CDR3 comprising an amino acid sequence selected from the group consisting of SEQ ID NO: 168 and 169. In some embodiments, the at least one B7H3 VHH domain comprises a CDR1, CDR2 and CDR3 set forth in SEQ ID NOS: 115, 146 and 168, respectively. In some embodiments, the at least one B7H3 VHH domain comprises a CDR1, CDR2 and CDR3 set forth in SEQ ID NOS: 115, 147 and 168, respectively. In some embodiments, the at least one B7H3 VHH domain comprises a CDR1, CDR2 and CDR3 set forth in SEQ ID NOS: 115, 148 and 168, respectively. In some embodiments, the at least one B7H3 VHH domain comprises a CDR1, CDR2 and CDR3 set forth in SEQ ID NOS: 115, 149 and 168, respectively. In some embodiments, the at least one B7H3 VHH domain comprises a CDR1, CDR2 and CDR3 set forth in SEQ ID NOS: 115, 150 and 168, respectively. In some embodiments, the at least one B7H3 VHH domain comprises a CDR1, CDR2 and CDR3 set forth in SEQ ID NOS: 116, 146 and 168, respectively. In some embodiments, the at least one B7H3 VHH domain comprises a CDR1, CDR2 and CDR3 set forth in SEQ ID NOS: 117, 146 and 168, respectively. In some embodiments, the at least one B7H3 VHH domain comprises a CDR1, CDR2 and CDR3 set forth in SEQ ID NOS: 118, 146 and 168, respectively. In some embodiments, the at least one B7H3 VHH domain comprises a CDR1, CDR2 and CDR3 set forth in SEQ ID NOS: 115, 146 and 169, respectively. In some embodiments, the at least one B7H3 VHH domain comprises a CDR1, CDR2 and CDR3 set forth in SEQ ID NOS: 119, 146 and 168, respectively. In some embodiments, the at least one B7H3 VHH domain comprises a CDR1, CDR2 and CDR3 set forth in SEQ ID NOS: 120, 146 and 168, respectively. In some embodiments, the at least one B7H3 VHH domain comprises a CDR1, CDR2 and CDR3 set forth in SEQ ID NOS: 115, 151 and 168, respectively. In some embodiments, the at least one B7H3 VHH domain comprises a CDR1, CDR2 and CDR3 set forth in SEQ ID NOS: 116, 147 and 168, respectively. In some embodiments, the at least one B7H3 VHH domain comprises a CDR1, CDR2 and CDR3 set forth in SEQ ID NOS: 118, 147 and 168, respectively. In some embodiments, the at least one B7H3 VHH domain comprises a CDR1, CDR2 and CDR3 set forth in SEQ ID NOS: 119, 147 and 168, respectively. In some embodiments, the at least one B7H3 VHH domain comprises a CDR1, CDR2 and CDR3 set forth in SEQ ID NOS: 116, 151 and 168, respectively. In some embodiments, the at least one B7H3 VHH domain comprises a CDR1, CDR2 and CDR3 set forth in SEQ ID NOS: 115, 146 and 168, respectively. In some embodiments, the at least one B7H3 VHH domain comprises a CDR1, CDR2 and CDR3 set forth in SEQ ID NOS: 121, 147 and 168, respectively. In some embodiments, the at least one B7H3 VHH domain comprises a CDR1, CDR2 and CDR3 set forth in SEQ ID NOS: 115, 146 and 168, respectively. In some embodiments, the at least one B7H3 VHH domain comprises a CDR1, CDR2 and CDR3 set forth in SEQ ID NOs: 119, 149 and 168, respectively. In some embodiments, the at least one B7H3 VHH domain comprises a CDR1, CDR2 and CDR3 set forth in SEQ ID NOS: 122, 151 and 168, respectively. In some embodiments, the at least one B7H3 VHH domain comprises the sequence of amino acids set forth in any one of SEQ ID NOs:2-34 and 467 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:2-34 and 467, and binds B7H3. In some embodiments, the at least one B7H3 VHH domain comprises the sequence of amino acids set forth in any one of SEQ ID NOS: 2-34 and 467. In some embodiments, the at least one B7H3 VHH domain comprises the sequence of amino acids set forth in any one of SEQ ID NOs:8-34, 467, 489-490, and 492-497 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 8-34, 467, 489-490, and 492-497, and binds B7H3. In some embodiments, the at least one B7H3 VHH domain comprises the sequence of amino acids set forth in any one of SEQ ID NOS: 8-34, 467, 489-490, and 492-497.

In some of any of the provided embodiments, the at least one B7H3 VHH domain comprises the sequence set forth in (i) SEQ ID NO:35, (ii) a humanized variant of SEQ ID NO:35, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:35, and binds B7H3. In some embodiments, the at least one B7H3 VHH domain comprises a CDR1 comprising an amino acid sequence set forth in SEQ ID NO: 123; a CDR2 comprising an amino acid sequence selected from the group consisting of SEQ ID NO: 152 and 153; and a CDR3 comprising an amino acid sequence selected from the group consisting of SEQ ID NO: 170 and 171. In some embodiments, the at least one B7H3 VHH domain comprises a CDR1, CDR2 and CDR3 set forth in SEQ ID NOS: 123, 152 and 170, respectively. In some embodiments, the at least one B7H3 VHH domain comprises a CDR1, CDR2 and CDR3 set forth in SEQ ID NOS: 123, 152 and 171, respectively. In some embodiments, the at least one B7H3 VHH domain comprises a CDR1, CDR2 and CDR3 set forth in SEQ ID NOS: 123, 153 and 170, respectively. In some embodiments, the at least one B7H3 VHH domain comprises a CDR1, CDR2 and CDR3 set forth in SEQ ID NOS: 123, 153 and 171, respectively. In some embodiments, the at least one B7H3 VHH domain comprises the sequence of amino acids set forth in any one of SEQ ID NOs:36-43 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:36-43, and binds B7H3. In some embodiments, the at least one B7H3 VHH domain comprises the sequence of amino acids set forth in any one of SEQ ID NOS: 36-43. In some embodiments, the at least one B7H3 VHH domain comprises the sequence of amino acids set forth in any one of SEQ ID NOs:40, 41, or 498-503 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 40, 41, or 498-503, and binds B7H3. In some embodiments, the at least one B7H3 VHH domain comprises the sequence of amino acids set forth in any one of SEQ ID NOS: 40, 41, or 498-503.

In some of any of the provided embodiments, the at least one B7H3 VHH domain comprises the sequence set forth in (i) SEQ ID NO:44 (ii) a humanized variant of SEQ ID NO:44, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:44, and binds B7H3. In some embodiments, the at least one B7H3 VHH domain comprises a CDR1 comprising an amino acid sequence selected from the group consisting of SEQ ID NO: 124, 125, 126, 127, 128, 129, 130, 131, 132, or 133; a CDR2 comprising an amino acid sequence set forth in SEQ ID NO: 154; and a CDR3 comprising an amino acid sequence selected from the group consisting of SEQ ID NO: 172, 173, 174, 175, 176, 177, 178, 179, 180, 181, 182 and 183. In some embodiments, the at least one B7H3 VHH domain comprises a CDR1, CDR2 and CDR3 set forth in SEQ ID NOS: 124, 154 and 172, respectively. In some embodiments, the at least one B7H3 VHH domain comprises a CDR1, CDR2 and CDR3 set forth in SEQ ID NOS: 124, 154 and 173, respectively. In some embodiments, the at least one B7H3 VHH domain comprises a CDR1, CDR2 and CDR3 set forth in SEQ ID NOS: 124, 154 and 174, respectively. In some embodiments, the at least one B7H3 VHH domain comprises a CDR1, CDR2 and CDR3 set forth in SEQ ID NOS: 124, 154 and 175, respectively. In some embodiments, the at least one B7H3 VHH domain comprises a CDR1, CDR2 and CDR3 set forth in SEQ ID NOS: 125, 154 and 173, respectively. In some embodiments, the at least one B7H3 VHH domain comprises a CDR1, CDR2 and CDR3 set forth in SEQ ID NOS: 126, 154 and 173, respectively. In some embodiments, the at least one B7H3 VHH domain comprises a CDR1, CDR2 and CDR3 set forth in SEQ ID NOS: 127, 154 and 173, respectively. In some embodiments, the at least one B7H3 VHH domain comprises a CDR1, CDR2 and CDR3 set forth in SEQ ID NOS: 128, 154 and 173, respectively. In some embodiments, the at least one B7H3 VHH domain comprises a CDR1, CDR2 and CDR3 set forth in SEQ ID NOS: 129, 154 and 173, respectively. In some embodiments, the at least one B7H3 VHH domain comprises a CDR1, CDR2 and CDR3 set forth in SEQ ID NOS: 130, 154 and 173, respectively. In some embodiments, the at least one B7H3 VHH domain comprises a CDR1, CDR2 and CDR3 set forth in SEQ ID NOS: 131, 154 and 173, respectively. In some embodiments, the at least one B7H3 VHH domain comprises a CDR1, CDR2 and CDR3 set forth in SEQ ID NOS: 124, 154 and 176, respectively. In some embodiments, the at least one B7H3 VHH domain comprises a CDR1, CDR2 and CDR3 set forth in SEQ ID NOS: 124, 154 and 177, respectively. In some embodiments, the at least one B7H3 VHH domain comprises a CDR1, CDR2 and CDR3 set forth in SEQ ID NOS: 124, 154 and 178, respectively. In some embodiments, the at least one B7H3 VHH domain comprises a CDR1, CDR2 and CDR3 set forth in SEQ ID NOS: 124, 154 and 179, respectively. In some embodiments, the at least one B7H3 VHH domain comprises a CDR1, CDR2 and CDR3 set forth in SEQ ID NOS: 124, 154 and 180, respectively. In some embodiments, the at least one B7H3 VHH domain comprises a CDR1, CDR2 and CDR3 set forth in SEQ ID NOS: 124, 154 and 181, respectively. In some embodiments, the at least one B7H3 VHH domain comprises a CDR1, CDR2 and CDR3 set forth in SEQ ID NOS: 124, 154 and 182, respectively. In some embodiments, the at least one B7H3 VHH domain comprises a CDR1, CDR2 and CDR3 set forth in SEQ ID NOS: 124, 154 and 183, respectively. In some embodiments, the at least one B7H3 VHH domain comprises a CDR1, CDR2 and CDR3 set forth in SEQ ID NOS: 126, 154 and 176, respectively. In some embodiments, the at least one B7H3 VHH domain comprises a CDR1, CDR2 and CDR3 set forth in SEQ ID NOS: 124, 154 and 179, respectively. In some embodiments, the at least one B7H3 VHH domain comprises a CDR1, CDR2 and CDR3 set forth in SEQ ID NOS: 124, 154 and 182, respectively. In some embodiments, the at least one B7H3 VHH domain comprises a CDR1, CDR2 and CDR3 set forth in SEQ ID NOS: 132, 154 and 176, respectively; or SEQ ID NOS: 133, 154 and 173, respectively. In some embodiments, the at least one B7H3 VHH domain comprises the sequence of amino acids set forth in any one of SEQ ID NOs:45-91 and 466 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:45-91 and 466, and binds B7H3. In some embodiments, the at least one B7H3 VHH domain comprises the sequence of amino acids set forth in any one of SEQ ID NOS: 45-91 and 466. In some embodiments, the at least one B7H3 VHH domain comprises the sequence of amino acids set forth in any one of SEQ ID NOs:56-91, 466, and 504-514 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 56-91, 466, and 504-514, and binds B7H3. In some embodiments, the at least one B7H3 VHH domain comprises the sequence of amino acids set forth in any one of SEQ ID NOS: 56-91, 466, and 504-514.

In some of any of the provided embodiments, the at least one B7H3 VHH domain comprises the sequence set forth in (i) SEQ ID NO:105 (ii) a humanized variant of SEQ ID NO:105, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:105, and binds B7H3. In some embodiments, the at least one B7H3 VHH domain comprises a CDR1 comprising an amino acid sequence set forth in SEQ ID NO: 145; a CDR2 comprising an amino acid sequence set forth in SEQ ID NO: 167; and a CDR3 comprising an amino acid sequence set forth in SEQ ID NO: 488. In some embodiments, the at least one B7H3 VHH domain comprises the sequence of amino acids set forth in any one of SEQ ID NOs:106-109 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:106-109, and binds B7H3. In some embodiments, the at least one B7H3 VHH domain comprises the sequence of amino acids set forth in any one of SEQ ID NOS: 106-109.

In some of any of the provided embodiments, the at least one B7H3 VHH domain comprises the sequence set forth in (i) SEQ ID NO:110 (ii) a humanized variant of SEQ ID NO:110, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:110, and binds B7H3. In some embodiments, the at least one B7H3 VHH domain comprises a CDR1 comprising an amino acid sequence set forth in SEQ ID NO: 139; a CDR2 comprising an amino acid sequence set forth in SEQ ID NO: 161; and a CDR3 comprising an amino acid sequence set forth in SEQ ID NO: 189. In some embodiments, the at least one B7H3 VHH domain comprises the sequence of amino acids set forth in any one of SEQ ID NOs:111-114 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:111-114, and binds B7H3. In some embodiments, the at least one B7H3 VHH domain comprises the sequence of amino acids set forth in any one of SEQ ID NOS: 111-114. In some embodiments, the at least one B7H3 VHH domain comprises the sequence of amino acids set forth in any one of SEQ ID NOs:515-518 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 515-518, and binds B7H3. In some embodiments, the at least one B7H3 VHH domain comprises the sequence of amino acids set forth in any one of SEQ ID NOS: 515-518.

In some of any of the provided embodiments, the at least one B7H3 VHH domain comprises the sequence set forth in (i) SEQ ID NO:92, 93, 94, 95, 96, 97, 98, 99, 100, 101, 102, 103 or 104 (ii) a humanized variant of SEQ ID NO: 92, 93, 94, 95, 96, 97, 98, 99, 100, 101, 102, 103 or 104, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 92, 93, 94, 95, 96, 97, 98, 99, 100, 101, 102, 103 or 104, and binds B7H3. In some embodiments, the at least one B7H3 VHH domain comprises a CDR1, CDR2 and CDR3 set forth in SEQ ID NOS:134, 155 and 184, respectively. In some embodiments, the at least one B7H3 VHH domain comprises a CDR1, CDR2 and CDR3 set forth in SEQ ID NOS:135, 156 and 168, respectively. In some embodiments, the at least one B7H3 VHH domain comprises a CDR1, CDR2 and CDR3 set forth in SEQ ID NOS:136, 157 and 185, respectively. In some embodiments, the at least one B7H3 VHH domain comprises a CDR1, CDR2 and CDR3 set forth in SEQ ID NOS:137, 158 and 186, respectively. In some embodiments, the at least one B7H3 VHH domain comprises a CDR1, CDR2 and CDR3 set forth in SEQ ID NOS: 138, 159 and 187, respectively. In some embodiments, the at least one B7H3 VHH domain comprises a CDR1, CDR2 and CDR3 set forth in SEQ ID NOS:138, 160 and 188, respectively. In some embodiments, the at least one B7H3 VHH domain comprises a CDR1, CDR2 and CDR3 set forth in SEQ ID NOS:139, 161 and 189, respectively. In some embodiments, the at least one B7H3 VHH domain comprises a CDR1, CDR2 and CDR3 set forth in SEQ ID NOS:140, 162 and 483, respectively. In some embodiments, the at least one B7H3 VHH domain comprises a CDR1, CDR2 and CDR3 set forth in SEQ ID NOS:141, 163 and 484, respectively. In some embodiments, the at least one B7H3 VHH domain comprises a CDR1, CDR2 and CDR3 set forth in SEQ ID NOS: 139, 161 and 189, respectively. In some embodiments, the at least one B7H3 VHH domain comprises a CDR1, CDR2 and CDR3 set forth in SEQ ID NOS:142, 164 and 485, respectively. In some embodiments, the at least one B7H3 VHH domain comprises a CDR1, CDR2 and CDR3 set forth in SEQ ID NOS:143, 165 and 486, respectively. In some embodiments, the at least one B7H3 VHH domain comprises a CDR1, CDR2 and CDR3 set forth in SEQ ID NOS:144, 166 and 487, respectively. In some embodiments, the at least one B7H3 VHH domain is set forth in SEQ ID NO: 92, 93, 94, 95, 96, 97, 98, 99, 100, 101, 102, 103 or 104.

In some embodiments, a B7H3 VHH domain comprises the VHH domain sequence set forth in SEQ ID NO: 1. In some embodiments, a B7H3 VHH domain comprises the VHH domain sequence set forth in SEQ ID NO:8. In some embodiments, a B7H3 VHH domain comprises the VHH domain sequence set forth in SEQ ID NO: 43. In some embodiments, a B7H3 VHH domain comprises the VHH domain sequence set forth in SEQ ID NO: 503. In some embodiments, a B7H3 VHH domain comprises the VHH domain sequence set forth in SEQ ID NO: 67. In some embodiments, a B7H3 VHH domain comprises the VHH domain sequence set forth in SEQ ID NO: 85. In some embodiments, a B7H3 VHH domain comprises the VHH domain sequence set forth in SEQ ID NO: 455. In some embodiments, a B7H3 VHH domain comprises the VHH domain sequence set forth in SEQ ID NO: 456. In some embodiments, a B7H3 VHH domain comprises the VHH domain sequence set forth in SEQ ID NO: 466. In some embodiments, a B7H3 VHH domain comprises the VHH domain sequence set forth in SEQ ID NO: 467.

In some of any of the provided embodiments, a B7H3 VHH domain may comprise additional amino acids at its N- and/or C-terminal, such as for linkage to another amino acid sequence, such as another polypeptide. In some of any of the provided embodiments, a B7H3 VHH domain may comprise a flexible linker, such as a glycine linker or a linker composed predominately of the amino acids Glycine and Serine, denoted as GS-linkers herein. Such linkers of the present disclosure can be of various lengths, for example, 5, 6, 7, 8, 9, 10, 11, 12, 13, 14, 15, 16, 17, 18, 19, 20 amino acids in length. In some embodiments, the linker comprises an amino acid sequence selected from the group consisting of GGSGGS, i.e., (GGS)₂ (SEQ ID NO: 191); GGSGGSGGS, i.e., (GGS)₃ (SEQ ID NO: 192); GGSGGSGGSGGS, i.e., (GGS)₄ (SEQ ID NO: 193); and GGSGGSGGSGGSGGS, i.e., (GGS)₅ (SEQ ID NO: 194), Gly-Gly (GG), GGG, GGGG (SEQ ID NO:195), GGGGG (SEQ ID NO: 196), and GGGGGG (SEQ ID NO: 197). In some embodiments, the linker is (GGGGS)n, wherein n is 1 to 5 (SEQ ID NO:123); (GGGGGS)n, wherein n is 1 to 4 (SEQ ID NO:124); GGGGS (SEQ ID NO:125); GGGGGS (SEQ ID NO:126); GGGGGSGGGGGSGGGGGS (SEQ ID NO:317); GGGGSGGGGSGGGGS (SEQ ID NO:318); GGSGGGGSGGGGSGGGGS (SEQ ID NO:319); or PGGGG (SEQ ID NO:444). In some embodiments, the linker is a GG linker. In some embodiments, the B7H3-binding polypeptide includes a combination of a GS-linker and a Glycine linker, In some embodiments, a B7H3 VHH domain may comprise the additional linker at its C-terminus, such as for linkage to another amino acid sequence, such as another polypeptide. In some of any of the provided embodiments, a B7H3 VHH domain may comprise the linker at its N-terminus, such as for linkage to another amino acid sequence, such as another polypeptide.

Provided herein is a multispecific polypeptide construct, comprising a first component comprising a heterodimeric Fc region comprising a first Fc polypeptide and a second Fc polypeptide and a second component comprising an anti-CD3 antibody or antigen-binding fragment comprising a variable heavy chain region (VH) and a variable light chain region (VL), wherein the VH and VL that comprise the anti-CD3 antibody or antigen binding fragment are linked to opposite polypeptides of the heterodimeric Fc; the first and second components are coupled by a linker, wherein the heterodimeric Fc region is positioned N-terminal to the anti-CD3 antibody; and one or both of the first and second components comprises at least one antigen binding domain comprising a single domain antibody that specifically binds B7H3 (B7H3 VHH domain). In particular embodiments, the B7H3 VHH domain can include any of the provided B7H3 VHH domain sequences, including any as described above or elsewhere herein.

In some embodiments, the multispecific polypeptide construct comprises at least (i) a first polypeptide comprising the first Fc polypeptide of the heterodimeric Fc region, the linker and the VH or VL domain of the anti-CD3 antibody or antigen binding fragment; and (ii) a second polypeptide comprising the second Fc polypeptide of the heterodimeric Fc region, the linker, optionally the same linker as present in the first polypeptide, and the other of the VH or VL domain of the anti-CD3 antibody or antigen binding fragment, wherein one or both of the first and second polypeptide comprise the at least one B7H3 VHH domain.

In some embodiments, one or both of the first and second Fc polypeptides of the heterodimeric Fc region comprises at least one modification to induce heterodimerization compared to a polypeptide of a homodimeric Fc region, optionally compared to the Fc polypeptide set forth in SEQ ID NO:198 or an immunologically active fragment thereof. In some embodiments, each of the first and second Fc polypeptides of the heterodimeric Fc region independently comprise at least one amino acid modification. In some embodiments, each of the first and second Fc polypeptides of the heterodimeric Fc region comprise a knob-into-hole modification or comprise a charge mutation to increase electrostatic complementarity of the polypeptides.

In some embodiments, the amino acid modification is a knob-into-hole modification. In one embodiment, the first Fc polypeptide of the heterodimeric Fc region comprises the modification selected from among Thr366Ser, Leu368Ala, Tyr407Val, and combinations thereof and the second Fc polypeptide of the heterodimeric Fc region comprises the modification Thr366Trp. In such an embodiment, the first and second Fc polypeptides can further comprises a modification of a non-cysteine residue to a cysteine residue, wherein the modification of the first Fc polypeptide is at one of the position Ser354 and Tyr349 and the modification of the second Fc polypeptide is at the other of the position Ser354 and Tyr349.

In some embodiments, the amino acid modification is a charge mutation to increase electrostatic complementarity of the polypeptides. In some embodiments, the first and/or second Fc polypeptides or each of the first and second Fc polypeptide comprise a modification in complementary positions, wherein the modification is replacement with an amino acid having an opposite charge to the complementary amino acid of the other polypeptide.

In some of any of the provided embodiments, one of the first or second Fc polypeptide of the heterodimeric Fc region further comprises a modification at residue Ile253. In some embodiments, the modification is Ile253Arg. In some embodiments, one of the first or second Fc polypeptide of the heterodimeric Fc region further comprises a modification at residue His435. In some embodiments, the modification is His435Arg.

In some embodiments, the Fc region of any of the provided polypeptides or constructs comprises a polypeptide that lacks Lys447.

In some embodiments, the Fc region of any of the provided polypeptides or constructs comprises at least one modification to enhance FcRn binding. In some embodiments, the modification is at a position selected from the group consisting of Met252, Ser254, Thr256, Met428, Asn434, and combinations thereof. In some embodiments, the modification is selected from the group consisting of Met252Y, Ser254T, Thr256E, Met428L, Met428V, Asn434S, and combinations thereof. In some embodiments the modification is at position Met252 and at position Met428. In some embodiments, the modification is Met252Y and Met428L. In some embodiments, the modification is Met252Y and Met428V.

In some of any of the provided embodiments, the first Fc polypeptide of the heterodimeric Fc region comprises the sequence of amino acids set forth in any of SEQ ID NOS: 293, 297, 305 or 307, and the second Fc polypeptide of the heterodimeric Fc region comprises the sequence of amino acids set forth in any of SEQ ID NOS: 294, 298, 301, 303, 309 or 311.

In some of any of the provided embodiments, the Fc region of a provided polypeptide or constrct comprises a polypeptide comprising at least one amino acid modification that reduces effector function and/or reduces binding to an effector molecule selected from an Fc gamma receptor or C1q. In some embodiments, the one or more amino acid modification is deletion of one or more of Glu233, Leu234 or Leu235.

In some of any of the provided embodiments, the first Fc polypeptide of the heterodimeric Fc region comprises the sequence of amino acids set forth in any of SEQ ID NOS: 295, 299, 306 or 308 and the second Fc polypeptide of the heterodimeric Fc region comprises the sequence of amino acids set forth in any of SEQ ID NOS: 296, 300, 302, 304, 310 or 312.

In some of any of the provided embodiments, the anti-CD3 antibody or antigen binding fragment is monovalent. In some embodimetns, the anti-CD3 antibody or antigen binding fragment is an Fv antibody fragment. In some embodiments, the Fv antibody fragment comprises a disulfide stabilized anti-CD3 binding Fv fragment (dsFv).

In some embodiments, the anti-CD3 antibody or antigen binding fragment is not a single chain antibody, for example is not a single chain variable fragment (scFv).

In some embodiments, the anti-CD3 antibody or antigen-binding fragment comprises a VH CDR1 comprising the amino acid sequence TYAMN (SEQ ID NO: 219); a VH CDR2 comprising the amino acid sequence RIRSKYNNYATYYADSVKD (SEQ ID NO: 220); a VH CDR3 comprising the amino acid sequence HGNFGNSYVSWFAY (SEQ ID NO: 221), a VL CDR1 comprising the amino acid sequence RSSTGAVTTSNYAN (SEQ ID NO: 222); a VL CDR2 comprising the amino acid sequence GTNKRAP (SEQ ID NO: 223); and a VL CDR3 comprising the amino acid sequence ALWYSNLWV (SEQ ID NO: 224). In some embodiments, the anti-CD3 antibody or antigen-binding fragment comprises: a VH having the amino acid sequence of any of SEQ ID NOS: 225-255, 480, 460, or 462 or a sequence that exhibits at least 90%, 91%, 92%, 93%, 94%, 95%, 96%, 97%, 98% or 99% sequence identity to any of SEQ ID NOS: 225-255, 480, 460, or 462; and a VL having the amino acid sequence of any of SEQ ID NOS: 256-274, 417,459, or 461 or a sequence that exhibits at least 90%, 91%, 92%, 93%, 94%, 95%, 96%, 97%, 98% or 99% sequence identity to any of SEQ ID NOS: 6256-274, 417,459, or 461. In some embodiments, the anti-CD3 antibody or antigen-binding fragment comprises the amino acid sequence of SEQ ID NO: 237 and the amino acid sequence of SEQ ID NO: 265. In some embodiments, the anti-CD3 antibody or antigen-binding fragment comprises the amino acid sequence of SEQ ID NO: 237 and the amino acid sequence of SEQ ID NO: 417. In some embodiments, the anti-CD3 antibody or antigen-binding fragment comprises the amino acid sequence of SEQ ID NO: 460 and the amino acid sequence of SEQ ID NO: 461. In some embodiments, the anti-CD3 antibody or antigen-binding fragment comprises the amino acid sequence of SEQ ID NO: 480 and the amino acid sequence of SEQ ID NO: 459.

In some embodiments, the VL of the anti-CD3 antibody or antigen binding fragment is linked to the first Fc polypeptide of the heterodimeric Fc and the VH of the anti-CD3 antibody or antigen binding fragment is linked to the second Fc polypeptide of the heterodimeric Fc.

In some embodiments, the CD3-binding region comprises a variable heavy chain region (VH) and a variable light chain region (VL), and the VL is C-terminal to the first Fc polypeptide of the heterodimeric Fc region and the VH is C-terminal to the second Fc polypeptide of the heterodimeric Fc region,wherein the first Fc polypeptide comprises a hole mutation and the second Fc polypeptide comprises a knob mutation. In some embodiments, the at least one B7H3 single domain antibody is positioned amino-terminally relative to the Fc region and/or carboxy-terminally relative to the CD3 binding region of the multispecific polypeptide construct.

In some embodiments, the multispecific polypeptide construct comprises a first B7H3 VHH domain that specifically bind B7H3 and a second B7H3 VHH domain that specifically binds B7H3. In some embodiments, the first or second B7H3 VHH domain is positioned amino-terminally relative to the Fc region of the multispecific construct and the other of the first or second B7H3 VHH domain is positioned carboxy-terminally relative to the CD3 binding region of the multispecific construct.

In some embodiments of any of the provided mutlispecific polypeptide constructs, the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second polypeptide comprises in order of N-terminus to C-terminus the second Fc polypeptide of the heterodimeric Fc region, the linker, optionally the same linker as present in the first component, and the other of the VH or VL domain of the anti-CD3 antibody or antigen binding fragment.

In some embodiments, the multispecific polypeptide construct comprises at least a first polypeptide comprising the first Fc polypeptide of the heterodimeric Fc region, the linker and the VH or VL domain of the anti-CD3 antibody or antigen binding fragment; and a second polypeptide comprising the second Fc polypeptide of the heterodimeric Fc region, the linker, optionally the same linker as present in the first polypeptide, and the other of the VH or VL domain of the anti-CD3 antibody or antigen binding fragment, wherein one or both of the first and second polypeptide comprise the at least one B7H3 VHH domain.

In some embodiments, the polypeptide construct comprises an immunoglobulin Fc region. In some embodiments, the immunoglobulin Fc region that links the at least one B7H3 VHH domain and the one or more additional binding domain. In some embodiments, the Fc region is a homodimeric Fc region. In some embodiments, the Fc region comprises the sequence of amino acids set forth in any of SEQ ID NOS: 198, 200, 201, 202 or 203, or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 198, 200, 201, 202 or 203. In some embodiments, the Fc region is a human IgG1. In some embodiments, the Fc region comprises the sequence of amino acids set forth in SEQ ID NO:198 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 198.

In some embodiments, the Fc region is a heterodimeric Fc reigon. In some embodiments, the Fc region exhibits effector function. In some embodiments, the Fc region comprises a polypeptide comprising one or more amino acid modification that reduces effector function and/or reduces binding to an effector molecule selected from an Fc gamma receptor or C1q. In some embodiments, the one or more amino acid modification is deletion of one or more of Glu233, Leu234 or Leu235. In some embodiments, the Fc region comprises the sequence of amino acids set forth in SEQ ID NO:199 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 199.

In some embodiments, one or both of the first and second Fc polypeptides of the heterodimeric Fc region comprises at least one modification to induce heterodimerization compared to a polypeptide of a homodimeric Fc region, optionally compared to the Fc polypeptide set forth in SEQ ID NO:198 or an immunologically active fragment thereof. In some embodiments, each of the first and second Fc polypeptides of the heterodimeric Fc region independently comprise at least one amino acid modification. In some embodiments, each of the first and second Fc polypeptides of the heterodimeric Fc region comprise a knob-into-hole modification or comprise a charge mutation to increase electrostatic complementarity of the polypeptides. In some embodiments, the amino acid modification is a knob-into-hole modification. In some embodiments, the amino acid modification is a charge mutation to increase electrostatic complementarity of the polypeptides. In some embodiments, the first and/or second Fc polypeptides or each of the first and second Fc polypeptide comprise a modification in complementary positions, wherein the modification is replacement with an amino acid having an opposite charge to the complementary amino acid of the other polypeptide. In some embodiments, the Fc region comprises a polypeptide comprising at least one modification to enhance FcRn binding. In some embodiments, the Fc region comprises a polypeptide comprising at least one amino acid modification that reduces effector function and/or reduces binding to an effector molecule selected from an Fc gamma receptor or C1q.

In some embodiments, one or both of the first and second components comprises at least one co-stimulatory receptor binding region (CRBR) binds to 41BB (CD137). In some embodiments, the multispecific polypeptide construct comprises only one co-stimulatory receptor binding region (CRBR). In some embodiments, the at least one co-stimulatory receptor binding region (CRBR) is positioned amino-terminally relative to the Fc region and/or carboxy-terminally relative to the CD3 binding region of the multispecific polypeptide construct.

In some embodiments of any of the provided multispecific polypeptide constructs, the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second component comprises the CRBR and comprises in order of N-terminus to C-terminus the second Fc polypeptide of the heterodimeric Fc region, the linker, optionally the same linker as present in the first component, the other of the VH or VL domain of the anti-CD3 antibody or antigen binding fragment, wherein the CRBR is positioned amino-terminally relative to the Fc region or carboxy-terminally relative to the anti-CD3 antibody or antigen binding fragment of the second component.

In some embodiments, the at least one co-stimulatory receptor binding region (CRBR) is or comprises the extracellular domain or binding fragment thereof of the native cognate binding partner of the co-stimulatory receptor, or a variant thereof that exhibits binding activity to the co-stimulatory receptor. In some embodiments, the at least one co-stimulatory receptor binding region (CRBR) is an antibody or antigen-binding fragment thereof selected from the group consisting of a Fab fragment, a F(ab′)2 fragment, an Fv fragment, a scFv, a scAb, a dAb, a single domain heavy chain antibody, and a single domain light chain antibody. In some embodiments, the antibody or antigen-binding fragment thereof is a Fv, a scFv, a Fab, a single domain antibody (VHH domain), a VNAR, or a VHH. In some embodiments, the antibody or antigen-binding fragment is an VHH domain. In some embodiments, the VHH domain is a human or humanized VHH domain.

In some of any of the provided embodiments, the at least one co-stimulatory receptor binding region (CRBR) binds a co-stimulatory receptor selected from among 41BB (CD137), OX40 (CD134), CD27, glucocorticoid-induced TNFR-related protein (GITR), CD28, ICOS, CD40, B-cell activating factor receptor (BAFF-R), B-cell maturation antigen (BCMA), Transmembrane activator and CAML interactor (TACI), and NKG2D. In some embodiments, the at least one co-stimulatory receptor binding region (CRBR) binds a co-stimulatory receptor selected from among 41BB (CD137), OX40 (CD134), and glucocorticoid-induced TNFR-related protein (GITR).

In some embodiments, the at least one co-stimulatory receptor binding region (CRBR) binds to 41BB (CD137).

In some embodiments, the at least one co-stimulatory receptor binding region (CRBR) comprises the sequence of amino acids set forth in SEQ ID NO:400 or a sequence that has at least 85%, 86%, 87%, 88%, 89%, 90%, 91%, 92%, 93%, 94%, 95%, 96%, 97%, 98%, or 99% sequence identity to the sequence set forth in SEQ ID NO:400 and binds 4-1BB. In some embodiments, the at least one co-stimulatory receptor binding region (CRBR) comprises the sequence of amino acids set forth in SEQ ID NO:481 or a sequence that has at least 85%, 86%, 87%, 88%, 89%, 90%, 91%, 92%, 93%, 94%, 95%, 96%, 97%, 98%, or 99% sequence identity to the sequence set forth in SEQ ID NO:481 and binds 4-1BB.

In some embodiments, the at least one co-stimulatory receptor binding region (CRBR) comprises the sequence of amino acids set forth in SEQ ID NO:400. In some embodiments, the at least one co-stimulatory receptor binding region (CRBR) comprises the sequence of amino acids set forth in SEQ ID NO:481.

In some embodiments, one or both of the first and second components comprises at least one inhibitory receptor binding region (IRBR) that binds an inhibitory receptor. In some embodiments, the at least one inhibitory receptor binding region (IRBR) is positioned amino-terminally relative to the Fc region and/or carboxy-terminally relative to the CD3 binding region of the multispecific polypeptide construct. In some embodiments, the multispecific polypeptide construct comprises only one inhibitory receptor binding region (IRBR).

In some embodiments of any of the provided multispecific polypeptide constructs, the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second component comprises the IRBR and comprises in order of N-terminus to C-terminus the second Fc polypeptide of the heterodimeric Fc region, the linker, optionally the same linker as present in the first component, the other of the VH or VL domain of the anti-CD3 antibody or antigen binding fragment, wherein the IRBR is positioned amino-terminally relative to the Fc region or carboxy-terminally relative to the anti-CD3 antibody or antigen-binding fragment of the second component.

In some embodiments, the at least one IRBR is or comprises the extracellular domain or binding fragment thereof of the native cognate binding partner of the inhibitory receptor, or a variant thereof that exhibits binding activity to the inhibitory receptor. In some embodiments, the at least one IRBR is an antibody or antigen-binding fragment thereof selected from the group consisting of a Fab fragment, a F(ab′)2 fragment, an Fv fragment, a scFv, a scAb, a dAb, a single domain heavy chain antibody, and a single domain light chain antibody. In some embodiments, the antibody or antigen-binding ″fragment thereof is a Fv, a scFv, a Fab, a single domain antibody (VHH domain), a VNAR, or a VHH. In some embodiments, the antibody or antigen-binding fragment is an VHH domain. In some embodiments, the VHH domain is a human or humanized VHH domain. In some embodiments, the at least one IRBR binds a inhibitory receptor selected from among PD-1, CTLA-4, TIGIT, VISTA and TIM3. In some embodiments, the at least one IRBR binds PD-1.

In some of any of the provided embodiments of a multispecific polypeptide construct, the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second component comprises comprises in order of N-terminus to C-terminus one of the IRBR or the CRBR, the second Fc polypeptide of the heterodimeric Fc region, the linker, optionally the same linker as present in the first component, the other of the VH or VL domain of the anti-CD3 antibody or antigen binding fragment, and the other of the CRBR or IRBR.

In some of any of the provided embodiments, the linker is a peptide or polypeptide linker In some embodiments, the linker is 3, 4, 5, 6, 7, 8, 9, 10, 11, 12, 13, 14, 15, 16, 17, 18, 19 or 20 amino acids in length.

In some embodiments, the linker is a non-cleavable linker, such as a linker that comprises GS, GGS, GGGGS (SEQ ID NO: 315), GGGGGS (SEQ ID NO: 316) and combinations thereof. In some embodiments, the linker is or comprises the sequence GGGGGSGGGGGSGGGGGS (SEQ ID NO: 317).

In some embodiments, the linker is a cleavable linker, such as a polypeptide that functions as a substrate for a protease. In some embodiments, the protease is produced by an immune effector cell, by a tumor, or by cells present in the tumor microenvironment. In some embodiments, the protease is produced by an immune effector cell and the immune effector cell is an activated T cell, a natural killer (NK) cell, or an NK T cell. In soe embodiments, the protease is a matriptase, a matrix metalloprotease (MMP), granzyme B, orcombinations thereof. In some embodiments, the cleavable linker comprises the amino acid sequence GGSGGGG [symbol] IGGSGGS (SEQ ID NO: 361).

Provided herein is an isolated single domain antibody that binds B7H3 and that contains any of the B7H3 VHH domain squences provided herein, including any as described above or elsewhere herein.

Provided herein is an isolated single domain antibody that binds B7H3, comprising a complementarity determining region 1 (CDR1) comprising an amino acid sequence selected from the group consisting of SEQ ID NO: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and 145; a complementarity determining region 2 (CDR2) comprising an amino acid sequence selected from the group consisting of SEQ ID NO: 146, 147, 148, 149, 150, 151, 152, 153, 154, 155, 156, 157, 158, 159, 160, 161, 162, 163, 164, 165, 166 and 167; and a complementarity determining region 3 (CDR3) comprising an amino acid sequence selected from the group consisting of SEQ ID NO: 168, 169, 170, 171, 172, 173, 174, 175, 176, 177, 178, 179, 180, 181, 182, 183, 184, 185, 186, 187, 188, 189, and 483-488.

In some embodiments, the heterodimeric Fc region comprises an Fc hole polypeptide and an Fc knob polypeptide, wherein the VL of the anti-CD3 antibody or antigen binding fragment is positioned C-terminal to the Fc hole and the VH of the anti-CD3 antibody or antigen binding fragment is positioned C-terminal to the Fc knob.

In some embodiments, the anti-CD3 antibody or antigen binding fragment is monovalent. In some embodiments, the anti-CD3 antibody or antigen binding fragment is not a single chain antibody, optionally is not a single chain variable fragment (scFv). In some embodiments, the anti-CD3 antibody or antigen binding fragment is an Fv antibody fragment. In some embodiments, the Fv antibody fragment comprises a disulfide stabilized anti-CD3 binding Fv fragment (dsFv). In some embodiments, the anti-CD3 antibody or antigen-binding fragment comprises the amino acid sequence of SEQ ID NO: 237 and the amino acid sequence of SEQ ID NO: 265. In some embodiments, the anti-CD3 antibody or antigen-binding fragment comprises the amino acid sequence of SEQ ID NO: 237 and the amino acid sequence of SEQ ID NO: 417. In some embodiments, the anti-CD3 antibody or antigen-binding fragment comprises the amino acid sequence of SEQ ID NO: 460 and the amino acid sequence of SEQ ID NO: 461. In some embodiments, the anti-CD3 antibody or antigen-binding fragment comprises the amino acid sequence of SEQ ID NO: 480 and the amino acid sequence of SEQ ID NO: 459.

Provided herein is a polynucleotide(s) encoding any of the provided B7H3-binding polypeptides. Provided herein is a polynucleotide(s) encoding any of the provided multispecific polypeptide constructs. Provided herein is a polynucleotide, comprising a first nucleic acid sequence encoding a first polypeptide of any of the provided multispecific constructs and a second nucleic acid sequence encoding a second polypeptide of any of the provided multispecific constructs, wherein the first and second nucleic acid sequence are separated by an internal ribosome entry site (IRES), or a nucleic acid encoding a self-cleaving peptide or a peptide that causes ribosome skipping.

Provided herein is a polynucleotide encoding any of the provided single domain antibodies. Provided herein is a vector comprising any of provided polynucleotides.

Provided herein is a cell comprising any of the provided polynucleotide or polynucleotides or any of the provided vector or vectors.

Provided herein is a method of producing a polypeptide including introducing into a cell any of the provided polynucleotide or polynucleotides or a vector or vectors and culturing the cell under conditions to produce the multispecific polypeptide construct. Provided herein is a polypeptide produced by any of the methods provided herein.

Provided herein is an engineered immune cell, comprising a chimeric antigen receptor comprising an extracellular domain comprising any of the provided single domain antibodies; a transmembrane domain; and an intracellular signaling domain.

Provided herein is a pharmaceutical composition comprising any of the provided B7H3-binding polypeptides, multispecific polypeptide constructs, single domain antibodies or engineered immune cells.

Provided herein is a method of stimulating or inducing an immune response in a subject, the method comprising administering, to a subject in need thereof, any of the provided B7H3-binding polypeptides, multispecific polypeptide constructs, single domain antibodies or engineered immune cells, or pharmaceutical compositions. Provided herein is a method of treating a disease or condition in a subject, the method comprising administering, to a subject in need thereof, a therapeutically effective amount of any of the provided B7H3-binding polypeptides, multispecific polypeptide constructs, single domain antibodies or engineered immune cells, or pharmaceutical compositions.

### BRIEF DESCRIPTION OF THE DRAWINGS

FIGS. 1A-1F set forth a series of graphs depicting the ability of various anti-B7H3 single domain antibodies (sdAb) to bind cell-surface B7H3. Binding was assessed by flow cytometry to B7H3-positive cell lines NCI-H460 (FIGS. 1A-1B) or A375 (FIG. 1C), or 293 cells transfected with B7H3 (FIGS. 1D, 1E, 1F).

FIGS. 2A-2V set forth a series of graphs depicting the ability of humanized sdAbs to bind cell surface B7H3. FIGS. 2A-2C show the binding of 57B04 and humanized variants thereof on NCI-H460. FIG. 2D shows the binding of 57B06 and humanized (hz) variants thereof to NCI-H460. FIG. 2E shows the binding of 1H5 and humanized (hz) variants thereof to 293FS cells expressing B7H3. FIGS. 2F-2I and 2X-Y show the binding of humanized (hz) 1A5 variants to NCI-H460 (FIG. 2F) or 293FS cells expressing B7H3 (FIGS. 2G, 2H, 2I, 2X, 2Y). FIGS. 2J-2W show the binding of 58E05 and humanized (hz) variants thereof to HCT-116 (FIGS. 2J, 2K, 2L), NCI-H460 (FIGS. 2M, 2N, 2O, 2P, 2R, 2S, 2T, 2U), A549 (FIG. 2Q) or 293FS cells expressing B7H3 (FIGS. 2V and 2W).

FIGS. 3A-3E depict a series of schematics representing various B7H3-targeted constrained CD3 engaging constructs. The basic components of the B7H3-targeted constrained CD3 engaging constructs of the present disclosure have constrained CD3 binding. The antigen binding domain(s) are positioned at the amino and/or carboxy termini. The Fc region, such as a heterodimeric Fc region, is positioned N-terminal to the CD3 binding region. This positioning of the Fc in close proximity to the CD3 binding region obstructs CD3 binding.

FIGS. 4A-4C depict the presence or absence of binding of cx3855 or B7H3xCD3 DART-Fc to B7H3 target cells or T cells. FIG. 4A depicts the presence or absence of binding to B7H3 positive cells, A375 cell line. FIG. 4B depicts the presence or absence of binding to primary human T-cells. FIG. 4C depicts a titration comparing binding to A375 and primary human T-cells.

FIGS. 5A-5C depict the presence or absence of binding of cx4137 to B7H3 target cells or T cells. FIG. 5A depicts the presence or absence of binding to B7H3 positive A375 cells. FIG. 5B depicts the presence or absence of binding to primary human T-cells. FIG. 5C depicts a titration comparing binding to A375 and primary human T-cells.

FIGS. 6A-6C depict the presence or absence of binding of cx3090 to B7H3 target cells or T cells. FIG. 6A depicts the presence or absence of binding to B7H3 positive cells, A375 cell line. FIG. 6B depicts the presence or absence of binding to primary human T-cells. FIG. 6C depicts a titration comparing binding to A375 and primary human T-cells.

FIGS. 7A-7C depict the presence or absence of binding of cx3243 to B7H3 target cells or T cells. FIG. 7A depicts the presence or absence of binding to B7H3 positive cells, A375 cell line. FIG. 7B depicts the presence or absence of binding to primary human T-cells. FIG. 7C depicts a titration comparing binding to A375 and primary human T-cells.

FIGS. 8A-8C depict the presence or absence of binding of cx4736 to B7H3 target cells or T cells. FIG. 8A depicts the presence or absence of binding to B7H3 positive cells, A375 cell line. FIG. 8B depicts the presence or absence of binding to primary human T-cells. FIG. 8C depicts a titration comparing binding to A375 and primary human T-cells.

FIGS. 9A-9C depicts the presence or absence of binding of cx4136 to B7H3 target cells or T cells. FIG. 9A depicts the presence or absence of binding to B7H3 positive cells, A375 cell line. FIG. 9B depicts the presence or absence of binding to primary human T-cells. FIG. 9C depicts a titration comparing binding to A375 and primary human T-cells.

FIGS. 10A-10C depict the presence or absence of binding of cx3072 to B7H3 target cells or T cells. FIG. 10A depicts the presence or absence of binding to B7H3 positive cells, A375 cell line.

FIG. 10B depicts the presence or absence of binding to primary human T-cells. FIG. 10C depicts a titration comparing binding to A375 and primary human T-cells.

FIGS. 11A-11C depict the presence or absence of binding of cx4641 to B7H3 target cells or T cells. FIG. 11A depicts the presence or absence of binding to B7H3 positive cells, A375 cell line. FIG. 11B depicts the presence or absence of binding to primary human T-cells. FIG. 11C depicts a titration comparing binding to A375 and primary human T-cells.

FIGS. 12A-12C depict the presence or absence of binding of cx4645 to B7H3 target cells or T cells. FIG. 12A depicts the presence or absence of binding to B7H3 positive cells, A375 cell line. FIG. 12B depicts the presence or absence of binding to primary human T-cells. FIG. 12C depicts a titration comparing binding to A375 and primary human T-cells.

FIGS. 13A-13C depict the presence or absence of binding of cx4736 (50 nM) to B7H3 target cells or T cells. FIG. 13A depicts the presence or absence of binding to B7H3 positive cells, A375 cell line. FIG. 13B depicts the presence or absence of binding to primary human T-cells. FIG. 13C depicts a titration comparing binding to A375 and primary human T-cells.

FIGS. 14A-14C depict the presence or absence of binding of cx4736 (12.5 nM) to B7H3 target cells or T cells. FIG. 14A depicts the presence or absence of binding to B7H3 positive cells, A375 cell line. FIG. 14B depicts the presence or absence of binding to primary human T-cells. FIG. 14C depicts a titration comparing binding to A375 and primary human T-cells.

FIGS. 15A-15C depict the presence or absence of binding of cx2846 to B7H3 target cells or T cells. FIG. 15A depicts the presence or absence of binding to B7H3 positive cells, A375 cell line. FIG. 15B depicts the presence or absence of binding to primary human T-cells. FIG. 15C depicts a titration comparing binding to A375 and primary human T-cells.

FIGS. 16A-16C depict the presence or absence of binding of cx3834 to B7H3 target cells or T cells. FIG. 16A depicts the presence or absence of binding to B7H3 positive cells, A375 cell line. FIG. 16B depicts the presence or absence of binding to primary human T-cells. FIG. 16C depicts a titration comparing binding to A375 and primary human T-cells.

FIGS. 17A-17C depict the presence or absence of binding of cx3960 to B7H3 target cells or T cells. FIG. 17A depicts the presence or absence of binding to B7H3 positive cells, A375 cell line. FIG. 17B depicts the presence or absence of binding to primary human T-cells. FIG. 17C depicts a titration comparing binding to A375 and primary human T-cells.

FIGS. 18A-18C depict the presence or absence of binding of cx4904 to B7H3 target cells or T cells. FIG. 18A depicts the presence or absence of binding to B7H3 positive cells, A375 cell line. FIG. 18B depicts the presence or absence of binding to primary human T-cells. FIG. 18C depicts a titration comparing binding to A375 and primary human T-cells.

FIGS. 19A-19C depict the presence or absence of binding of cx4908 to B7H3 target cells or T cells. FIG. 19A depicts the presence or absence of binding to B7H3 positive cells, A375 cell line.

FIG. 19B depicts the presence or absence of binding to primary human T-cells. FIG. 19C depicts a titration comparing binding to A375 and primary human T-cells.

FIGS. 20A-20B depict graphs demonstrating the ability of B7H3-targeted constrained CD3 engaging constructs to elicit B7H3-dependent T-cell activation. A Jurkat CD3 NFAT-GFP reporter cell line was used to monitor T-cell activation. A375 cells (FIG. 20A) and A375 cells wherein the B7H3 gene was disrupted by CRISPR (A375 B7H3−/−, FIG. 20B) were used as antigen positive and negative cell lines, respectively. A B7H3xCD3 bispecific in the DART-Fc format was used as a comparison.

FIGS. 21A-21B depict graphs demonstrating the ability of B7H3-targeted constrained CD3 engaging constructs to mediate antigen specific T-cell cytotoxicity toward the B7H3 positive cell line A375 (FIG. 21A) or an A375 cell line wherein the B7H3 gene was disrupted by CRISPR (A375 B7H3−/−, FIG. 21B), which were used as antigen positive and negative cell lines, respectively. A B7H3xCD3 bispecific in the DART-Fc format was used as a comparison.

FIGS. 22A-22B depict CD25 expression in CD4+ T cells in the presence of B7H3-targeted constrained CD3 engaging constructs in cocultures of T cells and either B7H3 positive cells (A375; FIG. 22A) or B7H3 negative cells (A375 B7H3−/−; FIG. 22B).

FIGS. 23A-23B depict CD69 expression in CD4+ T cells in the presence of B7H3-targeted constrained CD3 engaging constructs in cocultures of T cells and either B7H3 positive cells (A375; FIG. 23A) or B7H3 negative cells (A375 B7H3−/−; FIG. 23B).

FIGS. 24A-24B depict CD71 expression in CD4+ T cells in the presence of B7H3-targeted constrained CD3 engaging constructs in cocultures of T cells and either B7H3 positive cells (A375; FIG. 24A) or B7H3 negative cells (A375 B7H3−/−; FIG. 24B).

FIGS. 25A-25B depict CD25 expression in CD8+ T cells in the presence of B7H3-targeted constrained CD3 engaging constructs in cocultures of T cells and either B7H3 positive cells (A375; FIG. 25A) or B7H3 negative cells (A375 B7H3−/−; FIG. 25B).

FIGS. 26A-26B depict CD69 expression in CD8+ T cells in the presence of B7H3-targeted constrained CD3 engaging constructs in cocultures of T cells and either B7H3 positive cells (A375; FIG. 26A) or B7H3 negative cells (A375 B7H3−/−; FIG. 26B).

FIGS. 27A-27B depict CD71 expression in CD8+ T cells in the presence of B7H3-targeted constrained CD3 engaging constructs in cocultures of T cells and either B7H3 positive cells (A375; FIG. 27A) or B7H3 negative cells (A375 B7H3−/−; FIG. 27B).

FIGS. 28A-28D depict a series of graphs demonstrating the ability of B7H3-targeted constrained CD3 engaging constructs to elicit IFNγ (FIGS. 28A-28C) or TNFα (FIG. 28D) production from T cells or PBMCs in an antigen-dependent manner. Cytokine production was monitored using FluoroSpot assay in the presences or absence of a B7H3 positive cells line A375.

FIG. 29A depicts the ability of 58E05-Fc to induce ADCC of A375 target cells as assessed using a Jurkat reporter engineered to stably express CD16a with an NFAT-driven luciferase reporter gene. FIG. 29B depicts the ability of humanized variants of 58E05-Fc and 1A5-Fc to induce ADCC of SHP-77 target cells as assessed using a Jurkat reporter engineered to stably express CD16a with an NFAT-driven luciferase reporter gene. A conventional anti-B7H3 IgG1 antibody was used as a comparision and displayed no ability to mediate CD16a expression in the presence of SHP-77 a cell line that expresses low to intermediate levels of B7H3.

FIG. 30A is a schematic of various B7H3-targeting constrained CD3 constructs composed of two polypeptides, Chain 1 and Chain 2. Chain 1 contains either a heterodimeric Fc “hole,” linked via a non-cleavable linker to an anti-CD3 VL domain modified at G100C (top); a B7H3-targeting sdAb linked to a heterodimeric Fc “hole,” linked via a non-cleavable linker to an anti-CD3 VL domain (middle); or an B7H3-targeting sdAb linked to a heterodimeric Fc “hole,” linked via a non-cleavable linker to an anti-CD3 VL domain modified at G100C (bottom). Chain 2 contains either a B7H3-targeted sdAb, linked to a complementary heterodimeric Fc “knob,” linked via the linker as above to an anti-CD3 VH domain modified at G44C linked to second B7H3 sdAb (top); a B7H3-targeted sdAb, linked to a complementary heterodimeric Fc “knob,” linked via the linker as above to an anti-CD3 VH domain (middle); or a B7H3-targeted sdAb, linked to a complementary heterodimeric Fc “knob,” linked via the linker as above to an anti-CD3 VH domain modified by G44C (bottom). When co-expressed the CD3 binding domain is properly assembled via the association of the VL:VH on the hole and knob, respectively. Where denoted the VH:VL interaction is stabilized by an engineered disulfide bond between the modified residues G44C in the VH domain and G100C in the VL domain.

FIG. 30B is a schematic of various B7H3-targeting constrained CD3 constructs composed of two polypeptides, Chain 1 and Chain 2. Chain 1 contains a heterodimeric Fc “hole,” linked via a non-cleavable linker to an anti-CD3 VL domain modified at G100C linked to a co-stimulatory receptor targeting sdAb. Chain 2 contains either a B7H3-targeted sdAb, linked to a complementary heterodimeric Fc “knob,” linked via the linker as above to an anti-CD3 VH domain modified at G44C linked to second B7H3-targeted sdAb (top); a heterodimeric Fc “knob,” linked via the linker as above to an anti-CD3 VH domain modified at G44C linked to a B7H3-targeted sdAb (middle); or a B7H3-targeted sdAb, linked to a complementary heterodimeric Fc “knob,” linked via the linker as above to an anti-CD3 VH domain modified by G44C (bottom). When co-expressed the CD3 binding domain is properly assembled via the association of the VL:VH on the hole and knob, respectively. VH:VL interaction is stabilized by an engineered disulfide bond between the modified residues G44C in the VH domain and G100C in the VL domain. The resulting constructs engage B7H3 either in a bivalent (top) or a monovalent (middle and bottom) manner. All the constructs herein contain a co-stimulatory receptor targeting sdAb.

FIG. 30C is a schematic of various B7H3-targeting constrained CD3 constructs composed of three polypeptides, Chain 1, Chain 2 and Chain 3, wherein the B7H3 targeting domain is a FAB. Chain 1 contains a BH73-targeting VH, an IgG Constant Heavy 1 (CH1) linked via a hinge to a first member of a heterodimeric Fc (Fc-Het-1), linked via the linker as above to an anti-CD3 VL domain that either lacks (top) or contains the modification of G100C (middle). Chain 2 contains a BH73-targeting VH, an IgG Constant Heavy 1 (CH1) linked via a hinge to a second member of a heterodimeric Fc (Fc-Het-2), linked via the linker as above to an anti-CD3 VH domain that either lacks (top) or contains the modification of G44C (middle). Chain 3 contains a complementary B7H3-targeting VL domain linked to human Ig Constant Light (CL) region. When co-expressed the CD3 binding domain is properly assembled via the association of the VL: VH on the complimentary heterodimeric Fc regions. Where denoted the VH: VL interaction is stabilized by an engineered disulfide bond between the modified residues G44C in the VH domain and G100C in the VL domain.

FIGS. 31A-F depict cellular binding by representative B7H3-targeting constrained CD3 engaging constructs. FIGS. 31A, C, and E show binding to A375 cells (a B7H3 positive human melanoma cell line). FIGS. 31B, D, and F show the lack of binding to isolated T cells.

FIGS. 32A-D depict the ability of representative B7H3-targeting constrained CD3 engaging constructs to agonize CD3 in a target dependent manner. FIG. 32A and FIG. 32C depict the capacity to mediate CD3 signaling in the presence of B7H3 positive A375 cells, while FIG. 32B and FIG. 32D show the inability to mediate CD3 signaling in the presence of B7H3 negative CCRF-CEM cells. A Jurkat CD3 NFAT-GFP reporter cell line was used to assess CD3 agonism.

FIG. 33A depicts the ability of a representative B7H3-targeting constrained CD3 engaging construct (cx3072) to induce T-cell mediated cytotoxicity in a target dependent manner. Target cells were labeled with cytolD red label and dying cells were visualized by addition of Caspase 3/7 green reagent. Cytotoxicity was assessed by determining the overlap area of red target cells and green dying cells. A B7H3 negative A375 cell line, generated by CRISPR technology was used to test antigen specific T-cell mediated cytotoxicity. cx3072 was unable to elicit T-cell mediated cytotoxicity of these B7H3 deficient A375 cells. FIG. 33B shows the ability of cx5952 to induce T-cell mediated cytotoxicity in the presence of B7H3 positive A375 cells, but not in the presence of B7H3 negative CCRF-CEM cells.

FIGS. 34A and 34B depict the ability of representative B7H3-targeting constrained CD3 engaging constructs to induce T-cell mediated cytotoxicity in a target dependent manner. FIG. 34A depicts the capacity of these constructs to induce T-cell mediated cytotoxicity in the presence of B7H3 positive A375 cells, while FIG. 34B depicts the capacity of these constructs to induce T-cell mediated cytotoxicity in the presence of B7H3 negative CCRF-CEM cells. Cytotoxicity was assessed by determining the overlap area of red target cells and green dying cells.

FIGS. 34C and 34D depict the ability of representative B7H3-targeting constrained CD3 engaging constructs to induce T-cell mediated cytotoxicity in a target dependent manner. FIG. 34C depicts the capacity of these constructs to induce T-cell mediated cytotoxicity in the presence of B7H3 positive A375 cells, while FIG. 34C depicts the capacity of these constructs to induce T-cell mediated cytotoxicity in the presence of B7H3 negative CCRF-CEM cells. Cytotoxicity was assessed by determining the overlap area of red target cells and green dying cells.

FIGS. 35A-C depict the ability of a representative B7H3-targeting constrained CD3 engaging construct (cx5952) to induce T-cell mediated cytotoxicity and T-cell activation in a target dependent manner. Cytotoxicity was assessed by determining the overlap area of red target cells and green dying cells. FIG. 35A-C show the ability of cx5952 to activate CD4+ and CD8+ T-cells in a target dependent manner. T-cell activation was assessed by expression of the T cell activation markers CD25 (FIG. 35A), CD69 (FIG. 35B), and CD71 (FIG. 35C).

FIGS. 35D-35K depict the ability of representative B7H3-targeting constrained CD3 engaging constructs to induce T-cell activation in a target dependent manner. B7H3-target-dependent CD4+ T-cell activation is shown by expression of the T cell activation markers CD25 (FIG. 35D) and CD71 (FIG. 35F). B7H3-target-dependent CD8+ T-cell activation is shown by expression of the T cell activation markers CD25 (FIG. 35H) and CD71 (FIG. 35J). Non-target CD8+ T-cell activation was not observed based on T cell activation marker CD25 as shown in CD4+ T cells (FIG. 35E) or CD8+ T cells (FIG. 31) or based on T cell activation marker CD71 as shown in CD4+ T cells (FIG. 35G) or CD8+ T cells (FIG. 35K).

FIG. 36A depicts the ability of representative B7H3-targeting constrained CD3 engaging constructs to induce IFNγ production in a target dependent manner. FIG. 36A shows the production of IFNγ from T-cells cultured with B7H3 positive A375 cells or B7H3 negative CCRF-CEM cells in the presence of the B7H3-targeting CD3 engaging constructs.

FIG. 36B depicts the ability of representative B7H3-targeting constrained CD3 engaging constructs to induce IFNγ production in a target dependent manner. FIG. 36B shows the production of IFNγ from T-cells cultured with B7H3 positive A375 cells or B7H3 negative CCRF-CEM cells in the presence of the B7H3-targeting CD3 engaging constructs.

FIGS. 37A and 37B depict cellular binding of representative B7H3-targeting constrained CD3 engaging constructs. cx5187 and cx5823 each contain two B7H3 binding domains while constructs cx5873 and cx5965 each contain one B7H3 binding domain. FIG. 37A shows binding to B7H3 positive A375 cells. FIG. 37B shows the lack of binding to B7H3 negative CCRF-CEM cells and isolated T-cells.

FIGS. 37C and FIG. 37D depict the ability of representative B7H3-targeting constrained CD3 engaging constructs to agonize CD3 in a target dependent manner. FIG. 37C shows that engaging B7H3 positive A375 cells with a molecule that is bivalent and bi-epitopic to B7H3 (cx5187) induced more potent CD3 signaling than constructs that are monovalent to B7H3 (cx5873 and cx5965). FIG. 37D shows the lack of activation of T-cells in the presence of B7H3 negative CCRF-CEM cells. A Jurkat CD3 NFAT-GFP reporter cell line was used to assess CD3 agonism.

FIGS. 38A and FIG. 38B depict the ability of representative B7H3-targeting constrained CD3 engaging constructs to induce T-cell mediated cytotoxicity in a target dependent manner. FIG. 38A shows that targeting B7H3 positive A375 cells with a construct that is bivalent and bi-epitopic to B7H3 (cx5187) induced more potent T-cell mediated cytotoxicity than constructs that are monovalent to B7H3 (cx5873 and cx5965). FIG. 38B depicts the lack of T-cell mediated cytotoxicity against B7H3 negative CCRF-CEM cells.

FIGS. 39A-D depict the ability of representative B7H3-targeting constrained CD3 engaging constructs to activate T-cells in the presence of B7H3 positive A375 cells, but not in the presence of B7H3 negative CCRF-CEM cells. FIGS. 39A and 39B show that targeting B7H3 positive A375 cells with a construct that is bivalent and bi-epitopic to B7H3 (cx5187), induced more potent CD25 expression on CD4+ and CD8+ T-cells, respectively, than constructs that are monovalent to B7H3 (cx5873 and cx5965). FIGS. 39C and 39D show the lack of CD25 expression on CD4+ and CD8+ T-cells, respectively, in the presence of B7H3 negative CCRF-CEM cells.

FIGS. 40A and 40B demonstrate the ability of representative B7H3-targeting constrained CD3 engaging constructs to elicit T-cell mediated cytotoxicity in the presence of B7H3-positive A375 cells (FIG. 40A) but not in the presence of CCRF-CEM B7H3-negative cells (FIG. 40B).

FIGS. 40C-40J demonstrate the ability of representative B7H3-targeting constrained CD3 engaging constructs to elicit T cell activation in the presence of B7H3-positive A375 cells but not in the presence of CCRF-CEM B7H3-negative cells, as assessed by: expression of CD25 on CD4+ T cells (FIGS. 40C and 40D, respectively), CD25 expression on CD8+ T cells (FIGS. 40E and 40F, respectively), CD71 expression on CD4+ T cells (FIGS. 40G and 40H, respectively), CD71 expression on CD8+ T cells (FIGS. 40I and 40J, respectively).

FIGS. 40K and 40L demonstrate the ability of representative B7H3-targeting constrained CD3 engaging constructs to elicit T cell cytokine production in the presence of B7H3-positive A375 cells (FIG. 40K) but not in the presence of CCRF-CEM B7H3-negative cells (FIG. 40L).

FIGS. 41A-41B depict various representative B7H3-targeted constrained CD3 engagers with a 4-1BB binding domain as a CRBR. cx5841 and cx5187 have a B7H3-targeting sdAb positioned at the N and C-termini of one chain of the heterodimer, the Fc knob, and have 41BB-targeting sdAb positioned at the C-termini of the opposite chain of the heterodimer, the Fc hole, but have the VH and VL of the CD3 binding Fv positioned on opposite sides with respect to each other.

FIGS. 42A-D depict results of T cell reporter assays for exemplary constructs described in FIGS. 41A-B. FIGS. 42A and 42B depict mean fluorescence intensity (MFI) of the GFP reporter when the B7H3 positive cell line A375 or the B7H3 negative cell line CCRF-CEM, respectively, were co-cultured with Jurkat CD3 NFAT-GFP reporter cells. FIGS. 42C and 42D depict relative luminescent units (RLU) of a luciferase reporter when the B7H3 positive cell line A375 or the B7H3 negative cell line CCRF-CEM, respectively, were co-cultured with Jurkat CD3 NFAT-Luciferase reporter cells.

### DETAILED DESCRIPTION

Provided herein are polypeptides that specifically bind to B7H3, hereinafter also called B7H3-binding polypeptides. In some embodiments, the provided binding polypeptides comprise at least one VHH domain that binds B7H3. In some embodiments, a B7H3-binding polypeptide provided herein comprises one, two, three, four, five, six, seven, or eight VHH domains that each individually binds B7H3. In some embodiments, a B7H3-binding polypeptide provided herein comprises one, two, three, or four VHH domains that bind B7H3. In some embodiments, the B7H3-binding polypeptides are monospecific. In some embodiments, the B7H3-binding polypeptides are multispecific. For example, provided B7H3-binding polypeptides include polypeptides that may comprise at least one VHH domain that binds B7H3 and one or more additional binding domains, such as one or more additional VHH domains, that bind one or more target proteins other than B7H3.

In some embodiments, a B7H3-binding polypeptide comprises at least one VHH domain that binds B7H3 and an Fc domain. In some embodiments, a B7H3-binding polypeptide provided herein comprises one, two, three, or four VHH domains that bind B7H3 and an Fc domain. In some embodiments, an Fc domain mediates dimerization of the B7H3-binding polypeptide at physiological conditions such that a dimer is formed that doubles the number of B7H3 binding sites. For example, a B7H3-binding polypeptide comprising three VHH domains that bind B7H3 and an Fc region is trivalent as a monomer, but at physiological conditions, the Fc region may mediate dimerization, such that the B7H3-binding polypeptide exists as a hexavalent dimer under such conditions.

B7H3 (also called CD276) is a member of the B7 family of immune cell modulating molecules. It is expressed on the surface of a wide variety of tumor cells and tumor vasculature including, but not limited to, neuroblastoma, melanoma, renal cell cancer, prostate cancer, colorectal cancer, pancreatic cancer, gastric cancer, breast cancer, ovarian cancer and small cell lung cancer. In humans the B7H3 protein is expressed in two forms, 2Ig and 4Ig. The 2Ig form has an extracellular region containing only one V-like and one C-like Ig domain, similar to other B7 family members (Chapoval et al, 2001, Nat. Immunol. 2:269-274). The 4Ig form contains a duplication of the V-like and the C-like Ig domain in tandem (Steinberger et al, 2004, J. Immunol. 172:2352-2359; Sun et al, 2002, J. Immunol. 168:6294-6297), and, has been shown to be the dominant isoform induced on immune cells and tumor cells in humans (Zhou et al. (2007) Tissue Antigens, 70:96-104). It has been shown that, in some cases, the level of B7H3 expression on tumor tissue is strongly correlated with the extent as to which the tumor has metastasized, with an increased risk of clinical cancer recurrence and with cancer-specific death (Roth et al, 2007, Cancer Res. 67:7893-7900; Zang et al, 2007, Proc. Natl. Acad. Sci. U.S.A. 104: 19458-19463). Similarly, a high level of B7H3 expression on tumor tissue correlated with poor patient survival in clear cell renal cell carcinoma, urothelial cell carcinoma (Crispen et al, 2008, Clin. Cancer Res. 14:5150-5157; Boorjian et al., 2008, Clin. Cancer Res. 14:4800-4808), ovarian cancer (Zang et al, 2010, Mod. Pathol. 23: 1 104-1 112), glioblastoma (Lemke et al, 2012, Clin. Cancer Res. 18: 105-117) osteosarcoma (Wang et al, 2013, PLoS One 8:e70689), pancreatic cancer (Yamato et al, 2009, Br. J. Cancer 101 : 1709-1716) and neuroblastoma (Gregorio et al, 2008, Histopathology 53:73-80).

An exemplary sequence of human B7H3 (4ig) is set forth as follows:

MLRRRGSPGMGVHVGAALGALWFCLTGALE
VQVPEDPVVALVGTDATLCCSFSPEPGFSL
AQLNLIWQLTDTKQLVHSFAEGQDQGSAYA
NRTALFPDLLAQGNASLRLQRVRVADEGSF
TCFVSIRDFGSAAVSLQVAAPYSKPSMTLE
PNKDLRPGDTVTITCSSYQGYPEAEVFWQD
GQGVPLTGNVTTSQMANEQGLFDVHSILRV
VLGANGTYSCLVRNPVLQQDAHSSVTITPQ
RSPTGAVEVQVPEDPVVALVGTDATLRCSF
SPEPGFSLAQLNLIWQLTDTKQLVHSFTEG
RDQGSAYANRTALFPDLLAQGNASLRLQRV
RVADEGSFTCFVSIRDFGSAAVSLQVAAPY
SKPSMTLEPNKDLRPGDTVTITCSSYRGYP
EAEVFWQDGQGVPLTGNVTTSQMANEQGLF
DVHSVLRVVLGANGTYSCLVRNPVLQQDAH
GSVTITGQPMTFPPEALWVTVGLSVCLIAL
LVALAFVCWRKIKQSCEEENAGAEDQDGEG
EGSKTALQPLKHSDSKEDDGQEIA
(SEQ ID NO: 190, signal sequence underlined)

In some cases, the provided B7H3 binding polypeptides directly block or inhibit activity of B7H3, which, in some aspects, can be used as a therapeutic to inhibit or reduce tumor cell growth or survival.

A variety of B7H3 polypeptide binding formats are provided. In some examples, B7H3 binding polypeptides include B7H3 VHH-Fc polypeptides. In some embodiments, the Fc is an Fc that exhibits immune effector activity, such as one or more effector functions such as antibody-dependent cellular cytotoxicity (ADCC), antibody-dependent cellular phagocytosis (ADCP) and/or complement-dependent cytotoxicity (CDC).

In some embodiments, the provided B7H3-binding polypeptides can be used to stimulate an immune response in a subject, which, in some aspects, treats a disease or disorder, such as a cancer, in the subject. In some aspects, a B7H3-binding polypeptide provided herein, such as a B7H3-Fc, can bind to B7H3-expressing tumor cells and induce an active immune response against the tumor cells expressing B7H3. In some cases, the active immune response can cause the death of the cancerous cells (e.g., antibody binding to cancer cells inducing apoptotic cell death), or inhibit the growth (e.g., block cells cycle progression) of the cancerous cells. In other cases, a B7H3-binding polypeptide provided herein, such as a B7H3 VHH-Fc, can bind to cancerous cells and antibody dependent cellular cytotoxicity (ADCC) can eliminate cancerous cells to which the B7H3-binding polypeptide binds. In some cases, provided B7H3 VHH-binding polypeptides can also activate both cellular and humoral immune responses and recruit more natural killer cells or increased production of cytokines (e.g., IL-2, IFN-gamma, IL-12, TNF-alpha, TNF-beta, etc.) that further activate an individual's immune system to destroy cancerous cells. In yet another embodiment, B7H3 binding polypeptides, such as B7H3 VHH-Fc, can bind to cancerous cells, and macrophages or other phagocytic cell can opsonize the cancerous cells, such as via CDC or ADCP processes.

In other aspects, also provided herein are VHH-binding polypeptides that exhibit multispecific binding. In some cases, the binding polypeptides include polypeptides that exhibit dual affinity for B7H3 and a T cell antigen, such as CD3. In some aspects, such dual affinity molecules are capable of engaging or activating T cells at the site of a tumor upon binding of tumor-expressed B7H3. In particular, among such molecules provided herein are molecules that exhibit constrained CD3 binding. Also provided herein are engineered cells, such as engineered T cells, that express a chimeric antigen receptor containing a B7H3 binding polypeptide.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993); and updated versions thereof.

The section headings used herein are for organizational purposes only and are not to be construed as limiting the subject matter described.

### I. DEFINITIONS

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The term “isolated polynucleotide” as used herein shall mean a polynucleotide of genomic, cDNA, or synthetic origin or some combination thereof, which by virtue of its origin (1) is not associated with all or a portion of a polynucleotide found in nature, (2) is operably linked to a polynucleotide that it is not linked to in nature, or (3) does not occur in nature as part of a larger sequence.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The term “isolated protein” referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a protein with which the “isolated protein” is associated in nature, (6) is operably associated (by covalent or noncovalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially pure or substantially free from proteins or polypeptides or other contaminants that are found in its natural environment that would interfere with its use (therapeutic, diagnostic, prophylactic, research or otherwise).

As used herein, “substantially pure” means an object species is the predominant species present (i.e., on a molar basis it is more abundant than any other individual species in the composition), and a substantially purified fraction is a composition wherein the object species comprises at least about 50 percent (on a molar basis) of all macromolecular species present. Generally, a substantially pure composition will comprise more than about 80 percent of all macromolecular species present in the composition, for example, in some embodiments, more than about 85%, 90%, 95%, and 99%. In some embodiments, the object species is purified to essential homogeneity (contaminant species cannot be detected in the composition by conventional detection methods) wherein the composition consists essentially of a single macromolecular species.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a B7H3 epitope is a sdAb or VHH-containing polypeptide that binds this epitope with greater affinity, avidity, more readily, and/or with greater duration than it binds to other B7H3 epitopes or non-B7H3 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen-binding molecule specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule specific to the epitope binds. It is noted that, in some embodiments, not every one of the residues within the linear epitope need be directly bound (or involved in a bond) by the antigen-binding molecule. In some embodiments, linear epitopes can be from immunizations with a peptide that effectively consisted of the sequence of the linear epitope, or from structural sections of a protein that are relatively isolated from the remainder of the protein (such that the antigen-binding molecule can interact, at least primarily), just with that sequence section.

The terms “antibody” and “antigen-binding molecule” are used interchangeably in the broadest sense and encompass various polypeptides that comprise antibody-like antigen-binding domains, including but not limited to conventional antibodies (typically comprising at least one heavy chain and at least one light chain), single-domain antibodies (sdAbs, comprising just one chain, which is typically similar to a heavy chain), VHH-containing polypeptides (polypeptides comprising at least one heavy chain only antibody variable domain, or VHH), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc domains (comprising hinge, CH2, and CH3, where the hinge mediates dimerization).

The term antibody also includes, but is not limited to, chimeric antibodies, humanized antibodies, and antibodies of various species such as camelid (including llama), shark, mouse, human, cynomolgus monkey, etc.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single V_{H} or V_{L} domain may be sufficient to confer antigen-binding specificity, e.g. a single domain antibody, such as a VHH. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

An “antibody fragment” or “antigen-binding fragment” refers to a molecule other than a conventional or intact antibody that comprises a portion of an conventional or intact antibody containing at least a variable region that binds an antigen. Examples of antibody fragments include but are not limited to Fv, single chain Fvs (sdFvs), Fab, Fab′, Fab′-SH, F(ab′)₂; diabodies; linear antibodies; an single-domain antibodies comprising only the V_{H} region (VHH).

As used herein, “monovalent” with reference to a binding molecule refers to binding molecules that have a single antigen recognition site that is specific for a target antigen. Examples of monovalent binding molecules include, for example, a monovalent antibody fragment, a proteinaceous binding molecule with antibody-like binding properties or an MHC molecule. Examples of monovalent antibody fragments include, but are not limited to, a Fab fragment, an Fv fragment, and a single-chain Fv fragment (scFv).

The terms “single domain antibody”, “sdAb,” “VHH” are used interchangeably herein to refer to an antibody having a single monomeric domain antigen binding/recognition domain. Such antibodies include a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprise only a partial FR1 and/or FR4, or lacks one or both of those framework regions, so long as the VHH substantially maintains antigen binding and specificity.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc domain. In some such embodiments, the VHH polypeptide may form a dimer. Nonlimiting structures of VHH-containing polypeptides include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains).

As used herein, a B7H3-binding polypeptide is a polypeptide or protein that specifically binds B7H3. Typically, a B7H3-binding polypeptide herein is a VHH-containing polypeptide containing at least one VHH domain that binds B7H3. A B7H3-binding polypeptide includes conjugates, including fusion proteins. A B7H3-binding polypeptide includes fusion proteins, including those containing an Fc domain. In some embodiments, a B7H3-binding polypeptide contains two, three, or four or more VHH domains that each specifically bind to B7H3, wherein each VHH domain may be the same or different. In some embodiments, a B7H3-binidng polypeptide is multivalent. In some embodiments, a B7H3-binding polypeptide is multispecific. In some cases, a B7H3-binding polypeptide may contain one or more additional domains that bind to one or more further or additional antigens other than B7H3.

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3. Table 1, below, lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact |
|---|---|---|---|---|
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273,927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given VHH amino acid sequence, it is understood that such a CDR has a sequence of the corresponding CDR (e.g., CDR-H3) within the VHH, as defined by any of the aforementioned schemes. In some embodiments, specific CDR sequences are specified. Exemplary CDR sequences of provided antibodies are described using various numbering schemes (see e.g. Table 1), although it is understood that a provided antibody can include CDRs as described according to any of the other aforementioned numbering schemes or other numbering schemes known to a skilled artisan.

As used herein, “conjugate,” “conjugation” or grammatical variations thereof refers the joining or linking together of two or more compounds resulting in the formation of another compound, by any joining or linking methods known in the art. It can also refer to a compound which is generated by the joining or linking together two or more compounds. For example, a VHH domain linked directly or indirectly to one or more chemical moieties or polypeptide is an exemplary conjugate. Such conjugates include fusion proteins, those produced by chemical conjugates and those produced by any other methods.

An immunoglobulin Fc fusion (“Fc-fusion”), such as VHH-Fc, is a molecule comprising one or more VHH domains operably linked to an Fc region of an immunoglobulin. An immunoglobulin Fc region may be linked indirectly or directly to one or more VHH domains. Various linkers are known in the art and can optionally be used to link an Fc to a fusion partner to generate an Fc-fusion. In some such embodiments, the linker comprises 1-20 amino acids, preferably 1-20 amino acids predominantly composed of glycine and, optionally, serine. Fc-fusions of identical species can be dimerized to form Fc-fusion homodimers, or using non-identical species to form Fc-fusion heterodimers. In some embodiments, the Fc is a mammalian Fc such as human Fc.

The term “heavy chain constant region” as used herein refers to a region comprising at least three heavy chain constant domains, C_{H}1, hinge, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γconstant region is an IgG antibody, an antibody comprising a δ constant region is an IgD antibody, and an antibody comprising an α constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

A “functional Fc region” possesses an “effector function” of a native sequence Fc region. Exemplary “effector functions” include Fc receptor binding; C1q binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

In general, the numbering of the residues in an immunoglobulin heavy chain or portion thereof, such as an Fc region, is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

An “acceptor human framework” as used herein is a framework comprising the amino acid sequence of a heavy chain variable domain (V_{H}) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

As used herein, a “chimeric antigen receptor” or “CAR” refers to an engineered receptor, which introduces an antigen specificity, via an antigen binding domain, onto cells to which it is engineered (for example T cells such as naive T cells, central memory T cells, effector memory T cells or combination thereof) thus combining the antigen binding properties of the antigen binding domain with the T cell activity (e.g. lytic capacity and self renewal) of T cells. A CAR typically includes an extracellular antigen-binding domain (ectodomain), a transmembrane domain and an intracellular signaling domain. The intracellular signaling domain generally contains at least one ITAM signaling domain, e.g. derived from CD3zeta, and optionally at least one costimulatory signaling domain, e.g. derived from CD28 or 4-1BB. In a CAR provided herein, a VHH domain forms the antigen binding domain and is located at the extracellular side when expressed in a cell.

“Affinity” refers to the strength of the sum total of noncovalent interactions between a single binding site of a molecule (for example, an antibody or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation (such as T cell proliferation), and inducing or increasing expression of cytokines.

An “affinity matured” VHH-containing polypeptide refers to a VHH-containing polypeptide with one or more alterations in one or more CDRs compared to a parent VHH-containing polypeptide that does not possess such alterations, such alterations resulting in an improvement in the affinity of the VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize VHH or VHH-containing polypeptide performance. In some embodiments, a humanized VHH-containing polypeptide comprises a human Fc region. As will be appreciated, a humanized sequence can be identified by its primary sequence and does not necessarily denote the process by which the antibody was created.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 2. Amino acid substitutions may be introduced into an antibody of interest and the products screened for a desired activity, for example, retained/improved antigen binding, decreased immunogenicity, or improved ADCC or CDC.

TABLE 2

| Original | |
|---|---|
| Residue | Exemplary Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Also, included in this definition are cells having abnormal proliferation that is not impeded (e.g. immune evasion and immune escape mechanisms) by the immune system (e.g. virus infected cells). Exemplary cancers include, but are not limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The term “non-tumor cell” as used herein refers to a normal cells or tissue. Exemplary non-tumor cells include, but are not limited to: T-cells, B-cells, natural killer (NK) cells, natural killer T (NKT) cells, dendritic cells, monocytes, macrophages, epithelial cells, fibroblasts, hepatocytes, interstitial kidney cells, fibroblast-like synoviocytes, osteoblasts, and cells located in the breast, skeletal muscle, pancreas, stomach, ovary, small intestines, placenta, uterus, testis, kidney, lung, heart, brain, liver, prostate, colon, lymphoid organs, bone, and bone-derived mesenchymal stem cells. The term “a cell or tissue located in the periphery” as used herein refers to non-tumor cells not located near tumor cells and/or within the tumor microenvironment.

The term “cells or tissue within the tumor microenvironment” as used herein refers to the cells, molecules, extracellular matrix and/or blood vessels that surround and/or feed a tumor cell. Exemplary cells or tissue within the tumor microenvironment include, but are not limited to: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells (Treg cells); macrophages; neutrophils; myeloid-derived suppressor cells (MDSCs) and other immune cells located proximal to a tumor. Methods for identifying tumor cells, and/or cells/tissues located within the tumor microenvironment are well known in the art, as described herein, below.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, cancer or cancer metastasis). “An immune response” can encompass aspects of both the innate and adaptive immune systems.

As used herein, the terms “treating,” “treatment,” or “therapy” of a disease, disorder or condition is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

As used herein in the context of cancer, the terms “treatment” or, “inhibit,” “inhibiting” or “inhibition” of cancer refers to at least one of: a statistically significant decrease in the rate of tumor growth, a cessation of tumor growth, or a reduction in the size, mass, metabolic activity, or volume of the tumor, as measured by standard criteria such as, but not limited to, the Response Evaluation Criteria for Solid Tumors (RECIST), or a statistically significant increase in progression free survival (PFS) or overall survival (OS).

“Ameliorating” means a lessening or improvement of one or more symptoms as compared to not administering a therapeutic agent. “Ameliorating” also includes shortening or reduction in duration of a symptom.

“Preventing,” “prophylaxis,” or “prevention” of a disease or disorder refers to administration of a pharmaceutical composition, either alone or in combination with another compound, to prevent the occurrence or onset of a disease or disorder or some or all of the symptoms of a disease or disorder or to lessen the likelihood of the onset of a disease or disorder.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

The terms “effective amount” or “therapeutically effective amount” refer to a quantity and/or concentration of a composition containing an active ingredient (e.g. sdAb or VHH-containing polypeptide) that when administered into a patient either alone (i.e., as a monotherapy) or in combination with additional therapeutic agents, yields a statistically significant decrease in disease progression as, for example, by ameliorating or eliminating symptoms and/or the cause of the disease. An effective amount may be an amount that relieves, lessens, or alleviates at least one symptom or biological response or effect associated with a disease or disorder, prevents progression of the disease or disorder, or improves physical functioning of the patient. A therapeutically effective amount of a composition containing an active agent may vary according to factors such as the disease state, age, sex, and weight of the individual, and the ability of the active agent to elicit a desired response in the individual. A therapeutically effective amount is also one in which any toxic or detrimental effects of the active agent are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

As used herein, a composition refers to any mixture of two or more products, substances, or compounds, including cells. It may be a solution, a suspension, liquid, powder, a paste, aqueous, non-aqueous or any combination thereof.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Hence, it is a composition suitable for pharmaceutical use in a mammalian subject, often a human. A pharmaceutical composition typically comprises an effective amount of an active agent (e.g., sdAb or VHH-containing polypeptide) and a carrier, excipient, or diluent. The carrier, excipient, or diluent is typically a pharmaceutically acceptable carrier, excipient or diluent, respectively. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effect of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### II. VHH DOMAINS BINDING B7H3

Provided herein are B7H3-binding polypeptides that are VHH-containing polypeptides containing at least one VHH domain that specifically binds to B7H3. In some embodiments, the VHH domain binds human B7H3. In some of any of the provided embodiments, the VHH domain binds B7H3 having the sequence set forth in SEQ ID NO:190 or a mature form thereof lacking the signal sequence. In some embodiments, the VHH domain bind or recognizes 4IgB7H3. In some embodiments, the VHH domain binds or recognizes 2IgB7H3. In some embodiments, the VHH domain binds or recognizes 4IgB7H3 and 2IgB7H3. In some embodiments, the VHH-containing polypeptides incorporate multiple copies of a VHH domain provided herein. In such embodiments, the VHH-containing polypeptide may incorporate multiple copies of the same VHH domain. In some embodiments, the VHH-containing polypeptides may incorporate multiple copies of a VHH domain that are different but that recognize the same epitope on B7H3. The VHH-containing polypeptides can be formatted in a variety of formats, including any as described in Section III below.

A VHH domain is an antibody fragment that is a single monomeric variable antibody domain that is able to bind selectively to a specific antigen. With a molecular weight of only 12-15 kDa, VHH domains (also called single-domain antibodies) are much smaller than common antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain).

Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be derived from any species including, but not limited to mouse, human, camel, llama, alpaca, vicuna, guanaco, shark, goat, rabbit, and/or bovine. In some embodiments, a single domain antibody as used herein is a naturally occurring single domain antibody known as heavy chain antibody devoid of light chains. For clarity reasons, this variable domain derived from a heavy chain antibody naturally devoid of light chain is known herein as a VHH to distinguish it from the conventional VH of four chain immunoglobulins. Such a VHH molecule can be derived from antibodies raised in Camelidae species, for example in camel, llama, dromedary, alpaca, vicuna and guanaco. Other species besides Camelidae may produce heavy chain antibodies naturally devoid of light chain; such VHHs are within the scope of the disclosure.

Methods for the screening of VHH domains, including VHH-binding polypeptides, that possess the desired specificity for B7H3 include, but are not limited to, enzyme linked immunosorbent assay (ELISA), enzymatic assays, flow cytometry, and other immunologically mediated techniques known within the art.

Among the provided VHH domains provided herein are B7H3 VHH (llama-derived) and humanized sequences, such as any described below.

In some embodiments, a VHH domain that binds B7H3 may be humanized. Humanized antibodies (such as VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151 :2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

Provided herein is a VHH domain that binds B7H3 in which the VHH domain comprises a a CDR1, CDR2 and CDR3 contained in a VHH amino acid sequences selected from any of SEQ ID NO: 1-114 or an amino acid sequence that has at least 90%, 91%, 92%, 93%, 94%, 95%, 96%, 97%, 98%, or 99% sequence identity to the V_{H}H region amino acid selected from any one of SEQ ID NOs: 1-114 In some embodiments, a B7H3 VHH domain provided herein contains a CDR1 set forth in any one of SEQ ID NOS: 115-145, a CDR2 set forth in any one of SEQ ID NOS: 146-167 and a CDR3 set forth in any one of SEQ ID NOS: 168-189. Among the provided B7H3 VHH domain has the amino acid sequence set forth in any of SEQ ID NOS: 1-114or an amino acid sequence that has at least 90%, 91%, 92%, 93%, 94%, 95%, 96%, 97%, 98%, or 99% sequence identity to the V_{H}H region amino acid selected from any one of SEQ ID NOs: 1-114 In some embodimetns, the B7H3 VHH domain has the sequence of amino acids set forth in any one of SEQ ID NOS: 1-114.

Provided herein is a VHH domain that binds B7H3 in which the VHH domain comprises a a CDR1, CDR2 and CDR3 contained in a VHH amino acid sequences selected from any of SEQ ID NO: 1-114, 466, 467, 489, or 490, 492-518 or an amino acid sequence that has at least 90%, 91%, 92%, 93%, 94%, 95%, 96%, 97%, 98%, or 99% sequence identity to the V_{H}H region amino acid selected from any one of SEQ ID NOs: 1-114, 466, 467, 489, or 490, 492-518 In some embodiments, a B7H3 VHH domain provided herein contains a CDR1 set forth in any one of SEQ ID NOS: 115-145, a CDR2 set forth in any one of SEQ ID NOS: 146-167 and a CDR3 set forth in any one of SEQ ID NOS: 168-189 or 483-488. Among the provided B7H3 VHH domain has the amino acid sequence set forth in any of SEQ ID NOS: 1-114, 466, 467, 489, or 490, 492-518 or an amino acid sequence that has at least 90%, 91%, 92%, 93%, 94%, 95%, 96%, 97%, 98%, or 99% sequence identity to the V_{H}H region amino acid selected from any one of SEQ ID NOs: 1-114, 466, 467, 489, or 490, 492-518. In some embodimetns, the B7H3 VHH domain has the sequence of amino acids set forth in any one of SEQ ID NOS: 1-114, 466, 467, 489, or 490, 492-518.

In some embodiments, a B7H3 VHH domain provided herein contains a CDR1, CDR2, CDR3 contained in a VHH domain set forth in SEQ ID NO:1, or an amino acid sequence that has at least 90%, 91%, 92%, 93%, 94%, 95%, 96%, 97%, 98%, or 99% sequence identity to the VHH region amino acid selected set forth in SEQ ID NO:1. In some embodiments, the B7H3 VHH domain has the amino acid sequence set forth in SEQ ID NO:1 or an amino acid sequence that has at least 90%, 91%, 92%, 93%, 94%, 95%, 96%, 97%, 98%, or 99% sequence identity to the amino acid selected set forth in SEQ ID NO: 1. In some embodiments, the B7H3 VHH domain is a humanized variant of the amino acid sequence set forth in SEQ ID NO:1.

In some embodiments, a B7H3 VHH domain provided herein contains a CDR1 set forth in any one of SEQ ID NOS: 115, 116, 117, 118, 119, 120, 121 or 122, a CDR2 set forth in any one of SEQ ID NOS: 146, 147, 148, 149, 150, 151 and a CDR3 set forth in SEQ ID NO: 168.

In some embodiments, the B7H3 VHH domain provided herein contains a CDR1, CDR2, and CDR3 set forth in any one of SEQ ID NO: 115, 146 and 168, respectively. In some embodiments, the B7H3 VHH domain provided herein contains a CDR1, CDR2 and CDR3 set forth in any one of SEQ ID NO: 116, 146 and 168, respectively. In some embodiments, the B7H3 VHH domain provided herein contains a CDR1, CDR2 and CDR3 set forth in any one of SEQ ID NO: 117, 146 and 168, respectively. In some embodiments, the B7H3 VHH domain provided herein contains a CDR1, CDR2 and CDR3 set forth in any one of SEQ ID NO: 118, 146 and 168, respectively. In some embodiments, the B7H3 VHH domain provided herein contains a CDR1, CDR2 and CDR3 set forth in any one of SEQ ID NO: 119, 146 and 168, respectively. In some embodiments, the B7H3 VHH domain provided herein contains a CDR1, CDR2 and CDR3 set forth in any one of SEQ ID NO: 120, 146 and 168, respectively. In some embodiments, the B7H3 VHH domain provided herein contains a CDR1, CDR2 and CDR3 set forth in any one of SEQ ID NO: 115, 147 and 168, respectively. In some embodiments, the B7H3 VHH domain provided herein contains a CDR1, CDR2 and CDR3 set forth in any one of SEQ ID NO: 115, 148 and 168, respectively. In some embodiments, the B7H3 VHH domain provided herein contains a CDR1, CDR2 and CDR3 set forth in any one of SEQ ID NO: 115, 149 and 168, respectively. In some embodiments, the B7H3 VHH domain provided herein contains a CDR1, CDR2 and CDR3 set forth in any one of SEQ ID NO: 115, 150 and 168, respectively. In some embodiments, the B7H3 VHH domain provided herein contains a CDR1, CDR2 and CDR3 set forth in any one of SEQ ID NO: 115, 151 and 168, respectively. In some embodiments, the B7H3 VHH domain provided herein contains a CDR1, CDR2 and CDR3 set forth in any one of SEQ ID NO: 116, 147 and 168, respectively. In some embodiments, the B7H3 VHH domain provided herein contains a CDR1, CDR2 and CDR3 set forth in any one of SEQ ID NO: 118, 147 and 168, respectively. In some embodiments, the B7H3 VHH domain provided herein contains a CDR1, CDR2 and CDR3 set forth in any one of SEQ ID NO: 119, 147 and 168, respectively. In some embodiments, the B7H3 VHH domain provided herein contains a CDR1, CDR2 and CDR3 set forth in any one of SEQ ID NO: 116, 151 and 168, respectively. In some embodiments, the B7H3 VHH domain provided herein contains a CDR1, CDR2 and CDR3 set forth in any one of SEQ ID NO: 121, 147 and 168, respectively. In some embodiments, the B7H3 VHH domain provided herein contains a CDR1, CDR2 and CDR3 set forth in SEQ ID NOs: 119, 149 and 168, respectively. In some embodiments, the B7H3 VHH domain provided herein contains a CDR1, CDR2 and CDR3 set forth in any one of SEQ ID NO: 122, 151 and 168, respectively.

In some aspects, a VHH domain that binds B7H3 comprises a CDR1, CDR2 and CDR3 contained in a VHH amino acid sequences selected from any of SEQ ID NO: 2-34, 467,or an amino acid sequence that has at least 90%, 91%, 92%, 93%, 94%, 95%, 96%, 97%, 98%, or 99% sequence identity to the V_{H}H region amino acid selected from any one of SEQ ID NOs: 2-34, 467.

In some aspects, a VHH domain that binds B7H3 comprises a CDR1, CDR2 and CDR3 contained in a VHH amino acid sequences selected from any of SEQ ID NO: 2-34, 467, 489-490, and 492-497 or an amino acid sequence that has at least 90%, 91%, 92%, 93%, 94%, 95%, 96%, 97%, 98%, or 99% sequence identity to the V_{H}H region amino acid selected from any one of SEQ ID NOs: 2-34, 467, 489-490, and 492-497.

In some cases, the provided B7H3 VHH domain is a humanized variant that has the amino acid sequence set forth in any of SEQ ID NOS: 2-34, 467, or an amino acid sequence that has at least 90%, 91%, 92%, 93%, 94%, 95%, 96%, 97%, 98%, or 99% sequence identity to the V_{H}H region amino acid selected from any one of SEQ ID NOs: 2-34, 467, . In some embodimetns, the B7H3 humanized VHH domain has the sequence of amino acids set forth in any one of SEQ ID NOS: 2-34, 467.

In some cases, the provided B7H3 VHH domain is a humanized variant that has the amino acid sequence set forth in any of SEQ ID NOS: 2-34, 467, 489-490, and 492-497 or an amino acid sequence that has at least 90%, 91%, 92%, 93%, 94%, 95%, 96%, 97%, 98%, or 99% sequence identity to the V_{H}H region amino acid selected from any one of SEQ ID NOs: 2-34, 467, 489-490, and 492-497. In some embodimetns, the B7H3 humanized VHH domain has the sequence of amino acids set forth in any one of SEQ ID NOS: 2-34, 467, 489, and 490.

In some embodiments, a B7H3 VHH domain provided herein contains a CDR1, CDR2, CDR3 contained in a VHH domain set forth in SEQ ID NO:35, or an amino acid sequence that has at least 90%, 91%, 92%, 93%, 94%, 95%, 96%, 97%, 98%, or 99% sequence identity to the VHH region amino acid selected set forth in SEQ ID NO:35. In some embodiments, the B7H3 VHH domain has the amino acid sequence set forth in SEQ ID NO:35 or an amino acid sequence that has at least 90%, 91%, 92%, 93%, 94%, 95%, 96%, 97%, 98%, or 99% sequence identity to the amino acid selected set forth in SEQ ID NO: 35. In some embodiments, the B7H3 VHH domain is a humanized variant of the amino acid sequence set forth in SEQ ID NO:35.

In some embodiments, a B7H3 VHH domain provided herein contains a CDR1 set forth in SEQ ID NO: 123, a CDR2 set forth in SEQ ID NO: 152 or 153 and a CDR3 set forth in SEQ ID NO: 170 or 171.

In some embodiments, the B7H3 VHH domain provided herein contains a CDR1, CDR2, and CDR3 set forth in any one of SEQ ID NO: 123, 152 and 170, respectively. In some embodiments, the B7H3 VHH domain provided herein contains a CDR1, CDR2, and CDR3 set forth in any one of SEQ ID NO: 123, 153 and 170, respectively. In some embodiments, the B7H3 VHH domain provided herein contains a CDR1, CDR2, and CDR3 set forth in any one of SEQ ID NO: 123, 153 and 171, respectively.

In some aspects, a VHH domain that binds B7H3 comprises a a CDR1, CDR2 and CDR3 contained in a VHH amino acid sequences selected from any of SEQ ID NO:36-43or an amino acid sequence that has at least 90%, 91%, 92%, 93%, 94%, 95%, 96%, 97%, 98%, or 99% sequence identity to the V_{H}H region amino acid selected from any one of SEQ ID NOs: 36-43.

In some aspects, a VHH domain that binds B7H3 comprises a a CDR1, CDR2 and CDR3 contained in a VHH amino acid sequences selected from any of SEQ ID NO:36-43 and 498-503, or an amino acid sequence that has at least 90%, 91%, 92%, 93%, 94%, 95%, 96%, 97%, 98%, or 99% sequence identity to the V_{H}H region amino acid selected from any one of SEQ ID NOs: 36-43 and 498-503.

In some cases, the provided B7H3 VHH domain is a humanized variant that has the amino acid sequence set forth in any of SEQ ID NOS: 36-43 or an amino acid sequence that has at least 90%, 91%, 92%, 93%, 94%, 95%, 96%, 97%, 98%, or 99% sequence identity to the V_{H}H region amino acid selected from any one of SEQ ID NOs: 36-43. In some embodimetns, the B7H3 humaized VHH domain has the sequence of amino acids set forth in any one of SEQ ID NOS: 36-43.

In some cases, the provided B7H3 VHH domain is a humanized variant that has the amino acid sequence set forth in any of SEQ ID NOS: 36-43 and 498-503 or an amino acid sequence that has at least 90%, 91%, 92%, 93%, 94%, 95%, 96%, 97%, 98%, or 99% sequence identity to the V_{H}H region amino acid selected from any one of SEQ ID NOs: 36-43 and 498-503. In some embodimetns, the B7H3 humaized VHH domain has the sequence of amino acids set forth in any one of SEQ ID NOS: 36-43 and 498-503.

In some embodiments, a B7H3 VHH domain provided herein contains a CDR1, CDR2, CDR3 contained in a VHH domain set forth in SEQ ID NO:44, or an amino acid sequence that has at least 90%, 91%, 92%, 93%, 94%, 95%, 96%, 97%, 98%, or 99% sequence identity to the VHH region amino acid selected set forth in SEQ ID NO:44. In some embodiments, the B7H3 VHH domain has the amino acid sequence set forth in SEQ ID NO:44 or an amino acid sequence that has at least 90%, 91%, 92%, 93%, 94%, 95%, 96%, 97%, 98%, or 99% sequence identity to the amino acid selected set forth in SEQ ID NO: 44. In some embodiments, the B7H3 VHH domain is a humanized variant of the amino acid sequence set forth in SEQ ID NO:44.

In some embodiments, a B7H3 VHH domain provided herein contains a CDR1 set forth in any one of SEQ ID NOs: 124, 125, 126, 127, 128, 129, 130, 131, 132, 133, a CDR2 set forth in SEQ ID NO: 154 and a CDR3 set forth in any one of SEQ ID NOs:.172, 173, 174, 175, 176, 177, 178, 179, 180, 181, 182 or 183.

In some embodiments, the B7H3 VHH domain provided herein contains a CDR1, CDR2, and CDR3 set forth in any one of SEQ ID NO: 124, 154, 172, respectively. In some embodiments, the B7H3 VHH domain provided herein contains a CDR1, CDR2, and CDR3 set forth in any one of SEQ ID NO: 124, 154, 173, respectively. In some embodiments, the B7H3 VHH domain provided herein contains a CDR1, CDR2, and CDR3 set forth in any one of SEQ ID NO: 124, 154, 174, respectively. In some embodiments, the B7H3 VHH domain provided herein contains a CDR1, CDR2, and CDR3 set forth in any one of SEQ ID NO: 124, 154, 175, respectively. In some embodiments, the B7H3 VHH domain provided herein contains a CDR1, CDR2, and CDR3 set forth in any one of SEQ ID NO: 125, 154, 173, respectively. In some embodiments, the B7H3 VHH domain provided herein contains a CDR1, CDR2, and CDR3 set forth in any one of SEQ ID NO: 126, 154, 173, respectively. In some embodiments, the B7H3 VHH domain provided herein contains a CDR1, CDR2, and CDR3 set forth in any one of SEQ ID NO: 127, 154, 173, respectively. In some embodiments, the B7H3 VHH domain provided herein contains a CDR1, CDR2, and CDR3 set forth in any one of SEQ ID NO: 128, 154, 173, respectively. In some embodiments, the B7H3 VHH domain provided herein contains a CDR1, CDR2, and CDR3 set forth in any one of SEQ ID NO: 129, 154, 173, respectively. In some embodiments, the B7H3 VHH domain provided herein contains a CDR1, CDR2, and CDR3 set forth in any one of SEQ ID NO: 130, 154, 173, respectively. In some embodiments, the B7H3 VHH domain provided herein contains a CDR1, CDR2, and CDR3 set forth in any one of SEQ ID NO: 131, 154, 173, respectively. In some embodiments, the B7H3 VHH domain provided herein contains a CDR1, CDR2, and CDR3 set forth in any one of SEQ ID NO: 124, 154, 176, respectively. In some embodiments, the B7H3 VHH domain provided herein contains a CDR1, CDR2, and CDR3 set forth in any one of SEQ ID NO: 124, 154, 177, respectively. In some embodiments, the B7H3 VHH domain provided herein contains a CDR1, CDR2, and CDR3 set forth in any one of SEQ ID NO: 124, 154, 178, respectively. In some embodiments, the B7H3 VHH domain provided herein contains a CDR1, CDR2, and CDR3 set forth in any one of SEQ ID NO: 124, 154, 179, respectively. In some embodiments, the B7H3 VHH domain provided herein contains a CDR1, CDR2, and CDR3 set forth in any one of SEQ ID NO: 124, 154, 180, respectively. In some embodiments, the B7H3 VHH domain provided herein contains a CDR1, CDR2, and CDR3 set forth in any one of SEQ ID NO: 124, 154, 181, respectively. In some embodiments, the B7H3 VHH domain provided herein contains a CDR1, CDR2, and CDR3 set forth in any one of SEQ ID NO: 124, 154, 182, respectively. In some embodiments, the B7H3 VHH domain provided herein contains a CDR1, CDR2, and CDR3 set forth in any one of SEQ ID NO: 124, 154, 183, respectively. In some embodiments, the B7H3 VHH domain provided herein contains a CDR1, CDR2, and CDR3 set forth in any one of SEQ ID NO: 126, 154, 176, respectively. In some embodiments, the B7H3 VHH domain provided herein contains a CDR1, CDR2, and CDR3 set forth in any one of SEQ ID NO: 126, 154, 179, respectively. In some embodiments, the B7H3 VHH domain provided herein contains a CDR1, CDR2, and CDR3 set forth in any one of SEQ ID NO: 126, 154, 182, respectively. In some embodiments, the B7H3 VHH domain provided herein contains a CDR1, CDR2, and CDR3 set forth in any one of SEQ ID NO: 132, 154, 176, respectively. In some embodiments, the B7H3 VHH domain provided herein contains a CDR1, CDR2, and CDR3 set forth in any one of SEQ ID NO: 133, 154, 173, respectively.

In some aspects, a VHH domain that binds B7H3 comprises a a CDR1, CDR2 and CDR2 contained in a VHH amino acid sequences selected from any of SEQ ID NO: 45-91, 466, or an amino acid sequence that has at least 90%, 91%, 92%, 93%, 94%, 95%, 96%, 97%, 98%, or 99% sequence identity to the V_{H}H region amino acid selected from any one of SEQ ID NOs: 45-91, 466.

In some aspects, a VHH domain that binds B7H3 comprises a a CDR1, CDR2 and CDR2 contained in a VHH amino acid sequences selected from any of SEQ ID NO: 45-91, 466, and 504-514, or an amino acid sequence that has at least 90%, 91%, 92%, 93%, 94%, 95%, 96%, 97%, 98%, or 99% sequence identity to the V_{H}H region amino acid selected from any one of SEQ ID NOs: 45-91, 466, and 504-514.

In some cases, the provided B7H3 VHH domain is a humanized variant that has the amino acid sequence set forth in any of SEQ ID NOS: 45-91, 466, aor an amino acid sequence that has at least 90%, 91%, 92%, 93%, 94%, 95%, 96%, 97%, 98%, or 99% sequence identity to the V_{H}H region amino acid selected from any one of SEQ ID NOs: 45-91, 466. In some embodiments, the B7H3 humanized VHH domain has the sequence of amino acids set forth in any one of SEQ ID NOS: 45-91, 466.

In some cases, the provided B7H3 VHH domain is a humanized variant that has the amino acid sequence set forth in any of SEQ ID NOS: 45-91, 466, and 504-514or an amino acid sequence that has at least 90%, 91%, 92%, 93%, 94%, 95%, 96%, 97%, 98%, or 99% sequence identity to the V_{H}H region amino acid selected from any one of SEQ ID NOs: 45-91, 466, and 504-514. In some embodiments, the B7H3 humanized VHH domain has the sequence of amino acids set forth in any one of SEQ ID NOS: 45-91, 466, and 504-514.

In some embodiments, a B7H3 VHH domain provided herein contains a CDR1, CDR2, CDR3 contained in a VHH domain set forth in SEQ ID NO:105, or an amino acid sequence that has at least 90%, 91%, 92%, 93%, 94%, 95%, 96%, 97%, 98%, or 99% sequence identity to the VHH region amino acid selected set forth in SEQ ID NO:105. In some embodiments, the B7H3 VHH domain has the amino acid sequence set forth in SEQ ID NO:105 or an amino acid sequence that has at least 90%, 91%, 92%, 93%, 94%, 95%, 96%, 97%, 98%, or 99% sequence identity to the amino acid selected set forth in SEQ ID NO: 105. In some embodiments, the B7H3 VHH domain is a humanized variant of the amino acid sequence set forth in SEQ ID NO:105.

In some embodiments, a B7H3 VHH domain provided herein contains a CDR1 set forth in SEQ ID NO: 145, a CDR2 set forth in SEQ ID NO: 167 and a CDR3 set forth in SEQ ID NO:195.

In some embodiments, a B7H3 VHH domain provided herein contains a CDR1 set forth in SEQ ID NO: 145, a CDR2 set forth in SEQ ID NO: 167 and a CDR3 set forth in SEQ ID NO:488.

In some aspects, a VHH domain that binds B7H3 comprises a a CDR1, CDR2 and CDR2 contained in a VHH amino acid sequences selected from any of SEQ ID NO:106-109, or an amino acid sequence that has at least 90%, 91%, 92%, 93%, 94%, 95%, 96%, 97%, 98%, or 99% sequence identity to the V_{H}H region amino acid selected from any one of SEQ ID NOs: 106-109.

In some cases, the provided B7H3 VHH domain is a humanized variant that has the amino acid sequence set forth in any of SEQ ID NOS: 106-109 or an amino acid sequence that has at least 90%, 91%, 92%, 93%, 94%, 95%, 96%, 97%, 98%, or 99% sequence identity to the V_{H}H region amino acid selected from any one of SEQ ID NOs: 106-109. In some embodimetns, the B7H3 humanized VHH domain has the sequence of amino acids set forth in any one of SEQ ID NOS: 106-109.

In some embodiments, a B7H3 VHH domain provided herein contains a CDR1, CDR2, CDR3 contained in a VHH domain set forth in SEQ ID NO:110, or an amino acid sequence that has at least 90%, 91%, 92%, 93%, 94%, 95%, 96%, 97%, 98%, or 99% sequence identity to the VHH region amino acid selected set forth in SEQ ID NO:110. In some embodiments, the B7H3 VHH domain has the amino acid sequence set forth in SEQ ID NO:110 or an amino acid sequence that has at least 90%, 91%, 92%, 93%, 94%, 95%, 96%, 97%, 98%, or 99% sequence identity to the amino acid selected set forth in SEQ ID NO: 110. In some embodiments, the B7H3 VHH domain is a humanized variant of the amino acid sequence set forth in SEQ ID NO:110.

In some embodiments, a B7H3 VHH domain provided herein contains a CDR1 set forth in SEQ ID NO: 131, a CDR2 set forth in SEQ ID NO: 169 and a CDR3 set forth in SEQ ID NO:189.

In some embodiments, a B7H3 VHH domain provided herein contains a CDR1 set forth in SEQ ID NO: 131, a CDR2 set forth in SEQ ID NO: 161 and a CDR3 set forth in SEQ ID NO:189.

In some aspects, a VHH domain that binds B7H3 comprises a a CDR1, CDR2 and CDR2 contained in a VHH amino acid sequences selected from any of SEQ ID NO:111-114, or an amino acid sequence that has at least 90%, 91%, 92%, 93%, 94%, 95%, 96%, 97%, 98%, or 99% sequence identity to the V_{H}H region amino acid selected from any one of SEQ ID NOs: 111-114.

In some aspects, a VHH domain that binds B7H3 comprises a a CDR1, CDR2 and CDR2 contained in a VHH amino acid sequences selected from any of SEQ ID NO:111-114 and 515-528, or an amino acid sequence that has at least 90%, 91%, 92%, 93%, 94%, 95%, 96%, 97%, 98%, or 99% sequence identity to the V_{H}H region amino acid selected from any one of SEQ ID NOs: 111-114 and 515-518.

In some cases, the provided B7H3 VHH domain is a humanized variant that has the amino acid sequence set forth in any of SEQ ID NOS: 111-114 or an amino acid sequence that has at least 90%, 91%, 92%, 93%, 94%, 95%, 96%, 97%, 98%, or 99% sequence identity to the V_{H}H region amino acid selected from any one of SEQ ID NOs: 111-114. In some embodimetns, the B7H3 humanized VHH domain has the sequence of amino acids set forth in any one of SEQ ID NOS: 111-114.

In some cases, the provided B7H3 VHH domain is a humanized variant that has the amino acid sequence set forth in any of SEQ ID NOS: 111-114 and 515-518 or an amino acid sequence that has at least 90%, 91%, 92%, 93%, 94%, 95%, 96%, 97%, 98%, or 99% sequence identity to the V_{H}H region amino acid selected from any one of SEQ ID NOs: 111-114 and 515-518. In some embodimetns, the B7H3 humanized VHH domain has the sequence of amino acids set forth in any one of SEQ ID NOS: 111-114 and 515-518.

### III. FUSION PROTEINS AND CONJUGATES CONTAINING B7H3-BINDING POLYPEPTIDES

Provided herein are fusion proteins and conjugates containing B7H3-binding polypeptides containing at least one VHH domain that specifically binds B7H3 linked, directly or indirectly, to one or more additional domains or moieties. In some embodiments, the fusion protein or conjugate of the present disclosure is composed of a single polypeptide. In other embodiments, the fusion protein or conjugate of the present disclosure is composed of more than one polypeptide. In some embodiments, the B7H3-binding polypeptide of the present disclosure incorporates at least one VHH domain that specifically binds B7H3. In some aspects, the B7H3-binding polypeptide is multivalent. In some embodiments, the B7H3-binding polypeptides include two or more copies of a VHH domain that specifically binds B7H3, for example, three or more, four or more, five or more, or six or more copies of a VHH domain that specifically binds B7H3. In certain aspects, the B7H3-binding polypeptide is multispecific. For example, in some cases, the one or more additional domain may be one or more additional binding domain that binds to one or more further antigen or protein.

In some embodiments, the B7H3-binding polypeptides of the present disclosure include two or more polypeptide sequences that are operably linked via amino acid linkers. In some embodiments, these linkers are composed predominately of the amino acids Glycine and Serine, denoted as GS-linkers herein. The GS-linkers of the fusion proteins of the present disclosure can be of various lengths, for example, 5, 6, 7, 8, 9, 10, 11, 12, 13, 14, 15, 16, 17, 18, 19, 20 amino acids in length. In some embodiments, the GS-linker comprises an amino acid sequence selected from the group consisting of GGSGGS, i.e., (GGS)₂ (SEQ ID NO:191); GGSGGSGGS, i.e., (GGS)₃ (SEQ ID NO:192); GGSGGSGGSGGS, i.e., (GGS)₄ (SEQ ID NO:193); and GGSGGSGGSGGSGGS, i.e., (GGS)₅ (SEQ ID NO:194). In some embodiments, the linker is a flexible linker comprising Glycine residues, such as, by way of non-limiting example, GG, GGG, GGGG (SEQ ID NO:195), GGGGG (SEQ ID NO:196), and GGGGGG (SEQ ID NO: 197). In some embodiments, the linker is (GGGGS)n, wherein n is 1 to 5 (SEQ ID NO:313); (GGGGGS)n, wherein n is 1 to 4 (SEQ ID NO:314); GGGGS (SEQ ID NO:315); GGGGGS (SEQ ID NO:316); GGGGGSGGGGGSGGGGGS (SEQ ID NO:317); GGGGSGGGGSGGGGS (SEQ ID NO:318); or GGSGGGGSGGGGSGGGGS (SEQ ID NO:319). In some embodiments, the B7H3-binding polypeptide includes a combination of a GS-linker and a Glycine linker

### A. Fc Fusions

Provided herein is a B7H3-binding polypeptide that is a fusion protein containing at least one VHH domain that binds B7H3 provided herein and an Fc domain. In some embodiments, a B7H3-binding polypeptide provided herein comprises one, two, three, or four VHH domains that bind B7H3 and an Fc domain.

In some embodiments, incorporation of an immunoglobulin Fc region into the fusion protein can, in some aspects, be composed of two polypeptides that together form a dimer. In some embodiments, an Fc domain mediates dimerization of the B7H3-binding polypeptide at physiological conditions, such as when expressed from a cell, such that a dimer is formed that doubles the number of B7H3 binding sites. For example, a B7H3-binding polypeptide comprising three VHH domains that bind B7H3 and an Fc region is trivalent as a monomer, but the Fc region may mediate dimerization, such that the B7H3-binding polypeptide exists as a hexavalent dimer under such conditions. In some embodiments, a B7H3 VHH domain is fused to an IgG Fc region and in these embodiments, the fusion protein is bivalent having two B7H3 VHH domains per molecule. In some embodiments, two B7H3 binding domains (2×) are fused to an IgG Fc region and in these embodiments, the fusion protein is tetravalent having four B7H3 VHH domains per molecule. In some embodiments, three B7H3 VHH domain (3×) are fused to an IgG Fc region and in these embodiments, the fusion protein is hexavalent having six B7H3 VHH domains per molecule.

In some embodiments, the multivalent B7H3-binding polypeptide is bivalent. In some embodiments, the bivalent B7H3-binding polypeptide of the disclosure includes two copies of a B7H3-binding polypetide having the following structure: (B7H3 VHH)-Linker-Fc. In some embodiments, the multivalent B7H3-binding polypeptide is tetravalent. In some embodiments, the tetravalent B7H3-binding polypeptide of the disclosure includes two copies of a B7H3-polypeptide having the following structure: (B7H3 VHH)-Linker-(B7H3 VHH)-Linker-Fc. In some embodiments, the multivalent B7H3-binding polypeptide is hexavalent. In some embodiments, the hexavalent B7H3-binding polypeptide of the disclosure includes two copies of a B7H3-binding polypeptide having the following structure: (B7H3 VHH)-Linker-(B7H3 VHH)-Linker-(B7H3 VHH)-Linker-Fc.

In some cases, the CH3 domain of the Fc region can be used as homodimerization domain, such that the resulting fusion protein is formed from two identical polypeptides. In other cases, the CH3 dimer interface region of the Fc region can be mutated so as to enable heterodimerization. For example, a heterodimerization domain can be incorporated into the fusion protein such that the construct is an asymmetric fusion protein.

In any of the provided embodiments, a B7H3 VHH domain can be any as described above. In come embodiments, the B7H3 VHH domain is a humanized VHH domain that binds B7H3.

In various embodiments, an Fc domain included in a B7H3-bindng polypeptide is a human Fc domain, or is derived from a human Fc domain. In some embodiments, the fusion protein contains an immunoglobulin Fc region. In some embodiments, the immunoglobulin Fc region is an IgG isotype selected from the group consisting of IgG1 isotype, IgG2 isotype, IgG3 isotype, and IgG4 subclass.

In some embodiments, the immunoglobulin Fc region or immunologically active fragment thereof is an IgG isotype. For example, the immunoglobulin Fc region of the fusion protein is of human IgG1 isotype, having an amino acid sequence:

(SEQ ID NO: 198)
PAPELLGGPS VFLFPPKPKD TLMISRTPEV
TCVVVDVSHE DPEVKFNWYV DGVEVHNAKT
KPREEQYNST YRVVSVLTVL HQDWLNGKEY
KCKVSNKALP APIEKTISKA KGQPREPQVY
TLPPSRDELT KNQVSLTCLV KGFYPSDIAV
EWESNGQPEN NYKTTPPVLD SDGSFFLYSK
LTVDKSRWQQ GNVFSCSVMH EALHNHYTQK
SLSLSPGK

In some embodiments, the immunoglobulin Fc region or immunologically active fragment thereof comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 198.

In some embodiments where the fusion protein of the disclosure includes an Fc polypeptide, the Fc polypeptide is mutated or modified. In some cases, the mutations include one or more amino acid substitutions to reduce an effector function of the Fc polypeptide. Various examples of mutations to Fc polypeptides to alter, such as reduce, effector function are known, including any as described below. In some embodiments, reference to amino acid substitutions in an Fc region is by EU numbering by Kabat (also called Kabat numbering) unless described with reference to a specific SEQ ID NO. EU numbering is known and is according to the most recently updated IMGT Scientific Chart (IMGT®, the international ImMunoGeneTics information system®, http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

In some embodiments, an Fc region that exhibits reduced effector functions may be a desirable candidate for applications in which B7H3 or CD3 binding is desired yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the multispecific polypeptide constructs and/or cleaved components thereof lack FcγR binding (hence likely lacking ADCC activity), but retains FcRn binding ability. The primary cells for mediating ADCC, NK cells, express FcγRIII only, whereas monocytes express FcγRI, FcγRII and FcγRIII. Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the multispecific polypeptide construct or cleaved components thereof is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

In some embodiments, the human IgG Fc region is modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1):1-11.

Examples of mutations that enhance ADCC include modification at Ser239 and Ile332, for example Ser239Asp and Ile332Glu (S239D, 1332E). Examples of mutations that enhance CDC include modifications at Lys326 and Glu333. In some embodiments, the Fc region is modified at one or both of these positions, for example Lys326Ala and/or Glu333Ala (K326A and E333A) using the Kabat numbering system.

In some embodiments, the Fc region of the fusion protein is altered at one or more of the following positions to reduce Fc receptor binding: Leu 234 (L234), Leu235 (L235), Asp265 (D265), Asp270 (D270), Ser298 (S298), Asn297 (N297), Asn325 (N325), Ala327 (A327) or Pro329 (P329). For example, Leu 234Ala (L234A), Leu235Ala (L235A), Leu235Glu (L235E), Asp265Asn (D265N), Asp265Ala (D265A), Asp270Asn (D270N), Ser298Asn (S298N), Asn297Ala (N297A), Pro329Ala (P329A) or Pro239Gly (P329G), Asn325Glu (N325E) orAla327Ser (A327S). In preferred embodiments, modifications within the Fc region reduce binding to Fc-receptor-gamma receptors while have minimal impact on binding to the neonatal Fc receptor (FcRn).

In some embodiments, the human IgG1 Fc region is modified at amino acid Asn297 (Kabat Numbering) to prevent glycosylation of the fusion protein, e.g., Asn297Ala (N297A) or Asn297Asp (N297D). In some embodiments, the Fc region of the fusion protein is modified at amino acid Leu235 (Kabat Numbering) to alter Fc receptor interactions, e.g., Leu235Glu (L235E) or Leu235Ala (L235A). In some embodiments, the Fc region of the fusion protein is modified at amino acid Leu234 (Kabat Numbering) to alter Fc receptor interactions, e.g., Leu234Ala (L234A). In some embodiments, the Fc region of the fusion protein is modified at amino acid Leu234 (Kabat Numbering) to alter Fc receptor interactions, e.g., Leu235Glu (L235E). In some embodiments, the Fc region of the fusion protein is altered at both amino acids 234 and 235, e.g., Leu234Ala and Leu235Ala (L234A/L235A) or Leu234Val and Leu235Ala (L234V/L235A). In some embodiments, the Fc region of the fusion protein is altered at amino acids at 234, 235, and 297, e.g., Leu234Ala, Leu235Ala, Asn297Ala (L234A/L235A/N297A). In some embodiments, the Fc region of the fusion protein is altered at amino acids at 234, 235, and 329, e.g., Leu234Ala, Leu235Ala, Pro239Ala (L234A/L235A/P329A). In some embodiments, the Fc region of the fusion protein is modified at amino acid Asp265 (Kabat Numbering) to alter Fc receptor interactions, e.g Asp265Ala (D265A). In some embodiments, the Fc region of the fusion protein is modified at amino acid Pro329 (Kabat Numbering) to alter Fc receptor interactions, e.g Pro329Ala (P329A) or Pro329Gly (P329G). In some embodiments, the Fc region of the fusion protein is altered at both amino acids 265 and 329, e.g., Asp265Ala and Pro329Ala (D265A/P329A) or Asp265Ala and Pro329Gly (D265A/P329G). In some embodiments, the Fc region of the fusion protein is altered at amino acids at 234, 235, and 265, e.g., Leu234Ala, Leu235Ala, Asp265Ala (L234A/L235A/D265A). In some embodiments, the Fc region of the fusion protein is altered at amino acids at 234, 235, and 329, e.g., Leu234Ala, Leu235Ala, Pro329Gly (L234A/L235A/P329G). In some embodiments, the Fc region of the fusion protein is altered at amino acids at 234, 235, 265 and 329, e.g., Leu234Ala, Leu235Ala, Asp265Ala, Pro329Gly (L234A/L235A/D265A/P329G). In some embodiments, the Fc region of the fusion protein is altered at Gly235 to reduce Fc receptor binding. For example, wherein Gly235 is deleted from the fusion protein. In some embodiments, the human IgG1 Fc region is modified at amino acid Gly236 to enhance the interaction with CD32A, e.g., Gly236Ala (G236A). In some embodiments, the human IgG1 Fc region lacks Lys447 (EU index of Kabat et al 1991 Sequences of Proteins of Immunological Interest).

In some embodiments, the Fc region of the fusion protein is lacking an amino acid at one or more of the following positions to reduce Fc receptor binding: Glu233 (E233), Leu234 (L234), or Leu235 (L235). For example, an Fc region included in a B7H3-bindng polypeptide is derived from a human Fc domain, and comprises a three amino acid deletion in the lower hinge corresponding to IgG1 E233, L234, and L235. In some aspects, such Fc polypeptides do not engage FcγRs and thus are referred to as “effector silent” or “effector null.” For example, Fc deletion of these three amino acids reduces the complement protein C1q binding. In some embodiments, a polypeptide with an Fc region with Fc deletion of these three amino acids retains binding to FcRn and therefore has extended half-life and transcytosis associated with FcRn mediated recycling. Such a modified Fc region is referred to as “Fc xELL” or “Fc deletion” and has the following amino acid sequence:

(SEQ ID NO: 199)
PAPGGPSVFL FPPKPKDTLM ISRTPEVTCV
VVDVSHEDPE VKFNWYVDGV EVHNAKTKPR
EEQYNSTYRV VSVLTVLHQD WLNGKEYKCK
VSNKALPAPI EKTISKAKGQ PREPQVYTLP
PSRDELTKNQ VSLTCLVKGF YPSDIAVEWE
SNGQPENNYK TTPPVLDSDG SFFLYSKLTV
DKSRWQQGNV FSCSVMHEAL HNHYTQKSLS
LSPGK

In some embodiments, the immunoglobulin Fc region or immunologically active fragment thereof comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 199.

In some embodiments, the human IgG Fc region is modified to enhance FcRn binding. Examples of Fc mutations that enhance binding to FcRn are Met252Tyr, Ser254Thr, Thr256Glu (M252Y, S254T, T256E, respectively) (Kabat numbering, Dall'Acqua et al 2006, J. Biol Chem Vol. 281(33) 23514-23524), Met428Leu and Asn434Ser (M428L, N434S) (Zalevsky et al 2010 Nature Biotech, Vol. 28(2) 157-159), or Met252Ile, Thr256Asp, Met428Leu (M252I, T256D, M428L, respectively), (EU index of Kabat et al 1991 Sequences of Proteins of Immunological Interest).

In some embodiments, the Fc domain included in a B7H3-bindng polypeptide is derived from a human Fc domain and comprises mutations M252Y and M428V, herein referred to as “Fc-YV”. In some embodiments, the mutated or modified Fc polypeptide includes the following mutations: M252Y and M428L using the Kabat numbering system. In some embodiments, such mutations enhance binding to FcRn at the acidic pH of the endosome (near 6.5), while losing detectable binding at neutral pH (about 7.2), allowing for enhanced FcRn mediated recycling and extended half-life.

In some embodiments, the Fc domain included in a B7H3-binding polypeptide is derived from a human Fc domain and comprises mutations to induce heterodimerization. In some embodiments, such mutations include those referred to as “knob” and “hole” mutations. For example, having an amino acid modification within the CH3 domain at Thr366, which when replaced with a more bulky amino acid, e.g., Try (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). In some embodiments, the “knob” Fc domain comprises the mutation T366W. In some embodiments, the “hole” Fc domain comprises mutations T366S, L368A, and Y407V. Heterodimerization via CH3 modifications can be further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15). In some embodiments, Fc domains used for heterodimerization comprise additional mutations, such as the mutation S354C on a first member of a heterodimeric Fc pair that forms an asymmetric disulfide with a corresponding mutation Y349C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at H435. In various embodiments, the hole Fc domain comprises the modification H435R or H435K (referred to as “hole-R” in some instances when the modification is H435R), while the knob Fc domain does not. In some instances, the hole-R mutation improves purification of the heterodimer over homodimeric hole Fc domains that may be present.

In some embodiments, the human IgG Fc region is modified to prevent dimerization. In these embodiments, the fusion proteins of the present disclosure are monomeric. For example modification at residue Thr366 to a charged residue, e.g. Thr366Lys, Thr366Arg, Thr366Asp, or Thr366Glu (T366K, T366R, T366D, or T366E, respectively), prevents CH3-CH3 dimerization.

In some embodiments, the immunoglobulin Fc region or immunologically active fragment of the fusion protein is of human IgG2 isotype, having an amino acid sequence:

(SEQ ID NO: 200)
PAPPVAGPSV FLFPPKPKDT LMISRTPEVT
CVVVDVSHED PEVQFNWYVD GVEVHNAKTK
PREEQFNSTF RVVSVLTVVH QDWLNGKEYK
CKVSNKGLPA PIEKTISKTK GQPREPQVYT
LPPSREEMTK NQVSLTCLVK GFYPSDISVE
WESNGQPENN YKTTPPMLDS DGSFFLYSKL
TVDKSRWQQG NVFSCSVMHE ALHNHYTQKS
LSLSPGK

In some embodiments, the fusion or immunologically active fragment thereof comprises a human IgG2 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 200.

In some embodiments, the human IgG2 Fc region is modified at amino acid Asn297 (e.g. to prevent to glycosylation of the antibody, e.g., Asn297Ala (N297A) or Asn297Asp (N297D). In some embodiments, the human IgG2 Fc region is lacks Lys447 (EU index of Kabat et al 1991 Sequences of Proteins of Immunological Interest).

In some embodiments, the immunoglobulin Fc region or immunologically active fragment of the fusion protein is of human IgG3 isotype, having an amino acid sequence:

(SEQ ID NO: 201)
PAPELLGGPS VFLFPPKPKD TLMISRTPEV
TCVVVDVSHE DPEVQFKWYV DGVEVHNAKT
KPREEQYNST FRVVSVLTVL HQDWLNGKEY
KCKVSNKALP APIEKTISKT KGQPREPQVY
TLPPSREEMT KNQVSLTCLV KGFYPSDIAV
EWESSGQPEN NYNTTPPMLD SDGSFFLYSK
LTVDKSRWQQ GNIFSCSVMH EALHNRFTQK
SLSLSPGK

In some embodiments, the antibody or immunologically active fragment thereof comprises a human IgG3 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 201.

In some embodiments, the human IgG3 Fc region is modified at amino acid Asn297 (Kabat Numbering) to prevent to glycosylation of the antibody, e.g., Asn297Ala (N297A) or Asn297Asp (N297D). In some embodiments, the human IgG3 Fc region is modified at amino acid 435 to extend the half-life, e.g., Arg435His (R435H). In some embodiments, the human IgG3 Fc region is lacks Lys447 (EU index of Kabat et al 1991 Sequences of Proteins of Immunological Interest).

In some embodiments, the immunoglobulin Fc region or immunologically active fragment of the fusion protein is of human IgG4 isotype, having an amino acid sequence:

(SEQ ID NO: 202)
PAPEFLGGPS VFLFPPKPKD TLMISRTPEV
TCVVVDVSQE DPEVQFNWYV DGVEVHNAKT
KPREEQFNST YRVVSVLTVL HQDWLNGKEY
KCKVSNKGLP SSIEKTISKA KGQPREPQVY
TLPPSQEEMT KNQVSLTCLV KGFYPSDIAV
EWESNGQPEN NYKTTPPVLD SDGSFFLYSR
LTVDKSRWQE GNVFSCSVMH EALHNHYTQK
SLSLSLGK

In some embodiments, the antibody or immunologically active fragment thereof comprises a human IgG4 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 202.

In some embodiments, the immunoglobulin Fc region or immunologically active fragment of the fusion protein is of human IgG4 isotype, having an amino acid sequence:

(SEQ ID NO: 203)
PAPELLGGPS VFLFPPKPKD TLMISRTPEV
TCVVVDVSQE DPEVQFNWYV DGVEVHNAKT
KPREEQFNST YRVVSVLTVL HQDWLNGKEY
KCKVSNKGLP SSIEKTISKA KGQPREPQVY
TLPPSQEEMT KNQVSLTCLV KGFYPSDIAV
EWESNGQPEN NYKTTPPVLD SDGSFFLYSR
LTVDKSRWQE GNVFSCSVMH EALHNHYTQK
SLSLSLGK

In some embodiments, the antibody or immunologically active fragment thereof comprises a human IgG4 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 203.

In some embodiments, the human IgG4 Fc region is modified at amino acid 235 to alter Fc receptor interactions, e.g., Leu235Glu (L235E). In some embodiments, the human IgG4 Fc region is modified at amino acid Asn297 (Kabat Numbering) to prevent to glycosylation of the antibody, e.g., Asn297Ala (N297A) or Asn297Asp (N297D). In some embodiments, the human IgG4 Fc region is lacks Lys447 (EU index of Kabat et al 1991 Sequences of Proteins of Immunological Interest).

In some embodiments, the fusion protein contains a polypeptide derived from an immunoglobulin hinge region. The hinge region can be selected from any of the human IgG subclasses. For example, the fusion protein may contain a modified IgG1 hinge having the sequence of EPKSSDKTHTCPPC (SEQ ID NO: 204), where in the Cys220 that forms a disulfide with the C-terminal cysteine of the light chain is mutated to serine, e.g., Cys220Ser (C220S). In other embodiments, the fusion protein contains a truncated hinge having a sequence DKTHTCPPC (SEQ ID NO: 205).

In some embodiments, the fusion protein has a modified hinge from IgG4, which is modified to prevent or reduce strand exchange, e.g., Ser228Pro (S228P), having the sequence ESKYGPPCPPC (SEQ ID NO: 206). In some embodiments, the fusion protein contains linker polypeptides. In other embodiments, the fusion protein contains linker and hinge polypeptides.

In some embodiments, the Fc region lacks or has reduced Fucose attached to the N-linked glycan-chain at N297. There are numerous ways to prevent fucosylation, including but not limited to production in a FUT8 deficient cell line; addition inhibitors to the mammalian cell culture media, for example Castanospermine; and metabolic engineering of the production cell line.

In some embodiments, the Fc region is engineered to eliminate recognition by pre-existing antibodies found in humans. In some embodiments, VHH-containing polypeptides of the present disclosure are modified by mutation of position Leu11, for example Leu11Glu (L11E) or Leu11Lys (L11K). In other embodiments, single domain antibodies of the present disclosure are modified by changes in carboxy-terminal region, for example the terminal sequence has the sequence GQGTLVTVKPGG (SEQ ID NO: 207) or GQGTLVTVEPGG (SEQ ID NO: 208) or modification thereof. In some embodiments, single domain antibodies of the present disclosure are modified by changes in carboxy-terminal region, for example the terminal sequence has the sequence “GG” or modification thereof. In some embodiments, the VHH-containing polypeptides of the present disclosure are modified by mutation of position 11 and by changes in carboxy-terminal region.

In some embodiments, the one or more polypeptides of the fusion proteins of the present disclosure are operably linked via amino acid linkers. In some embodiments, these linkers are composed predominately of the amino acids Glycine and Serine, denoted as GS-linkers herein. The GS-linkers of the fusion proteins of the present disclosure can be of various lengths, for example, 5, 6, 7, 8, 9, 10, 11, 12, 13, 14, 15, 16, 17, 18, 19, 20 amino acids in length.

In some embodiments, the GS-linker comprises an amino acid sequence selected from the group consisting of GGSGGS, i.e., (GGS)₂ (SEQ ID NO: 191); GGSGGSGGS, i.e., (GGS)₃ (SEQ ID NO: 192); GGSGGSGGSGGS, i.e., (GGS)₄ (SEQ ID NO: 193); and GGSGGSGGSGGSGGS, i.e., (GGS)₅ (SEQ ID NO: 194). In some embodiments, the linker is a flexible linker comprising Glycine residues, such as, by way of non-limiting example, GG, GGG, GGGG (SEQ ID NO: 195), GGGGG (SEQ ID NO: 196), and GGGGGG (SEQ ID NO: 197). In some embodiments, the fusion proteins can include a combination of a GS-linker and a Glycine linker

### B. Conjugates

Provided herein are conjugates containing at least one VHH domain that specifically binds B7H3 provided herein and one or more further moiety. The further moiety can be a therapeutic agent, such as a cytotoxic agent, or can be a detection agent. In some embodiments, the moiety can be a targeting moiety, a small molecule drug (non-polypeptide drug of less than 500 Daltons molar mass), a toxin, a cytostatic agent, a cytotoxic agent, an immunosuppressive agent, a radioactive agent suitable for diagnostic purposes, a radioactive metal ion for therapeutic purposes, a prodrug-activating enzyme, an agent that increases biological half-life, or a diagnostic or detectable agent.

In some embodiments, the conjugate is an antibody drug conjugate (ADC, also called immunoconjugates) containing one or more B7H3 VHH domain provided herein conjugated to a therapeutic agent, which is either cytotoxic, cytostatic or otherwise provides some therapeutic benefit. In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In some embodiments, provided antibody drug conjugates of the present disclosure allow targeted-delivery of the drug moiety to tumors. In some cases, this can result in targeted killing of the tumor cell.

In some embodiments, there is provided a B7H3-binding conjugate comprising at least one B7H3 VHH domain provided herein conjugated with a therapeutic agent. In some embodiments, the therapeutic agent includes, for example, daunomycin, doxorubicin, methotrexate, and vindesine (Rowland et al., Cancer Immunol. Immunother. 21:183-187, 1986). In some embodiments, the therapeutic agent has an intracellular activity. In some embodiments, the B7H3-binding conjugate is internalized and the therapeutic agent is a cytotoxin that blocks the protein synthesis of the cell, therein leading to cell death. In some embodiments, the therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where the therapeutic agent is a cytotoxin comprising a polypeptide having a ribosome-inactivating activity, the B7H3-binding conjugate must be internalized upon binding to the target cell in order for the protein to be cytotoxic to the cells.

In some embodiments, there is provided a B7H3-binding conjugate comprising at least one B7H3 VHH domain provided herein conjugated with a toxin. In some embodiments, the toxin includes, for example, bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition.

In some embodiments, there is provided a B7H3-binding conjugate comprising at least one B7H3 VHH domain provided herein conjugated with a label, which can generate a detectable signal, indirectly or directly. These IgSF conjugates can be used for research or diagnostic applications, such as for the in vivo detection of cancer. The label is preferably capable of producing, either directly or indirectly, a detectable signal. For example, the label may be radio-opaque or a radioisotope, such as 3H, 14C, 32P, 35S, 123I, 125I, 131I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example 99Tc or 123I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

The B7H3-binding conjugates may be prepared using any methods known in the art. See, e.g., WO 2009/067800, WO 2011/133886, and U.S. Patent Application Publication No. 2014322129, incorporated by reference herein in their entirety.

In some embodiments, the attachment can be covalent or non-covalent, e.g., via a biotin-streptavidin non-covalent interaction. In some embodiments, 1, 2, 3, 4, 5 or more moieties, which can be the same or different, are conjugated, linked or fused to a B7H3 VHH domain to form a B7H3-binding conjugate. In some embodiments, such moieties can be attached to the VHH domain using various molecular biological or chemical conjugation and linkage methods known in the art and described below. In some embodiments, linkers such as peptide linkers, cleavable linkers, non-cleavable linkers or linkers that aid in the conjugation reaction, can be used to link or conjugate the effector moieties to the variant polypeptide or immunomodulatory protein.

In some embodiments, a B7H3 VHH domain is conjugated to one or more moieties, e.g. about 1 to about 20 drug moieties per VHH, through a linker (L). In some embodiments, the B7H3-binding conjugate comprises the following components: (VHH domain), (L)_{q} and (moiety)ₘ, wherein the VHH domain is any of the described VHH domains capable of specifically binding B7H3 as described; L is a linker for linking the protein or polypeptide to the moiety; m is at least 1; q is 0 or more; and the resulting B7H3-binding conjugate binds to B7H3. In particular embodiments, m is 1 to 4 and q is 0 to 8.

The linker may be composed of one or more linker components. For covalent attachment of the antibody and the drug moiety the linker typically has two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p 234-242).

Exemplary linker components include 6-maleimidocaproyl (“MC”), maleimidopropanoyl (“MP”), valine-citrulline (“val-cit”), a alanine-phenylalanine (“ala-phe”), p-aminobenzyloxycarbonyl (“PAB”), N-Succinimidyl 4-(2-pyridylthio)pentanoate (“SPP”), N-Succinimidyl 4-(N-maleimidomethyl)cyclohexane-I carboxylate (“SMCC”), and N-Succinimidyl (4-iodo-acetyl)aminobenzoate (“SIAB”).

In some embodiments, the linker may comprise amino acid residues. Exemplary amino acid linker components include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline Amino acid linker components can be designed and optimized in their selectivity for enzymatic cleavage by a particular enzymes, for example, a tumor-associated protease, cathepsin B, C and D, at a plasmin protease.

Conjugates of a VHH domain and cytotoxic agent can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl substrate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

The antibody drug conjugate can be prepared by a variety of methods, such as organic chemistry reactions, conditions, and reagents known to those skilled in the art. In one embodiments, methods include: (1) reaction of a nucleophilic group of a VHH domain with a bivalent linker reagent, to form VHH-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of a VHH domain.

Nucleophilic groups on antibodies, including VHH domains, include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues).

Conjugates, such as antibody drug conjugates, may also be produced by modification of an antibody, such as a VHH domain, to introduce electrophilic moieties, which can react with nucleophilic substituents on the linker reagent or drug. The sugars of glycosylated antibodies may be oxidized, e.g., with periodate oxidizing reagents, to form aldehyde or ketone groups which may lead with the amine group of linker reagents or drug moieties. The resulting imine Schiff base groups may form a stable linkage, or may be reduced, e.g., by borohydride reagents to form stable amine linkages In one embodiment, reaction of the carbohydrate portion of a glycosylated antibody with either glactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid. Such aldehyde can be reacted with a drug moiety or linker nucleophile.

Likewise, nucleophilic groups on a drug moiety include, but are not limited to amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBi esters, haloformates, and acid halides; (ii) alkyl and benzyl halides such as haloacetamides; (iii) aldehydes, ketones, carboxyl, and maleimide groups.

Alternatively, a fusion protein containing a VHH domain and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

### C. Multispecific Formats

Provided herein are B7H3-binding polypeptides that are multispecific containing at least one VHH domain that binds B7H3 and one or more additional binding domains. Typically, the one or more additional domains bind to a second antigen or protein other than B7H3. In some embodiments, the one or more additional domain is an antibody or antigen-binding fragment specific for the second antigen or protein. In some embodiments, the additional domain is a VHH domain.

In some embodiments, a multispecific B7H3-binding polypeptide comprises at least one VHH domain that binds B7H3 and at least one additional binding domain that binds a second antigen or protein. In some embodiments, this second antigen is a tumor associated antigen (TAA) or tumor microenvironment associated antigen (TMEAA). In some embodiments, this second antigen is an immunomodulatory antigen, wherein said antigen is involved with enhancing or dampening a signaling pathway in an immune cell.

In some cases, a multispecific B7H3-binding polypeptide can further contain an Fc domain, such as any described above. In some embodiments, a multispecific B7H3-binding polypeptide provided herein at least one VHH domains that bind B7H3, at least one additional binding domain that binds a second antigen or protein, and an Fc domain. In some embodiments, an Fc domain mediates dimerization of the multispecific B7H3-binding polypeptide at physiological conditions such that a dimer is formed that doubles the number of binding sites for B7H3 and for the additional antigen or protein.

Non-limiting exemplary multispecific B7H3-binding polypeptides are described below.

### 1. Bispecific T Cells Engager

In some embodiments, the B7H3-binding polypeptide is a bispecific construct that is or comprises at least one B7H3 VHH domain provided herein and at least one additional binding molecule capable of binding to a surface molecule expressed on a T cell. In some embodiments, the surface molecule is an activating component of a T cell, such as a component of the T cell receptor complex. In particular aspects, the surface molecule is an activating T cell antigen that is expressed on a T cell and is capable of inducing T cell activation upon interaction with an antigen binding molecule. For example, in some aspects, interaction of an antigen binding molecule with an activating T cell antigen may induce T cell activation by triggering the signaling cascade of the T cell receptor complex. Suitable assays to measure T cell activation are known, and include any assay to measure or assess proliferation, differentiation, cytokine secretion, cytotoxic activity and/or expression of one or more activation marker. In some embodiments, the simultaneous or near simultaneous binding of such a B7H3-binding polypeptide to both of its targets, B7H3 expressed on target cell and a T cell molecule expressed on a T cell, e.g. activating T cell antigen, can result in a temporary interaction between the target cell and T cell, thereby resulting in activation, e.g. cytotoxic activity, of the T cell and subsequent lysis of the target cell.

In some embodiments, the T surface molecule, such as activating T cell antigen, is CD3 or is CD2. Specifically, a provided bispecific B7H3-binding polypeptide is capable of specifically binding an activating T cell antigen expressed on a human T cell, such as human CD3 or human CD3. In particular aspects, the additional binding domain that is specific to the activating T cell antigen (e.g. CD3 or CD2) is an antibody or antigen-binding fragment. In some embodiments, a B7H3-binding polypeptide can be a bispecific antibody T cell-engager containing at least one B7H3 VHH domain that specifically binds to B7H3 and an additional binding molecule that is an antibody or antigen-binding fragment specific for an activating component of a T cell (e.g. a T cell surface molecule, e.g. CD3 or CD2).

Among bispecific antibody T cell-engagers are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010). Similar formats of any of the above molecules can be generated using any of the B7H3 VHH domains provided herein.

In some embodiments, the additional binding domain specific to an activating T cell antigen is an antigen-binding fragment selected from a Fab fragment, a F(ab′)₂ fragment, an Fv fragment, a scFv, disulfide stabilized Fv fragment (dsFv), a scAb, a dAb, a single domain heavy chain antibody (VHH), or a single domain light chain antibody. In some embodiments, the additional binding domain is monovalent for binding the activating T cell antigen, such as CD2 or CD3.

In some embodiments, the additional binding domain is capable of binding to CD3 or a CD3 complex. A CD3 complex is a complex of at least five membrane-bound polypeptides in mature T-lymphocytes that are non-covalently associated with one another and with the T-cell receptor. The CD3 complex includes the gamma, delta, epsilon, zeta, and eta chains (also referred to as subunits). In some embodiments, the additional binding molecule is an antibody or antigen-binding fragment capable of specifically binding to CD3 or a CD3 complex, also called a CD3-binding domain. In some embodiments, the CD3-binding domain capable of binding CD3 or a CD3 complex includes one or more copies of an anti-CD3 Fab fragment, an anti-CD3 F(ab′)₂ fragment, an anti-CD3 Fv fragment, an anti-CD3 scFv, an anti-CD3 dsFv, an anti-CD3 scAb, an anti-CD3 dAb, an anti-CD3 single domain heavy chain antibody (VHH), and an anti-CD3 single domain light chain antibody. In some embodiments, the anti-CD3 binding domain is monovalent for binding CD3.

In some cases, the CD3-binding domain recognizes the CD3ε-chain. In some embodiments, the anti-CD3ε binding domain includes one or more copies of an anti-CD3ε Fab fragment, an anti-CD3ε F(ab′)₂ fragment, an anti-CD3ε Fv fragment, an anti-CD3ε scFv, an anti-CD3ε dsFv, an anti-CD3ε scAb, an anti-CD3ε dAb, an anti-CD3ε single domain heavy chain antibody (VHH), and an anti-CD3ε single domain light chain antibody. In some embodiments, the anti-CD3ε binding domain is monovalent for binding CD3ε.

Exemplary monoclonal antibodies against CD3 or a CD3 complex include, but are not limited to, OKT3, SP34, UCHT1 or 64.1, or an antigen-binding fragment thereof (See e.g., June, et al., J. Immunol. 136:3945-3952 (1986); Yang, et al., J. Immunol. 137:1097-1100 (1986); and Hayward, et al., Immunol. 64:87-92 (1988)). In some aspects, clustering of CD3 on T cells, e.g., by immobilized or cell-localized or tethered anti-CD3-antibodies, leads to T cell activation similar to the engagement of the T cell receptor but independent from its clone typical specificity. In one embodiment, the CD3-binding domain monovalently and specifically binds a CD3 antigen, and is derived from OKT3 (ORTHOCLONE-OKT3™ (muromonab-CD3); humanized OKT3 (U.S. Pat. No. 7,635,475 and published international application No. WO2005040220); SP34 (Pessano et ai. The EMBO Journal. 4: 337-344, 1985); humanized variant of SP34 (WO2015001085); Teplizumab™ (MGA031, Eli Lilly); an anti-CD3 binding molecule described in US2011/0275787; UCHT1 (Pollard et al. 1987 J Histochem Cytochem. 35(11):1329-38; WO2000041474); NI0401 (WO2007/033230); visilizumab (U.S. Pat. No. 5,834,597); BC-3 (Anasetti et al., Transplantation 54: 844 (1992); H2C (described in PCT publication no. WO2008/119567); V9 (described in Rodrigues et al., Int J Cancer Suppl 7, 45-50 (1992) and U.S. Pat. No. 6,054,297)). Other anti-CD3 antibodies also can be used in the constructs provided herein, including any described in International published PCT application Nos. WO199404679, WO2008119567, WO2015095392, WO2016204966, WO2019133761; published patent application Nos. US20170369563, US20180194842, US20180355038; U.S. Pat. Nos. 7,728,114, 7,381,803, 7,994,289.

In some embodiments, the CD3-binding domain contains a variable heavy (VH) chain set forth in SEQ ID NO:209 and/or a variable light chain set forth in SEQ ID NO:210, or VH and/or VL sequences having at least 60%, 70%, 80%, 90%, 95%, 97%, 98%, 99% identity with these sequences and specifically binds CD3. In some embodiments, the CD3-binding domain contains a CDRH1, CDRH2 and CDRH3 of the variable heavy (VH) chain set forth in SEQ ID NO:209 and a CDRL1, CDRL2 and CDRL3 variable light chain set forth in SEQ ID NO:210. In some cases, the CD3-binding region comprisies a humanized version of the VH sequence set forth in SEQ ID NO:209 and a humanized version of the VL sequence set forth in SEQ ID NO:210. In some embodiments a CD3-binding region can contain a humanized OKT3 derived VH domain sequence set forth in any one of SEQ ID NOs 211; 212; 213 and/or a VL domain sequence set forth in any one of SEQ ID NOs 214, 215, 216, or VH and/or VL sequences having at least 60%, 70%, 80%, 90%, 95%, 97%, 98%, 99% identity with these sequences and specifically binds CD3. In some embodiments, the CD3-binding domain is a Fab, scFv, Fv or dsFv, in which is contained any combination of the above VH and VL sequence, particularly any combination of a VH sequence set forth in any of SEQ ID NOS: 211, 212 or 213 and a VL sequence set forth in any of SEQ ID NOS: 214, 215, or 216.

In some embodiments, the anti-CD3ε binding domain includes a VH CDR1 sequence that includes at least the amino acid sequence TYAMN (SEQ ID NO: 219); a VH CDR2 sequence that includes at least the amino acid sequence RIRSKYNNYATYYADSVKD (SEQ ID NO: 220); a VH CDR3 sequence that includes at least the amino acid sequence HGNFGNSYVSWFAY (SEQ ID NO: 221), a VL CDR1 sequence that includes at least the amino acid sequence RSSTGAVTTSNYAN (SEQ ID NO: 222); a VL CDR2 sequence that includes at least the amino acid sequence GTNKRAP (SEQ ID NO: 223); and a VL CDR3 sequence that includes at least the amino acid sequence ALWYSNLWV (SEQ ID NO: 224). In some embodiments, the CD3-binding domain is a Fab, scFv, Fv or dsFv, in which is contained a VH CDR1 sequence that includes at least the amino acid sequence TYAMN (SEQ ID NO: 219); a VH CDR2 sequence that includes at least the amino acid sequence RIRSKYNNYATYYADSVKD (SEQ ID NO: 220); a VH CDR3 sequence that includes at least the amino acid sequence HGNFGNSYVSWFAY (SEQ ID NO: 221), a VL CDR1 sequence that includes at least the amino acid sequence RSSTGAVTTSNYAN (SEQ ID NO: 222); a VL CDR2 sequence that includes at least the amino acid sequence GTNKRAP (SEQ ID NO: 223); and a VL CDR3 sequence that includes at least the amino acid sequence ALWYSNLWV (SEQ ID NO: 224).

In some embodiments, the CD3-binding domain contains a variable heavy (VH) chain set forth in SEQ ID NO:217 and/or a variable light chain set forth in SEQ ID NO:218, or VH and/or VL sequences having at least 60%, 70%, 80%, 90%, 95%, 97%, 98%, 99% identity with these sequences and specifically binds to CD3. In some embodiments, the CD3-binding domain contains a CDRH1, CDRH2 and CDRH3 of the variable heavy (VH) chain set forth in SEQ ID NO:217 and a CDRL1, CDRL2 and CDRL3 variable light chain set forth in SEQ ID NO:218. In some embodiments, the CD3-binding domain contains a CDRH1, CDRH2 and CDRH3 set forth in SEQ ID NOs:219, 220 and 221, respectively and a CDRL1, CDRL2 and CDRL3 variable light chain set forth in SEQ ID NO:222, 223 and 224, repectively. In some cases, the CD3-binding region comprises a humanized version of the VH sequence set forth in SEQ ID NO:217 and a humanized version of the VL sequence set forth in SEQ ID NO:218. In some embodiments a CD3-binding region can contain a humanized VH domain sequence set forth in any one of SEQ ID NOs 225-255, 460, 462, or 480 and/or a VL domain sequence set forth in any one of SEQ ID Nos 256-274, 417, 459, or 461, or VH and/or VL sequences having at least 60%, 70%, 80%, 90%, 95%, 97%, 98%, 99% identity with these sequences and specifically binds to CD3. In some embodiments, the anti-CD3ε binding domain thereof includes a variable heavy chain (Hv) comprising the amino acid sequence of SEQ ID NO: 237 and a variable light chain (Lv) comprising the amino acid sequence of SEQ ID NO: 265. In some embodiments, the CD3-binding domain is a Fab, scFv, Fv or dsFv, in which is contained any combination of the above VH and VL sequence, particularly any combination of a VH sequence set forth in any of SEQ ID NOS: 225-255, 460, 462, or 480 and a VL sequence set forth in any of SEQ ID NOS: 256-274, 417, 459, or 461. In some embodiments, the anti-CD3 binding domain is a Fab, scFv, Fv or dsFv, in which is contained a variable heavy chain (Hv) comprising the amino acid sequence of SEQ ID NO: 237 and a variable light chain (Lv) comprising the amino acid sequence of SEQ ID NO: 265.

In some embodiments, the CD3-binding domain contains a variable heavy (VH) chain set forth in any one of SEQ ID NO:537, 538, 541, or 542. In some embodiments, the CD3-binding domain contains a variable light (VL) chain set forth in any one of SEQ ID NO:539, 540, 543, or 544.

The provided bispecific constructs can be formatted in any of a number of formats containing the at least one B7H3 VHH domain and the at least one additional domain specific to an activating T cell antigen, such as a CD3-binding domain.

In one embodiment, the bispecific construct is a bispecific single-domain antibody-linked Fab (S-Fab) containing at least one B7H3 VHH domain as described linked, directly or indirectly to a Fab antigen binding fragment specific to a T cell activating antigen, e.g. CD3, such as an anti-CD3 Fab. The Fab against a T cell activating antigen, e.g. anti-CD3 Fab, can contain any of the VH and VL sequences as described. In some embodiments, the B7H3 VHH domain is linked to the C-terminus of the VH or VL chain of an anti-CD3 Fab. In some embodiments, the S-Fab can be further modified, such as by conjugation with polyethylene glycol (PEG), N-(2-hydroxypropyl) methacrylamide (HPMA) copolymers, proteins (such as albumin), polyglutamic acid or PASylation (Pan et al. (2018) International Jounral of Nanomedicine, 2018: 3189-3201).

In another embodiment, the bispecific construct is a scFv-single domain antibody in which the construct contains at least one B7H3 VHH as described linked, directly or indirectly, to an scFv containing a VH and a VL of an antigen binding domain specific to a T cell activating antigen, e.g. CD3. The scFv against a T cell activating antigen, e.g. anti-CD3 scFv, can contain any of the VH and VL sequences as described. In some embodiments, the VHH domain and the scFv are connected by a linker, such as a peptide linker. In some embodiments, the peptide linker can be a peptide linker as described herein. In some embodiments, the VHH domain and the scFv are each connected, optionally through a hinge region or a linker (e.g. peptide linker), to an Fc region, such as an N-terminus of an Fc region. The Fc region can be any described herein, such as a human Fc region or a variant thereof, e.g. a human IgG1 Fc region or a variant thereof. In particular examples, the Fc region is formed by variant Fc domains, e.g. variant human IgG1 domains, that are mutated or modified to promote heterodimerization in which different polypeptides can be dimerized to yield a heterodimer.

In a further embodiment, the CD3-binding domain is a single domain antibody, such as is a VHH domain that specifically binds to CD3. Single domain antibodies, including VHH domains that bind to CD3 are known, see e.g. published U.S. patent application No. US20160280795. In some embodiments, the CD3-binding domain is an anti-CD3 VHH set forth in SEQ ID NO:275, or a sequence that exhibits at least 60%, 70%, 80%, 90%, 95%, 97%, 98%, 99% identity with SEQ ID NO:275 and specifically binds to CD3. In such aspects, a bispecific construct provided herein can include at least one B7H3 VHH domain and at least one CD3 VHH domain.

For formatting the constructs, in some cases, each VHH domain is connected, optionally through a hinge region or linker (e.g. peptide linker), to an Fc region, such as an N-terminus of an Fc region. The Fc region can be any described herein, such as a human Fc region or a variant thereof, e.g. a human IgG1 Fc region or a variant thereof. In particular examples, the Fc region is formed by variant Fc domains, e.g. variant human IgG1 domains, that are mutated or modified to promote heterodimerization in which different polypeptides can be dimerized to yield a heterodimer.

In the above embodiments, exemplary modifications of an Fc region to promote heterodimerization are known, including any as described below, e.g. Table 3. In some embodiments, one Fc polypeptide of a heterodimeric Fc comprises the sequence of amino acids set forth in any of SEQ ID NOS:293, 297, 305, 307,445, or 451 and the other Fc polypeptide of the heterodimeric Fc contains the sequence of amino acids set forth in any of SEQ ID NOS:294, 298, 301, 303, 309, 311, 446, 449, or 453. In some embodiments, one Fc polypeptide of a heterodimeric Fc comprises the sequence of amino acids set forth in any of SEQ ID NOS: 295, 299, 306, 308, 447, or 452 and the other Fc polypeptide of the heterodimeric Fc comprises the sequence of amino acids set forth in any of SEQ ID NOS: 296, 300, 302, 304, 310, 312, 448, 450, or 454.

### 2. Constrained CD3 Multispecific Construct

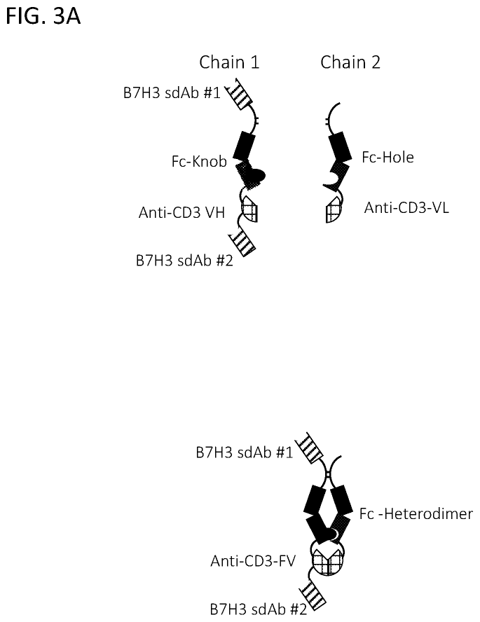

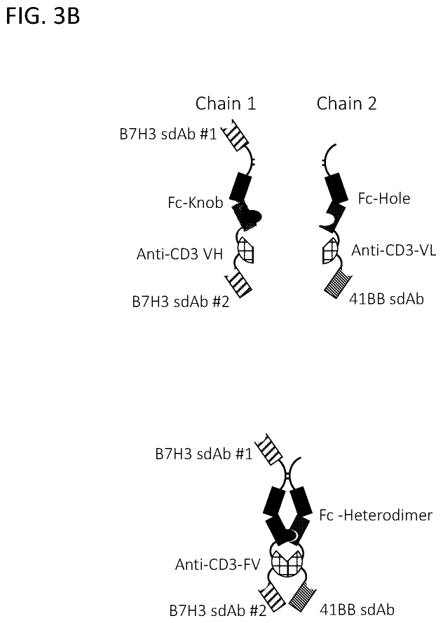

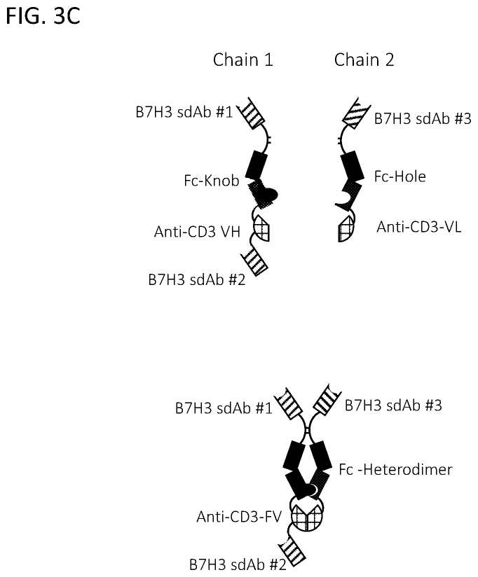

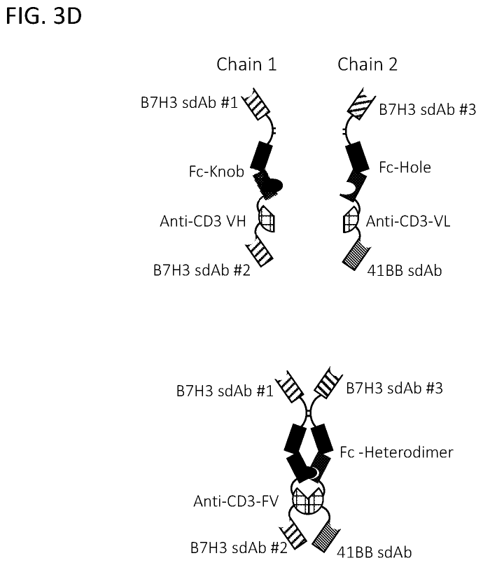

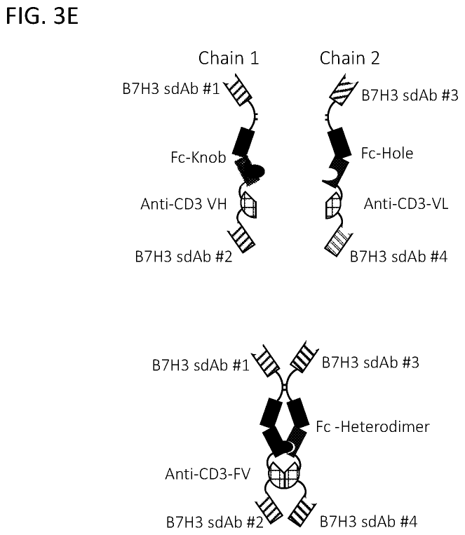

In some embodiments, the B7H3-binding polypeptide is a multispecific polypeptide construct that is a constrained T-cell engaging fusion protein. In particular aspects, the constrained multispecific constructs provided herein bind an activating T cell antigen, such as a CD3, and B7H3. The constrained multispecific polypeptide constructs provided herein include at least a first component that includes an immunoglobulin Fc region, a second component that includes one or more copies of at least a binding domain that binds CD3 (referred to herein as an anti-CD3 binding domain or a CD3 binding domain, which are terms that are used interchangeably herein), and a linker, such as a polypeptide linker, that joins the first component and the second component. In the provided multispecific polypeptide constructs, one or both of the first and second components contain at least one B7H3 VHH domain, which, when engaged upon binding to antigen, render the constrained CD3 binding region substantially able to bind CD3. FIGS. 3A-3E depict exemplary formats of a constrained multispecific construct.

In some embodiments, the constrained multispecific polypeptide constructs provided herein exist in two states in terms of capacity to bind CD3 and subsequently activate T-cells: (1) the “inactive” state occurs when there is no binding of any or all of the antigen binding domain(s) to B7H3, such that the CD3 binding is constrained and T-cell interaction is obviated or reduced, and (2) the “active” state occurs upon antigen binding by any or all of the antigen binding domain(s), such that the CD3 binding region is able to bind CD3 and the T-cell interaction is allowed.

In some embodiments, the Fc region is linked to the CD3 binding domain via a linker or linkers. In some embodiments, the Fc region is linked to the CD3 binding region via a non-cleavable linker or linkers. In some embodiments, the Fc region is linked to the CD3 binding region via a cleavable linker or an otherwise labile linker or linkers. In some embodiments, cleavable linker is a linker that can be specifically cleaved in the presence of a protease. In some aspects, enhanced CD3 binding occurs following cleavage of the cleavable linker. In some such aspects, the “active” state can be further amplified via several mechanisms, including via cleavage of the linker joining the CD3 binding region and the Fc region. In some embodiments, the cleavable linker is a linker that contains a substrate recognition site for a protease. In some embodiments, wherein the Fc region and the CD3 binding region are linked by a cleavable linker, enhanced CD3 binding may occur following cleavage within the linker(s).

Further, in aspects wherein the Fc region and the CD3 binding region are operably linked by a cleavable linker, cleavage of the linker(s) between the Fc region and the CD3 binding region may separate the constrained multispecific polypeptide constructs into a first and second component. Depending on the composition of the constrained multispecific polypeptide construct, the first and second component may have distinct functionalities. In some embodiments, the Fc region is a region that exhibits one or more effector functions, such as ADCC, CDC or ADCP functions. In such examples, the constrained multispecific polypeptide constructs of the disclosure can be used to produce a self-amplifying system. For example, in some aspects, the incorporation of a protease cleavable linker between the Fc and the components of the CD3 binding domain enables for amplification of the T-cell activating capacity by allowing full exposure of the CD3 binding domain. Depending on the specific linker included, the amplification step can be mediated by tumor associated proteases or by granzymes released following antigen dependent-T-cell activation. If a tumor protease cleavable linker is included the amplification is mediated by the tumor or tumor-microenvironment. Whereas, if a granzyme B cleavable linker is included the amplification may be self-mediated by T-cells following antigen-dependent activation. Furthermore, in cases wherein an effector enabled Fc is included in the construct, amplification may be mediated by granzymes released from NK cell that occurs through an ADCC mechanism.

The provided constrained multispecific polypeptide constructs include a configuration in which the first component containing the Fc region is N-terminal to the second component containing the CD3 binding region. In such an embodiment, the first and second components are joined via a linker that is C-terminal to the end of the Fc region. In some embodiments, the at least one B7H3 VHH domain is positioned on the amino-terminal (N-term) region of the multispecific polypeptide construct. In some embodiments, the at least one B7H3 VHH domain is positioned on the carboxy-terminal (C-term) region of the multispecific polypeptide construct. In some embodiments, the constrained multispecific polypeptide construct contains at least two B7H3 VHH domains that are positioned on both the N- and C-terminal regions of the multispecific polypeptide construct.

In some embodiments, the constrained multispecific polypeptide construct is a dimer, in which dimerization is formed by covalent or non-covalent interactions between two polypeptide chains. In some embodiments, the two polypeptide chains are covalently bonded to each other by, for example, interchain disulfide bonds. In some embodiments, the Fc region mediates dimerization via interchain disulfide bonds. In particular embodiments, a constrained multispecific polypeptide construct contains a heterodimeric Fc region in which, in some cases, the polypeptide chains of the multispecific polypeptide construct are different (heterodimer). In particular examples of a heterodimeric multispecific polypeptide construct, the CD3-binding region is a two chain polypeptide containing a VH and a VL chain, such as is an Fv antibody fragment containing the VH and VL. In some embodiments, the Fv antibody fragment includes a disulfide stabilized anti-CD3 binding Fv fragment (dsFv).

In particular embodiments, the Fv is a disulfide stabilized Fv fragment (dsFv) in which the the V_{H}-V_{L} heterodimer is stabilized by an interchain disulfide bond. In some embodiments, the interchain disulfide bond is engineered by mutation of position in framework positions of the VH and/or VL chain. In some embodiments, the VH chain contains the mutation G44C and the VL chain contains the mutation G100C, each by kabat numbering. In some embodiments, the disulfide stabilized anti-CD3 Fv comprises an anti-CD3 VH with the mutation at position 105 to Cys and an anti-CD3 VL with the mutation position 43 to Cys by Kabat numbering.

In some embodiments, a constrained multispecific polypeptide construct is formed from or includes two polypeptides, including a first polypeptide comprising a first Fc polypeptide of a heterodimeric Fc region, a linker (e.g. cleavable or non-cleavable linker), a VH domain of an anti-CD3 antibody or antigen binding fragment (e.g. Fv); and a second polypeptide comprising a second Fc polypeptide of the heterodimeric Fc region, the linker (e.g. the cleavable or non-cleavable linker), a VL domain of the anti-CD3 antibody or antigen binding fragment (e.g. Fv). In some embodiments, the first polypeptide contains one or two VHH domains that bind to B7H3. In some embodiments, the second polypeptide contains one or two VHH domains that bind to B7H3. In some embodiments, a constrained multispecific polypeptide construct contains at least two B7H3 VHH domains. In some cases, at least one B7H3 VHH domain is located N-terminally to the Fc polypeptide and at least one B7H3 VHH domain is located C-terminally to the chain of the CD3-binding region.

In some embodiments, the first polypeptide or second polypeptide or both the first and second polypeptide further include a co-stimulatory receptor binding region (CRBR) that binds a co-stimulatory receptor. In some embodiments, the CRBR of the first and/or second polypeptide can be located N-terminally to the Fc polypeptide and/or C-terminally to the chain of the CD3-binding region.

In some embodiments, a constrained multispecific polypeptide construct contains at least two VHH domains that bind B7H3 and at least one co-stimulatory receptor binding region (CRBR) that binds a co-stimulatory receptor. In some embodiments, a constrained multispecific polypeptide construct contains (1) a first polypeptide comprising in order of N-terminus to C-terminus: a first B7H3 VHH domain, the first Fc polypeptide of a heterodimeric Fc region, a linker (e.g. a cleavable linker), a chain (e.g. VH or VL) of an anti-CD3 antibody or antigen binding fragment (e.g. Fv or dsFv), and a second B7H3 VHH domain; and (2) a second polypeptide comprising in order of N-terminus to C-terminus: the second Fc polypeptide of the heterodimeric Fc region, the same linker (e.g. same cleavable linker), the other chain (other of the VH or VL) of the anti-CD3 antibody or antigen binding fragment, and a co-stimulatory receptor binding region (CRBR) that binds a co-stimulatory receptor.

In some embodiments, the first polypeptide or second polypeptide or both the first and second polypeptide further include an inhibitory receptor binding region (IRBR) that binds an inhibitory receptor. In some embodiments, the IRBR of the first and/or second polypeptide can be located N-terminally to the Fc polypeptide and/or C-terminally to the chain of the CD3-binding region.

In some embodiments, a constrained multispecific polypeptide construct contains at least two VHH domains that bind B7H3 and at least one inhibitory receptor binding region (IRBR) that binds an inhibitory receptor. In some embodiments, a constrained multispecific polypeptide construct contains (1) a first polypeptide comprising in order of N-terminus to C-terminus: a first B7H3 VHH domain, the first Fc polypeptide of a heterodimeric Fc region, a linker (e.g. a cleavable or non-cleavable linker), a chain (e.g. VH or VL) of an anti-CD3 antibody or antigen binding fragment (e.g. Fv or dsFv), and a second B7H3 VHH domain; and (2) a second polypeptide comprising in order of N-terminus to C-terminus: the second Fc polypeptide of the heterodimeric Fc region, the same linker (e.g. same cleavable linker), the other chain (other of the VH or VL) of the anti-CD3 antibody or antigen binding fragment, and an inhibitory receptor binding region (IRBR) that binds an inhibitory receptor.

In some embodiments, at least one of the first polypeptide or second polypeptide further include a co-stimulatory receptor binding region (CRBR) that binds a co-stimulatory receptor and at least one of the first polypeptide or second polypeptide further includes an inhibitory receptor binding region (IRBR) that binds an inhibitory receptor. In some embodiments, the CRBR of the first and/or second polypeptide can be located N-terminally to the Fc polypeptide and/or C-terminally to the chain of the CD3-binding region. In some embodiments, the IRBR of the first and/or second polypeptide can be located N-terminally to the Fc polypeptide and/or C-terminally to the chain of the CD3-binding region.

In some embodiments, a constrained multispecific polypeptide construct contains at least two VHH domains that bind B7H3, a co-stimulatory receptor binding region (CRBR) that binds a co-stimulatory receptor and an inhibitory receptor binding region (IRBR) that binds an inhibitory receptor. In some embodiments, a constrained multispecific polypeptide construct contains (1) a first polypeptide comprising in order of N-terminus to C-terminus: a first B7H3 VHH domain, the first Fc polypeptide of a heterodimeric Fc region, a linker (e.g. a cleavable or non-cleavable linker), a chain (e.g. VH or VL) of an anti-CD3 antibody or antigen binding fragment (e.g. Fv or dsFv), and a second B7H3 VHH domain; and (2) a second polypeptide comprising in order of N-terminus to C-terminus: one of an IRBR or CRBR, the second Fc polypeptide of the heterodimeric Fc region, the same linker (e.g. same cleavable or non-cleavable linker), the other chain (other of the VH or VL) of the anti-CD3 antibody or antigen binding fragment, and the other of the IRBR or CRBR.

Each of the components of the multispecific polypeptide constructs of the disclosure is described in more detail below.

a. B7H3 VHH Antigen Binding Comain

A constrained multispecific polypeptide construct of the disclosure includes at least one B7H3 VHH domain from among any provided herein. In some embodiments, the B7H3 VHH domain comprises the sequence of amino acids set forth in any of SEQ ID NOS:1-114, 466, 467.

A constrained multispecific polypeptide construct of the disclosure includes at least one B7H3 VHH domain from among any provided herein. In some embodiments, the B7H3 VHH domain comprises the sequence of amino acids set forth in any of SEQ ID NOS:1-114, 466, 467, 489, 490, or 492-518.

In particular embodiments, a constrained multispecific polypeptide construct contains at least two B7H3 domain. In some cases, at least one B7H3 VHH domain is positioned amino terminally relative to an Fc polypeptide of the heterodimeric Fc and at least one B7H3 VHH domain is positioned carboxy-terminally relative to VH or VL chain of the CD3 binding region.

In aspects of a constrained multispecific polypeptide construct containing at least two or containing two B7H3 VHH domains, each of the B7H3 VHH domains can bind to the same or an overlapping epitope on B7H3.

In aspects of a constrained multispecific polypeptide construct containing at least two or containing two B7H3 VHH domains, each of the B7H3 VHH domains can bind to a different or a non-overlapping epitope on B7H3.

In some embodiments, the first sdAb VHH domain and second sdAb VHH domain comprises the amino acid sequences set forth in SEQ ID NO: 67 and SEQ ID NO:43. In some embodiments, the first sdAb VHH domain and second sdAb VHH domain comprises the amino acid sequences set forth in SEQ ID NO: 67 and SEQ ID NO:503. In some embodiments, the first sdAb VHH domain and second sdAb VHH domain comprises the amino acid sequences set forth in SEQ ID NO: 67 and SEQ ID NO:67. In some embodiments, the first sdAb VHH domain and second sdAb VHH domain comprises the amino acid sequences set forth in SEQ ID NO: 67 and SEQ ID NO:1. In some embodiments, the first sdAb VHH domain and second sdAb VHH domain comprises the amino acid sequences set forth in SEQ ID NO: 67 and SEQ ID NO:8. In some embodiments, the first sdAb VHH domain and second sdAb VHH domain comprises the amino acid sequences set forth in SEQ ID NO: 467 and SEQ ID NO:466. In some embodiments, the first sdAb VHH domain and second sdAb VHH domain comprises the amino acid sequences set forth in SEQ ID NO: 467 and SEQ ID NO:85.

In some embodiments, the antigen binding domain, such as a B7H3 VHH domain, is linked, directly or indirectly via a linker, to the Fc region and/or to the CD3 binding region. In some embodiments, linkage is via a linker. In some embodiments, the linker is a linking peptide (LP), which can include any flexible or rigid linker as described. In some embodiments, the linker is selected from the group consisting of GGSGGS, i.e., (GGS)₂ (SEQ ID NO: 191); GGSGGSGGS, i.e., (GGS)₃ (SEQ ID NO: 192); GGSGGSGGSGGS, i.e., (GGS)₄ (SEQ ID NO: 193); and GGSGGSGGSGGSGGS, i.e., (GGS)₅ (SEQ ID NO: 194). In some embodiments, the linker is a flexible linker comprising Glycine residues, such as, by way of non-limiting example, GG, GGG, GGGG (SEQ ID NO: 195), GGGGG (SEQ ID NO: 196), and GGGGGG (SEQ ID NO: 197). In some embodiments, the linker includes a combination of a GS-linker and a Glycine linker.

b. Fc Region

A constrained multispecific polypeptide construct includes an immunoglobulin Fc region. Generally, the constrained multispecific polypeptide construct is a dimer formed by polypeptides, each containing an Fc. The Fc polypeptide can be any as set forth above.

In particular embodiments, the Fc region is formed by Fc domains that are mutated or modified to promote heterodimerization in which different polypeptides can be dimerized to yield a heterodimer. Thus, in some embodiments, the dimer is a heterodimer in which two polypeptide chains of the multispecific polypeptide construct are different.

Various methods are known for promoting heterodimerization of complementary Fc polypeptides, see e.g. Ridgway et al, Protein Eng. 9:617-621 (1996); Merchant et al, Nat. Biotechnol. 16(7): 677-81 (1998); Moore et al. (2011) MAbs, 3:546-57; Von Kreudenstein et al. MAbs, (2013) 5:646-54; Gunasekaran et al. (2010) J. Biol. Chem., 285:19637-46; Leaver-Fay et al. (2016) Structure, 24:641-51; Ha et al. (2016) Frontiers in Immunology, 7:1; Davis et al. (2010) Protein Eng Des Sel, 23:195-202; published international PCT Appl. No. WO 1998/050431, WO 2009/089004, WO2011143545 WO 2014/067011, WO 2012/058768, WO2018027025; published U.S. patent Appl. No. US20140363426, US20150307628, US20180016354, US20150239991; and U.S. Pat. Nos. 5,731,168, 7,183,076, 9,701,759, 9,605,084, and 9,650,446. Methods to promote heterodimerization of Fc chains include mutagenesis of the Fc region, such as by including a set of “knob-into-hole” mutations or including mutations to effect electrostatic steering of the Fc to favor attractive interactions among different polypeptide chains. For example, in some embodiments, the Fc polypeptides of a heterodimer includes a mutation to alter charge polarity across the Fc dimer interface such that co-expression of electrostatically matched Fc chains support favorable attractive interactions thereby promoting desired Fc heterodimer formation, whereas unfavorable repulsive charge interactions suppress unwanted Fc homodimer formation (Guneskaran et al. (2010) JBC, 285: 19637-19646). When co-expressed in a cell, association between the chains is possible but the chains do not substantially self-associate due to charge repulsion. Other strategies for generating a heterodimeric Fc include mixing human IgG and IgA CH3 domain segments to create a complementary CH3 heterodimer, which is referred to as a SEED Fc.

Methods and variants for heterodimerization also include those described in published international PCT App. WO2014/145806, including “knobs and holes” mutations (also called “skew” variants), mutations that relate to “electrostatic steering” or “charge pairs,” and pI variants. Heterodimeric variants also include any as described in U.S. published Appl. No. US2012/0149876 or US2018/011883.

In some embodiments, to promote heterodimerization both polypeptides of the Fc heterodimer contain paired or complementary amino acid modifications. Exemplary paired amino acid modification of polypeptides of an Fc fusion are set forth in Table 3.

TABLE 3
Paired amino acids of Heterodimeric Fc

| First Fc polypeptide | Second Fc Polypeptide |
|---|---|
| T366W | T366S/L368W/Y407V |
| T366W/S354C | T366S/L368A/Y407V/Y349C |
| S364H/F405A | Y349T/Y349F |
| T350V/L351Y/F405A/Y407V | T350V/T366L/K392L/T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/399V/F405T/S354C |
| K370E/K409W | E357N/D399V/F405T |

In some embodiments, modifications include introduction of a protuberance (knob) into a first Fc polypeptide and a cavity (hole) into a second Fc polypeptide such that the protuberance is positionable in the cavity to promote complexing of the first and second Fc-containing polypeptides. Amino acids targeted for replacement and/or modification to create protuberances or cavities in a polypeptide are typically interface amino acids that interact or contact with one or more amino acids in the interface of a second polypeptide.

In some embodiments, a first Fc polypeptide that is modified to contain protuberance (hole) amino acids include replacement of a native or original amino acid with an amino acid that has at least one side chain which projects from the interface of the first Fc polypeptide and is therefore positionable in a compensatory cavity (hole) in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. In some embodiments, the replacement residues for the formation of a protuberance are naturally occurring amino acid residues and include, for example, arginine (R), phenylalanine (F), tyrosine (Y), or tryptophan (W). In some examples, the original residue identified for replacement is an amino acid residue that has a small side chain such as, for example, alanine, asparagine, aspartic acid, glycine, serine, threonine, or valine.

In some embodiments, a second Fc polypeptide that is modified to contain a cavity (hole) is one that includes replacement of a native or original amino acid with an amino acid that has at least one side chain that is recessed from the interface of the second polypeptide and thus is able to accommodate a corresponding protuberance from the interface of a first polypeptide. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T) and valine (V). In some examples, the original amino acid identified for replacement is an amino acid that has a large side chain such as, for example, tyrosine, arginine, phenylalanine, or tryptophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9: 617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain.

For example, in some embodiments the heterodimeric Fc includes a polypeptide having an amino acid modification within the CH3 domain at Thr366, which when replaced with a more bulky amino acid, e.g., Try (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Tyr349 to Cys (Y349C) on opposite CH3 domains (Reviewed in Carter, 2001 Journal of Immunological Methods, 248: 7-15).

In particular embodiments, a multispecific polypeptide construct contains a first and second Fc₌able to mediate Fc heterodimerization contains a first Fc polypeptide containing mutations T366W and S354C and a second Fc polypeptide containing mutations T366S, L368A, Y407V and Y349C. In some embodiments, the first Fc polypeptide is selected from an Fc polypeptide comprising the sequence set forth in SEQ ID NO: 445 or 451 and the second Fc polypeptide is selected from an Fc polypeptide comprising the sequence set forth in SEQ ID NO: 446, 449 or 453. In some embodiments, the first Fc polypeptide is or comprises the sequence of amino acids set forth in any of SEQ ID NOS: 293, 297, 305 or 307 and the second Fc polypeptide is or comprises the sequence of amino acids set forth in any of SEQ ID NOS: 294, 298, 301, 303, 309 or 311.

In some embodiments, the Fc polypeptide exhibits features providing Fc-mediated effector functions. In particular examples, the first Fc polypeptide is or comprises the sequence set forth in SEQ ID NOs:445 and a second Fc polypeptide that is or comprises SEQ ID NO: 446 or 449. In some embodiments, the first Fc polypeptide is or comprises the sequence set forth in SEQ ID NO: 293 and the second Fc polypeptide is or comprises the sequence set forth in SEQ ID NO: 294 or 301. In some embodiments, the first Fc polypeptide is or comprises the sequence set forth in SEQ ID NO: 297 and the second Fc polypeptide is or comprises the sequence set forth in SEQ ID NO: 298 or 303. The first and second Fc polypeptide can be formatted on either polypeptide chain of the construct.

In some embodiments, one or both of the first and second Fc polypeptides can further include one or more amino acid mutations to further reduce one or more Fc effector functions, such as reduced Fc receptor binding. Exemplary mutations to reduce Fc effector functions include any as described. In some embodiments, the modification can be a deletion of one or more positions,Glu233 (E233), Leu234 (L234), or Leu235 (L235), such as a deletion of amino acids Glu233 (E233), Leu234 (L234), and Leu235 (L235). In some embodiments, the first Fc polypeptide is selected from an Fc polypeptide comprising the sequence set forth in SEQ ID NO: 447 or 452 and the second Fc polypeptide is selected from an Fc polypeptide comprising the sequence set forth in SEQ ID NO: 448, 450 or 454. In some embodiments, the first Fc polypeptide is or comprises the sequence of amino acids set forth in any of SEQ ID NOS: 295, 299, 306 or 308 and the second Fc polypeptide is or comprises the sequence of amino acids set forth in any of SEQ ID NOS: 296, 300, 302, 304, 310 or 312.

In particular examples, the first Fc polypeptide is or comprises the sequence set forth in SEQ ID NOs:447 and a second Fc polypeptide that is or comprises SEQ ID NO: 448 or 450. In some embodiments, the first Fc polypeptide is or comprises the sequence set forth in SEQ ID NO: 295 and the second Fc polypeptide is or comprises the sequence set forth in SEQ ID NO: 296 or 302. In some embodiments, the first Fc polypeptide is or comprises the sequence set forth in SEQ ID NO: 299 and the second Fc polypeptide is or comprises the sequence set forth in SEQ ID NO: 300 or 304. The first and second Fc polypeptide can be formatted on either polypeptide chain of the construct.

In some embodimetns, the first Fc polypeptide or second Fc polypeptide further includes mutations M252Y and/or M428V. In particular examples, the first Fc polypeptide is or comprises the sequence set forth in SEQ ID NO:451 and the second Fc polypeptide is or comprises the sequence set forth in SEQ ID NO:453. In some embodiments, the first Fc polypeptide is or comprises the sequence set forth in SEQ ID NO:305 and the second Fc polypeptide is or comprises the sequence set forth in SEQ ID NO: 309. In some embodiments, the first Fc polypeptide is or comprises the sequence set forth in SEQ ID NO:307and the second Fc polypeptide is or comprises the sequence set forth in SEQ ID NO: 311. In other examples, the first Fc polypeptide is or comprises the sequence set forth in SEQ ID NO:452 and the second Fc polypeptide is or comprises the sequence set forth in SEQ ID NO:454. In some embodiments, the first Fc polypeptide is or comprises the sequence set forth in SEQ ID NO:306 and the second Fc polypeptide is or comprises the sequence set forth in SEQ ID NO: 310. In some embodiments, the first Fc polypeptide is or comprises the sequence set forth in SEQ ID NO:308 and the second Fc polypeptide is or comprises the sequence set forth in SEQ ID NO: 312. The first and second Fc polypeptide can be formatted on either polypeptide chain of the construct.

Additional examples of variants that can facilitate the promotion of heterodimers are any combination or pair of steric variants (e.g. skew variants) of a first Fc polypeptide and a second Fc polypeptide from among: S364K/E357Q and L368D/K370S; L368D/K370S and S364K; L368E/K370S and S364K; T411T/E360E/Q362E and D401K; L368D/K370S and S364K/E357L, K370S and S364K/E357Q and T366S/L368A/Y407V and T366W or 366S/L368A/Y407V/Y349C and T366W/S354C), where each pair represents mutations in the first Fc polypeptide and second Fc polypeptide. In particular embodiments, a provided construct contains a first and second Fc polypeptide containing the pair of mutations L368D/K370S and S364K and E357Q.

An additional mechanism that can be used in the generation of heterodimers is sometimes referred to as “electrostatic steering” as described in Gunasekaran et al., J. Biol. Chem. 285(25):19637 (2010). This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “steric variants”. In one embodiments, a first Fc polypeptide can contain mutations D221E/P228E/L368E and a second Fc polypeptide can contain mutations D221R/P228R/K409R. In another embodiments, a first Fc polypeptide can contain mutations C220E/P228E/368E and a second Fc polypeptide can contain mutations C220R/E224R/P228R/K409R.

In some embodiments, heterodimerization can be facilitated by pI variants. In some aspects, a pI variant can include those that increase the pI of the protein (basic changes). In other aspects, the pI variant can include those that decrease the pI of the protein (acidic changes). In some cases, all combinations of these variants can be done, including combinations in which one Fc polypeptide may be wild type, or a variant that does not display a significantly different pI from wild-type, and the other Fc polypeptide can be either more basic or more acidic. Alternatively, each Fc polypeptide can be changed, one to more basic and one to more acidic. In some embodiments, at least one Fc polypeptide is a negative pI variant Fc containing mutations Q295E/N384D/Q418E/N421D.

In some embodiments, a combination of steric heterodimerization variants (e.g. knob and hole) and pI or charge pair variants can be used.

In particular embodiments, the provided constructs contains (a) a first Fc polypeptide comprising the skew variants S364K/E357Q; and b) a second Fc polypeptide containing skew variants L368D/K370S and the pI variants N208D/Q295E/N384D/Q418E/N421D. In some embodiments, one or both of the first and second polypeptide can contain further mutations to reduce Fc effector activity, such as the exemplary mutations E233P/L234V/L235A/G236del/S267K. An example of such a first Fc polypeptide and a second Fc polypeptide able to mediate Fc heterodimeriztion comprise the sequences set forth in SEQ ID NOs:457 and 458. The first and second Fc polypeptide can be formatted on either polypeptide chain of the construct.

The resulting constrained multispecific polypeptide constructs can be purified by any suitable method such as, for example, by affinity chromatography over Protein A or Protein G columns Where two nucleic acid molecules encoding different polypeptides are transformed into cells, formation of homo- and heterodimers will occur. Conditions for expression can be adjusted so that heterodimer formation is favored over homodimer formation.

Techniques for recovery of heterodimers from homodimers based on a differential affinity of the heterodimers for an affinity reagent are known. In some aspects, such techniques include designing a heterodimer so that one of the Fc polypeptide chains does not bind to the affinity reagent protein A. In some cases, one of the polypeptide chain can contain one or more amino acid substitution to abrogate or reduce affinity for the protein A reagent in one of the polypeptides of the Fc heterodimer, see e.g. WO2017134440, WO2010151792, Jendeberg et al. (Jendeberg et al., (1997) J. Immunol. Methods, 201(1): 25-34. In some of these embodiments, the Fc region may be modified at the protein-A binding site on one member of the heterodimer so as to prevent protein-A binding and thereby enable more efficient purification of the heterodimeric fusion protein. An exemplary modification within this binding site is Ile253, for example Ile253Arg (I253R). In some embodiments, the modification may be H435R or H435R/Y436F. In some embodiments, an Fc polypeptide of an Fc heterodimer can contain a modification so that it is capable of binding protein A but not protein G (pA+/pG−). Exemplary pA+/pG− amino acid modifications include an Fc containing serine at position 428, serine at position 434 and optionally histidine at position 436, with reference to human IgG1 or comprising these residues at the corresponding positions in human IgG 2, 3, or 4. In some aspects, such amino acid modifications in one IgG Fc polypeptide at positions 428, 434 and optionally 436 reduces or prevents the binding of protein G, enhancing the purification of the protein.

In some embodiments, any of such modifications to confer differential affinity to an affinity reagent can be combined with any one or more other amino acid modifications described above. For example, the I253R modification maybe combined with either the T366S/L368A/Y407V modifications or with the T366W modifications. The T366S/L368A/Y407V modified Fc is capable of forming homodimers as there is no steric occlusion of the dimerization interface as there is in the case of the T336W modified Fc. Therefore, in some embodiments, the I253R modification is combined with the T366S/L368A/Y407V modified Fc to disallow purification any homodimeric Fc that may have formed. Similar modifications can be employed by combining T366S/L368A/Y407V and H453R.

In some embodiments, the Fc regions of the heterodimeric molecule additionally can contain one or more other Fc mutation, such as any described above. In some embodiments, the heterodimer molecule contains an Fc region with a mutation that reduces effector function. In some embodiments, the Fc region is altered to provide reduced Fc-mediated effector functions, such as via reduced Fc receptor binding, e.g. binding to FcγR binding but generally not FcRn binding.

In some embodiments, the Fc region is mutated in one or more of the following positions to reduce Fc receptor binding: Glu233 (E233), Leu234 (L234), or Leu235 (L235). The one or more mutations can include E233P, L234V and/or L235A.

In particular embodiments, the mutations of the Fc region to reduce Fc effector function, e.g. via reducing Fc receptor binding to FcγR, include mutations from among any of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G, E233P/L234V/L235A/G236del, D265A/P329A, D265A/P329G, D265A/N297A, L234V/L235A/D265A, L234V/L235A/N297A, L234V/L235A/P329A, or L234V/L235A/P329G. In some embodiments, one Fc polypeptide of a heterodimeric Fc comprises the sequence of amino acids set forth in any of SEQ ID NOS: 445 (eg SEQ ID NO:293 or 297), 451 (eg SEQ ID NO:305 or 307), and the other Fc polypeptide of the heterodimeric Fc contains the sequence of amino acids set forth in any of SEQ ID NOS: 446 (e.g. SEQ ID NO:294 or 298), 449 (e.g. SEQ ID NO:301 or 303), 453 (e.g. SEQ ID NO:309 or 311). In some embodiments, one Fc polypeptide of a heterodimeric Fc comprises the sequence of amino acids set forth in any of SEQ ID NOS: 447 (e.g. SEQ ID NO:295 or 299), 452 (eg SEQ ID NO:306 or 308) and the other Fc polypeptide of the heterodimeric Fc comprises the sequence of amino acids set forth in any of SEQ ID NOS: 448 (e.g. SEQ ID NO:296 or 300), 450 (e.g. SEQ ID NO:302 or 304), 454 (e.g. SEQ ID NO:310 or 312).

In some embodiments, the Fc region of the provided multispecific polypeptide constructs exhibit one or more effector functions. In some cases, the Fc region is capable of providing Fc-mediated effector functions, such as for example, ADCC (e.g., release of granzyme B by NK cells), ADCP, and/or CDC. In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments in which the multispecific polypeptide constructs contain a cleavable linker, cleavage of the linker can produce two components that each have biological activity: the CD3-binding region that is able to bind and engage CD3 on a T cell, which, in some aspects, also can contain a CRBR for inducing a costimulatory signal on the T cell and/or an IRBR for inducing an inhibitory signal on the T cell; and the Fc region linked to the B7H3 VHH domain that can exhibit target-specific effector function. In particular embodiments provided herein, the multispecific polypeptide constructs contain a non-cleavable linker and may, in some aspects, not exhibit an independent Fc-mediated effector function.

In some embodiments, the Fc region includes an Fc polypeptide that is mutated or modified to alter one or more effector functions. Thus, in some cases, effector functions such as on or more of ADCC, ADCP and/or CDC can be altered, such as reduced or enhanced, in an Fc for use with the provided constrained multispecific polypeptide constructs. Exemplary mutations to reduce effector function include any as described above.

In some embodiments, an IgG1 Fc polypeptide or a variant thereof such as any described below can be made in a G1 m1 or G1 m3 allotype. In some embodiments, the Fc region can contain amino acids of the human G1 m1 allotype, such as residues containing Asp (D) and Leu (L) at positions 356 and 358, e.g. as set forth in SEQ ID NO:198. In some cases, an Fc polypeptide can contain amino acid substitutions E356D and M358L to reconstitute residues of allotype G1 m1. In other embodiments, the Fc region can contain amino acids of the human G1 m3 allotype, such as residues Glu (E) and Met (M) at positions 356 and 358 by EU numbering, e.g. as set forth in SEQ ID NOS: 457 and 458. In some cases, an Fc polypeptide can contain amino acid substitutions D356E and L358M to reconstitute residues of allotype G1 m3.

c. CD3 Binding Domain

A constrained multispecific polypeptide construct includes one or more copies of an anti-CD3 binding domain. The anti-CD3 binding domains of the disclosure activate T cells via engagement of CD3 or a member of the CD3 complex on the T cells. In preferred embodiments, the anti-CD3 binding domains of the disclosure specifically bind the epsilon chain of CD3, also known as CD3ε. The anti-CD3ε binding domains of the disclosure activate T cells via engagement of CD3ε on the T cells. The anti-CD3 binding domains of the disclosure agonize, stimulate, activate, and/or otherwise augment CD3-mediated T cell activation. Biological activities of CD3 include, for example, T cell activation and other signaling through interaction between CD3 and the antigen-binding subunits of the T-Cell Receptor (TCR). For example, the anti-CD3 binding domains of the disclosure completely or partially activate T cells via engagement of CD3ε on T cells by partially or completely modulating, e.g., agonizing, stimulating, activating or otherwise augmenting CD3-mediated T cell activation.

The CD3 binding domain can be any as described above. In particular embodiments, the CD3 binding domain is an Fv antibody fragment that binds CD3ε (referred to herein as an anti-CD3ε Fv fragment). In some embodiments, the anti-CD3ε Fv antibody fragment is a disulfide stabilized anti-CD3 binding Fv fragment (dsFv). In some embodiments, the anti-CD3 binding domain is monovalent for binding CD3.

In some embodiments, the CD3 binding region is an Fv antibody fragment containing a variable heavy chain (Hv, also called VH) and variable light chain (Lv, also called VL), such as any as described. In aspects of such embodiments, the immunoglobulin Fc region is a heterodimeric Fc region containing two different Fc polypeptides capable of heterodimeric association between both polypeptides of the Fc heterodimer, such as any as described. In such embodiments, the variable heavy chain (VH) and variable light chain (VL) of the CD3 binding region are linked on opposite chains of the heterodimeric Fc.

In some embodiments, the CD3 binding region is an Fv or dsFv of SP34 (Pessano et ai. The EMBO Journal. 4: 337-344, 1985) or of a humanized variant of SP34 (WO2015001085).

In some embodiments, the anti-CD3ε binding domain thereof is an Fv or dsFv fragment that includes a combination of heavy chain variable amino acid sequence and a light chain variable amino acid sequence. In some embodiments, the CD3-binding domain is an Fv or dsFv fragment in which is contained a VH CDR1 sequence that includes at least the amino acid sequence TYAMN (SEQ ID NO: 219); a VH CDR2 sequence that includes at least the amino acid sequence RIRSKYNNYATYYADSVKD (SEQ ID NO: 220); a VH CDR3 sequence that includes at least the amino acid sequence HGNFGNSYVSWFAY (SEQ ID NO: 221), a VL CDR1 sequence that includes at least the amino acid sequence RSSTGAVTTSNYAN (SEQ ID NO: 222); a VL CDR2 sequence that includes at least the amino acid sequence GTNKRAP (SEQ ID NO: 223); and a VL CDR3 sequence that includes at least the amino acid sequence ALWYSNLWV (SEQ ID NO: 224).

In some embodiments, the anti-CD3ε binding domain includes a VH CDR1 sequence that is at least 90%, 91%, 92%, 93%, 94%, 95%, 96%, 97%, 98%, 99% or more identical to the amino acid sequence TYAMN (SEQ ID NO: 219); a VH CDR2 sequence that is at least 90%, 91%, 92%, 93%, 94%, 95%, 96%, 97%, 98%, 99% or more identical to the amino acid sequence RIRSKYNNYATYYADSVKD (SEQ ID NO: 220); a VH CDR3 sequence that is at least 90%, 91%, 92%, 93%, 94%, 95%, 96%, 97%, 98%, 99% or more identical to the amino acid sequence HGNFGNSYVSWFAY (SEQ ID NO: 221), a VL CDR1 sequence that is at least 90%, 91%, 92%, 93%, 94%, 95%, 96%, 97%, 98%, 99% or more identical to the amino acid sequence RSSTGAVTTSNYAN (SEQ ID NO: 222); a VL CDR2 sequence that is at least 90%, 91%, 92%, 93%, 94%, 95%, 96%, 97%, 98%, 99% or more identical to the amino acid sequence GTNKRAP (SEQ ID NO: 223); and a VL CDR3 sequence that is at least 90%, 91%, 92%, 93%, 94%, 95%, 96%, 97%, 98%, 99% or more identical to the amino acid sequence ALWYSNLWV (SEQ ID NO: 224).

In some embodiments, the anti-CD3ε binding domain includes a VH CDR1 sequence that includes at least the amino acid sequence GFTFNTYAMN (SEQ ID NO: 471); a VH CDR2 sequence that includes at least the amino acid sequence RIRSKYNNYATY (SEQ ID NO: 472); a VH CDR3 sequence that includes at least the amino acid sequence HGNFGNSYVSWFAY (SEQ ID NO: 221), a VL CDR1 sequence that includes at least the amino acid sequence RSSTGAVTTSNYAN (SEQ ID NO: 19); a VL CDR2 sequence that includes at least the amino acid sequence GTNKRAP (SEQ ID NO: 20); and a VL CDR3 sequence that includes at least the amino acid sequence ALWYSNLWV (SEQ ID NO: 21).

In some embodiments, the anti-CD3ε binding domain includes a VH CDR1 sequence that includes at least the amino acid sequence GFTFNTYAMN (SEQ ID NO: 471); a VH CDR2 sequence that includes at least the amino acid sequence RIRSKYNNYATY (SEQ ID NO: 472); a VH CDR3 sequence that includes at least the amino acid sequence HGNFGNSYVSWFAY (SEQ ID NO: 221), a VL CDR1 sequence that includes at least the amino acid sequence RSSTGAVTTSNYAN (SEQ ID NO: 222); a VL CDR2 sequence that includes at least the amino acid sequence GTNKRAP (SEQ ID NO: 223); and a VL CDR3 sequence that includes at least the amino acid sequence ALWYSNLWV (SEQ ID NO: 224).

In some embodiments, the anti-CD3ε binding domain includes a VH CDR1 sequence that is at least 90%, 91%, 92%, 93%, 94%, 95%, 96%, 97%, 98%, 99% or more identical to the amino acid sequence GFTFNTYAMN (SEQ ID NO: 471); a VH CDR2 sequence that is at least 90%, 91%, 92%, 93%, 94%, 95%, 96%, 97%, 98%, 99% or more identical to the amino acid sequence RIRSKYNNYATY (SEQ ID NO: 472); a VH CDR3 sequence that is at least 90%, 91%, 92%, 93%, 94%, 95%, 96%, 97%, 98%, 99% or more identical to the amino acid sequence HGNFGNSYVSWFAY (SEQ ID NO: 221), a VL CDR1 sequence that is at least 90%, 91%, 92%, 93%, 94%, 95%, 96%, 97%, 98%, 99% or more identical to the amino acid sequence RSSTGAVTTSNYAN (SEQ ID NO: 222); a VL CDR2 sequence that is at least 90%, 91%, 92%, 93%, 94%, 95%, 96%, 97%, 98%, 99% or more identical to the amino acid sequence GTNKRAP (SEQ ID NO: 223); and a VL CDR3 sequence that is at least 90%, 91%, 92%, 93%, 94%, 95%, 96%, 97%, 98%, 99% or more identical to the amino acid sequence ALWYSNLWV (SEQ ID NO: 224).

In some embodiments, the anti-CD3ε binding domain includes a VH CDR1 sequence that includes at least the amino acid sequence GFTFNTYAMN (SEQ ID NO: 471); a VH CDR2 sequence that includes at least the amino acid sequence RIRSKYNNYATY (SEQ ID NO: 472); a VH CDR3 sequence that includes at least the amino acid sequence HGNFGNSYVSWFAY (SEQ ID NO: 221), a VL CDR1 sequence that includes at least the amino acid sequence GSSTGAVTTSNYAN (SEQ ID NO: 478); a VL CDR2 sequence that includes at least the amino acid sequence GTNKRAP (SEQ ID NO: 479); and a VL CDR3 sequence that includes at least the amino acid sequence ALWYSNHWV (SEQ ID NO: 474).

In some embodiments, the anti-CD3ε binding domain includes a VH CDR1 sequence that includes at least the amino acid sequence GFTFNTYAMN (SEQ ID NO: 471); a VH CDR2 sequence that includes at least the amino acid sequence RIRSKYNNYATY (SEQ ID NO: 472); a VH CDR3 sequence that includes at least the amino acid sequence HGNFGNSYVSWFAY (SEQ ID NO: 221), a VL CDR1 sequence that includes at least the amino acid sequence GSSTGAVTTSNYAN (SEQ ID NO: 478); a VL CDR2 sequence that includes at least the amino acid sequence GTNKRAP (SEQ ID NO: 223); and a VL CDR3 sequence that includes at least the amino acid sequence ALWYSNHWV (SEQ ID NO: 474).

In some embodiments, the anti-CD3ε binding domain includes a VH CDR1 sequence that is at least 90%, 91%, 92%, 93%, 94%, 95%, 96%, 97%, 98%, 99% or more identical to the amino acid sequence GFTFNTYAMN (SEQ ID NO: 471); a VH CDR2 sequence that is at least 90%, 91%, 92%, 93%, 94%, 95%, 96%, 97%, 98%, 99% or more identical to the amino acid sequence RIRSKYNNYATY (SEQ ID NO: 472); a VH CDR3 sequence that is at least 90%, 91%, 92%, 93%, 94%, 95%, 96%, 97%, 98%, 99% or more identical to the amino acid sequence HGNFGNSYVSWFAY (SEQ ID NO: 221), a VL CDR1 sequence that is at least 90%, 91%, 92%, 93%, 94%, 95%, 96%, 97%, 98%, 99% or more identical to the amino acid sequence GSSTGAVTTSNYAN (SEQ ID NO: 478); a VL CDR2 sequence that is at least 90%, 91%, 92%, 93%, 94%, 95%, 96%, 97%, 98%, 99% or more identical to the amino acid sequence GTNKRAP (SEQ ID NO: 223); and a VL CDR3 sequence that is at least 90%, 91%, 92%, 93%, 94%, 95%, 96%, 97%, 98%, 99% or more identical to the amino acid sequence ALWYSNHWV (SEQ ID NO: 474). \In some embodiments, the anti-CD3ε binding domain includes a VH CDR1 sequence that is at least 90%, 91%, 92%, 93%, 94%, 95%, 96%, 97%, 98%, 99% or more identical to the amino acid sequence GFTFNTYAMN (SEQ ID NO: 471); a VH CDR2 sequence that is at least 90%, 91%, 92%, 93%, 94%, 95%, 96%, 97%, 98%, 99% or more identical to the amino acid sequence RIRSKYNNYATY (SEQ ID NO: 472); a VH CDR3 sequence that is at least 90%, 91%, 92%, 93%, 94%, 95%, 96%, 97%, 98%, 99% or more identical to the amino acid sequence HGNFGNSYVSWFAY (SEQ ID NO: 221), a VL CDR1 sequence that is at least 90%, 91%, 92%, 93%, 94%, 95%, 96%, 97%, 98%, 99% or more identical to the amino acid sequence GSSTGAVTTSNYAN (SEQ ID NO: 478); a VL CDR2 sequence that is at least 90%, 91%, 92%, 93%, 94%, 95%, 96%, 97%, 98%, 99% or more identical to the amino acid sequence GTNKRAP (SEQ ID NO: 479); and a VL CDR3 sequence that is at least 90%, 91%, 92%, 93%, 94%, 95%, 96%, 97%, 98%, 99% or more identical to the amino acid sequence ALWYSNHWV (SEQ ID NO: 474).

In some embodiments, the anti-CD3ε binding domain includes a VH CDR1 sequence that includes at least the amino acid sequence GFTFSTYAMN (SEQ ID NO: 476); a VH CDR2 sequence that includes at least the amino acid sequence RIRSKYNNYATY (SEQ ID NO: 477); a VH CDR3 sequence that includes at least the amino acid sequence HGNFGDSYVSWFAY (SEQ ID NO: 473), a VL CDR1 sequence that includes at least the amino acid sequence GSSTGAVTTSNYAN (SEQ ID NO: 478); a VL CDR2 sequence that includes at least the amino acid sequence GTNKRAP (SEQ ID NO: 223); and a VL CDR3 sequence that includes at least the amino acid sequence ALWYSNHWV (SEQ ID NO: 474).

In some embodiments, the anti-CD3ε binding domain includes a VH CDR1 sequence that is at least 90%, 91%, 92%, 93%, 94%, 95%, 96%, 97%, 98%, 99% or more identical to the amino acid sequence GFTFSTYAMN (SEQ ID NO: 476); a VH CDR2 sequence that is at least 90%, 91%, 92%, 93%, 94%, 95%, 96%, 97%, 98%, 99% or more identical to the amino acid sequence RIRSKYNNYATY (SEQ ID NO: 477); a VH CDR3 sequence that is at least 90%, 91%, 92%, 93%, 94%, 95%, 96%, 97%, 98%, 99% or more identical to the amino acid sequence HGNFGDSYVSWFAY (SEQ ID NO: 473), a VL CDR1 sequence that is at least 90%, 91%, 92%, 93%, 94%, 95%, 96%, 97%, 98%, 99% or more identical to the amino acid sequence GSSTGAVTTSNYAN (SEQ ID NO: 478); a VL CDR2 sequence that is at least 90%, 91%, 92%, 93%, 94%, 95%, 96%, 97%, 98%, 99% or more identical to the amino acid sequence GTNKRAP (SEQ ID NO: 223); and a VL CDR3 sequence that is at least 90%, 91%, 92%, 93%, 94%, 95%, 96%, 97%, 98%, 99% or more identical to the amino acid sequence ALWYSNHWV (SEQ ID NO: 474).

In some embodiments, the anti-CD3ε binding domain includes a CDR3 that includes at least amino acids VLWYSNRWV (SEQ ID NO:475). In some embodiments, the anti-CD3ε binding domain includes a CDR3 that is at least 90%, 91%, 92%, 93%, 94%, 95%, 96%, 97%, 98%, 99% or more identical to the amino acids VLWYSNRWV (SEQ ID NO:475).

In some embodiments, the anti-CD3ε binding domain includes one or more copies of an antibody or an antigen-binding fragment thereof selected from the group consisting of a Fab fragment, a F(ab′)₂ fragment, an Fv fragment, a scFv, a scAb, a dAb, a single domain heavy chain antibody, and a single domain light chain antibody. In some embodiments, the anti-CD3 binding domain includes an Fv antibody fragment that binds CD3ε (referred to herein as an anti-CD3ε Fv fragment). In some embodiments, the anti-CD3ε Fv antibody fragment is a disulfide stabilized anti-CD3 binding Fv fragment (dsFv). In some embodiments, the anti-CD3 binding domain is monovalent for binding CD3.

In some embodiments, the CD3 binding region is not a single chain antibody. For example, in some aspects, the CD3 binding region is not a single chain variable fragment (scFv).

In some embodiments, the CD3 binding region is an Fv antibody fragment containing a variable heavy chain (Hv, also called VH) and variable light chain (Lv, also called VL), such as any as described. In aspects of such embodiments, the immunoglobulin Fc region is a heterodimeric Fc region containing two different Fc polypeptides capable of heterodimeric association between both polypeptides of the Fc heterodimer, such as any as described in Section III.C.2.b. In such embodiments, the variable heavy chain (VH) and variable light chain (VL) of the CD3 binding region are linked on opposite chains of the heterodimeric Fc.

In some embodiments, the anti-CD3ε binding domain thereof includes a combination of a heavy chain variable region amino acid sequence and a light chain variable region amino acid sequence comprising an amino acid sequence selected from the group of SEQ ID NO: 225-274, 417, and 459-462. In some embodiments, the anti-CD3ε binding domain thereof includes a combination of a heavy chain variable region amino acid sequence selected from the group of SEQ ID NO: 225-255,460, and 462 and a light chain variable region amino acid sequence comprising an amino acid sequence selected from the group of SEQ ID NO: 256-274, 417, 459, and 461.

In some embodiments, the anti-CD3ε binding domain thereof includes a combination of a heavy chain variable region amino acid sequence and a light chain variable region amino acid sequence comprising an amino acid sequence selected from the group of SEQ ID NO: 217, 218, 225-274, 417, 459-462 and 480. In some embodiments, the anti-CD3ε binding domain thereof includes a combination of a heavy chain variable region amino acid sequence selected from the group of SEQ ID NO: 217, 225-255, 460, 462 and 480 and a light chain variable region amino acid sequence comprising an amino acid sequence selected from the group of SEQ ID NO: 218, 256-274, 417, 459, and 461.

In some embodiments, the anti-CD3ε binding domain thereof is an Fv fragment that includes a combination of heavy chain variable amino acid sequence and a light chain variable amino acid sequence. In some embodiments, the anti-CD3ε binding domain thereof includes a combination of a heavy chain variable region amino acid sequence and a light chain variable region amino acid sequence comprising an amino acid sequence selected from the group of SEQ ID NO: 225-274, 417, and 459-462. In some embodiments, the anti-CD3ε binding domain thereof is an Fv fragment that includes a combination of heavy chain variable amino acid sequence and a light chain variable amino acid sequence comprising an amino acid sequence that is at least 90%, 91%, 92%, 93%, 94%, 95%, 96%, 97%, 98%, 99% or more identical to an amino acid sequence selected from the group consisting of SEQ ID NOs: 225-274, 417, and 459-462.

In some embodiments, the anti-CD3ε binding domain thereof is an Fv fragment that includes a combination of heavy chain variable amino acid sequence and a light chain variable amino acid sequence. In some embodiments, the anti-CD3ε binding domain thereof includes a combination of a heavy chain variable region amino acid sequence and a light chain variable region amino acid sequence comprising an amino acid sequence selected from the group of SEQ ID NO: 217, 218, 225-274, 417, 459-462 and 480. In some embodiments, the anti-CD3ε binding domain thereof is an Fv fragment that includes a combination of heavy chain variable amino acid sequence and a light chain variable amino acid sequence comprising an amino acid sequence that is at least 90%, 91%, 92%, 93%, 94%, 95%, 96%, 97%, 98%, 99% or more identical to an amino acid sequence selected from the group consisting of SEQ ID NOs: 217, 218, 225-274, 417, 459-462 and 480.

In some embodiments, the anti-CD3ε binding domain thereof is an Fv, such as a dsFv fragment, that includes a heavy chain variable amino acid sequence that is at least 90%, 91%, 92%, 93%, 94%, 95%, 96%, 97%, 98%, 99% or more identical to an amino acid sequence selected from the group consisting of SEQ ID NO: 225-255,460, and 462 and a light chain variable amino acid sequence that is at least 90%, 91%, 92%, 93%, 94%, 95%, 96%, 97%, 98%, 99% or more identical to an amino acid sequence selected from the group consisting of SEQ ID NO: 256-274, 417, 459, and 461. In some embodiments, the anti-CD3ε binding domain thereof includes a combination of a heavy chain variable region amino acid sequence selected from the group of SEQ ID NO: 225-255,460, and 462 and a light chain variable region amino acid sequence comprising an amino acid sequence selected from the group of SEQ ID NO: 256-274, 417, 459, and 461. In some embodiments, the anti-CD3ε binding domain thereof is an Fv, such as a dsFv fragment, that includes a heavy chain variable amino acid sequence selected from the group of SEQ ID NO: 225-255, 460 and 462 and a light chain variable amino acid sequence selected from the group consisting of SEQ ID NO: 256-274, 417, 459 and 461. In some embodiments, the anti-CD3 binding domain is an Fv, such as a dsFv, in which is contained a variable heavy chain (VH) comprising the amino acid sequence of SEQ ID NO: 237 and a variable light chain (VL) comprising the amino acid sequence of SEQ ID NO: 265. In some embodiments, the anti-CD3 binding domain is an Fv or dsFv, in which is contained a variable heavy chain (VH) comprising the amino acid sequence of SEQ ID NO: 237 and a variable light chain (VL) comprising the amino acid sequence of SEQ ID NO: 417.

In some embodiments, the anti-CD3ε binding domain thereof is an Fv fragment that includes a combination of heavy chain variable amino acid sequence selected from the group of SEQ ID NO: 217, 225-255,460, 462 and 480 and light chain variable amino acid sequence selected from the group consisting of SEQ ID NO: 218, 256-274, 417, 459, and 461. In some embodiments, the anti-CD3ε binding domain thereof is an Fv fragment that includes a combination of heavy chain variable amino acid sequence that is at least 90%, 91%, 92%, 93%, 94%, 95%, 96%, 97%, 98%, 99% or more identical to an amino acid sequence selected from the group consisting of SEQ ID NO: 217, 225-255,460, 462 and 480 and a light chain variable amino acid sequence that is at least 90%, 91%, 92%, 93%, 94%, 95%, 96%, 97%, 98%, 99% or more identical to an amino acid sequence selected from the group consisting of SEQ ID NO: 218, 256-274, 417, 459, and 461.

In some embodiments, the anti-CD3ε Fv antibody fragment includes a combination of a heavy chain variable amino acid sequence that is at least 90%, 91%, 92%, 93%, 94%, 95%, 96%, 97%, 98%, 99% or more identical to an amino acid sequence selected from the group consisting of SEQ ID NO: 217, 225-236, 238-240, 460, and 241 and a light chain variable amino acid sequence that is at least 90%, 91%, 92%, 93%, 94%, 95%, 96%, 97%, 98%, 99% or more identical to an amino acid sequence selected from the group consisting of SEQ ID NO: 218, 256, 258-264, 266, 268, 270, and 461. In some embodiments, the anti-CD3ε Fv antibody fragment includes a combination of a heavy chain variable amino acid sequence selected from the group of SEQ ID NO: 217, 225-236, 238-240, 460, and 241 and a light chain variable amino acid sequence selected from the group consisting of SEQ ID NO: 218, 256, 258-264, 266, 268, 270, 459 and 461.

In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO:217. In some embodiments, the anti-CD3ε binding domain includes a variable light chain (VL) comprising an amino acid sequence that is at least 90%, 91%, 92%, 93%, 94%, 95%, 96%, 97%, 98%, 99% or more identical to the amino acid sequence of SEQ ID NO: 218. In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 217 and a variable light chain (VL) comprising an amino acid sequence that is at least 90%, 91%, 92%, 93%, 94%, 95%, 96%, 97%, 98%, 99% or more identical to the amino acid sequence of SEQ ID NO: 218. In some embodiments, the anti-CD3ε binding domain thereof includes a variable heavy chain (VH) comprising the amino acid sequence of SEQ ID NO: 217. In some embodiments, the anti-CD3ε binding domain includes a variable light chain (VL) comprising the amino acid sequence of SEQ ID NO: 218. In some embodiments, the anti-CD3ε binding domain thereof includes a variable heavy chain (VH) comprising the amino acid sequence of SEQ ID NO: 217 and a variable light chain (VL) comprising the amino acid sequence of SEQ ID NO: 218.

In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 460. In some embodiments, the anti-CD3ε binding domain includes a variable light chain (VL) comprising an amino acid sequence that is at least 90%, 91%, 92%, 93%, 94%, 95%, 96%, 97%, 98%, 99% or more identical to the amino acid sequence of SEQ ID NO: 461. In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 460 and a variable light chain (VL) comprising an amino acid sequence that is at least 90%, 91%, 92%, 93%, 94%, 95%, 96%, 97%, 98%, 99% or more identical to the amino acid sequence of SEQ ID NO: 461. In some embodiments, the anti-CD3ε binding domain thereof includes a variable heavy chain (VH) comprising the amino acid sequence of SEQ ID NO: 460. In some embodiments, the anti-CD3ε binding domain includes a variable light chain (VL) comprising the amino acid sequence of SEQ ID NO: 461. In some embodiments, the anti-CD3ε binding domain thereof includes a variable heavy chain (VH) comprising the amino acid sequence of SEQ ID NO: 460 and a variable light chain (VL) comprising the amino acid sequence of SEQ ID NO: 461.

In particular embodiments, the Fv is a disulfide stabilized Fv fragment (dsFv) in which the the V_{H}-V_{L} heterodimer is stabilized by an interchain disulfide bond. In some embodiments, the interchain disulfide bond is engineered by mutation of position in framework positions of the VH and/or VL chain. In some embodiments, the disulfide stabilized anti-CD3 Fv comprises an anti-CD3 VH with the mutation 44 to Cys and an anti-CD3 VL with the mutation 100 to Cys by Kabat numbering. For example, in some embodiments, the VH chain contains the mutation G44C and the VL chain contains the mutation G100C, each by kabat numbering. In some embodiments, the disulfide stabilized anti-CD3 Fv comprises an anti-CD3 VH with the mutation at position 105 to Cys and an anti-CD3 VL with the mutation position 43 to Cys by Kabat numbering.

In some embodiments, the anti-CD3ε Fv antibody fragment includes a combination of a variable heavy chain amino acid sequence that is at least 90%, 91%, 92%, 93%, 94%, 95%, 96%, 97%, 98%, 99% or more identical to an amino acid sequence selected from the group consisting of SEQ ID NO: 237 and 242-255, and 462 and a variable light chain amino acid sequence that is at least 90%, 91%, 92%, 93%, 94%, 95%, 96%, 97%, 98%, 99% or more identical to an amino acid sequence selected from the group consisting of SEQ ID NO: 257, 265, 267, 269, 271-274, 417, and 459. In some of any such embodiments, the anti-CD3 Fv is a dsFv that has a VH chain containing the mutation G44C and a VL chain containing the mutation G100C, each by kabat numbering. In some embodiments, the anti-CD3ε Fv antibody fragment includes a combination of a variable heavy chain amino acid sequence selected from the group of SEQ ID NO: 237 and 242-255, and 462 and a variable light chain amino acid sequence selected from the group consisting of SEQ ID NO: 257, 265, 267, 269, 271-274, 417, and 459.

In some embodiments, the anti-CD3ε Fv antibody fragment includes a combination of a variable heavy chain amino acid sequence that is at least 90%, 91%, 92%, 93%, 94%, 95%, 96%, 97%, 98%, 99% or more identical to an amino acid sequence selected from the group consisting of SEQ ID NO: 237 and 242-255, 462 and 480 and a variable light chain amino acid sequence that is at least 90%, 91%, 92%, 93%, 94%, 95%, 96%, 97%, 98%, 99% or more identical to an amino acid sequence selected from the group consisting of SEQ ID NO: 257, 265, 267, 269, 271-274, 417, and 459. In some of any such embodiments, the anti-CD3 Fv is a dsFv that has a VH chain containing the mutation G44C and a VL chain containing the mutation G100C, each by kabat numbering. In some embodiments, the anti-CD3ε Fv antibody fragment includes a combination of a variable heavy chain amino acid sequence selected from the group of SEQ ID NO: 237 and 242-255, 462 and 480 and a variable light chain amino acid sequence selected from the group consisting of SEQ ID NO: 257, 265, 267, 269, 271-274, 417, and 459.

In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 237 In some embodiments, the anti-CD3ε binding domain includes a variable light chain (VL) comprising an amino acid sequence that is at least 90%, 91%, 92%, 93%, 94%, 95%, 96%, 97%, 98%, 99% or more identical to the amino acid sequence of SEQ ID NO: 265. In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 237 and a variable light chain (VL) comprising an amino acid sequence that is at least 90%, 91%, 92%, 93%, 94%, 95%, 96%, 97%, 98%, 99% or more identical to the amino acid sequence of SEQ ID NO: 265. In some of any such embodiments, the anti-CD3 Fv is a dsFv that has a VH chain containing the mutation G44C and a VL chain containing the mutation G100C, each by kabat numbering. In some embodiments, the anti-CD3ε binding domain thereof includes a variable heavy chain (VH) comprising the amino acid sequence of SEQ ID NO: 237. In some embodiments, the anti-CD3ε binding domain includes a variable light chain (VL) comprising the amino acid sequence of SEQ ID NO: 265. In some embodiments, the anti-CD3ε binding domain thereof includes a variable heavy chain (VH) comprising the amino acid sequence of SEQ ID NO: 237 and a variable light chain (VL) comprising the amino acid sequence of SEQ ID NO: 265.

In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 462. In some embodiments, the anti-CD3ε binding domain includes a variable light chain (VL) comprising an amino acid sequence that is at least 90%, 91%, 92%, 93%, 94%, 95%, 96%, 97%, 98%, 99% or more identical to the amino acid sequence of SEQ ID NO: 459. In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 462 and a variable light chain (VL) comprising an amino acid sequence that is at least 90%, 91%, 92%, 93%, 94%, 95%, 96%, 97%, 98%, 99% or more identical to the amino acid sequence of SEQ ID NO: 459. In some of any such embodiments, the anti-CD3 Fv is a dsFv that has a VH chain containing the mutation G44C and a VL chain containing the mutation G100C, each by kabat numbering. In some embodiments, the anti-CD3ε binding domain thereof includes a variable heavy chain (VH) comprising the amino acid sequence of SEQ ID NO: 462. In some embodiments, the anti-CD3ε binding domain includes a variable light chain (VL) comprising the amino acid sequence of SEQ ID NO: 459. In some embodiments, the anti-CD3ε binding domain thereof includes a variable heavy chain (VH) comprising the amino acid sequence of SEQ ID NO: 462 and a variable light chain (VL) comprising the amino acid sequence of SEQ ID NO: 459.

In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 480. In some embodiments, the anti-CD3ε binding domain includes a variable light chain (VL) comprising an amino acid sequence that is at least 90%, 91%, 92%, 93%, 94%, 95%, 96%, 97%, 98%, 99% or more identical to the amino acid sequence of SEQ ID NO: 459. In some embodiments, the anti-CD3ε binding domain thereof includes a variable heavy chain (VH) comprising an amino acid sequence that is at least 90%, 91%, 92%, 93%, 94%, 95%, 96%, 97%, 98%, 99% or more identical to the amino acid sequence of SEQ ID NO: 480 and a variable light chain (VL) comprising an amino acid sequence that is at least 90%, 91%, 92%, 93%, 94%, 95%, 96%, 97%, 98%, 99% or more identical to the amino acid sequence of SEQ ID NO: 459. In some of any such embodiments, the anti-CD3 Fv is a dsFv that has a VH chain containing the mutation G44C and a VL chain containing the mutation G100C, each by kabat numbering. In some embodiments, the anti-CD3ε binding domain thereof includes a variable heavy chain (VH) comprising the amino acid sequence of SEQ ID NO: 480. In some embodiments, the anti-CD3ε binding domain includes a variable light chain (VL) comprising the amino acid sequence of SEQ ID NO: 459. In some embodiments, the anti-CD3ε binding domain thereof includes a variable heavy chain (VH) comprising the amino acid sequence of SEQ ID NO: 480 and a variable light chain (VL) comprising the amino acid sequence of SEQ ID NO: 459.

d. Linker

A constrained multispecific polypeptide constructs contain a linker that joins or couples the first component containing the immunoglobulin Fc region and the second component containing the CD3 binding region. In some embodiments, the linker is positioned at the end of the C-terminal region of the Fc region, such that the Fc region is N-terminal to the CD3 binding region. It is understood that because the provided constrained multispecific polypeptide constructs are multimers, such as dimers containing a first and second polypeptide that together form the first and second component, the provided constructs include a linker joining the Fc portion and the CD3 binding region of the first and a linker joining the Fc portion and the CD3 binding region of the second polypeptide. In some embodiments, the first polypeptide includes a first Fc polypeptide of a heterodimeric Fc region, a linker, and a first domain (e.g. VH) of a CD3 binding region, and the second polypeptide includes a second Fc polypeptide of the heterodimeric Fc region, a linker and second domain (e.g. VL) of the CD3 binding region. Typically, the linkers present in the first and second polypeptides of the constrained multispecific polypeptide construct are the same. Thus, in some embodiments, each domain of the CD3 binding domain is linked via a linker, such as the same linker, to opposite polypeptides of the Fc, such as heterodimeric Fc.

Various polypeptide linkers for use in fusion proteins are known (see e.g. Chen et al. (2013) Adv. Drug. Deliv. 65:1357-1369; and International PCT publication No. WO 2014/099997, WO2000/24884; U.S. Pat. Nos. 5,258,498; 5,525,491; 5,525,491, 6,132,992).

In some embodiments, the linker is chosen so that, when the CD3 binding region is joined to the Fc region of the multispecific polypeptide conjugate, the CD3 binding region is constrained and not able to, or not substantially able to, bind or engage CD3 on the surface of a cell, e.g. T cell, upon contact of the multispecific polypeptide construct with the cell. Various assays can be employed to assess binding or engagement of CD3 by the multispecific polypeptide construct, including assays to assess T cell binding, NFAT activation using a reporter system, cytolytic T cell activity, cytokine production and/or expression of T cell activation markers. Exemplary assays are shown in the provided Examples. Typically, the linker also is one that ensures correct folding of the polypeptide construct, does not exhibit a charge that would be inconsistent with the activity or function of the linked polypeptides or form bonds or other interactions with amino acid residues in one or more of the domains that would impede or alter activity of the linked polypeptides. In some embodiments, the linker is a polypeptide linker The polypeptide linker can be a flexible linker or a rigid linker or a combination of both. In some aspects, the linker is a short, medium or long linker. In some embodiments, the linker is up to 40 amino acids in length. In some embodiments, the linker is up to 25 amino acids in length. In some embodiments, the linker is at least or is at least about 2 amino acids in length. In some aspects, a suitable length is, e.g., a length of at least one and typically fewer than about 40 amino acid residues, such as 2-25 amino acid residues, 5-20 amino acid residues, 5-15 amino acid residues, 8-12 amino acid . In some embodiments, the linker is from or from about 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 24 amino acids, 8 to 20 amino acids, 8 to 18 amino acids, 8 to 14 amino acids, 8 to 12 amino acids, 8 to 10 amino acids, 10 to 24 amino acids, 10 to 20 amino acids, 10 to 18 amino acids, 10 to 14 amino acids, 10 to 12 amino acids, 12 to 24 amino acids, 12 to 20 amino acids, 12 to 18 amino acids, 12 to 14 amino acids, 14 to 24 amino acids, 14 to 20 amino acids, 14 to 18 amino acids, 18 to 24 amino acids, 18 to 20 amino acids or 20 to 24 amino acids. In some embodiments, the linker is 2, 3, 4, 5, 6, 7, 8, 9, 10, 11, 12, 13, 14, 15, 16, 17, 18, 19 or 20 amino acids in length.

In certain aspects, the longer the linker length, the greater the CD3 binding when the multispecific polypeptide conjugate is bounds to its antigen, e.g. TAA. Thus, in some aspects, the linker is greater than 12 amino acids in length, such as greater than 13, 14, 15, 16, 17 or 18 amino acids in length. In some embodiments, the linker is 12 to 40 amino acids in length, 12 to 30 amino acids, 12 to 24 amino acids, 12 to 18 acids, 12 to 15 amino acids, 15 to 40 amino acids, 15 to 30 amino acids, 15 to 24 amino acids, 15 to 18 amino acids, 18 to 40 amino acids, 18 to 30 amino acids, 18 to 24 amino acids, 24 to 40 amino acids, 24 to 30 amino acids or 30 to 40 amino acids.

The linkers can be naturally occurring, synthetic or a combination of both. Particularly suitable linker polypeptides predominantly include amino acid residues selected from Glycine (Gly), Serine (Ser), Alanine (Ala), and Threonine (Thr). For example, the linker may contain at least 75% (calculated on the basis of the total number of residues present in the peptide linker), such as at least 80%, at least 85%, or at least 90% of amino acid residues selected from Gly, Ser, Ala, and Thr. The linker may also consist of Gly, Ser, Ala and/or Thr residues only. In some embodiments, the linker contains 1-25 glycine residues, 5-20 glycine residues, 5-15 glycine residues, or 8-12 glycine residues. In some aspects, suitable peptide linkers typically contain at least 50% glycine residues, such as at least 75% glycine residues. In some embodiments, a peptide linker comprises glycine residues only. In some embodiments, a peptide linker comprises glycine and serine residues only.

In some embodiments, these linkers are composed predominately of the amino acids Glycine and Serine, denoted as GS-linkers herein. In some embodiments, the linker contains (GGS)n, wherein n is 1 to 10, such as 1 to 5, for example 1 to 3, such as GGS(GGS)n (SEQ ID NO:491), wherein n is 0 to 10. In particular embodiments, the linker contains the sequence (GGGGS)n (SEQ ID NO: 313), wherein n is 1 to 10 or n is 1 to 5, such as 1 to 3. In further embodiments, the linker contains (GGGGGS)n (SEQ ID NO:314), wherein n is 1 to 4, such as 1 to 3. The linker can include combinations of any of the above, such as repeats of 2, 3, 4, or 5 GS, GGS, GGGGS, and/or GGGGGS linkers may be combined. In some embodiments, such a linker is 2, 3, 4, 5, 6, 7, 8, 9, 10, 11, 12, 13, 14, 15, 16, 17, 18 or 19 amino acids in length.

In some embodiments, the linker is (in one-letter amino acid code): GGS, GGGGS (SEQ ID NO: 315), or GGGGGS (SEQ ID NO: 316). In some embodiments, the GS-linker comprises an amino acid sequence of GGSGGS, i.e., (GGS)₂ (SEQ ID NO: 191); GGSGGSGGS, i.e., (GGS)₃ (SEQ ID NO: 192); GGSGGSGGSGGS, i.e., (GGS)₄ (SEQ ID NO: 193); GGSGGSGGSGGSGGS, i.e., (GGS)₅ (SEQ ID NO: 194); GGGGGSGGGGGSGGGGGS, i.e., (G5S)₃ (SEQ ID NO: 317), GGSGGGGSGGGGSGGGGS (SEQ ID NO: 319) and GGGGSGGGGSGGGGS (SEQ ID NO:318). In some embodiments, the linker is GGGGG (SEQ ID NO:196). In some embodiments, the linker is PGGGG (SEQ ID NO:444). In some embodiments, the linker is GGGG (SEQ ID NO:195). In some of any of the above examples, serine can be replaced with alanine (e.g., (Gly4Ala) or (Gly3Ala)).

In some embodiments, the linker includes a peptide linker having the amino acid sequence GlyₓXaa-Gly_{y}-Xaa-Gly_{z} (SEQ ID NO:320), wherein each Xaa is independently selected from Alanine (Ala), Valine (Val), Leucine (Leu), Isoleucine (Ile), Methionine (Met), Phenylalanine (Phe), Tryptophan (Trp), Proline (Pro), Glycine (Gly), Serine (Ser), Threonine (Thr), Cysteine (Cys), Tyrosine (Tyr), Asparagine (Asn), Glutamine (Gln), Lysine (Lys), Arginine (Arg), Histidine (His), Aspartate (Asp), and Glutamate (Glu), and wherein x, y, and z are each integers in the range from 1-5. In some embodiments, each Xaa is independently selected from the group consisting of Ser, Ala, and Thr. In a specific variation, each of x, y, and z is equal to 3 (thereby yielding a peptide linker having the amino acid sequence Gly-Gly-Gly-Xaa-Gly-Gly-Gly-Xaa-Gly-Gly-Gly (SEQ ID NO:321), wherein each Xaa is selected as above.

In some embodiments, the linker is serine-rich linkers based on the repetition of a (SSSSG)n (SEQ ID NO:322) motif where n is at least 1, though y can be 2, 3, 4, 5, 6, 7, 8 and 9.

In some cases, it may be desirable to provide some rigidity into the peptide linker. This may be accomplished by including proline residues in the amino acid sequence of the peptide linker. Thus, in some embodiments, a linker comprises at least one proline residue in the amino acid sequence of the peptide linker For example, a peptide linker can have an amino acid sequence wherein at least 25% (e.g., at least 50% or at least 75%) of the amino acid residues are proline residues. In one particular embodiment, the peptide linker comprises proline residues only.

In some aspects, a peptide linker comprises at least one cysteine residue, such as one cysteine residue. For example, in some embodiments, a linker comprises at least one cysteine residue and amino acid residues selected from the group consisting of Gly, Ser, Ala, and Thr. In some such embodiments, a linker comprises glycine residues and cysteine residues, such as glycine residues and cysteine residues only. Typically, only one cysteine residue will be included per peptide linker. One example of a specific linker comprising a cysteine residue includes a peptide linker having the amino acid sequence Glyₘ-Cys-Glyₙ, wherein n and m are each integers from 1-12, e.g., from 3-9, from 4-8, or from 4-7. In a specific variation, such a peptide linker has the amino acid sequence GGGGG-C-GGGGG (SEQ ID NO:323).

In some embodiments, the linker of the fusion protein is a structured or constrained linker. In particular embodiments, the structured linker contains the sequence (AP)n or (EAAAK)n (SEQ ID NO:134), wherein n is 2 to 20, preferably 4 to 10, including but not limited to, AS-(AP)n-GT (SEQ ID NO:135) or AS-(EAAAK)n-GT (SEQ ID NO:136), wherein n is 2 to 20, such as 2, 3, 4, 5, 6, 7, 8, 9, 10, 11, 12, 13, 14 or 15. In other embodiments, the linker comprises the sequences (GGGGA)n (SEQ ID NO:137), (PGGGS)n (SEQ ID NO:138), (AGGGS)n (SEQ ID NO:139) or GGS-(EGKSSGSGSESKST)n-GGS (SEQ ID NO:140, wherein n is 2 to 20), (ADAAP)n (SEQ ID NO:545, wherein n is 2 to 20), (ADAAP)n-G (SEQ ID NO:546, wherein n is 2 to 20), (GEPQG)n (SEQ ID NO:547, wherein n is 2 to 20), (GEPQG)n-G (SEQ ID NO:548, wherein n is 2 to 20), (AGGEP)n (SEQ ID NO:549, wherein n is 2 to 20), (AGGEP)n-G (SEQ ID NO:550, wherein n is 2 to 20), (AGSEP)n (SEQ ID NO:551, wherein n is 2 to 20), (AGSEP)n-G (SEQ ID NO:552, wherein n is 2 to 20), (GGGEQ)n (SEQ ID NO:553, wherein n is 2 to 20), (GGGEQ)n-G (SEQ ID NO:554, wherein n is 2 to 20). In some embodiments, the linker is SSSASASSA (SEQ ID NO:141), GSPGSPG (SEQ ID NO:142), ATTTGSSPGPT (SEQ ID NO:143), ADAAPADAAPG (SEQ ID NO:555), GEPQGGEPQGG (SEQ ID NO:556), AGGEPAGGEPG (SEQ ID NO:557), AGSEPAGSEPG (SEQ ID NO:558), or GGGEQGGGEQG (SEQ ID NO:559). In some embodiments, such linkers, by virtue of their structure, may be more resistant to proteolytic degradation, thereby offering an advantage when injected in vivo. In some embodiments, such linkers are negatively charged and may be better suited for dampening the binding of the CD3 binding domain to CD3.

In some embodiments, the linker is not a cleavable linker, also called non-cleavable linker. In some embodiments, the linker is not a cleavable by a protease. In some embodiments, a linker that is not a cleavable linker or that is not cleavable by a protease is one that is generally stable for in vivo delivery or recombinant production. In some aspects, a linker that is not cleavable by a protease includes those that do not contain at least one peptide bond which preferably lies within a cleavable peptide sequence or recognition site of a protease. In particular embodiments, a non-cleavable linker is not a target substrate for a protease, such that it is not preferentially or specifically cleaved by a protease compared to a linker that contains a substrate recognition site for the same protease.

In some embodiments, the linker does not contains a substrate recognition site or cleavage site for a particular protease, which is the sequence recognized by the active site of a protease that is cleaved by a protease. Typically, for example, for a serine protease, a cleavage sequence is made up of the P1-P4 and P1′-P4′ amino acids in a substrate, where cleavage occurs after the P1 position. Typically, a cleavage sequence for a serine protease is six residues in length to match the extended substrate specificity of many proteases, but can be longer or shorter depending upon the protease. Typically, the linker does not include a P1-P1′ scissile bond sequence that is recognized by a protease. In some aspects, a non-cleavable linker or a linker that does not contain a substrate recognition site that is specifically recognized for cleavage by a protease is one whose cleavage by a protease is substantially less than cleavage of a target substrate of the protease.

In some embodiments, the linker is a cleavable linker In some aspects, a cleavable linker is a linker, such as any described above, that further includes a sequence that is a substrate for a protease due to the presence of at least one bond that can be broken under physiological conditions. In some cases, a cleavable linker is susceptible to or sensitive to cleavage under specific conditions that exist in vivo, such as following exposure to an extracellular protease, including those present in cellular environments in vivo. In some cases, the protease may be present in a particular physiological microenvironment, such as the tumor microenvironment, thereby restricting the sites at which cleavage may occur.

A protease typically exhibits specificity or preference for cleavage of a particular target substrate compared to another non-target substrate. Such a degree of specificity can be determined based on the rate constant of cleavage of a sequence, e.g. linker, which is a measure of preference of a protease for its substrate and the efficiency of the enzyme. Any method to determine the rate of increase of cleavage over time in the presence of various concentrations of substrate can be used to calculate the specificity constant. For example, a substrate is linked to a fluorogenic moiety, which is released upon cleavage by a protease. By determining the rate of cleavage at different protease concentrations the specificity constant for cleavage (k_{cat}/Kₘ) can be determined for a particular protease towards a particular linker. In some embodiments, a cleavable linker is a linker that is capable of being specifically cleaved by a protease at a rate of about at least 1×10⁴ M⁻¹S⁻¹, or at least 5×10⁴ M⁻¹S⁻¹, at least 10×10⁴ M⁻¹S⁻¹. at least 10×10⁵ M⁻¹S⁻¹ or more.

In some embodiments, a constrained multispecific polypeptide constructs of the disclosure include a cleavable linker that joins the first and second components. In some embodiments, the cleavable linker includes an amino acid sequence that can serve as a substrate for a protease, usually an extracellular protease. For example, the cleavable linker may include a cleavage sequence containing at least one peptide bond which preferably lies within a cleavable peptide sequence of a protease. Suitable proteases include, for example, matrix metalloproteases (MMP), cysteine proteases, serine proteases and plasmin activators, which are formed or activated in intensified manner in diseases such as rheumatoid arthritis or cancer, leading to excessive tissue degradation, inflammations and metastasis. In particular embodiments, the protease is a protease that is produced by a tumor, an activated immune effector cell (e.g. a T cell or a NK cell), or a cell in a tumor microenvironment. In some embodiments, the protease is a granzyme B, a matriptase or an MMP, such as MMP-2.

The cleavable linker may be selected based on a protease that is produced by a tumor that is in proximity to cells that express the target and/or produced by a tumor that is co-localized in tissue with the desired target of the multispecific polypeptide constructs. There are reports in the literature of increased levels of proteases having known substrates in a number of cancers, e.g., solid tumors. See, e.g., La Rocca et al, (2004) British J. of Cancer 90(7): 1414-1421.

In some embodiments, the cleavable linker that joins the first and second component of a constrained multispecific polypeptide construct is cleaved by a protease produced by an immune effector cell that is activated by one of the components. For example, multispecific polypeptide constructs that encompass an effector enabled or enhanced IgG Fc region are capable of eliciting ADCC when engaged with the target antigen. Central to ADCC is the release of granzyme B and perforin from the effector cells, namely NK cells and cytotoxic T-cells. Upon release granzyme B enters the target cell in a perforin dependent manner wherein it mediates apoptosis. Importantly, granzyme B is active within the extracellular synapse between the effector cell and the target cell. In some embodiments, the cleavable linker that joins the first and second component multispecific polypeptide construct is cleaved by granzyme B. Granzyme B is released during effector cell activation mediated by one of the components of the multispecific polypeptide construct. In some embodiments, granzyme B and other proteases can be produced by immune effector cells, including activated T cells or NK cells. In some embodiments, activation of T cells by CD3 engagement upon binding of a TAA by a multispecific polypeptide construct may release such proteases, which then can cleave a specific cleavable linker thereby potentiating or increasing activity of the CD3 binding molecule to engage CD3. In some embodiments, the cleavage can amplify or increase the activity achieved by the multispecific construct when bound to TAA in an uncleaved state.

Exemplary substrates include but are not limited to substrates cleavable by one or more of the following enzymes or proteases: ADAMS, ADAMTS, e.g. ADAM8; ADAM9; ADAM10; ADAM12; ADAM15; ADAM17/TACE; ADAMDEC1; ADAMTS1; ADAMTS4; ADAMTS5; aspartate proteases, e.g., BACE or Renin; aspartic cathepsins, e.g., Cathepsin D or Cathepsin E; Caspases, e.g., Caspase 1, Caspase 2, Caspase 3, Caspase 4, Caspase 5, Caspase 6, Caspase 7, Caspase 8, Caspase 9, Caspase 10, or Caspase 14; cysteine cathepsins, e.g., Cathepsin B, Cathepsin C, Cathepsin K, Cathepsin L, Cathepsin S, Cathepsin V/L2, Cathepsin X/Z/P; Cysteine proteinases, e.g., Cruzipain; Legumain; Otubain-2; KLKs, e.g., KLK4, KLK5, KLK6, KLK7, KLK8, KLK10, KLK11, KLK13, or KLK14; Metallo proteinases, e.g., Meprin; Neprilysin; PSMA; BMP-1; MMPs, e.g., MMP1, MMP2, MMP3, MMP7, MMP8, MMP9, MMP10, MMP11, MMP12, MMP13, MMP14, MMP15, MMP16, MMP17, MMP19, MMP20, MMP23, MMP24, MMP26, or MMP27, serine proteases, e.g., activated protein C, Cathepsin A, Cathepsin G, Chymase, coagulation factor proteases (e.g., FVIIa, FIXa, FXa, FXIa, FXIIa), Elastase, granzyme B, Guanidinobenzoatase, HtrA1, Human Neutrophil Elastase, Lactoferrin, Marapsin, NS3/4A, PACE4, Plasmin, PSA, tPA, Thrombin, Tryptase, uPA; Type II Transmembrane Serine Proteases (TTSPs), e.g., DESC1, DPP-4, FAP, Hepsin, Matriptase-2, Matriptase, TMPRSS2, TMPRSS3, or TMPRSS4; and any combination thereof.

In some embodiments, the cleavable linker is cleaved by multiple proteases, e.g., 2 or more proteases, 3 or more proteases, 4 or more proteases, and so on.

In some embodiments, the cleavable linker is selected for use with a specific protease, for example a protease that is known to be produced by a tumor that is in proximity to cells that express the target and/or produced by a tumor that is co-localized with the target of the multispecific polypeptide construct.

In some embodiments, the cleavable linker contains a substrate recognition site or cleavage site for a particular protease, which is the sequence recognized by the active site of a protease that is cleaved by a protease. Typically, for example, for a serine protease, a cleavage sequence is made up of the P1-P4 and P1′-P4′ amino acids in a substrate, where cleavage occurs after the P1 position. Typically, a cleavage sequence for a serine protease is six residues in length to match the extended substrate specificity of many proteases, but can be longer or shorter depending upon the protease. Typically, the cleavable linker includes a P1-P1′ scissile bond sequence that is recognized by a protease. In some aspects, the cleavable linker is engineered to introduce a peptide bond able to be cleaved by a specific protease, for example by introducing a substrate recognition site sequence or cleavage sequence of the protease.

In some embodiments, the cleavable linker includes a combination of two or more substrate sequences. In some embodiments, each substrate sequence is cleaved by the same protease. In some embodiments, at least two of the substrate sequences are cleaved by different proteases. In some embodiments, the cleavable linker comprises an amino acid that is a substrate for granzyme B. In some embodiments, a granzyme B cleavable linker contains an amino acid sequence having the general formula P4 P3 P2 P1↓P1′ (SEQ ID NO: 334), wherein P4 is amino acid I, L, Y, M, F, V, or A; P3 is amino acid A, G, S, V, E, D, Q, N, or Y; P2 is amino acid H, P, A, V, G, S, or T; P1 is amino acid D or E; and P1′ is amino acid I, L, Y, M, F, V, T, S, G or A. In some embodiments, a granzyme B cleavable linker contains an amino acid sequence having the general formula P4 P3 P2 P1 ↓ P1′ (SEQ ID NO: 335), wherein P4 is amino acid I or L; P3 is amino acid E; P2 is amino acid P or A; P1 is amino acid D; and P1′ is amino acid I, V, T, S, or G.

In some embodiments, the substrate for granzyme B comprises the amino acid sequence LEAD (SEQ ID NO: 336), LEPD (SEQ ID NO: 337), or LEAE (SEQ ID NO:338). In some embodiments, the cleavable linker contains the amino acid sequence the cleavable linker comprises the amino acid sequence IEPDI (SEQ ID NO:339), LEPDG (SEQ ID NO:340), LEADT (SEQ ID NO:341), IEPDG (SEQ ID NO:342), IEPDV (SEQ ID NO:343), IEPDS (SEQ ID NO:344), IEPDT (SEQ ID NO:345), IEPDP (SEQ ID NO:482), LEPDG (SEQ ID NO:340) or LEADG (SEQ ID NO:334).

In some embodiments, the cleavable linker comprises an amino acid that is a substrate for matriptase. In some embodiments, the cleavable linker comprises the sequence P1QAR↓(A/V) (SEQ ID NO: 346), wherein P1 is any amino acid. In some embodiments, the cleavable linker comprises the sequence RQAR(A/V) (SEQ ID NO: 347). In some embodiments, the substrate for matriptase comprises the amino acid sequence RQAR (SEQ ID NO: 348). In some embodiments, the cleavable linker comprises the amino acid sequence RQARV (SEQ ID NO: 349).

In some embodiments, the cleavable linker comprises an amino acid that is a substrate for one or more matrix metalloproteases (MMPs). In some embodiments, the MMP is MMP-2. In some embodiments, the cleavable linker contains. the general formula P3 P2 P1↓P1′ (SEQ ID NO: 350), wherein P3 is P, V or A; P2 is Q or D; P1 is A or N; and P1′ is L, I or M. In some embodiments, the cleavable linker contains the general formula P3 P2 P1 ↓ P1′ (SEQ ID NO: 351), wherein P3 is P; P2 is Q or D; P1 is A or N; and P1′ is L or I. In some embodiments, the substrate for MMP comprises the amino acid sequence PAGL (SEQ ID NO: 352).

In some embodiments, the cleavable linker comprises a combination of an amino acid sequence that is a substrate for granzyme B and an amino acid sequence that is a substrate for matriptase. In some embodiments, the cleavable linker comprises a combination of the amino acid sequence LEAD (SEQ ID NO: 336) and the amino acid sequence RQAR (SEQ ID NO: 348).

In some embodiments, the cleavable linker comprises a combination of an amino acid sequence that is a substrate for granzyme B and an amino acid sequence that is a substrate for MMP. In some embodiments, the cleavable linker comprises a combination of the amino acid sequence LEAD (SEQ ID NO: 336) and the amino acid sequence PAGL (SEQ ID NO: 352).

In some embodiments, the cleavable linker comprises a combination of an amino acid sequence that is a substrate for matriptase and an amino acid sequence that is a substrate for MMP. In some embodiments, the cleavable linker comprises a combination of the amino acid sequence RQAR (SEQ ID NO: 348) and the amino acid sequence PAGL (SEQ ID NO: 352).

In some embodiments, the cleavable linker comprises a combination of an amino acid sequence that is a substrate for granzyme B, an amino acid sequence that is a substrate for matriptase, and an amino acid sequence that is a substrate for MMP. In some embodiments, the cleavable linker comprises a combination of an amino acid sequence that is a substrate for granzyme B and an amino acid sequence that is a substrate for MMP. In some embodiments, the cleavable linker comprises a combination of the amino acid sequence LEAD (SEQ ID NO: 336), the amino acid sequence RQAR (SEQ ID NO: 348), and the amino acid sequence PAGL (SEQ ID NO: 352).

The cleavable linker can include any known linkers. Examples of cleavable linkers are described in Be'liveau et al. (2009) FEBS Journal, 276; U.S. published application Nos. US20160194399; US20150079088; US20170204139; US20160289324; US20160122425; US20150087810; US20170081397; U.S. Pat. No. 9,644,016.

In some embodiments, the cleavable linker comprises an amino acid sequence selected from the group consisting of TGLEADGSPAGLGRQARVG (SEQ ID NO: 353); TGLEADGSRQARVGPAGLG (SEQ ID NO: 354); TGSPAGLEADGSRQARVGS (SEQ ID NO: 355); TGPAGLGLEADGSRQARVG (SEQ ID NO: 356); TGRQARVGLEADGSPAGLG (SEQ ID NO: 357); TGSRQARVGPAGLEADGS (SEQ ID NO: 358); and TGPAGLGSRQARVGLEADGS (SEQ ID NO: 359); GPAGLGLEPDGSRQARVG (SEQ ID NO: 360); GGSGGGGIEPDIGGSGGS (SEQ ID NO: 361); GGSGGGGLEADTGGSGGS (SEQ ID NO: 362); GSIEPDIGS (SEQ ID NO: 363); GSLEADTGS (SEQ ID NO: 364); GGSGGGGIEPDGGGSGGS (SEQ ID NO: 365); GGSGGGGIEPDVGGSGGS (SEQ ID NO: 366); GGSGGGGIEPDSGGSGGS (SEQ ID NO: 367); GGSGGGGIEPDTGGSGGS (SEQ ID NO: 368); GGGSLEPDGSGS (SEQ ID NO: 369); GPAGLGLEADGSRQARVG (SEQ ID NO: 370), GGEGGGGSGGSGGGS (SEQ ID NO: 371); GSSAGSEAGGSGQAGVGS (SEQ ID NO: 372); GGSGGGGLEAEGSGGGGS (SEQ ID NO: 373); GGSGGGGIEPDPGGSGGS(SEQ ID NO: 374); TGGSGGGGIEPDIGGSGGS (SEQ ID NO: 375).

e. Costimulatory Binding Domain

Multispecific polypeptide constructs of the present disclosure include one or more co-stimulatory receptor binding region (CRBR) that binds a costimulatory receptor. In some embodiments, the one or more CRBR of the provided multispecific polypeptide constructs bind a co-stimulatory receptor expressed on T cells. In some embodiments, the co-stimulatory receptor is upregulated, induced, or expressed on the surface of an activated T cell. In some aspects, the CRBR binds a co-stimulatory receptor and stimulates the co-stimulatory receptor. In some embodiments, agonistic binding of the co-stimulatory receptor to the CRBR of the multispecific polypeptide induces downstream signaling in the T cell to potentiate or enhance T cell activation or functionalities following engagement of CD3. In some embodiments, the CRBR, or independently each of the CRBRs, is an antibody or antigen binding fragment, a natural cognate binding partner of the co-stimulatory receptor, an Anticalin (engineered lipocalin), a Darpin, a Fynomer, a Centyrin (engineered fibroneticin III domain), a cystine-knot domain, an Affilin, an Affibody, or an engineered CH3 domain.

In some embodiments, the CRBR, or independently each of the CRBRs, such as the first CRBR and the second CRBRs, includes one or more copies of an antibody or an antigen-binding fragment thereof. In some embodiments, the CRBR or independently each of the CRBRs, such as the first antigen-binding domain and the second CRBRs, includes one or more copies of an antibody or an antigen-binding fragment thereof selected from the group consisting of a Fab fragment, a F(ab′)₂ fragment, an Fv fragment, a scFv, a scAb, a dAb, a single domain heavy chain antibody, and a single domain light chain antibody.

In some embodiments, the CRBR, or independently each of the CRBRs, such as the first CRBR and the second CRBRs, is a single chain antibody. In some examples, the single chain is an scFv, a scAb, a single domain heavy chain antibody, or a single domain light chain antibody.

In some embodiments, the CRBR, or independently each of the CRBRs, such as the first CRBR and the second CRBR, includes one or more single domain antibody (sdAb) fragments, for example V_{H}H, V_{NAR}, engineered V_{H} or V_{K} domains. V_{H}Hs can be generated from natural camelid heavy chain only antibodies, genetically modified rodents that produce heavy chain only antibodies, or naive/synthetic camelid or humanized camelid single domain antibody libraries. V_{NAR}_{^{S}} can be generated from cartilaginous fish heavy chain only antibodies. Various methods have been implemented to generate monomeric sdAbs from conventionally heterodimeric V_{H} and V_{K} domains, including interface engineering and selection of specific germline families.

In some embodiments, the CRBR, or independently each of the CRBRs such as the first CRBR and/or the second CRBR, of the multispecific polypeptide constructs contains at least one sdAb or an scFv that binds a costimulatory receptor. In some embodiments, the at least one scFv or sdAb that binds a costimulatory receptor is positioned amino-terminally relative to the Fc region and/or carboxy-terminally relative to the CD3 binding region of the multispecific polypeptide construct. In some embodiments, the multispecific polypeptide construct contains only one scFv or sdAb that binds to a costimulatory receptor, which can be positioned either amino-terminally relative to the Fc region and/or carboxy-terminally relative to the CD3 binding region. In some embodiments, the multispecific polypeptide construct contains two scFv or sdAb that bind to a costimulatory receptor, positioned amino-terminally relative to the Fc region and/or carboxy-terminally relative to the CD3 binding region.

In some embodiments, the multispecific polypeptide construct is formed from or includes two polypeptides, including a first polypeptide comprising a first Fc polypeptide of a heterodimeric Fc region, a linker, a VH domain of an anti-CD3 antibody or antigen binding fragment (e.g. Fv), and an scFv or sdAb that binds to a costimulatory receptor; and a second polypeptide comprising a second Fc polypeptide of the heterodimeric Fc region, the linker, a VL domain of the anti-CD3 antibody or antigen binding fragment (e.g. Fv) and, optionally, another, the same or different, scFv or sdAb that binds to a costimulatory receptor. The scFv or sdAb that binds the costimulatory receptor can be positioned amino terminally relative to an Fc polypeptide of the heterodimeric Fc and/or carboxy-terminally relative to a VH or VL chain of the CD3 binding region. At least one of the first and/or second polypeptide of the multispecific polypeptide construct also includes an antigen binding domain that binds a TAA or a chain thereof as described in Section II.4. In some embodiments, the antigen binding domain that binds a TAA is a scFv or sdAb and is included as part of the first and/or second polypeptide of the multispecific polypeptide construct. In some embodiments, the antigen binding domain that binds a TAA is a Fab, and the multispecific polypeptide construct is additionally formed from a third polypeptide where at least the first and second polypeptide include a chain of the Fab that binds TAA (e.g. VH-CH1 or VL-CL of a Fab) and the third polypeptide contains the other chain of the Fab that binds TAA (e.g. the other of VH-CH1 or VL-CL of a Fab).

In some embodiments, the CRBR or independently each of the CRBRs, such as the first CRBR and/or the second CRBRs, contains more than one chain. In some embodiments, the CRBR or independently each of the CRBRs, such as the first CRBR and/or the second CRBRs, of the multispecific polypeptide constructs contains VH and VL sequences assembled as FABs.

In some embodiments, the CRBR antigen binding domain or independently each of the CRBR antigen binding domains, such as the first antigen-binding domain and/or the second antigen binding domains, of the multispecific polypeptide constructs contains a VH-CH1 (Fd) and a VL-CL of a Fab antibody that binds a costimulatory receptor. In some embodiments, the Fab antibody containing a VH-CH1 (Fd) and a VL-CL is positioned amino-terminally relative to the Fc region and/or carboxy-terminally relative to the CD3 binding region of the multispecific polypeptide construct. In some embodiments, the multispecific polypeptide construct contains only one Fab antibody, containing a VH-CH1 (Fd) or VL-CL, that binds to a costimulatory receptor, which can be positioned either amino-terminally relative to the Fc region and/or carboxy-terminally relative to the CD3 binding region. In some embodiments, the multispecific polypeptide construct contains two Fab antibody fragments, each containing a VH-CH1 (Fd) and VL-CL, that binds to a costimulatory receptor, in which one is positioned amino-terminally relative to the Fc region and the other is positioned carboxy-terminally relative to the CD3 binding region.

In some embodiments, the multispecific polypeptide construct is formed from or includes three or more polypeptides, including a first polypeptide comprising a first Fc polypeptide of a heterodimeric Fc region, a linker and a VH-CH1 (Fd) or VL-CL of a Fab antibody fragment that binds to a costimulatory receptor; a second polypeptide comprising a second Fc polypeptide of the heterodimeric Fc region, the linker and, optionally, the same VH-CH1 (Fd) or VL-CL of the Fab antibody fragment that binds to a costimulatory receptor, and a third polypeptide comprising the other of the VH-CH1 (Fd) or VL-CL of the Fab antibody fragment that binds to the costimulatory receptor. The first, second and/or third polypeptide of the multispecific polypeptide construct also can include a B7H3 VHH domain, such as any as described.

In some embodiments, the CRBR, or independently each of the CRBRs, is or includes a natural (native) cognate binding partner of the co-stimulatory receptor (e.g. a natural ligand), or a variant thereof that exhibits binding activity to the co-stimulatory receptor.

In some embodiments, the one or more CRBR of the provided multispecific polypeptide constructs bind a co-stimulatory receptor expressed on T cells. In some embodiments, there are more than one CRBR that binds to a costimulatory receptor and each of the CRBRs, such as the first CRBR and the second CRBR, bind the same co-stimulatory receptor. In some embodiments, each of the CRBRs, such as the first CRBR and the CRBRs, bind a different co-stimulatory receptor. In some embodiments, each of the CRBRs, such as the first CRBR and the second CRBR bind a different epitope on the same co-stimulatory receptor. In some embodiments, each of the CRBRs, such as the first antigen-CRBR and the CRBR, bind the same epitope on the same co-stimulatory receptor.

In some embodiments, the CRBR, or independently each of the CRBRs that binds a co-stimulatory receptor results in monovalent, bivalent, trivalent, or tetravalent binding to the co-stimulatory receptor.

In some embodiments, the antigen binding domains results in monovalent, bivalent, trivalent, or tetravalent binding to the TAA. In some embodiments, bivalent binding to the TAA comprises two antigen binding domains that bind the same epitope of the same antigen (e.g. mono-epitopic). In some embodiments, bivalent binding to the TAA comprises two antigen binding domains that bind different epitopes of the same antigen (e.g. bi-epitopic). In some embodiments, monovalent binding to the TAA comprises one antigen binding domain that binds one epitope of the antigen (e.g.mono-epitopic).

In some embodiments, the co-stimulatory receptor is expressed on T cells, such as primary T cells obtained from a subject. In some embodiments, the co-stimulatory receptor is expressed on human T cells, such as primary human T cells obtained from a human subject.

In some embodiments, the co-stimulatory receptor is a member of the tumor necrosis factor (TNF) receptor family. In some embodiments, the costimulatory receptor is a member of the immunoglobulin superfamily (IgSF). In some embodiments, the costimulatory receptor is a member of the B7 family of receptors.

In some embodiments, the co-stimulatory receptor is selected from the group consisting of 41BB (CD137), OX40 (CD134), CD27, glucocorticoid-induced TNFR-related protein (GITR), CD28, ICOS, CD40, B-cell activating factor receptor (BAFF-R), B-cell maturation antigen (BCMA), Transmembrane activator and CAML interactor (TACI), and NKG2D. In some embodiments, the co-stimulatory receptor is selected from 41BB, OX40, GITR, ICOS, or CD28. In some embodiments, the co-stimulatory receptor is selected from 41BB, OX40, or GITR.

In some embodiments, the costimulatory receptor is 41BB. In some embodiments, the costimulatory receptor is OX40. In some embodiments, the costimulatory receptor is GITR. In some embodiments, the costimulatory receptor is ICOS. In some embodiments, the costimulatory receptor is CD28.

In some embodiments, the CRBR of the multispecific polypeptide is or comprises an agonistic binding molecule to the co-stimulatory receptor. The CRBR can bind to the co-stimulatory receptor and initiate, induce, or stimulate a reaction or activity that is similar to or the same as that initiated, induced, or stimulated by the receptor's natural ligand. In some aspects, the binding of the CRBR to the co-stimulatory receptor induces or stimulates a downstream signal that is more than 5%, more than 10%, more than 20%, more than 30%, more than 40%, more than 50%, more than 60%, more than 70%, more than 80%, more than 90%, or more than 100% of the signal that is initiated, induced, or stimulated by the receptor's natural ligand.

In some embodiments, the one or more CRBR is an antibody or fragment thereof that binds to the co-stimulatory receptor 41BB (CD137), OX40 (CD134), CD27, glucocorticoid-induced TNFR-related protein (GITR), CD28, ICOS, CD40, B-cell activating factor receptor (BAFF-R), B-cell maturation antigen (BCMA). In some embodiments, the one or more CRBR is an antibody or fragment thereof that binds to the co-stimulatory receptor 41BB, OX40, GITR, ICOS, or CD28. In some embodiments, the one or more CRBR is an antibody or fragment thereof that binds to the co-stimulatory receptor 41BB, OX40, or GITR. Exemplary polypeptides for binding 41BB, OX40 and GITR are described in PCT publication. No. WO2017123650, WO2017123673, and WO2017015623, respectively. In some embodiments, the one or more CRBR is a single domain antibody (sdAb) that binds the co-stimulatory receptor, such as those described in PCT publication. No. WO2017123650, WO2017123673, and WO2017015623.

In some examples, the co-stimulatory receptor binding region (CRBR) binds or comprises a natural cognate binding partner of 41BB (CD137), OX40 (CD134), CD27, glucocorticoid-induced TNFR-related protein (GITR), CD28, ICOS, CD40, B-cell activating factor receptor (BAFF-R), B-cell maturation antigen (BCMA), Transmembrane activator and CAML interactor (TACI), NKG2D. In some embodiments, the natural cognate binding partner is selected from 41BB ligand (41BBL), OX40L (CD252), CD70, GITR Ligand/TNFSF18, CD80 (B7-1), CD86 (B7-2), ICOS Ligand (ICOSL), CD154 (CD40L), B-cell activating factor (BAFF), A proliferation-inducing ligand (APRIL), NKG2D ligands, or a functional fragment thereof.

In some embodiments, the co-stimulatory receptor binding region (CRBR) is an antibody or antigen binding fragment that binds 41BB. In particular examples, the CRBR that binds 4-1BB is a single domain antibody. In some embodiments, the sdAb contains a CDR1 GFSFSINAMG (set forth in SEQ ID NO:468), a CDR2 AIESGRNTV (set forth in SEQ ID NO:469) and a CDR3 LKGNRVVSPSVAY (set forth in SEQ ID NO: 470). Examples of sdAb that target 41BB are described in PCT publication. No. WO2017123650.

Exemplary sequences of CRBRs are set forth in Table 4.

In some embodiments, at least one CRBR, or independently each CRBR, binds the co-stimulatory receptor 41BB. In some examples, the CRBR is or contains an antibody or antigen binding fragment specific to or that binds 41BB, such as a sdAb or fragments containing a VH and VL (e.g. scFv). In some embodiments, at least on CRBR, or independently each CRBR, is a natural ligand of 41BB or is a functional binding fragment thereof. Exemplary 41BB-binding CRBRs are set forth in any of SEQ ID NOS: 376-400 and 481. In some emobidments, a 41BB-binding CRBR is a functional fragment of 41BB ligand (41BBL) containing the extracellular domain or a truncated portion thereof, such as corresponding to amino acids 50-254 of UniProt No. P41273, e.g. as set forth in SEQ ID NO:376, or a truncated portion or fragment thereof set forth in any of SEQ ID NOS:392-399. In some embodiments, at least one CRBR, or independently each CRBR, is an anticalin set forth in any one of SEQ ID NOS: 383-391. In some embodiments, a sdAb, such as a VHH, contains a CDR1, a CDR2, and a CDR3 having a sequence set forth in SEQ ID NO: 468, 469, and 470, respectively. A 41BB-binding CRBR, such as a sdAb, can include the sequence set forth in SEQ ID NO:400. A 41BB-binding CRBR, such as a sdAb, can include the sequence set forth in SEQ ID NO:481. In some embodiments, the 4-1BB-binding domain contains an antigen binding antibody fragment containing a VH and a VL, such as a single chain fragment in which the VH and VL are separated by a linker, for example an scFv. In some embodiments, the 41BB binding CRBR contains a VH set forth in any of SEQ ID NOS: 377, 379 and 381 and a VL set forth in any of SEQ ID NO: 378, 380 or 382. The CRBRs, or independently each CRBR, in a provided multispecific polypeptide construct can have at least 85%, 85%, 87%, 88%, 89%, 90%, 91%, 92%, 93%, 94%, 95%, 96%, 97%, 98% or 99% sequence identity to any of the foregoing SEQ ID Nos and bind 41BB.

In some embodiments, at least one CRBR, or independently each CRBR, binds the co-stimulatory receptor OX40. In some examples, the CRBR is or contains an antibody or antigen binding fragment specific to or that binds OX40, such as a sdAb or fragments containing a VH and VL (e.g. scFv). In some embodiments, at least on CRBR, or independently each CRBR, is a natural ligand of OX40 or is a functional binding fragment thereof. Exemplary of such OX40-binding CRBRs are set forth in any of SEQ ID NOS: 401-410. In some embodiments, the OX40-binding CRBR contains an VH set forth in any of SEQ ID NOS: 406 and 408 and a VL set forth in any of SEQ ID NO: 407 and 409. The CRBRs, or independently each CRBR, in a provided multispecific polypeptide construct can have at least 85%, 85%, 87%, 88%, 89%, 90%, 91%, 92%, 93%, 94%, 95%, 96%, 97%, 98% or 99% sequence identity to any of the foregoing SEQ ID Nos and bind OX40.

In some embodiments, at least one CRBR, or independently each CRBR, binds the co-stimulatory receptor GITR. In some examples, the CRBR is or contains an antibody or antigen binding fragment specific to or that binds GITR, such as a sdAb or fragments containing a VH and VL (e.g. scFv). In some embodiments, at least one CRBR, or independently each CRBR, is a natural ligand of GITR or is a functional binding fragment thereof. Exemplary of such GITR-binding CRBRs are set forth in any of SEQ ID NOS: 411-416. In some embodiments, the GITR binding CRBR contains a VH set forth in any of SEQ ID NOS: 412, 414, 289 and 291 and a VL set forth in any of SEQ ID NO: 413, 415, 290, 292. The CRBRs, or independently each CRBR, in a provided multispecific polypeptide construct can have at least 85%, 85%, 87%, 88%, 89%, 90%, 91%, 92%, 93%, 94%, 95%, 96%, 97%, 98% or 99% sequence identity to any of the foregoing SEQ ID Nos and bind GITR.

In some embodiments, at least one CRBR, or independently each CRBR, binds the co-stimulatory receptor CD27. In some examples, the CRBR is or contains an antibody or antigen binding fragment specific to or that binds CD27, such as a sdAb or fragments containing a VH and VL (e.g. scFv). In some embodiments, at least one CRBR, or independently each CRBR, is a natural ligand of CD27 or is a functional binding fragment thereof. Exemplary of such CD27-binding CRBRs are set forth in any of SEQ ID NOS: 276-278. In some embodiments, the CD27 binding CRBR contains a VH set forth SEQ ID NO: 277 and a VL set forth in SEQ ID NO: 278. The CRBRs, or independently each CRBR, in a provided multispecific polypeptide construct can have at least 85%, 85%, 87%, 88%, 89%, 90%, 91%, 92%, 93%, 94%, 95%, 96%, 97%, 98% or 99% sequence identity to any of the foregoing SEQ ID Nos and bind CD27.

In some embodiments, at least one CRBR, or independently each CRBR, binds the co-stimulatory receptor ICOS. In some examples, the CRBR is or contains an antibody or antigen binding fragment specific to or that binds ICOS, such as a sdAb or fragments containing a VH and VL (e.g. scFv). In some embodiments, at least one CRBR, or independently each CRBR, is a natural ligand of ICOS or is a functional binding fragment thereof. An exemplary ICOS-binding CRBR sequence is set forth in SEQ ID NO: 279.

In some embodiments, at least one CRBR, or independently each CRBR, binds the co-stimulatory receptor CD28. In some examples, the CRBR is or contains an antibody or antigen binding fragment specific to or that binds CD28, such as a sdAb or fragments containing a VH and VL (e.g. scFv). In some embodiments, at least one CRBR, or independently each CRBR, is a natural ligand of CD28 or is a functional binding fragment thereof. An exemplary CD28-binding CRBR sequence is set forth in SEQ ID NO: 280.

TABLE 4
Exemplary CRBR Sequences

| | | | |
|---|---|---|---|
| CRBR | Format | Reference | SEQ ID NO |

41BB binding CRBR Sequences

| | | | |
|---|---|---|---|
| 41BBL | Natural Ligand | UniProt accession no. P41273 | 376 |
| PF-05082566 | VH | US 2012/0237498 (SEQ ID NO: 43) | 377 |
| | VL | US 2012/0237498 (SEQ ID NO: 45) | 378 |
| BMS663513 | VH | WO 2005/035584 (SEQ ID NO: 9) | 379 |
| | VL | WO 2005/035584 (SEQ ID NO: 6) | 380 |
| MSB7 | VH | US 2017/0226215 (SEQ ID NO: 138) | 381 |
| | VL | US 2017/0226215 (SEQ ID NO: 28) | 382 |
| 41BB Anticalin | Anticalin | WO 2016/177762 (SEQ ID NO: 12) | 383 |
| 41BB Anticalin | Anticalin | WO 2016/177762 (SEQ ID NO: 13) | 384 |
| 41BB Anticalin | Anticalin | WO 2016/177762 (SEQ ID NO: 14) | 385 |
| 41BB Anticalin | Anticalin | WO 2016/177762 (SEQ ID NO: 15) | 386 |
| 41BB Anticalin | Anticalin | WO 2016/177762 (SEQ ID NO: 16) | 387 |
| 41BB Anticalin | Anticalin | WO 2016/177762 (SEQ ID NO: 17) | 388 |
| 41BB Anticalin | Anticalin | WO 2016/177762 (SEQ ID NO: 18) | 389 |
| 41BB Anticalin | Anticalin | WO 2016/177762 SEQ ID NO: 19) | 390 |
| 41BB Anticalin | Anticalin | WO 2016/177762 (SEQ ID NO: 20) | 391 |
| 71-254 of human | 41BB ligand | WO 2017/167672 (SEQ ID NO: 3) | 392 |
| 41BBL | | | |
| 85-254 of human | 41BB ligand | WO 2017/167672 (SEQ ID NO: 4) | 393 |
| 41BBL | | | |
| 80-254 of human | 41BB ligand | WO 2017/167672 (SEQ ID NO: 5) | 394 |
| 41BBL | | | |
| 52-254 of human | 41BB ligand | WO 2017/167672 (SEQ ID NO: 6) | 395 |
| 41BBL | | | |
| 71-248 of human | 41BB ligand | WO 2017/167672 (SEQ ID NO: 7) | 396 |
| 41BBL | | | |
| 85-248 of human | 41BB ligand | WO 2017/167672 (SEQ ID NO: 8 | 397 |
| 41BBL | | | |
| 80-248 of human | 41BB ligand | WO 2017/167672 (SEQ ID NO: 9) | 398 |
| 41BBL | | | |
| 52-248 of human | 41BB ligand | WO 2017/167672 (SEQ ID NO: 10) | 399 |
| 41BBL | | | |
| 41BB sdAb | sdAb | US 2017/0198050 | 400 |
| 41BB sdAb | sdAb | | 481 |

OX40-binding CRBR Sequences

| | | | |
|---|---|---|---|
| OX40 ligand | Natural Ligand | UniProt accession no. P23510 | 401 |
| OX40 ligand | Natural Ligand | U.S. Pat. No. 7,959,925 (SEQ ID NO: 2) | 402 |
| human OX40L: | Natural Ligand | WO 2017/167672 (SEQ ID NO: 11) | 403 |
| 51-183 | | | |
| Human OX40L: | Natural Ligand | WO 2017/167672 (SEQ ID NO: 12) | 404 |
| 51-183 N90D | | | |
| Human OX40L: | Natural Ligand | WO 2017/167672 (SEQ ID NO :13) | 405 |
| 52-183 | | | |
| 1A07 | VH | US 2015/0307617 (SEQ ID NO: 56) | 406 |
| | VL | US 2015/0307617 (SEQ ID NO: 59) | 407 |
| 1949 | VH | WO 2016/179517 (SEQ ID NO: 16) | 408 |
| | VL | WO 2016/179517 | 409 |
| ID10v1 | sdAb | U.S. Pat. No, 9,006,399 | 410 |

GITR-binding CRBR Sequences

| | | | |
|---|---|---|---|
| GITR ligand | Natural Ligand | UniProt no. Q9UNG2 | 411 |
| 36E5 | VH | US 2014/0348841 (SEQ ID NO: 104) | 412 |
| | VL | US 2014/0348841 (SEQ ID NO: 105) | 413 |
| TRX-518 | VH | US 2013/0183321 (SEQ ID NO: 54) | 414 |
| | VL | US 2013/0183321 (SEQ ID NO: 44) | 415 |
| 5H7v2 | VH | US 2015/0064204 (SEQ ID NO: 282) | 289 |
| | VL | US 2015/0064204 (SEQ ID NO: 134) | 290 |
| 41G5v2 | VH | US 2015/0064204 (SEQ ID NO: 312) | 291 |
| | VL | US 2015/0064204 (SEQ ID NO: 124) | 292 |
| C06v3 | sdAb | US 2017/0022284 (SEQ ID NO: 59) | 416 |

CD27-binding CRBR Sequences

| | | | |
|---|---|---|---|
| CD70-ECD | Natural Ligand | UniProt no. P32970 | 276 |
| 1F5 | VH | US 2011/0274685 | 277 |
| | VL | US 2011/0274685 | 278 |

CD28-binding CRBR Sequences

| | | |
|---|---|---|
| CD28 sdAb | sdAb | 280 |

ICOS-binding CRBR Sequences

| | | |
|---|---|---|
| ICOS sdAb | sdAb | 279 |

In some embodiments, the one or more CRBR is linked, directly or indirectly via a linker, to the Fc region and/or to the CD3 binding region. In some embodiments, linkage is via a linker. In some embodiments, the linker is a linking peptide (LP), which can include any flexible or rigid linker as described herein, although generally the peptide linking the CRBR or regions is not a cleavable liker.

In some embodiments, the multispecific polypeptide construct comprises a linking peptide (LP) between the CRBR and the Fc region. In some embodiments, the multispecific polypeptide construct comprises a linking peptide (LP) between the CD3 binding region and the CRBR.

f. Inhibitory Receptor Binding Regions (IRBR)

The multispecific polypeptide constructs of the present disclosure include one or more inhibitor receptor binding region (IRBR) that binds an inhibitory receptor. In some embodiments, the one or more IRBR of the provided multispecific polypeptide constructs binds an inhibitory receptor expressed on T cells. In some embodiments, the inhibitory receptor is upregulated, induced, or expressed on the surface of an activated T cell. In some aspects, the IRBR blocks an interaction between the inhibitory receptor and its ligand, thereby reducing, suppressing or decreasing an inhibitory signal in the cell to which the IRBR binds, e.g. T cell. In some embodiments, the IRBR, or independently each of the IRBRs, is an antibody or antigen binding fragment, a natural cognate binding partner of the co-stimulatory receptor, an Anticalin (engineered lipocalin), a Darpin, a Fynomer, a Centyrin (engineered fibroneticin III domain), a cystine-knot domain, an Affilin, an Affibody, or an engineered CH3 domain.

In some embodiments, the IRBR, or independently each of the IRBRs, such as the first IRBR and the second IRBR, includes one or more copies of an antibody or an antigen-binding fragment thereof. In some embodiments, the IRBR or independently each of the IRBRs, such as the first IRBR and the second IRBR, includes one or more copies of an antibody or an antigen-binding fragment thereof selected from the group consisting of a Fab fragment, a F(ab′)₂ fragment, an Fv fragment, a scFv, a scAb, a dAb, a single domain heavy chain antibody, and a single domain light chain antibody.

In some embodiments, the IRBR, or independently each of the IRBRs, such as the first IRBR and the second IRBR, is a single chain antibody. In some examples, the single chain is an scFv, a scAb, a single domain heavy chain antibody, or a single domain light chain antibody.

In some embodiments, the IRBR, or independently each of the IRBRs, such as the first IRBR and the second IRBR, includes one or more single domain antibody (sdAb) fragments, for example V_{H}H, V_{NAR}, engineered V_{H} or V_{K} domains. V_{H}Hs can be generated from natural camelid heavy chain only antibodies, genetically modified rodents that produce heavy chain only antibodies, or naive/synthetic camelid or humanized camelid single domain antibody libraries. V_{NAR}_{^{S}} can be generated from cartilaginous fish heavy chain only antibodies. Various methods have been implemented to generate monomeric sdAbs from conventionally heterodimeric V_{H} and V_{K} domains, including interface engineering and selection of specific germline families.

In some embodiments, the IRBR, or independently each of the IRBRs such as the first IRBR and/or the second IRBR, of the multispecific polypeptide constructs contains at least one sdAb or an scFv that binds an inhibitory receptor. In some embodiments, the at least one scFv or sdAb that binds an inhibitory receptor is positioned amino-terminally relative to the Fc region and/or carboxy-terminally relative to the CD3 binding region of the multispecific polypeptide construct. In some embodiments, the multispecific polypeptide construct contains only one scFv or sdAb that binds to an inhibitory receptor, which can be positioned either amino-terminally relative to the Fc region and/or carboxy-terminally relative to the CD3 binding region. In some embodiments, the multispecific polypeptide construct contains two scFv or sdAb that bind to an inhibitory receptor, positioned amino-terminally relative to the Fc region and/or carboxy-terminally relative to the CD3 binding region.

In some embodiments, the multispecific polypeptide construct is formed from or includes two polypeptides, including a first polypeptide comprising a first Fc polypeptide of a heterodimeric Fc region, a linker, a VH domain of an anti-CD3 antibody or antigen binding fragment (e.g. Fv), and an scFv or sdAb that binds to an inhibitory receptor; and a second polypeptide comprising a second Fc polypeptide of the heterodimeric Fc region, the linker, a VL domain of the anti-CD3 antibody or antigen binding fragment (e.g. Fv) and, optionally, another, the same or different, scFv or sdAb that binds to an inhibitory receptor. The scFv or sdAb that binds the inhibitory receptor can be positioned amino terminally relative to an Fc polypeptide of the heterodimeric Fc and/or carboxy-terminally relative to a VH or VL chain of the CD3 binding region. At least one of the first and/or second polypeptide of the multispecific polypeptide construct also includes an antigen binding domain that binds a TAA or a chain thereof as described in Section II.4. In some embodiments, the antigen binding domain that binds a TAA is a scFv or sdAb and is included as part of the first and/or second polypeptide of the multispecific polypeptide construct. In some embodiments, the antigen binding domain that binds a TAA is a Fab, and the multispecific polypeptide construct is additionally formed from a third polypeptide where at least the first and second polypeptide include a chain of the Fab that binds TAA (e.g. VH-CH1 or VL-CL of a Fab) and the third polypeptide contains the other chain of the Fab that binds TAA (e.g. the other of VH-CH1 or VL-CL of a Fab).

In some embodiments, the multispecific polypeptide construct is formed from or includes two polypeptides, including a first polypeptide comprising in order: a first antigen binding domain specific for a TAA, a first Fc polypeptide of a heterodimeric Fc region, a linker, a VH domain of an anti-CD3 antibody or antigen binding fragment (e.g. Fv), and a second antigen binding domain specific for a TAA; and a second polypeptide containing the IRBR and comprising in order: a second Fc polypeptide of the heterodimeric Fc region, the linker, a VL domain of the anti-CD3 antibody or antigen binding fragment (e.g. Fv) , wherein the IRBR is positioned amino terminally to the Fc region and/or C-terminally to the CD3 binding region. In some embodiments, the IRBR is positioned on the second polypeptide carboxy-terminally to the CD3 binding region. In some embodiments, the IRBR is positioned on the second polypeptide amino-terminally to the Fc region. In some embodiments, the IRBR is positioned amino terminally to the Fc region and C-terminally to the CD3 binding region. In some embodiments, the first and second antigen binding domain is specific to a TAA are the same. In some embodiments, the first and second antigen binding domain is specific to a TAA are different. In some embodiments, the first antigen binding domain and the second antigen binding domain bind a different TAA. In some embodiments, the first antigen binding domain and the second antigen binding domain bind a distinct or non-overlapping epitope of the same TAA and/or compete for binding to the same TAA.

In some embodiments, the IRBR or independently each of the IRBRs, such as the first IRBR and/or the second IRBR, contains more than one chain. In some embodiments, the IRBR or independently each of the IRBRs, such as the first IRBR and/or the second IRBR, of the multispecific polypeptide constructs contains VH and VL sequences assembled as FABs.

In some embodiments, the antigen binding domain or independently each of the antigen binding domains, such as the first antigen-binding domain and/or the second antigen binding domains, of the multispecific polypeptide constructs contains a VH-CH1 (Fd) and a VL-CL of a Fab antibody that binds an inhibitory receptor. In some embodiments, the Fab antibody containing a VH-CH1 (Fd) and a VL-CL is positioned amino-terminally relative to the Fc region and/or carboxy-terminally relative to the CD3 binding region of the multispecific polypeptide construct. In some embodiments, the multispecific polypeptide construct contains only one Fab antibody, containing a VH-CH1 (Fd) or VL-CL, that binds to a inhibitory receptor, which can be positioned either amino-terminally relative to the Fc region and/or carboxy-terminally relative to the CD3 binding region. In some embodiments, the multispecific polypeptide construct contains two Fab antibody fragments, each containing a VH-CH1 (Fd) and VL-CL, that binds to a inhibitory receptor, in which one is positioned amino-terminally relative to the Fc region and the other is positioned carboxy-terminally relative to the CD3 binding region.

In some embodiments, the multispecific polypeptide construct is formed from or includes three or more polypeptides, including a first polypeptide comprising a first Fc polypeptide of a heterodimeric Fc region, a linker and a VH-CH1 (Fd) or VL-CL of a Fab antibody fragment that binds to an inhibitory receptor; a second polypeptide comprising a second Fc polypeptide of the heterodimeric Fc region, the linker and, optionally, the same VH-CH1 (Fd) or VL-CL of the Fab antibody fragment that binds to a inhibitory receptor, and a third polypeptide comprising the other of the VH-CH1 (Fd) or VL-CL of the Fab antibody fragment that binds to the inhibitory receptor. The first, second and/or third polypeptide of the multispecific polypeptide construct also can include an antigen binding domain that binds a TAA or a chain thereof as described in Section II.4. In some embodiments, the antigen binding domain that binds a TAA is a scFv or sdAb and is included as part of the first and/or second polypeptide of the multispecific polypeptide construct. In some embodiments, the antigen binding domain that binds a TAA is a Fab, and the multispecific polypeptide construct is additionally formed from a fourth polypeptide where at least a first and second polypeptide includes a chain of the Fab that binds TAA (e.g. VH-CH1 or VL-CL of a Fab) and the fourth polypeptide contains the other chain of the Fab that binds TAA (e.g. the other of VH-CH1 or VL-CL of a Fab).

In some embodiments, the IRBR, or independently each of the IRBRs, is or includes a natural (native) cognate binding partner of the inhibitory receptor (e.g. a natural ligand), or a variant thereof that exhibits binding activity to the inhibitory receptor.

In some embodiments, the one or more IRBR of the provided multispecific polypeptide constructs binds an inhibitory receptor expressed on T cells. In some embodiments, there are more than one IRBR that bind to an inhibitory receptor and each of the IRBRs, such as the first IRBR and the second IRBR, binds the same co-stimulatory receptor. In some embodiments, each of the IRBRs, such as the first IRBR and the second IRBR, binds a different inhibitory receptor. In some embodiments, each of the IRBRs, such as the first IRBR and the second IRBR binds a different epitope on the same inhibitory receptor. In some embodiments, each of the IRBRs, such as the first IRBR and the second IRBR, binds the same epitope on the same inhibitory receptor.

In some embodiments, the IRBR, or independently each of the IRBRs that bind an inhibitory receptor results in monovalent, bivalent, trivalent, or tetravalent binding to the inhibitory receptor.

In some embodiments, the inhibitory receptor is expressed on T cells, such as primary T cells of a subject. In some embodiments, the inhibitory receptor is expressed on human T cells, such as primary human T cells of a human subject.

In some embodiments, the inhibitory receptor is a member of the tumor necrosis factor (TNF) receptor family. In some embodiments, the inhibitory receptor is a member of the immunoglobulin superfamily (IgSF).

In some embodiments, the inhibitory receptor is Programmed cell death protein 1 (PD-1), cytotoxic T-lymphocyte associated protein 4 (CTLA-4), T cell immunoreceptor with Ig and ITIM domains (TIGIT), V-domain immunoglobulin suppressor of T cell activation (VISTA), T cell immunoglobulin and mucin-domain containing-3 (TIM3), or lymphocyte activation gene 3 (LAG3). In some embodiments, the one or more IRBR is an antibody or fragment thereof that binds to the inhibitor receptor PD-1, CTLA-4, TIGIT, VISTA, TIM3 or LAG3. In particular embodiments, the antibody or antigen-binding fragment is humanized or is human.

In some examples, the inhibitory receptor binding region (IRBR) binds or comprises a natural cognate binding partner of PD-1, CTLA-4, TIGIT, VISTA, or TIM3. In some embodiments, the natural cognate binding partner is selected from PD-L1, PD-L2, CD80, CD86, CD155, CD112, or VSIG-3/IGSF11, or a functional fragment thereof.

In some examples, the IRBR contains an antibody fragment, such as an scFv, that contains a variable light (VL) chain and a variable heavy (VH) chain of an antibody that that binds an inhibitory receptor, such as PD-1, CTLA-4, TIGIT, VISTA, or TIM3. In some examples, the IRBR contains a single domain antibody or a VHH domain that specifically binds an inhibitory receptor, such as a PD-1, CTLA-4, TIGIT, VISTA, or TIM3, see e.g. described in PCT publication No. WO2018068695 or WO2018068201.

In some embodiments, the inhibitory receptor is PD-1. In come embodiments, the one or more IRBR is an antibody fragment that binds to PD-1.

In some embodiments, the IRBR is or contains a VHH domain that binds PD-1 comprising a CDR1, CDR2 and CDR3 contained in a VHH amino acid sequences selected from any of SEQ ID NO: 421-426, 443, or 519-536 or an amino acid sequence that has at least 90%, 91%, 92%, 93%, 94%, 95%, 96%, 97%, 98%, or 99% sequence identity to the VHH region amino acid selected from any one of SEQ ID NOs: 421-426, 443, or 519-536 and binds PD-1.

In some embodiments, the IRBR is or contains a VHH domain that contains a CDR1, CDR2, CDR3 contained in a VHH domain set forth in SEQ ID NO: 443, or an amino acid sequence that has at least 90%, 91%, 92%, 93%, 94%, 95%, 96%, 97%, 98%, or 99% sequence identity to the VHH region amino acid selected set forth in SEQ ID NO: 443 and that binds PD-1. In some embodiments, the IRBR is or contains a VHH domain that has the amino acid sequence set forth in SEQ ID NO: 443 or an amino acid sequence that has at least 90%, 91%, 92%, 93%, 94%, 95%, 96%, 97%, 98%, or 99% sequence identity to the amino acid selected set forth in SEQ ID NO: 443 or 519 and that binds PD-1. In some embodiments, IRBR is or contains a VHH domain that is a humanized variant of the amino acid sequence set forth in SEQ ID NO: 443.

In some embodiments, an IRBR that binds PD-1 has a VHH domain that comprises a CDR1 set forth in any one of SEQ ID NOS: 438, 439 or 440, a CDR2 set forth in SEQ ID NO: 441 and a CDR3 set forth in SEQ ID NO: 442.

In some embodiments, an IRBR that binds PD-1 has a VHH domain that contains a CDR1, CDR2, and CDR3 set forth in SEQ ID NOs: 439, 441, and 442, respectively. In some embodiments, an IRBR that binds PD-1 has a VHH domain that contains a CDR1, CDR2 and CDR3 set forth in SEQ ID NOs: 438, 441, and 442, respectively. In some embodiments, the an IRBR that binds PD-1 has a VHH domain that contains a CDR1, CDR2 and CDR3 set forth in SEQ ID NOs: 440, 441, and 442, respectively.

In some aspects, the IRBR is or contains a VHH domain that contains a CDR1, CDR2 and CDR3 contained in a VHH amino acid sequence selected from any of SEQ ID NO:421-437, or an amino acid sequence that has at least 90%, 91%, 92%, 93%, 94%, 95%, 96%, 97%, 98%, or 99% sequence identity to the V_{H}H region amino acid selected from any one of SEQ ID NOs: 421-437 and that binds PD-1.

In some cases, the IRBR contains a VHH domain that is a humanized variant that has the amino acid sequence set forth in any of SEQ ID NOS: 421-437 or an amino acid sequence that has at least 90%, 91%, 92%, 93%, 94%, 95%, 96%, 97%, 98%, or 99% sequence identity to the VHH region amino acid selected from any one of SEQ ID NOs: 421-437 and that binds PD-1. In some embodimetns, the IRBR is or contains a VHH domain seqeunce that is a humanized VHH domain having the sequence of amino acids set forth in any one of SEQ ID NOS: 421-437.

In some embodiments, the IRBR is or contains a VHH domain that contains a CDR1, CDR2, CDR3 contained in a VHH domain set forth in SEQ ID NO: 519, or an amino acid sequence that has at least 90%, 91%, 92%, 93%, 94%, 95%, 96%, 97%, 98%, or 99% sequence identity to the VHH region amino acid selected set forth in SEQ ID NO: 519 and that binds PD-1. In some embodiments, the IRBR is or contains a VHH domain that has the amino acid sequence set forth in SEQ ID NO: 519 or an amino acid sequence that has at least 90%, 91%, 92%, 93%, 94%, 95%, 96%, 97%, 98%, or 99% sequence identity to the amino acid selected set forth in SEQ ID NO: 519 or 519 and that binds PD-1. In some embodiments, IRBR is or contains a VHH domain that is a humanized variant of the amino acid sequence set forth in SEQ ID NO: 519.

In some embodiments, an IRBR that binds PD-1 has a VHH domain that comprises a CDR1 set forth in any one of SEQ ID NOS: 438, 439 or 440, a CDR2 set forth in SEQ ID NO: 441 and a CDR3 set forth in SEQ ID NO: 442.

In some embodiments, an IRBR that binds PD-1 has a VHH domain that contains a CDR1, CDR2, and CDR3 set forth in SEQ ID NOs: 439, 441, and 442, respectively. In some embodiments, an IRBR that binds PD-1 has a VHH domain that contains a CDR1, CDR2 and CDR3 set forth in SEQ ID NOs: 438, 441, and 442, respectively. In some embodiments, the an IRBR that binds PD-1 has a VHH domain that contains a CDR1, CDR2 and CDR3 set forth in SEQ ID NOs: 440, 441, and 442, respectively.

In some aspects, the IRBR is or contains a VHH domain that contains a CDR1, CDR2 and CDR3 contained in a VHH amino acid sequence selected from any of SEQ ID NO: 520-536, or an amino acid sequence that has at least 90%, 91%, 92%, 93%, 94%, 95%, 96%, 97%, 98%, or 99% sequence identity to the V_{H}H region amino acid selected from any one of SEQ ID NOs: 520-536 and that binds PD-1.

In some cases, the IRBR contains a VHH domain that is a humanized variant that has the amino acid sequence set forth in any of SEQ ID NOS: 520-536 or an amino acid sequence that has at least 90%, 91%, 92%, 93%, 94%, 95%, 96%, 97%, 98%, or 99% sequence identity to the VHH region amino acid selected from any one of SEQ ID NOs: 520-536 and that binds PD-1. In some embodimetns, the IRBR is or contains a VHH domain seqeunce that is a humanized VHH domain having the sequence of amino acids set forth in any one of SEQ ID NOS: 520-536.

In some embodiments, the one or more IRBR is linked, directly or indirectly via a linker, to the Fc region and/or to the CD3 binding region. In some embodiments, linkage is via a linker. In some embodiments, the linker is a linking peptide (LP), which can include any flexible or rigid linker as described, such as in Section II.3, although generally the peptide linking the IRBR or regions is not a cleavable liker.

In some embodiments, the multispecific polypeptide construct comprises a linking peptide (LP) between the IRBR and the Fc region. In some embodiments, the multispecific polypeptide construct comprises a linking peptide (LP) between the CD3 binding region and the IRBR.

In some embodiments, the multispecific polypeptide construct comprises more than one IRBR. In some embodiments, the multispecific polypeptide construct comprises a first linking peptide (LP1) between the first IRBR and the Fc region. In some embodiments, the multispecific polypeptide construct comprises a second linking peptide (LP2) between the CD3 binding region and the second IRBR. In some embodiments, the multispecific polypeptide construct comprises a first linking peptide (LP1) between the first IRBR and the Fc region and a second linking peptide (LP2) between the CD3 binding region and the second CRBR. In some aspects, the multispecific polypeptide construct has the structural arrangement from N-terminus to C-terminus as follows: IRBR and/or antigen binding domain-LP1-Fc region-linker-CD3 binding region-LP2-IRBR and/or antigen binding domain. In some embodiments, the two linking peptides are not identical to each other.

In some embodiments, the LP (e.g., LP1 or LP2) is independently a peptide of about 1 to 20 amino acids in length. In some embodiments, the LP1 or LP2 is independently a peptide that is or comprises any Gly-Ser linker as set forth in SEQ ID NOs: 191-194, 313-319, 332, 465, or GGS.

In some embodiments, the multispecific polypeptide construct contains both a CRBR and an IRBR. In some embodiments, one of the CRBR or IRBR is positioned amino-terminally relative to the Fc region and the other of the CRBR or IRBR is positioned carboxy-terminally relative to the CD3 binding region of the multispecific polypeptide construct. In some embodiments, the CRBR and IRBR are present on different polypeptide of a heterodimeric multispecific polypeptide construct, in which at least one of the polypeptides also contains the at least one antigen binding domain specific to a TAA. In some embodiments, the CRBR and IRBR are present on the same polypeptide (first polypeptide) of a heterodimeric multispecific polypeptide construct and the at least one antigen binding domain specific to a TAA is on the other (or second) polypeptide of the heterodimeric multispecific polypeptide construct.

In some embodiments, the multispecific polypeptide construct is formed from or includes two polypeptides. In some aspects, the first polypeptide comprises in order: a first antigen binding domain specific for a TAA, a first Fc polypeptide of a heterodimeric Fc region, a linker, a VH domain of an anti-CD3 antibody or antigen binding fragment (e.g. Fv), and a second antigen binding domain specific for a TAA; and a second polypeptide comprising in order: one of the IRBR or CRBR, a second Fc polypeptide of the heterodimeric Fc region, the linker, a VL domain of the anti-CD3 antibody or antigen binding fragment (e.g. Fv) , and the other of the IRBR or CRBR. In some embodiments, the IRBR is positioned on the second polypeptide carboxy-terminally to the CD3 binding region and the CRBR is positioned on the second polypeptide amino-terminally to the Fc region. In some embodiments, the IRBR is positioned on the second polypeptide amino-terminally to the Fc region and the CRBR is positioned on the second polypeptide carboxy-terminally to the CD3 binding region. In some embodiments, the first and second antigen binding domain is specific to a TAA are the same. In some embodiments, the first and second antigen binding domain is specific to a TAA are different. In some embodiments, the first antigen binding domain and the second antigen binding domain bind a different TAA. In some embodiments, the first antigen binding domain and the second antigen binding domain bind a distinct or non-overlapping epitope of the same TAA and/or compete for binding to the same TAA.

### 3. NK Recruitment

In some embodiments, the B7H3-binding polypeptide is a bispecific construct that is or comprises at least one B7H3 VHH domain provided herein and at least one additional binding molecule capable of binding to a surface molecule expressed on a Natural Killer (NK) cells and/or recruiting NK cells. In particular aspects, the multispecific construct is bispecific for B7H3 and the NK cell surface molecule. In some embodiments, the surface molecule is CD16 (FcγRIII). Specifically, a provided bispecific B7H3-binding polypeptide is capable of specifically binding an an NK activating receptor expressed on a human NK cells cell, such as human CD16a.

CD16, a low affinity receptor for the Fc portion of some IgGs known to be involved in antibody-dependent cellular cytotoxicity (ADCC), is the best-characterized membrane receptor responsible for triggering of target cell lysis by NK cells (Mandelboim et al., 1999, PNAS 96:5640-5644). Generally, a large majority (approximately 90%) of human NK cells express CD56 at low density (CD56dim) and FcγRIII (CD16) at a high level (Cooper et al., 2001, Trends Immunol. 22:633-640). Human FcγRIII exists as two isoforms, CD16a (FcγRIIIA) and CD16b (FcγRIIIB), that share 96% sequence identity in their extracellular immunoglobulin-binding regions (van de Winkel and Capel, 1993, Immunol. Today 14(5):215-221). In particular embodiments, the additional binding molecule is capable of specifically binding CD16a.

CD16a is expressed on macrophages, mast cells, and NK cells as a transmembrane receptor. On NK cells, the alpha chain of CD16a associates with the immunoreceptor tyrosine-based activation motif (ITAM) containing FcεRI γ-chain and/or the T-cell receptor (TCR)/CD3 ζ-chain to mediate signalling (Wirthmueller et al., 1992, J. Exp. Med. 175:1381-1390). The interaction of CD16a with different combinations of homo- and hetero-dimers of the γ and ζ chains has been observed in NK cells, indicating the ability to mediate signaling via different signaling pathways via variations of the CD16a complex in NK cells (Anderson et al., 1990, PNAS 87(6):2274-2278; Ackerly et al., 1992, Int. J. Cancer Suppl. 7:11-14). FcγR-expressing effector cells have been shown to be involved in destroying tumor cells via ADCC. For example, engagement of CD16a, such as with an agonist binding molecule capable of specifically binding CD16a can result in activating of NK cells expressing CD16a, thereby eliciting a biological response, in particular a signaling response. In some cases, the binding molecule is capable of triggering cell killing, in a manner analogous to antibody-dependent cellular cytotoxicity (ADCC), by virtue of its binding to such cells.

In particular example, B7H3-binding polypeptides include bispecific molecules that can specifically bind to B7H3 and to CD16a may target NK cells to cells bearing such antigen, so that the cell bearing the antigen may be eradicated via NK cell mediated cell killing For example, a binding molecule that specifically binds B7H3 expressed on a tumor cell may target NK-cells to the tumor cell. In some cases, activation of the NK cell caused by the binding molecule binding to CD16a can lead to killing of the tumor cells.

In some embodiments, the additional binding domain specific to an activating NK cell receptor, such as CD16a, is an antigen-binding fragment selected from a Fab fragment, a F(ab′)₂ fragment, an Fv fragment, a scFv, disulfide stabilized Fv fragment (dsFv), a scAb, a dAb, a single domain heavy chain antibody (VHH), or a single domain light chain antibody. In some embodiments, the additional binding domain is monovalent for binding the activating T NK cell receptor, such as CD16a.

In some cases, the additional binding domain recognizes CD16a. In some embodiments, the anti-CD16a binding domain includes one or more copies of an anti-CD16a Fab fragment, an anti-CD16a F(ab′)₂ fragment, an anti-CD16a Fv fragment, an anti-CD16a scFv, an anti-CD16a dsFv, an anti-CD16a scAb, an anti-CD16a dAb, an anti-CD16a single domain heavy chain antibody (VHH), and an anti-CD16a single domain light chain antibody. In some embodiments, the anti-CD16a binding domain is monovalent for binding CD16a. In some embodiments, the BH73-binding polypeptide is a bispecific construct that binds BH73 and agonizes the activity of CD16a.

Antibodies and antigen-binding fragments thereof specific for CD16a are known and include, for example, NM3E2 (McCall et al. (1999) Mol. Immunol., 36:433-045. Other anti-CD16a antibodies also can be used in the constructs provided herein, including any described in published U.S. patent application No. US10160280795; U.S. Pat. No. 9,701,750; Behar et al. (2008) Protein Eng Des Sel. 21:1-10; Arndt et al., (1999) Blood 94:2562-2568. In particular examples, the anti-CD16a is an anti-CD16a scFv. In some embodiments, the anti-CD16a is an anti-CD16a antibody included in a TandAb molecule (see e.g. Reush et al. (2014) Mabs, 6:727-738). In some apects, the anti-CD16a is an anti-CD16a or antigen binding fragment, such as scFv, described in U.S. Pat. No. 9,035,026.

The provided bispecific constructs can be formatted in any of a number of formats containing the at least one B7H3 VHH domain and the at least one additional domain specific to an activating NK cell receptor, such as a CD16a-binding domain.

In one embodiment, the bispecific construct is a bispecific single-domain antibody-linked Fab (S-Fab) containing at least one B7H3 VHH domain as described linked, directly or indirectly to a Fab antigen binding fragment specific to an NK cell activating receptor, e.g. CD16a, such as an anti-CD16a Fab. In some embodiments, the B7H3 VHH domain is linked to the C-terminus of the VH or VL chain of an anti-C16a Fab. In some embodiments, the S-Fab can be further modified, such as by conjugation with polyethylene glycol (PEG), N-(2-hydroxypropyl) methacrylamide (HPMA) copolymers, proteins (such as albumin), polyglutamic acid or PASylation (Pan et al. (2018) International Jounral of Nanomedicine, 2018:3189-3201).

In another embodiment, the bispecific construct is a scFv-single domain antibody in which the construct contains at least one B7H3 VHH as described linked, directly or indirectly, to an scFv containing a VH and a VL of an antigen binding domain specific to an NK cell activating receptor, e.g. CD16a. The scFv against an NK cell activating receptor, e.g. anti-CD16a scFv, can contain any of the VH and VL sequences as described. In some embodiments, the VHH domain and the scFv are connected by a linker, such as a peptide linker. In some embodiments, the peptide linker can be a peptide linker as described herein. In some embodiments, the VHH domain and the scFv are each connected, optionally through a hinge region or a linker (e.g. peptide linker), to an Fc region, such as an N-terminus of an Fc region. The Fc region can be any described herein, such as a human Fc region or a variant thereof, e.g. a human IgG1 Fc region or a variant thereof. In particular examples, the Fc region is formed by variant Fc domains, e.g. variant human IgG1 domains, that are mutated or modified to promote heterodimerization in which different polypeptides can be dimerized to yield a heterodimer.

In a further embodiment, the antigen binding domain specific to an NK cell activating receptor, e.g. CD16a, is a single domain antibody, such as is a VHH domain that specifically binds to CD16a. Single domain antibodies, including VHH domains that bind to CD16a are known, see e.g. published U.S. patent application No. US20160280795. In such aspects, a bispecific construct provided herein can include at least one B7H3 VHH domain and at least one CD16a VHH domain. For formatting the constructs, in some cases, each VHH domain is connected, optionally through a hinge region or linker (e.g. peptide linker), to an Fc region, such as an N-terminus of an Fc region. The Fc region can be any described herein, such as a human Fc region or a variant thereof, e.g. a human IgG1 Fc region or a variant thereof. In particular examples, the Fc region is formed by variant Fc domains, e.g. variant human IgG1 domains, that are mutated or modified to promote heterodimerization in which different polypeptides can be dimerized to yield a heterodimer.

In the above embodiments, exemplary modifications of an Fc region to promote heterodimerization are known, including any as described below, e.g. Table 3. In some embodiments, one Fc polypeptide of a heterodimeric Fc comprises the sequence of amino acids set forth in any of SEQ ID NOS:293, 297, 305, 307, 445, or 451 and the other Fc polypeptide of the heterodimeric Fc contains the sequence of amino acids set forth in any of SEQ ID NOS:294, 298, 301, 303, 309, 311, 446, 449, or 453. In some embodiments, one Fc polypeptide of a heterodimeric Fc comprises the sequence of amino acids set forth in any of SEQ ID NOS: 295, 299, 306, 308, 447, or 452 and the other Fc polypeptide of the heterodimeric Fc comprises the sequence of amino acids set forth in any of SEQ ID NOS: 296, 300, 302, 304, 310, 312, 448, 450, or 454.

4. Cytokine Fusion and/or Cytokine Receptor Targeting

In some embodiments, the B7H3-binding polypeptide is a multispecific polypeptide construct that is a cytokine-antibody fusion protein (also called a B7H3 VHH-cytokine fusion). In some aspects, at least one B7H3 VHH domain provided herein is linked, directly or indirectly, to at least one cytokine, such as to an interferon. In particular embodiments, the cytokine is an interferon capable of exhibiting anti-proliferative activity, apoptotic activity and/or anti-viral activity. In some embodiments, the interferon of a B7H3 VHH-cytokine fusion provided herein is capable of binding to a receptor composed of IFNAR1 and/or 2. Any of a variety of assays can be used to assess the effect of such fusion proteins on binding IFNAR1 and/or 2, reducing or decreasing the growth rather and/or proliferation rate of a cancer cell, reducing tumor size, eliminating tumors or inducing the death of a cancer cell (e.g. via apoptosis). Such assays in include in vitro assays with various cancer cell lines known to express B7H3 or in vivo assays employing animal tumor models.

In some embodiments, the interferon is a type I interferon, such as a human type I interferon or a variant thereof. In some aspects, the human type I interferon is a variant that is a truncated human type I interferon or a human mutant type I interferon. In some embodiments, the type I interferon or variant thereof is a wild-type human IFN-alpha (IFN-alpha; alpha2 and natural higher affinity variants such as alpha 14), interferon beta (IFN-beta) as well as mutants and/or truncated forms thereof. In some embodiments, the interferon is a type II interferon, such as a human type II interferon or a variant thereof. In some aspects, the human type II interferon is a variant that is a truncated human type II interferon or a human mutant type II interferon. In some embodiments, the type II interferon or variant thereof is a wild-type human interferon gamma (IFN-gamma) as well as mutants and/or truncated forms thereof. In some embodiments, the provided cytokine-antibody fusion proteins can be used to inhibit the growth and/or proliferation of target cells (e.g. cancer cells) that express or overexpress B7H3.

In some embodiments, the B7H3 VHH-cytokine fusion protein is similar in format to any as described in International PCT published application No. WO2014194100; U.S. Pat. No. 9,803,021; Valedkarimi et al. (2017) Biomed Pharmacother., 95:731-742; or Young et al. (2014) Semin Oncol., 41:623-636.

In particular embodiments, the interferon, e.g. a type I interferon, such as a human type I interferon (e.g. IFN-alpha, IFN-beta, or IFN-gamma) is one that possesses the endogenous binding affinity and/or activity of the native or wild-type interferon, preferably at a level of at least 60%, or of at least or at least about 80%, such as at least 90%, 95%, 98%, 99%, 100%, or a level greater than the native wild-type interferon (in its isolated form).

Interferons and interferon mutants are a well known and well characterized group of cytokines (see e.g., WO 2002/095067; WO 2002/079249; WO 2002/101048; WO 2002/095067; WO 2002/083733; WO 2002/086156; WO 2002/083733; WO 2003/000896; WO 2002/101048; WO 2002/079249; WO 2003/000896; WO 2004/022593; WO2004/022747; WO 2003/023032; WO 2004/022593 and also in Kim et al. (2003) Cancer Lett. 189(2): 183-188; Hussain et a/. (2000) J. Interferon Cytokine Res. 20(9): 763-768; Hussain et al. (1998) J. Interferon Cytokine Res. 18(7): 469-477; Nyman et al. (1988) Biochem. J. 329 (Pt 2): 295-302; Golovleva et al. (1997) J. Interferon Cytokine Res. 17(10): 637-645; Hussain et al. (1997) J. Interferon Cytokine Res. 17(9): 559-566; Golovleva et al. (1997) Hum. Hered. 47(4): 185-188; Kita et al. (1991) J. Interferon Cytokine Res. 17(3): 135-140; Golovleva et al. (1996) Am. J. Hum. Genet. 59(3): 570-578; Hussain et al. (1996) J. Interferon Cytokine Res. 16(7): 523-529; Linge et al. (1995) Biochim Biophys Acta. Any of such can be used in the provided cytokine-antibody fusion proteins.

In some embodiments, the interferon is a human type I interferon. Alleles of the human interferon family of genes/proteins are known, see e.g. Pestka (10983) Arch Biochem Biophys., 221:1-37; Diaz et al. (1994) Genomics, 22:540-52; Pestka (1986) Meth. Enzymol, 199: 3-4; and Krause et al. (2000) J. Biol. Chem., 275:22995-3004.

In some embodiments, the interferon is a full-length IFN-alpha (e.g. human IFN-alpha), a full-length IFN-beta (e.g. human IFN-beta) or a full-length IFN-gamma (e.g. human IFN-gamma). In some embodiments, the interferon is a biologically active truncated IFN-alpha (e.g. human IFN-alpha), a biologically active truncated IFN-beta (e.g. human IFN-beta) or a biologically active truncated IFN-gamma (e.g. human IFN-gamma). In some embodiments, a biologically active truncated interferon contains a contiguous sequence of amino acids of a wild-type or native interferon that is truncated at the N- and/or C-terminus and comprises a length that is at least or at least about 50%, 55%, 60%, 65%, 70%, 75%, 80%, 85%, 90% or more the length of the native or wild-type interferon. Any of a variety of standard assays for assessing biological activity of an interferon can be used. For example, IFN-alpha activity can be assayed by measuring antiviral activity against a particular test virus. Kits for assaying for IFN-alpha activity are commercially available (see, e.g., ILITE™ alphabeta kit by Neutekbio, Ireland). In some aspects, the IFN-alpha is an IFN-ata (e.g. Acc. No. CAA23805), IFN-a-c (Acc. No. P01566), IFN-a-d (Acc. No. AAB59403); IFNa-5 (Acc. No. CAA26702); IFNa-6 (Acc. No. AA26704); IFNa-4 (Acc. No. NP_066546); IFNa-4b (Acc. No. CAA26701); IFNa-I (Acc. No. AAA52725); IFNa-J (Acc. No. CAA23792); IFNa-H (Acc. No. CAA23794); IFNa-F (Acc. No. AAA52718); IFNa-7 (Acc. No. CAA26903), or is a biologically active fragment thereof. In some aspects, the IFN-beta is IFN-beta set forth in Acc. No. AAC41702 or is a biologically active fragment thereof. In some aspects, the IFN-gamma is IFN-gamma set forth in Acc. No. P01579 or is a biologically active fragment thereof.

In some embodiments, a provided B7H3 VHH-cytokine fusion contains a variant or mutant interferon alpha 2 (IFNa2) is contemplated. Certain mutants include a mutation of the His at position 57, and/or the E at position 58, and/or the Q at position 61. In certain embodiments the mutants include the mutation H57Y, and/or E58N, and/or Q61S. In certain embodiments the mutants include a mutated IFNa2 having the mutations H57Y, E58N, and Q61S (YNS) (see, e.g., Kalie et al. (2007) J. Biol. Chem., 282: 11602-11611). In other embodiments mutants include a mutation of the His at position 57, and/or the E at position 58, and/or the Q at position 61 to A (alanine). In certain embodiments the mutants include a mutated IFNa2 having the mutations H57A, E58A, and Q61A (HEQ) (see, e.g., Jaitin et al. (2006) Mo/. Cellular Biol, 26(5): 1888-1897). In certain embodiments the mutant interferon comprises a mutation of His at position 57 to A, Y, or M, and/or a mutation of E at position 58 to A, or N, or D, or L, and/or a mutation of Q at position 61 to A, or S, or L, or D. [0244] In certain embodiments mutant include mutants of interferon alpha 8 (IFN-a8), such as variants with amino acid replacement corresponding to R145 to V, I, or L, and/or A146 to N, or S, and/or M149 to Y, e.g. R145V/A146N/M149Y), R145I,/A146S/M149Y or R145L/A146S/M149Y (see, e.g,. Yamamoto et.al.. (2009) J. Interferon & cytokine Res, 29: 161-170.

In some embodiments, a provided B7H3 VHH-cytokine fusion contains a mutant or variant IFN-beta containing a serine substituted for the naturally occurring cysteine at amino acid 17 (see, e.g., Hawkins et al. (1985) Cancer Res., 45, 5914-5920).

In some embodiments, a provided B7H3 VHH-cytokine fusion contains a truncated interferon. In one embodiment, a truncated interferon includes a human IFN-alpha with deletions of up to the first 15 amino-terminal amino acid residues and/or up to the last 10-13 carboxyl-terminal amino acid residues, which has been shown to retain activity of the native or wild-type human IFN-alpha (see e.g. Ackerman (1984) Proc. Natl. Acad. Sci, USA, 81 : 1045-1047). In some embodiments, a truncated human IFN-alpha has 1, 2, 3, 4, 5, 6, 7, 8, 9, 10, 11, 12 or 13 carboxyl terminal amino acid residues deleted and/or 1, 2, 3, 4, 5, 6, 7, 8, 9, 10, 11, 12, 13, 14, or 15 amino terminal amino acid residues deleted.

In some embodiments, a provided B7H3 VHH-cyokine fusion contains a truncated interferon, such as described in published U.S. patent appl. No. US2009/0025106. In some embodiments, a provided B7H3 VHH-cytokine fusion contains a truncated IFN-gamma containing N- and/or C-terminal deletions, such as described in Lundell et al. (1991) Protein Neg., 4:335-341; Pan et al. (1987 (Eur. J. Biochem., 166:145-149); WO.

In some embodiments, the interferon, e.g. human interferon, is a mutant interferon that is resistant to proteolysis compared to the unmodified, typically wild-type protein, see e.g. U.S. Pat. Nos. 7,998,469; 8,052,964; 4,832,959; 6,120,762; WO1992/008737; and EP219781.

In aspects of the provided B7H3 VHH-cytokine fusion proteins, the antibody and the cytokine, e.g. interferon, are attached directly or are attached indirectly via a linker, such as a peptide linker. The attachment can be to the N- or C-terminus of the VHH domain, so long as the attachment does not interfere with binding of the antibody to B7H3. Any linker, e.g. peptide linker, described herein can be used. In some embodiments, the linker is a GS-linker that comprises an amino acid sequence selected from the group consisting of GGSGGS, i.e., (GGS)₂ (SEQ ID NO: 191); GGSGGSGGS, i.e., (GGS)₃ (SEQ ID NO: 192); GGSGGSGGSGGS, i.e., (GGS)₄ (SEQ ID NO: 193); and GGSGGSGGSGGSGGS, i.e., (GGS)₅ (SEQ ID NO: 194). In some embodiments, the linker is a flexible linker comprising Glycine residues, such as, by way of non-limiting example, GG, GGG, GGGG (SEQ ID NO: 195), GGGGG (SEQ ID NO: 196), and GGGGGG (SEQ ID NO: 197). In some embodiments, the fusion proteins can include a combination of a GS-linker and a Glycine linker.

### D. Chimeric Receptors and Engineered Cells

Provided herein are chimeric antigen receptors (CARs) having an extracellular domain comprising one or more of the B7H3 VHH domains provided herein, such as any of the sequences of a B7H3 VHH domain provided herein. CAR constructs provided herein include an extracellular domain containing the one or more B7H3 VHH, a transmembrane domain and an intracellular signaling region. The one or more B7H3 VHH domain which form the antigen binding unit of the CAR “binds” or is “capable of binding”, i.e. targets, B7H3 with sufficient affinity such the CAR is useful in therapy in targeting a cell or tissue expressing B7H3.

CARs are synthetic receptors typically containing an extracellular targeting/binding moiety that is associated with one or more signaling domains in a single fusion molecule, and that is expressed on the surface of a cell, such as a T cell. Thus, CARs combine antigen-specificity and T cell activating properties in a single fusion molecule. First generation CARs typically included the cytoplasmic region of the CD3zeta or Fc 1 receptor y chain as their signaling domain. First generation CARs have been tested in phase I clinical studies in patients with ovarian cancer, renal cancer, lymphoma, and neuroblastoma, where they have induced modest responses (reviewed in Sadelain et al., Curr Opin Immunol, 21 (2): 215-223, 2009). Second generation CARs, which contain the signalling domains of a costimulatory molecule, such as CD28, and CD3zeta, provide dual signalling to direct combined activating and co-stimulatory signals. Third generation CARs are more complex with three or more signaling domains (reviewed in Sadelain et al., Cancer Discovery (3), 388-398, 2013 and Dotti et al, Immuno. Rev, 257 (1), 1-36, 2014).

In some embodiments, a provided CAR contains at least one antigen binding domain comprising a B7H3 VHH domain that targets or is capable of specifically binding B7H3. In some embodiments, the CAR contains at least two antigen binding domains (where at least one comprises a B7H3 VHH domain) which target one or more antigen. In one embodiment, the antigen binding domain of a CAR comprises two or at least two B7H3 VHH domains that are specific for B7H3, thus providing a bivalent binding molecule. In one embodiment, the antigen binding domain comprises two or at least two B7H3 VHH domains that are specific for B7H3, but bind to different epitopes on said antigen. In such cases, the antigen binding domain comprises a first B7H3 VHH domain that binds to a first epitope of B7H3 and a second VHH domain that binds to a second epitope of B7H3. The epitopes may be overlapping. Thus, in some embodiments, the antigen binding domain is biparatopic and the CAR is a biparatopic CAR. In yet another embodiment, the antigen binding domain comprises two B7H3 VHH domains that are specific for B7H3 and bind to the same epitopes on B7H3.

The transmembrane domain of a CAR provided herein is a domain that typically crosses or is capable of crossing or spanning the plasma membrane and is connected, directly or indirectly (e.g. via a spacer, such as an immunoglobulin hinge sequence) to the extracellular antigen binding domain and the endoplasmic portion containing the intracellular signaling domain. In one embodiment, the transmembrane domain of the CAR is a transmembrane region of a transmembrane protein (for example Type I transmembrane proteins), an artificial hydrophobic sequence or a combination thereof. In one embodiment, the transmembrane domain comprises the CD3zeta domain or CD28 transmembrane domain. Other transmembrane domains will be apparent to those of skill in the art and may be used in connection with embodiments of a CAR provided herein.

The intracellular signaling region of a CAR provided herein contains one or more intracellular signaling domain that transmits a signal to a T cell upon engagement of the antigen binding domain of the CAR, such as upon binding antigen. In some embodiments, the intracellular region contains an intracellular signaling domain that is or contains an ITAM signaling domain. Exemplary intracellular signaling domains include, for example, a signaling domain derived from ζ chain of the T-cell receptor complex or any of its homologs (e.g., η chain, FcsRIy and β chains, MB 1 (Iga) chain, B29 (Ig) chain, etc.), human CD3zeta chain, CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CDS, OX40 and CD28. In particular embodiments, the intracellular signaling region contains an intracellular signaling domain derived from the human CD3 zeta chain.

In some embodiments, the endodomain comprises at CD3-zeta signaling domain. In some embodiments, the CD3-zeta signaling domain comprises the sequence of amino acids set forth in SEQ ID NO: 281 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO:281 and retains the activity of T cell signaling.

In some embodiments, the intracellular signaling region of a CAR can further contain an intracellular signaling domain derived from a costimulatory molecule. In such examples, such a signaling domain may enhance CAR-T cell activity, such as via enhancement of proliferation, survival and/or development of memory cells, after antigen specific engagement, for example, compared to a CAR that only contains an ITAM containing signaling domain, e.g. CD3 zeta. In some embodiments, the co-stimulatory domain is a functional signaling domain obtained from a protein selected from: CD28, CD137 (4-IBB), CD134 (OX40), Dap10, CD27, CD2, CDS, ICAM-1 , LFA-1 (CD11a/CD18), Lck, TNFR-I, TNFR-II, Fas, CD30, CD40 or combinations thereof. In particular embodiments, the costimulatory signaling domain is derived or obtained from a human protein. In some aspects, the costimulatory signaling domain is derived or obtained from human CD28 or human CD137 (4-IBB).

In some embodiments, the costimulatory signaling domain is a derived from CD28 or 4-1BB and comprises the sequence of amino acids set forth in any of SEQ ID NOS: 282-285 or a sequence of amino acids that exhibits at least 85%, 86%, 87%, 88%, 89%, 90%, 91%, 92%, 93%, 94%, 95%, 96%, 97%, 98% or 99% or more sequence identity to SEQ ID NO:282-285 and retains the activity of T cell costimulatory signaling.

In particular embodiments, the CAR further comprises a hinge or spacer region which connects the extracellular antigen binding domain and the transmembrane domain. This hinge or spacer region can be used to achieve different lengths and flexibility of the resulting CAR. Examples of the a hinge or spacer region that can be used include, but are not limited to, Fc fragments of antibodies or fragments or derivatives thereof, hinge regions of antibodies, or fragments or derivatives thereof, C_{H}2 regions of antibodies, C_{H}3 regions of antibodies, artificial spacer sequences, for example peptide sequences, or combinations thereof. Other hinge or spacer region will be apparent to those of skill in the art and may be used. In one embodiment, the hinge is an lgG4 hinge or a CD8A hinge.

In some embodiments, the spacer and transmembrane domain are the hinge and transmembrane domain derived from CD8, such as having an exemplary sequence set forth in SEQ ID NO: 286-288 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:286-288.

Also provided herein is an isolated nucleic acid construct comprising at least one nucleic acid encoding a CAR as provided herein. In some aspects, the construct is an expression vector for expression of the CAR in a cell. The expression vector may be a viral vector. Viral vector technology is well known in the art and is described, for example, in Sambrook et al. (Molecular Cloning: A Laboratory Manual, Cold Spring Harbor Laboratory, New York, 2013). A number of viral based systems have been developed for gene transfer into mammalian cells. For example, retroviruses such as, adenovirus vectors are used. In one embodiment, a lentivirus vector is used.

In a further aspect, also provided is an isolated cell or cell population comprising one or more nucleic acid construct as described above. Also provided is an isolated cell or cell population that has been genetically modified to express a CAR provided herein. Thus, provided herein are genetically engineered cells which comprise, such as stably express, a CAR provided herein. In one embodiment, the cell is selected from the group consisting of a T cell, a Natural Killer (NK) cell, a cytotoxic T lymphocyte (CTL), a regulatory T cell, hematopoietic stem cells and/or pluripotent embryonic/induced stem cells. In some cases, the cell is a T cell, such as a CD4 and/or CD8 T cell. In some embodiments, the cells are autologous to the subject. For example, in some embodiments, T cells may be isolated from a patient (also called primary T cells) for engineering, e.g. transfection or transduction, with a CAR nucleic acid construct.

In an exemplary example, primary T-cells can be purified ex vivo (CD4 cells or CD8 cells or both) and stimulated with a TCR/CD28 agonists, such as anti-CD3/anti-CD28 coated beads. After a 2 or 3 day activation process, a recombinant expression vector encoding the CAR can be stably introduced into the primary T cells through standard lentiviral or retroviral transduction protocols or plasmid electroporation strategies. Cells can be monitored for CAR expression by, for example, flow cytometry using anti-epitope tag or antibodies that cross-react with native parental molecule. T-cells that express the CAR can be enriched through sorting with anti-epitope tag antibodies or enriched for high or low expression depending on the application.

The CAR engineered T-cells can be assayed for appropriate function by a variety of means. In some cases, in vitro cytotoxicity, proliferation, or cytokine assays (e.g., IFN-gamma expression) can be used to assess the function of engineered T-cells. Exemplary standard endpoints are percent lysis of a tumor line, proliferation of the engineered T-cell, or IFN-gamma protein expression in culture supernatant. In some cases, the ability to stimulate activiation of T cells upon stimulation of the CAR, e.g. via antigen, can be assessed, such as by monitoring expression of activation markers such as CD69, CD44, or CD62L, proliferation and/or cytokine production.

Also provided herein are methods for the prevention and/or treatment of a disease or condition in a subject, such as a cancer, that includes administering to a subject engineered cells comprising a CAR provided herein. Generally, the subject is in need of treatment for the disease or condition. pharmaceutically active amount of a cell and/or of a pharmaceutical composition of the invention.

### IV. POLYPEPTIDE EXPRESSION AND PRODUCTION

Nucleic acid molecules comprising polynucleotides that encode any of the provided sdAb and B7H3-binding polypeptides are provided. In some embodiments, the provided nucleic acid sequences and particularly DNA sequences encode fusion proteins as provided herein. In any of the foregoing embodiments, the nucleic acid molecule may also encode a leader sequence that directs secretion of the B7H3-binding polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the B7H3-binding polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In particular, a DNA vector that encodes a desired B7H3-binding polypeptide, such as a fusion protein, can be used to facilitate the methods of preparing the B7H3-binding polypeptides described herein and to obtain significant quantities. The DNA sequence can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used.

The disclosure also provides methods of producing a B7H3-binding polypeptide by culturing a cell under conditions that lead to expression of the polypeptide, wherein the cell comprises an isolated nucleic acid molecule encoding a B7H3-binding polypeptide described herein, and/or vectors that include these isolated nucleic acid sequences.

In some embodiments, a B7H3-binding polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the B7H3-binding polypeptides may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses an B7H3-binding polypeptide described herein is provided. The B7H3-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a B7H3-binding polypeptide that comprises an Fc region. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the B7H3-binding polypeptide is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, B7H3-binding polypeptides prepared by the methods described above are provided. In some embodiments, the B7H3-binding polypeptide is prepared in a host cell. In some embodiments, the B7H3-binding polypeptide is prepared in a cell-free system. In some embodiments, the B7H3-binding polypeptide is purified. In some embodiments, a cell culture media comprising an B7H3-binding polypeptide is provided.

In some embodiments, compositions comprising antibodies prepared by the methods described above are provided. In some embodiments, the composition comprises a B7H3-binding polypeptide prepared in a host cell. In some embodiments, the composition comprises a B7H3-binding polypeptide prepared in a cell-free system. In some embodiments, the composition comprises a purified B7H3-binding polypeptide.

### V. PHARMACEUTICAL COMPOSITIONS AND FORMULATIONS

Provided herein are pharmaceutical compositions containing any of the B7H3-binding polypeptides provided herein or engineered cells expressing the same. In some embodiments, B7H3-binding polypeptides, such as fusion proteins of the disclosure (also referred to herein as “active compounds”), and derivatives, fragments, analogs and homologs thereof, can be incorporated into pharmaceutical compositions suitable for administration. In some embodiments, engineered cells expressing a chimeric receptor, such as a chimeric antigen receptor, containing a B7H3-binding polypeptide provided herein can be incorporated into pharmaceutical compositions suitable for administration.

Such compositions typically contain a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, intratumoral, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a kit, container, pack, or dispenser together with instructions for administration. These pharmaceutical compositions can be included in diagnostic kits with instructions for use.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated. In some embodiments, the pharmaceutical composition may be administered in an amount in the range of about 50 μg/kg body weight to about 50 mg/kg body weight per dose. In some embodiments, the pharmaceutical composition may be administered in an amount in the range of about 100 μg/kg body weight to about 50 mg/kg body weight per dose. In some embodiments, the pharmaceutical composition may be administered in an amount in the range of about 100 μg/kg body weight to about 20 mg/kg body weight per dose. In some embodiments, the pharmaceutical composition may be administered in an amount in the range of about 0.5 mg/kg body weight to about 20 mg/kg body weight per dose.

In some embodiments, the pharmaceutical composition may be administered in an amount in the range of about 10 mg to about 1,000 mg per dose. In some embodiments, the pharmaceutical composition may be administered in an amount in the range of about 20 mg to about 500 mg per dose. In some embodiments, the pharmaceutical composition may be administered in an amount in the range of about 20 mg to about 300 mg per dose. In some embodiments, the pharmaceutical composition may be administered in an amount in the range of about 20 mg to about 200 mg per dose.

The pharmaceutical composition may be administered as needed to subjects. In some embodiments, an effective dose of the pharmaceutical composition is administered to a subject one or more times. In various embodiments, an effective dose of the pharmaceutical composition is administered to the subject once a month, less than once a month, such as, for example, every two months, every three months, or every six months. In other embodiments, an effective dose of the pharmaceutical composition is administered more than once a month, such as, for example, every two weeks, every week, twice per week, three times per week, daily, or multiple times per day. An effective dose of the pharmaceutical composition is administered to the subject at least once. In some embodiments, the effective dose of the pharmaceutical composition may be administered multiple times, including for periods of at least a month, at least six months, or at least a year. In some embodiments, the pharmaceutical composition is administered to a subject as-needed to alleviate one or more symptoms of a condition.

### VI. METHODS OF TREATMENT AND USES

The B7H3-binding polypeptides or engineered cells expressing the same described herein are useful in a variety of therapeutic, diagnostic and prophylactic indications. For example, the B7H3-binding polypeptides or engineered cells are useful in treating a variety of diseases and disorders in a subject. Such methods and uses include therapeutic methods and uses, for example, involving administration of the molecules or engineered cells, or compositions containing the same, to a subject having a disease, condition, or disorder, such as a tumor or cancer. In some embodiments, the molecule ore engineered cell is administered in an effective amount to effect treatment of the disease or disorder. Uses include uses of molecules containing the B7H3-binding polypeptides or engineered cells in such methods and treatments, and in the preparation of a medicament in order to carry out such therapeutic methods. In some embodiments, the methods are carried out by administering the B7H3-binding polypeptides or engineered cells, or compositions comprising the same, to the subject having or suspected of having the disease or condition. In some embodiments, the methods thereby treat the disease or condition or disorder in the subject.

In one embodiment, a B7H3-binding polypeptide or engineered cell of the disclosure may be used as therapeutic agents. Such agents will generally be employed to diagnose, prognose, monitor, treat, alleviate, and/or prevent a disease or pathology in a subject. A therapeutic regimen is carried out by identifying a subject, e.g., a human patient or other mammal suffering from (or at risk of developing) a disorder using standard methods. In some cases, a subject is selected that is known, suspected or that has been identified as having a tumor expressing B7H3. A B7H3-binding polypeptide or engineered cell is administered to the subject. A B7H3-binding polypeptide or engineered cell is administered to the subject and will generally have an effect due to its binding with the target(s).

In some embodiments, a provided B7-H3 polypeptide multi-specific polypeptide construct or engineered cell is capable of modulating, e.g. increasing, an immune response when administered to a subject, such as by engagement of CD3 and/or a CD3 signal in a cell. In some embodiments, provided herein is a method of modulating an immune response in a subject by administering a therapeutically effective amount of a provided multispecific construction or engineered cell, or pharmaceutical compositions thereof. In some embodiments, the method of modulating an immune response increases or enhances an immune response in a subject. For example, the increase or enhanced response may be an increase in cell-mediated immunity In some examples, the method increases T-cell activity, such as cytolytic T-cell (CTL) activity. In some embodiments, the modulated (e.g., increased) immune response is against a tumor or cancer.

In some embodiments, administration of a B7H3-binding polypeptide, such as an B7H3-Fc fusion protein or a multispecific construction containing an Fc region, may activate innate immune cells via engagement of FcγRs through the Fc-region of the multispecific polypeptide construct. Administration of such multispecific polypeptide constructs may agonize, stimulate, activate, and/or augment innate immune cell effector functions, including ADCC, cytokine release, degranulation and/or ADCP. In the case of a constrained multispecific polypeptide construct, administration of such multispecific polypeptide constructs may activate T-cells once the linker(s) joining the first and second component is cleaved by a protease and/or upon binding of B7H3 on a target cell (e.g. tumor cell), thereby allowing the anti-CD3 binding portion to bind CD3ε on the T cells. In some cases, administration of the multispecific polypeptide constructs may agonize, stimulate, activate, and/or augment CD3-mediated T cell activation, cytotoxicity, cytokine release and/or proliferation.

In some embodiments, the provided methods are for treating a disease or condition in a subject by administering a therapeutically effective amount of any of the provided B7H3-binding polypeptides or engineered cells or pharmaceutical compositions thereof. In some embodiments, the disease or condition is a tumor or a cancer. Generally, alleviation or treatment of a disease or disorder involves the lessening of one or more symptoms or medical problems associated with the disease or disorder. For example, in the case of cancer, the therapeutically effective amount of the drug can accomplish one or a combination of the following: reduce the number of cancer cells; reduce the tumor size; inhibit (i.e., to decrease to some extent and/or stop) cancer cell infiltration into peripheral organs; inhibit tumor metastasis; inhibit, to some extent, tumor growth; and/or relieve to some extent one or more of the symptoms associated with the cancer. In some embodiments, a composition of this disclosure can be used to prevent the onset or reoccurrence of the disease or disorder in a subject, e.g., a human or other mammal, such as a non-human primate, companion animal (e.g., cat, dog, horse), farm animal, work animal, or zoo animal. The terms subject and patient are used interchangeably herein.

In some embodiments, the B7H3-binding polypeptides or engineered cells, or pharmaceutical compositions thereof, can be used to inhibit growth of mammalian cancer cells (such as human cancer cells). A method of treating cancer can include administering an effective amount of any of the pharmaceutical compositions described herein to a subject with cancer. The effective amount of the pharmaceutical composition can be administered to inhibit, halt, or reverse progression of cancers. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient.

Non-liming examples of disease include: all types of cancers (breast, lung, colorectal, prostate, melanomas, head and neck, pancreatic, etc.), rheumatoid arthritis, Crohn's disuse, SLE, cardiovascular damage, ischemia, etc. For example, indications would include leukemias, including T-cell acute lymphoblastic leukemia (T-ALL), lymphoblastic diseases including multiple myeloma, and solid tumors, including lung, colorectal, prostate, pancreatic, and breast, including triple negative breast cancer. For example, indications include bone disease or metastasis in cancer, regardless of primary tumor origin; breast cancer, including by way of non-limiting example, ER/PR+ breast cancer, Her2+ breast cancer, triple-negative breast cancer; colorectal cancer; endometrial cancer; gastric cancer; glioblastoma; head and neck cancer, such as esophageal cancer; lung cancer, such as by way of non-limiting example, non-small cell lung cancer; multiple myeloma ovarian cancer; pancreatic cancer; prostate cancer; sarcoma, such as osteosarcoma; renal cancer, such as by way of nonlimiting example, renal cell carcinoma; and/or skin cancer, such as by way of nonlimiting example, squamous cell cancer, basal cell carcinoma, or melanoma. In some embodiments, the cancer is a squamous cell cancer. In some embodiments, the cancer is a skin squamous cell carcinoma. In some embodiments, the cancer is an esophageal squamous cell carcinoma. In some embodiments, the cancer is a head and neck squamous cell carcinoma. In some embodiments, the cancer is a lung squamous cell carcinoma.

In some embodiments, the B7H3-binding polypeptides or engineered cells, or pharmaceutical compositions thereof, or are useful in treating, alleviating a symptom of, ameliorating and/or delaying the progression of a cancer or other neoplastic condition. In some embodiments, the cancer is bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer. In certain embodiments, the cancer is a metastatic cancer, refractory cancer, or recurrent cancer.

In some embodiments, a therapeutically effective amount of a B7H3-binding polypeptide, such as a fusion protein or multispecific polypeptide construct, of the disclosure relates generally to the amount needed to achieve a therapeutic objective. Typically, precise amount of the compositions of the present disclosure to be administered can be determined by a physician with consideration of individual differences in age, weight, tumor size, extent of infection or metastasis, and condition of the patient (subject).

In some embodiments, a therapeutically effective dose may be, by way of nonlimiting example, from about 0.01 μg/kg body weight to about 10 mg/kg body weight. In some embodiments, the therapeutically effective dose may be, by way of nonlimiting example, from about 0.01 mg/kg body weight to about 5-10 mg/kg body weight. Common dosing frequencies may range, for example, from twice daily to once a week.

In some embodiments, a therapeutic amount of an engineered cell composition of the present disclosure is administered. It can generally be stated that a pharmaceutical composition comprising engineered cells, e.g., T cells, as described herein may be administered at a dosage of 10⁴ to 10⁹ cells/kg body weight, such as 10⁵ to 10⁶ cells/kg body weight, including all integer values within those ranges. Engineered cell compositions, such as T cell compositions, may also be administered multiple times at these dosages. The cells can be administered by using infusion techniques that are commonly known in immunotherapy (see, e.g., Rosenberg et al, New Eng. J. of Med. 319: 1676, 1988). The optimal dosage and treatment regime for a particular patient can readily be determined by one skilled in the art of medicine by monitoring the patient for signs of disease and adjusting the treatment accordingly.

Efficaciousness of treatment is determined in association with any known method for diagnosing or treating the particular disorder. Methods for the screening of B7H3-binding polypeptides, or engineered cells containing the same, that possess the desired specificity include, but are not limited to, enzyme linked immunosorbent assay (ELISA) and other immunologically mediated techniques known within the art. A variety of means are known for determining if administration of the provided B7H3-binding polypeptides or engineered cells sufficiently modulates immunological activity by eliminating, sequestering, or inactivating immune cells mediating or capable of mediating an undesired immune response; inducing, generating, or turning on immune cells that mediate or are capable of mediating a protective immune response; changing the physical or functional properties of immune cells; or a combination of these effects. Examples of measurements of the modulation of immunological activity include, but are not limited to, examination of the presence or absence of immune cell populations (using flow cytometry, immunohistochemistry, histology, electron microscopy, polymerase chain reaction (PCR)); measurement of the functional capacity of immune cells including ability or resistance to proliferate or divide in response to a signal (such as using T-cell proliferation assays and pepscan analysis based on 3H-thymidine incorporation following stimulation with anti-CD3 antibody, anti-T-cell receptor antibody, anti-CD28 antibody, calcium ionophores, PMA (phorbol 12-myristate 13-acetate) antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (e.g., by flow cytometry, enzyme-linked immunosorbent assays, Western blot analysis, protein microarray analysis, immunoprecipitation analysis); measurement of biochemical markers of activation of immune cells or signaling pathways within immune cells (e.g., Western blot and immunoprecipitation analysis of tyrosine, serine or threonine phosphorylation, polypeptide cleavage, and formation or dissociation of protein complexes; protein array analysis; DNA transcriptional, profiling using DNA arrays or subtractive hybridization); measurements of cell death by apoptosis, necrosis, or other mechanisms (e.g., annexin V staining, TUNEL assays, gel electrophoresis to measure DNA laddering, histology; fluorogenic caspase assays, Western blot analysis of caspase substrates); measurement of the genes, proteins, and other molecules produced by immune cells (e.g., Northern blot analysis, polymerase chain reaction, DNA microarrays, protein microarrays, 2-dimensional gel electrophoresis, Western blot analysis, enzyme linked immunosorbent assays, flow cytometry); and measurement of clinical symptoms or outcomes such as improvement of autoimmune, neurodegenerative, and other diseases involving self-proteins or self-polypeptides (clinical scores, requirements for use of additional therapies, functional status, imaging studies) for example, by measuring relapse rate or disease severity.

The provided B7H3-binding polypeptides are also useful in a variety of diagnostic and prophylactic formulations. In one embodiment, a B7H3-binding polypeptide is administered to patients that are at risk of developing one or more of the aforementioned disorders. A patient's or organ's predisposition to one or more of the disorders can be determined using genotypic, serological or biochemical markers.

In another embodiment of the disclosure, a B7H3-binding polypeptide or engineered cell is administered to human individuals diagnosed with a clinical indication associated with one or more of the aforementioned disorders. Upon diagnosis, such a therapeutic agent is administered to mitigate or reverse the effects of the clinical indication.

### Combination Therapy

B7H3-binding polypeptides or engineered cells of the present disclosure can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

In some embodiments, a B7H3-binding polypeptide provided herein is given concurrently with one or more chemotherapeutic agent, CAR-T (chimeric antigen receptor T-cell) therapy, oncolytic virus therapy, cytokine therapy, and/or agents that target other checkpoint molecules, such as VISTA, gpNMB, B7H4, HHLA2, CD73, CTLA4, TIGIT, etc.

In some embodiments, the B7H3-binding polypeptide or engineered cells of the present disclosure is used in combination with other anti-tumor agents, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib)(TARCEVA®), platelet derived growth factor inhibitors (e.g., GLEEVEC® (Imatinib Mesylate)), a COX-2 inhibitor (e.g., celecoxib), interferons, CTLA4 inhibitors (e.g., anti-CTLA antibody ipilimumab (YERVOY®)), PD-1 inhibitors (e.g., anti-PD1 antibodies, BMS-936558), PDL1 inhibitors (e.g., anti-PDL1 antibodies, MPDL3280A), PDL2 inhibitors (e.g., anti-PDL2 antibodies), cytokines, antagonists (e.g., neutralizing antibodies) that bind to one or more of the following targets ErbB2, ErbB3, ErbB4, PDGFR-beta, BlyS, APRIL, BCMA, PD-1, PDL1, PDL2, CTLA4, or VEGF receptor(s), TRAIL/Apo2, and other bioactive and organic chemical agents, etc.

In some embodiments, a B7H3-binding polypeptide or engineered cell provided herein is given concurrently with a PD-1/PD-L1 therapy. Examples of PD-1/PD-L1 therapy include nivolumab (BMS); pidilizumab (CureTech, CT-011), pembrolizumab (Merck); durvalumab (Medimmune/AstraZeneca); atezolizumab (Genentech/Roche); avelumab (Pfizer); AMP-224 (Amplimmune); BMS-936559; AMP-514 (Amplimmune); MDX-1105 (Merck); TSR-042 (Tesaro/AnaptysBio, ANB-011); STI-A1010 (Sorrento Therapeutics); STI-A1110 (Sorrento Therapeutics); and other agents that are directed against programmed death-1 (PD-1) or programmed death ligand 1 (PD-L1).

In some embodiments, the B7H3-binding polypeptide or engineered cell of the present disclosure may be used in combination with a chemotherapeutic agent. Examples of chemotherapeutic agents include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Illinois), and TAXOTERE® doxetaxel (Rhone- Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib)) (TARCEVA®)) and VEGF-A that reduce cell proliferation and pharmaceutically acceptable salts, acids or derivatives of any of the above.

Further nonlimiting exemplary chemotherapeutic agents include anti-hormonal agents that act to regulate or inhibit hormone action on cancers such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® (aldesleukin) rIL-2; LURTOTECAN® topoisomerase 1 inhibitor; ABARELIX® GnRH agoninst; and pharmaceutically acceptable salts, acids or derivatives of any of the above.

In some embodiments, the B7H3-binding polypeptide and the additional agent are formulated into a single therapeutic composition, and the B7H3-binding polypeptide and additional agent are administered simultaneously. Alternatively, the B7H3-binding polypeptide or engineered cell and the additional agent are separate from each other, e.g., each is formulated into a separate therapeutic composition, and the B7H3-binding polypeptide or engineered cell and the additional agent are administered simultaneously, or the B7H3-binding polypeptide or engineered cell and the additional agent are administered at different times during a treatment regimen. For example, the B7H3-binding polypeptide or engineered cell is administered prior to the administration of the additional agent, the B7H3-binding polypeptide or engineered cell is administered subsequent to the administration of the additional agent, or the B7H3-binding polypeptide or engineered cell and the additional agent are administered in an alternating fashion. The B7H3-binding polypeptide and additional agent may be administered in single doses or in multiple doses.

In some embodiments, the B7H3-binding polypeptide or engineered cell and the additional agent(s) are administered simultaneously. For example, the B7H3-binding polypeptide and the additional agent(s) can be formulated in a single composition or administered as two or more separate compositions. In some embodiments, the B7H3-binding polypeptide or engineered cell and the additional agent(s) are administered sequentially, or the B7H3-binding polypeptide or engineered cell and the additional agent are administered at different times during a treatment regimen.

### VII. EXEMPLARY EMBODIMENTS

Among the provided embodiments are:

1. A B7H3-binding polypeptide construct, comprising at least one heavy chain only variable domain (B7H3 VHH domain) that specifically binds B7H3 and one or more additional binding domain that binds to a target other than B7H3.

2. The B7H3-binding polypeptide construct of embodiment 1, wherein the at least one B7H3 VHH domain comprises a complementarity determining region 1 (CDR1) comprising an amino acid sequence selected from the group consisting of SEQ ID NO: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and 145; a complementarity determining region 2 (CDR2) comprising an amino acid sequence selected from the group consisting of SEQ ID NO: 146, 147, 148, 149, 150, 151, 152, 153, 154, 155, 156, 157, 158, 159, 160, 161, 162, 163, 164, 165, 166 and 167; and a complementarity determining region 3 (CDR3) comprising an amino acid sequence selected from the group consisting of SEQ ID NO: 168, 169, 170, 171, 172, 173, 174, 175, 176, 177, 178, 179, 180, 181, 182, 183, 184, 185, 186, 187, 188, 189, and 483-488.

3. A B7H3-binding polypeptide construct, comprising at least one heavy chain only variable domain (B7H3 VHH domain) comprising a complementarity determining region 1 (CDR1) comprising an amino acid sequence selected from the group consisting of SEQ ID NO: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and 145; a complementarity determining region 2 (CDR2) comprising an amino acid sequence selected from the group consisting of SEQ ID NO: 146, 147, 148, 149, 150, 151, 152, 153, 154, 155, 156, 157, 158, 159, 160, 161, 162, 163, 164, 165, 166 and 167; and a complementarity determining region 3 (CDR3) comprising an amino acid sequence selected from the group consisting of SEQ ID NO: 168, 169, 170, 171, 172, 173, 174, 175, 176, 177, 178, 179, 180, 181, 182, 183, 184, 185, 186, 187, 188, 189, and 483-488.

4. The B7H3-binding polypeptide construct of any of embodiments 1-3, wherein the B7H3 is a human B7H3.

5. The B7H3-binding polypeptide construct of any of embodiments 1-4, wherein the at least one B7H3 VHH domain is humanized.

6. The B7H3-binding polypeptide construct of any of embodiments 1, 2, 4 and 5, wherein the one or more additional binding domains binds to an activating receptor on an immune cell.

7. The B7H3-binding polypeptide construct of embodiment 6, wherein the immune cell is a T cell.

8. The B7H3-binding polypeptide construct of embodiment 6 or embodiment 7, wherein the activating receptor is CD3 (CD3ε).

9. The B7H3-binding polypeptide construct of embodiment 8 that is bispecific for B7H3 and CD3.

10. The B7H3-binding polypeptide construct of embodiment 9, wherein the immune cell is a Natural Killer (NK) cell.

11. The B7H3-binding polypeptide construct of embodiment 6 or embodiment 10, wherein the activating receptor is CD16 (CD16a).

12. The B7H3-binding polypeptide construct of embodiment 11 that is bispecific for B7H3 and CD16a.

13. The B7H3-binding polypeptide construct of any of embodiments 1, 2, 4 and 5, wherein the one or more additional binding domain binds to a cytokine receptor.

14. The B7H3-binding polypeptide construct of any of embodiments 1, 2 and 4-13, wherein the one or more additional binding domain comprises an antibody or antigen-binding fragment thereof.

15. The B7H3-binding polypeptide construct of any of embodiments 1, 2 and 4-14, wherein the one or more additional binding domain is monovalent.

16. The B7H3-binding polypeptide construct of embodiment 14 or embodiment 15, wherein the antibody or antigen-binding fragment thereof is an Fv, a disulfide-stabilized Fv (dsFv), scFv, a Fab, a single domain antibody (sdAb), a VNAR, or a VHH.

17. The B7H3-binding polypeptide construct of embodiment 13, wherein the one or more additional binding domain is a cytokine or is a truncated fragment or variant thereof capable of binding to the cytokine receptor.

18. The B7H3-binding polypeptide construct of embodiment 17, wherein the cytokine is an interferon, or is a truncated fragment or variant of an interferon.

19. The B7H3-binding polypeptide construct of embodiment 18, wherein the interferon is a type I interferon, a type II interferon, a truncated fragment or variant of a type I interferon, or a truncated fragment of variant of a type II interferon.

20. The B7H3-binding polypeptide construct of embodiment 19, wherein the interferon is selected from: a type I interferon that is an IFN-alpha or an IFN-beta, or is a truncated fragment or variant thereof; or a type II interferon that is an or IFN-gamma or a truncated fragment or variant thereof.

21. The B7H3-binding polypeptide construct of any of embodiments 1-20, wherein the polypeptide construct comprises an immunoglobulin Fc region.

22. The B7H3-binding polypeptide construct of any of embodiments 1, 2 and 4-21, wherein the polypeptide construct comprises an immunoglobulin Fc region that links the at least one B7H3 VHH domain and the one or more additional binding domain.

23. The B7H3-binding polypeptide construct of any of embodiments 1-22 that is a dimer.

24. The B7H3-binding polypeptide construct of any of embodiments 21-23, wherein the Fc region is a homodimeric Fc region.

25. The B7H3-binding polypeptide construct of any of embodiments 21-24, wherein the Fc region comprises the sequence of amino acids set forth in any of SEQ ID NOS: 198, 200, 201, 202 or 203, or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 198, 200, 201, 202 or 203.

26. The B7H3-binding polypeptide construct of any of embodiments 21-24, wherein the Fc region is a human IgG1.

27. The B7H3-binding polypeptide construct of embodiment 26, wherein the Fc region comprises the sequence of amino acids set forth in SEQ ID NO:198 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 198.

28. The B7H3-binding polypeptide construct of any of embodiments 21-23, wherein the Fc region is a heterodimeric Fc region.

29. The B7H3-binding polypeptide construct of any of embodiments 21-28, wherein the Fc region exhibits effector function.

30. The B7H3-binding polypeptide construct of any of embodiments 21-29, wherein the Fc region comprises a polypeptide comprising one or more amino acid modification that reduces effector function and/or reduces binding to an effector molecule selected from an Fc gamma receptor or C1q.

31. The B7H3-binding polypeptide construct of embodiment 30, wherein the one or more amino acid modification is deletion of one or more of Glu233, Leu234 or Leu235.

32. The B7H3-binding polypeptide construct of embodiment 30 or embodiment 31, wherein the Fc region comprises the sequence of amino acids set forth in SEQ ID NO:199 or a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 199.

33. The B7H3-binding polypeptide construct of any of embodiments 1-32, wherein the at least one B7H3 VHH domain comprises the VHH domain sequence set forth in any of SEQ ID NOS: 1, 8-35, 40, 41, 44, 56-110, 466, 467, 489, 490, or 492-518 or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 1, 8-35, 40, 41, 44, 56-110, 466, 467, 489, 490, or 492-518 and binds B7H3.

34. The B7H3-binding polypeptide construct of any of embodiments 1-33, wherein the at least one B7H3 VHH domain comprises the sequence set forth in (i) SEQ ID NO:1, (ii) a humanized variant of SEQ ID NO:1, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:1, and binds B7H3.

35. The B7H3-binding polypeptide construct of any of embodiments 1-34, wherein the at least one B7H3 VHH domain comprises a CDR1 comprising an amino acid sequence selected from the group consisting of SEQ ID NO: 115, 116, 117, 118, 119, 120 and 121; a CDR2 comprising an amino acid sequence selected from the group consisting of SEQ ID NO: 146, 147, 148, 149, 150 and 151; and a CDR3 comprising an amino acid sequence selected from the group consisting of SEQ ID NO: 168 and 169.

36. The B7H3-binding polypeptide construct of any of embodiments 1-35, wherein the at least one B7H3 VHH domain comprises a CDR1, CDR2 and CDR3 set forth in SEQ ID NOS: 115, 146 and 168, respectively; SEQ ID NOS: 115, 147 and 168, respectively; SEQ ID NOS: 115, 148 and 168, respectively; SEQ ID NOS: 115, 149 and 168, respectively; SEQ ID NOS: 115, 150 and 168, respectively; SEQ ID NOS: 116, 146 and 168, respectively; SEQ ID NOS: 117, 146 and 168, respectively; SEQ ID NOS: 118, 146 and 168, respectively; SEQ ID NOS: 115, 146 and 169, respectively; SEQ ID NOS: 119, 146 and 168, respectively; SEQ ID NOS: 120, 146 and 168, respectively; SEQ ID NOS: 115, 151 and 168, respectively; SEQ ID NOS: 116, 147 and 168, respectively; SEQ ID NOS: 118, 147 and 168, respectively; SEQ ID NOS: 119, 147 and 168, respectively; SEQ ID NOS: 116, 151 and 168, respectively; SEQ ID NOS: 115, 146 and 168, respectively; SEQ ID NOS: 121, 147 and 168, respectively; SEQ ID NOS: 115, 146 and 168, respectively; SEQ ID NOs: 119, 149 and 168, respectively; or SEQ ID NOS: 122, 151 and 168, respectively.

37. The B7H3-binding polypeptide construct of any of embodiments 1-36, wherein the at least one B7H3 VHH domain comprises the sequence of amino acids set forth in any one of SEQ ID NOs:8-34, 467, 489-490, and 492-497 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 8-34, 467, 489-490, and 492-497, and binds B7H3.

38. The B7H3-binding polypeptide construct of embodiments 1-37, wherein the at least one B7H3 VHH domain comprises the sequence of amino acids set forth in any one of SEQ ID NOS: 8-34, 467, 489-490, and 492-497.

39. The B7H3-binding polypeptide construct of any of embodiments 1-33, wherein the at least one B7H3 VHH domain comprises the sequence set forth in (i) SEQ ID NO:35, (ii) a humanized variant of SEQ ID NO:35, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:35, and binds B7H3.

40. The B7H3-binding polypeptide of any of embodiments 1-33 and 39, wherein the at least one B7H3 VHH domain comprises a CDR1 comprising an amino acid sequence set forth in SEQ ID NO: 123; a CDR2 comprising an amino acid sequence selected from the group consisting of SEQ ID NO: 152 and 153; and a CDR3 comprising an amino acid sequence selected from the group consisting of SEQ ID NO: 170 and 171.

41. The B7H3-binding polypeptide construct of any of embodiments 1-33, 39 and 40, wherein the at least one B7H3 VHH domain comprises a CDR1, CDR2 and CDR3 set forth in SEQ ID NOS: 123, 152 and 170, respectively; SEQ ID NOS: 123, 152 and 171, respectively; SEQ ID NOS: 123, 153 and 170, respectively; or SEQ ID NOS: 123, 153 and 171, respectively.

42. The B7H3-binding polypeptide construct of any of embodiments 1-33 and 39-41, wherein the at least one B7H3 VHH domain comprises the sequence of amino acids set forth in any one of SEQ ID NOs:40, 41, or 498-503 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 40, 41, or 498-503, and binds B7H3.

43. The B7H3-binding polypeptide construct of embodiments 1-33 and 39-42, wherein the at least one B7H3 VHH domain comprises the sequence of amino acids set forth in any one of SEQ ID NOS: 40, 41, or 498-503.

44. The B7H3-binding polypeptide construct of any of embodiments 1-33, wherein the at least one B7H3 VHH domain comprises the sequence set forth in (i) SEQ ID NO:44 (ii) a humanized variant of SEQ ID NO:44, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:44, and binds B7H3.

45. The B7H3-binding polypeptide construct of any of embodiments 1-33 and 44, wherein the at least one B7H3 VHH domain comprises a CDR1 comprising an amino acid sequence selected from the group consisting of SEQ ID NO: 124, 125, 126, 127, 128, 129, 130, 131, 132, or 133; a CDR2 comprising an amino acid sequence set forth in SEQ ID NO: 154; and a CDR3 comprising an amino acid sequence selected from the group consisting of SEQ ID NO: 172, 173, 174, 175, 176, 177, 178, 179, 180, 181, 182 and 183.

46. The B7H3-binding polypeptide construct of any of embodiments 1-33, 44 and 45, wherein the at least one B7H3 VHH domain comprises a CDR1, CDR2 and CDR3 set forth in SEQ ID NOS: 124, 154 and 172, respectively; SEQ ID NOS: 124, 154 and 173, respectively; SEQ ID NOS: 124, 154 and 174, respectively; SEQ ID NOS: 124, 154 and 175, respectively; SEQ ID NOS: 125, 154 and 173, respectively; SEQ ID NOS: 126, 154 and 173, respectively; SEQ ID NOS: 127, 154 and 173, respectively; SEQ ID NOS: 128, 154 and 173, respectively; SEQ ID NOS: 129, 154 and 173, respectively; SEQ ID NOS: 130, 154 and 173, respectively; SEQ ID NOS: 131, 154 and 173, respectively; SEQ ID NOS: 124, 154 and 176, respectively; SEQ ID NOS: 124, 154 and 177, respectively; SEQ ID NOS: 124, 154 and 178, respectively; SEQ ID NOS: 124, 154 and 179, respectively; SEQ ID NOS: 124, 154 and 180, respectively; SEQ ID NOS: 124, 154 and 181, respectively; SEQ ID NOS: 124, 154 and 182, respectively; SEQ ID NOS: 124, 154 and 183, respectively; SEQ ID NOS: 126, 154 and 176, respectively; SEQ ID NOS: 124, 154 and 179, respectively; SEQ ID NOS: 124, 154 and 182, respectively; SEQ ID NOS: 132, 154 and 176, respectively; or SEQ ID NOS: 133, 154 and 173, respectively.

47. The B7H3-binding polypeptide construct of any of embodiments 1-33 and 44-46, wherein the at least one B7H3 VHH domain comprises the sequence of amino acids set forth in any one of SEQ ID NOs:56-91, 466, and 504-514 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 56-91, 466, and 504-514, and binds B7H3.

48. The B7H3-binding polypeptide construct of embodiments 1-33 and 44-47, wherein the at least one B7H3 VHH domain comprises the sequence of amino acids set forth in any one of SEQ ID NOS: 56-91, 466, and 504-514.

49. The B7H3-binding polypeptide construct of any of embodiments 1-33, wherein the at least one B7H3 VHH domain comprises the sequence set forth in (i) SEQ ID NO:105 (ii) a humanized variant of SEQ ID NO:105, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:105, and binds B7H3.

50. The B7H3-binding polypeptide construct of any of embodiments 1-33 and 49, wherein the at least one B7H3 VHH domain comprises a CDR1 comprising an amino acid sequence set forth in SEQ ID NO: 145; a CDR2 comprising an amino acid sequence set forth in SEQ ID NO: 167; and a CDR3 comprising an amino acid sequence set forth in SEQ ID NO: 488.

51. The B7H3-binding polypeptide construct of any of embodiments 1-33, 49 and 50, wherein the at least one B7H3 VHH domain comprises the sequence of amino acids set forth in any one of SEQ ID NOs:106-109 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:106-109, and binds B7H3.

52. The B7H3-binding polypeptide construct of embodiments 1-33 and 49-51, wherein the at least one B7H3 VHH domain comprises the sequence of amino acids set forth in any one of SEQ ID NOS: 106-109.

53. The B7H3-binding polypeptide construct of any of embodiments 1-33, wherein the at least one B7H3 VHH domain comprises the sequence set forth in (i) SEQ ID NO:110 (ii) a humanized variant of SEQ ID NO:110, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:110, and binds B7H3.

54. The B7H3-binding polypeptide of any of embodiments 1-33 and 53, wherein the at least one B7H3 VHH domain comprises a CDR1 comprising an amino acid sequence set forth in SEQ ID NO: 139; a CDR2 comprising an amino acid sequence set forth in SEQ ID NO: 161; and a CDR3 comprising an amino acid sequence set forth in SEQ ID NO: 189.

55. The B7H3-binding polypeptide construct of any of embodiments 1-33, 53 and 54, wherein the at least one B7H3 VHH domain comprises the sequence of amino acids set forth in any one of SEQ ID NOs:515-518 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 515-518, and binds B7H3.

56. The B7H3-binding polypeptide construct of embodiments 1-33 and 53-55, wherein the at least one B7H3 VHH domain comprises the sequence of amino acids set forth in any one of SEQ ID NOS: 515-518.

57. The B7H3-binding polypeptide construct of any of embodiments 1-33, wherein the at least one B7H3 VHH domain comprises the sequence set forth in (i) SEQ ID NO:92, 93, 94, 95, 96, 97, 98, 99, 100, 101, 102, 103 or 104 (ii) a humanized variant of SEQ ID NO: 92, 93, 94, 95, 96, 97, 98, 99, 100, 101, 102, 103 or 104, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 92, 93, 94, 95, 96, 97, 98, 99, 100, 101, 102, 103 or 104, and binds B7H3.

58. The B7H3-binding polypeptide construct of any of embodiments 1-33 and 57, wherein the at least one B7H3 VHH domain comprises a CDR1, CDR2 and CDR3 set forth in SEQ ID NOS:134, 155 and 184, respectively; 135, 156 and 168, respectively; 136, 157 and 185, respectively; 137, 158 and 186, respectively; 138, 159 and 187, respectively; 138, 160 and 188, respectively; 139, 161 and 189, respectively; 140, 162 and 483, respectively; 141, 163 and 484, respectively; 139, 161 and 189, respectively; 142, 164 and 485, respectively; 143, 165 and 486, respectively; 144, 166 and 487, respectively.

59. The B7H3-binding polypeptide construct of any of embodiments 1-33, 57 and 58, wherein the at least one B7H3 VHH domain is set forth in SEQ ID NO: 92, 93, 94, 95, 96, 97, 98, 99, 100, 101, 102, 103 or 104.

60. A multispecific polypeptide construct, comprising: (a) a first component comprising a heterodimeric Fc region comprising a first Fc polypeptide and a second Fc polypeptide and (b) a second component comprising an anti-CD3 antibody or antigen-binding fragment comprising a variable heavy chain region (VH) and a variable light chain region (VL), wherein: the VH and VL that comprise the anti-CD3 antibody or antigen binding fragment are linked to opposite polypeptides of the heterodimeric Fc; the first and second components are coupled by a linker, wherein the heterodimeric Fc region is positioned N-terminal to the anti-CD3 antibody; and one or both of the first and second components comprises at least one antigen binding domain comprising a single domain antibody that specifically binds B7H3 (B7H3 VHH domain).

61. The multispecific polypeptide construct of embodiment 60, wherein the multispecific polypeptide construct comprises at least (i) a first polypeptide comprising the first Fc polypeptide of the heterodimeric Fc region, the linker and the VH or VL domain of the anti-CD3 antibody or antigen binding fragment; and (ii) a second polypeptide comprising the second Fc polypeptide of the heterodimeric Fc region, the linker, optionally the same linker as present in the first polypeptide, and the other of the VH or VL domain of the anti-CD3 antibody or antigen binding fragment, wherein one or both of the first and second polypeptide comprise the at least one B7H3 VHH domain.

62. The multispecific polypeptide construct of embodiment 60 or embodiment 61, wherein one or both of the first and second Fc polypeptides of the heterodimeric Fc region comprises at least one modification to induce heterodimerization compared to a polypeptide of a homodimeric Fc region, optionally compared to the Fc polypeptide set forth in SEQ ID NO:198 or an immunologically active fragment thereof.

63. The multispecific polypeptide construct of embodiment 62, wherein each of the first and second Fc polypeptides of the heterodimeric Fc region independently comprise at least one amino acid modification.

64. The multispecific polypeptide construct of embodiment 63, wherein each of the first and second Fc polypeptides of the heterodimeric Fc region comprise a knob-into-hole modification or comprise a charge mutation to increase electrostatic complementarity of the polypeptides.

65. The multispecific polypeptide construct of embodiment 64, wherein the amino acid modification is a knob-into-hole modification.

66. The multispecific polypeptide construct of any of embodiments 60-65, wherein the first Fc polypeptide of the heterodimeric Fc region comprises the modification selected from among Thr366Ser, Leu368Ala, Tyr407Val, and combinations thereof and the second Fc polypeptide of the heterodimeric Fc region comprises the modification Thr366Trp.

67. The multispecific polypeptide of embodiment 66, wherein the first and second Fc polypeptides further comprises a modification of a non-cysteine residue to a cysteine residue, wherein the modification of the first polypeptide is at one of the position Ser354 and Tyr349 and the modification of the second Fc polypeptide is at the other of the position Ser354 and Tyr349.

68. The multispecific polypeptide construct of any of embodiments 62-64, wherein the amino acid modification is a charge mutation to increase electrostatic complementarity of the polypeptides.

69. The multispecific polypeptide construct of any of embodiments 60-64 and 68, wherein the first and/or second Fc polypeptides or each of the first and second Fc polypeptide comprise a modification in complementary positions, wherein the modification is replacement with an amino acid having an opposite charge to the complementary amino acid of the other polypeptide.

70. The multispecific polypeptide construct of any of embodiments 60-69, wherein one of the first or second Fc polypeptide of the heterodimeric Fc region further comprises a modification at residue Ile253.

71. The multispecific polypeptide construct of embodiment 70, wherein the modification is Ile253Arg.

72. The multispecific polypeptide construct of any of embodiments 60-71, wherein one of the first or second Fc polypeptide of the heterodimeric Fc region further comprises a modification at residue His435.

73. The multispecific polypeptide construct of embodiment 72, wherein the modification is His435Arg.

74. The multispecific polypeptide construct of any of embodiments 60-73, wherein the Fc region comprises a polypeptide that lacks Lys447.

75. The multispecific polypeptide construct of any of embodiments 60-74, wherein the Fc region comprises a polypeptide comprising at least one modification to enhance FcRn binding.

76. The multispecific polypeptide construct of embodiment 75, wherein the modification is at one or more position selected from the group consisting of Met252, Ser254, Thr256, Met428, Asn434, and combinations thereof.

77. The multispecific polypeptide construct of embodiment 76, wherein the modification is selected from the group consisting of Met252Y, Ser254T, Thr256E, Met428L, Met428V, Asn434S, and combinations thereof.

78. The multispecific polypeptide construct of embodiment 75 or embodiment 76, wherein the modification is at position Met252 and at position Met428.

79. The multispecific polypeptide construct of embodiment 78, wherein the modification is Met252Y and Met428L.

80. The multispecific polypeptide construct of embodiment 78, wherein the modification is Met252Y and Met428V.

81. The multispecific polypeptide construct of any of embodiments 60-80, wherein the first Fc polypeptide of the heterodimeric Fc region comprises the sequence of amino acids set forth in any of SEQ ID NOS:293, 297, 305 or 307, and the second Fc polypeptide of the heterodimeric Fc region comprises the sequence of amino acids set forth in any of SEQ ID NOS:294, 298, 301, 303, 309 or 311.

82. The multispecific polypeptide construct of any of embodiments 21-81, wherein the Fc region comprises a polypeptide comprising at least one amino acid modification that reduces effector function and/or reduces binding to an effector molecule selected from an Fc gamma receptor or C1q.

83. The multispecific polypeptide construct of embodiment 82, wherein the at least one amino acid modification is deletion of one or more of Glu233, Leu234 or Leu235.

84. The multispecific polypeptide construct of any of embodiments 60-83, wherein the first Fc polypeptide of the heterodimeric Fc region comprises the sequence of amino acids set forth in any of SEQ ID NOS: 295, 299, 306 or 308 and the second Fc polypeptide of the heterodimeric Fc region comprises the sequence of amino acids set forth in any of SEQ ID NOS: 296, 300, 302, 304, 310 or 312.

85. The multispecific polypeptide construct of any of embodiment 60-84, wherein the anti-CD3 antibody or antigen binding fragment is monovalent.

86. The multispecific polypeptide construct of any of embodiments 60-85, wherein the anti-CD3 antibody or antigen binding fragment is not a single chain antibody, optionally is not a single chain variable fragment (scFv).

87. The multispecific polypeptide construct of any of embodiments 60-86, wherein the anti-CD3 antibody or antigen binding fragment is an Fv antibody fragment.

88. The multispecific polypeptide construct of embodiment 87, wherein the Fv antibody fragment comprises a disulfide stabilized anti-CD3 binding Fv fragment (dsFv).

89. The multispecific polypeptide construct of 60-88, wherein the anti-CD3 antibody or antigen-binding fragment comprises a VH CDR1 comprising the amino acid sequence TYAMN (SEQ ID NO: 219); a VH CDR2 comprising the amino acid sequence RIRSKYNNYATYYADSVKD (SEQ ID NO: 220); a VH CDR3 comprising the amino acid sequence HGNFGNSYVSWFAY (SEQ ID NO: 221), a VL CDR1 comprising the amino acid sequence RSSTGAVTTSNYAN (SEQ ID NO: 222); a VL CDR2 comprising the amino acid sequence GTNKRAP (SEQ ID NO: 223); and a VL CDR3 comprising the amino acid sequence ALWYSNLWV (SEQ ID NO: 224).

90. The multispecific polypeptide construct of any of embodiments 60-89, wherein the anti-CD3 antibody or antigen-binding fragment comprises: a VH having the amino acid sequence of any of SEQ ID NOS: 225-255, 480, 460, or 462 or a sequence that exhibits at least 90%, 91%, 92%, 93%, 94%, 95%, 96%, 97%, 98% or 99% sequence identity to any of SEQ ID NOS: 225-255,460, or 462 and binds CD3; and a VL having the amino acid sequence of any of SEQ ID NOS: 256-274, 417,459, or 461 or a sequence that exhibits at least 90%, 91%, 92%, 93%, 94%, 95%, 96%, 97%, 98% or 99% sequence identity to any of SEQ ID NOS: 256-274, 417,459, or 461 and binds CD3.

91. The multispecific polypeptide construct of any of embodiments 60-90, wherein the anti-CD3 antibody or antigen-binding fragment comprises the amino acid sequence of SEQ ID NO: 237 and the amino acid sequence of SEQ ID NO: 265.

92. The multispecific polypeptide construct of any of embodiments 60-90, wherein the anti-CD3 antibody or antigen-binding fragment comprises the amino acid sequence of SEQ ID NO: 237 and the amino acid sequence of SEQ ID NO: 417.

93. The multispecific polypeptide construct of any of embodiments 60-90, wherein the anti-CD3 antibody or antigen-binding fragment comprises the amino acid sequence of SEQ ID NO: 460 and the amino acid sequence of SEQ ID NO: 461.

94. The multispecific polypeptide construct of any of embodiments 60-90, wherein the anti-CD3 antibody or antigen-binding fragment comprises the amino acid sequence of SEQ ID NO: 480 and the amino acid sequence of SEQ ID NO: 459.

95. The multispecific polypeptide construct of any of embodiment 60-94, wherein the at least one B7H3 single domain antibody is positioned amino-terminally relative to the Fc region and/or carboxy-terminally relative to the CD3 binding region of the multispecific polypeptide construct.

96. The multispecific polypeptide construct of any of embodiments 60-95, wherein the multispecific polypeptide construct comprises a first B7H3 VHH domain that specifically bind B7H3 and a second B7H3 VHH domain that specifically binds B7H3.

97. The multispecific polypeptide construct of embodiment 96, wherein the first or second B7H3 VHH domain is positioned amino-terminally relative to the Fc region of the multispecific construct and the other of the first or second B7H3 VHH domain is positioned carboxy-terminally relative to the CD3 binding region of the multispecific construct.

98. The multispecific polypeptide construct of embodiment 96 or embodiment 97, wherein the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second component comprises in order of N-terminus to C-terminus the second Fc polypeptide of the heterodimeric Fc region, the linker, optionally the same linker as present in the first component, and the other of the VH or VL domain of the anti-CD3 antibody or antigen binding fragment.

99. The multispecific polypeptide construct of any of embodiments 96-98, wherein the first and second B7H3 VHH domain are the same.

100. The multispecific polypeptide construct of any of embodiments 96-98, wherein the first and second B7H3 VHH domain are different.

101. The multispecific polypeptide construct of embodiment 100, wherein the first and second B7H3 VHH domain bind a distinct or non-overlapping epitope of B7H3 and/or do not compete for binding to B7H3.

102. The multispecific polypeptide construct of any of embodiments 60-101, wherein the at least one B7H3 VHH domain, or each of the first and second B7H3 VHH domain, independently comprises the VHH domain sequence set forth in any of SEQ ID NOS: 1, 8-35, 40, 41, 44, 56-110, 466, 467, 489, 490, or 492-518 or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 1, 8-35, 40, 41, 44, 56-110, 466, 467, 489, 490, or 492-518 and binds B7H3.

103. The multispecific polypeptide construct of any of embodiments 60-102, wherein the at least one B7H3 VHH domain, or each of the first and second B7H3 VHH domain, independently comprises the sequence set forth in (i) SEQ ID NO:1, (ii) a humanized variant of SEQ ID NO:1, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:1, and binds B7H3.

104. The multispecific polypeptide construct of any of embodiments 60-103, wherein the at least one B7H3 VHH domain, or each of the first and second B7H3 VHH domain, independently comprises a CDR1 comprising an amino acid sequence selected from the group consisting of SEQ ID NO: 115, 116, 117, 118, 119, 120 and 121; a CDR2 comprising an amino acid sequence selected from the group consisting of SEQ ID NO: 146, 147, 148, 149, 150 and 151; and a CDR3 comprising an amino acid sequence selected from the group consisting of SEQ ID NO: 168 and 169.

105. The multispecific polypeptide construct of any of embodiments 60-104, wherein the at least one B7H3 VHH domain, or each of the first and second B7H3 VHH domain, independently comprises a CDR1, CDR2 and CDR3 set forth in SEQ ID NOS: 115, 146 and 168, respectively; SEQ ID NOS: 115, 147 and 168, respectively; SEQ ID NOS: 115, 148 and 168, respectively; SEQ ID NOS: 115, 149 and 168, respectively; SEQ ID NOS: 115, 150 and 168, respectively; SEQ ID NOS: 116, 146 and 168, respectively; SEQ ID NOS: 117, 146 and 168, respectively; SEQ ID NOS: 118, 146 and 168, respectively; SEQ ID NOS: 115, 146 and 169, respectively; SEQ ID NOS: 119, 146 and 168, respectively; SEQ ID NOS: 120, 146 and 168, respectively; SEQ ID NOS: 115, 151 and 168, respectively; SEQ ID NOS: 116, 147 and 168, respectively; SEQ ID NOS: 118, 147 and 168, respectively; SEQ ID NOS: 119, 147 and 168, respectively; SEQ ID NOS: 116, 151 and 168, respectively; SEQ ID NOS: 115, 146 and 168, respectively; SEQ ID NOS: 121, 147 and 168, respectively; SEQ ID NOS: 115, 146 and 168, respectively; SEQ ID NOs: 119, 149 and 168, respectively; or SEQ ID NOS: 122, 151 and 168, respectively.

106. The multispecific polypeptide construct of any of embodiments 60-105, wherein the at least one B7H3 VHH domain, or each of the first and second B7H3 VHH domain, independently comprises the sequence of amino acids set forth in any one of SEQ ID NOs: 8-34, 467, 489-490, and 492-497 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 8-34, 467, 489-490, and 492-497, and binds B7H3.

107. The multispecific polypeptide construct of any of embodiments 60-106, wherein the at least one B7H3 VHH domain, or each of the first and second B7H3 VHH domain, independently comprises the sequence of amino acids set forth in any one of SEQ ID NOS: 8-34, 467, 489-490, and 492-497.

108. The multispecific polypeptide construct of any of embodiments 60-102, wherein the at least one B7H3 VHH domain, or each of the first and second B7H3 VHH domain, independently comprises the sequence set forth in (i) SEQ ID NO:35, (ii) a humanized variant of SEQ ID NO:35, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:35, and binds B7H3.

109. The multispecific polypeptide construct of any of embodiments 60-102 and 108, wherein the at least one B7H3 VHH domain, or each of the first and second B7H3 VHH domain, independently comprises a CDR1 comprising an amino acid sequence set forth in SEQ ID NO: 123; a CDR2 comprising an amino acid sequence selected from the group consisting of SEQ ID NO: 152 and 153; and a CDR3 comprising an amino acid sequence selected from the group consisting of SEQ ID NO: 170 and 171.

110. The multispecific polypeptide construct of any of embodiments 60-102, 108 and 109, wherein the at least one B7H3 VHH domain, or each of the first and second B7H3 VHH domain, independently comprises a CDR1, CDR2 and CDR3 set forth in SEQ ID NOS: 123, 152 and 170, respectively; SEQ ID NOS: 123, 152 and 171, respectively; SEQ ID NOS: 123, 153 and 170, respectively; or SEQ ID NOS: 123, 153 and 171, respectively.

111. The multispecific polypeptide construct of any of embodiments 60-102 and 108-110, wherein the at least one B7H3 VHH domain, or each of the first and second B7H3 VHH domain, independently comprises the sequence of amino acids set forth in any one of SEQ ID NOs: 40, 41, or 498-503 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 40, 41, or 498-503, and binds B7H3.

112 The multispecific polypeptide construct of embodiments 60-102 and 108-111, wherein the at least one B7H3 VHH domain, or each of the first and second B7H3 VHH domain, independently comprises the sequence of amino acids set forth in any one of SEQ ID NOS: 40, 41, or 498-503.

113. The multispecific polypeptide construct of any of embodiments 60-102, wherein the at least one B7H3 VHH domain, or each of the first and second B7H3 VHH domain, independently comprises the sequence set forth in (i) SEQ ID NO:44 (ii) a humanized variant of SEQ ID NO:44, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:44, and binds B7H3.

114. The multispecific polypeptide construct of any of embodiments 60-102 and 113, wherein the at least one B7H3 VHH domain, or each of the first and second B7H3 VHH domain, independently comprises a CDR1 comprising an amino acid sequence selected from the group consisting of SEQ ID NO: 124, 125, 126, 127, 128, 129, 130, 131, 132, or 133; a CDR2 comprising an amino acid sequence set forth in SEQ ID NO: 154; and a CDR3 comprising an amino acid sequence selected from the group consisting of SEQ ID NO: 172, 173, 174, 175, 176, 177, 178, 179, 180, 181, 182 and 183.

115. The multispecific polypeptide construct of any of embodiments 60-102, 113 and 114, wherein the at least one B7H3 VHH domain, or each of the first and second B7H3 VHH domain, independently comprises a CDR1, CDR2 and CDR3 set forth in SEQ ID NOS: 124, 154 and 172, respectively; SEQ ID NOS: 124, 154 and 173, respectively; SEQ ID NOS: 124, 154 and 174, respectively; SEQ ID NOS: 124, 154 and 175, respectively; SEQ ID NOS: 125, 154 and 173, respectively; SEQ ID NOS: 126, 154 and 173, respectively; SEQ ID NOS: 127, 154 and 173, respectively; SEQ ID NOS: 128, 154 and 173, respectively; SEQ ID NOS: 129, 154 and 173, respectively; SEQ ID NOS: 130, 154 and 173, respectively; SEQ ID NOS: 131, 154 and 173, respectively; SEQ ID NOS: 124, 154 and 176, respectively; SEQ ID NOS: 124, 154 and 177, respectively; SEQ ID NOS: 124, 154 and 178, respectively; SEQ ID NOS: 124, 154 and 179, respectively; SEQ ID NOS: 124, 154 and 180, respectively; SEQ ID NOS: 124, 154 and 181, respectively; SEQ ID NOS: 124, 154 and 182, respectively; SEQ ID NOS: 124, 154 and 183, respectively; SEQ ID NOS: 126, 154 and 176, respectively; SEQ ID NOS: 124, 154 and 179, respectively; SEQ ID NOS: 124, 154 and 182, respectively; SEQ ID NOS: 132, 154 and 176, respectively; or SEQ ID NOS: 133, 154 and 173, respectively.

116. The multispecific polypeptide construct of any of embodiments 60-102 and 113-115, wherein the at least one B7H3 VHH domain, or each of the first and second B7H3 VHH domain, independently comprises the sequence of amino acids set forth in any one of SEQ ID NOs: 56-91, 466, and 504-514 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 56-91, 466, and 504-514, and binds B7H3.

117. The multispecific polypeptide construct of embodiments 60-102 and 113-116, wherein the at least one B7H3 VHH domain, or each of the first and second B7H3 VHH domain, independently comprises the sequence of amino acids set forth in any one of SEQ ID NOS: 56-91, 466, and 504-514.

118. The multispecific polypeptide construct of any of embodiments 60-102, wherein the at least one B7H3 VHH domain, or each of the first and second B7H3 VHH domain, independently comprises the sequence set forth in (i) SEQ ID NO:105 (ii) a humanized variant of SEQ ID NO:105, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:105, and binds B7H3.

119 The multispecific polypeptide construct of any of embodiments 60-102 and 118, wherein the at least one B7H3 VHH domain, or each of the first and second B7H3 VHH domain, independently comprises a CDR1 comprising an amino acid sequence set forth in SEQ ID NO: 145; a CDR2 comprising an amino acid sequence set forth in SEQ ID NO: 167; and a CDR3 comprising an amino acid sequence set forth in SEQ ID NO: 488.

120. The multispecific polypeptide construct of any of embodiments 60-102, 118 and 119, wherein the at least one B7H3 VHH domain, or each of the first and second B7H3 VHH domain, independently comprises the sequence of amino acids set forth in any one of SEQ ID NOs:106-109 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:106-109, and binds B7H3.

121. The multispecific polypeptide construct of any of embodiments 60-102 and 118-120, wherein the at least one B7H3 VHH domain, or each of the first and second B7H3 VHH domain, independently comprises the sequence of amino acids set forth in any one of SEQ ID NOS: 106-109.

122. The multispecific polypeptide construct of any of embodiments 60-102, wherein the at least one B7H3 VHH domain, or each of the first and second B7H3 VHH domain, independently comprises the sequence set forth in (i) SEQ ID NO:110 (ii) a humanized variant of SEQ ID NO:110, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:110, and binds B7H3.

123. The multispecific polypeptide construct of any of embodiments 60-102 and 122, wherein the at least one B7H3 VHH domain, or each of the first and second B7H3 VHH domain, independently comprises a CDR1 comprising an amino acid sequence set forth in SEQ ID NO: 139; a CDR2 comprising an amino acid sequence set forth in SEQ ID NO: 161; and a CDR3 comprising an amino acid sequence set forth in SEQ ID NO: 189.

124. The multispecific polypeptide construct of any of embodiments 60-102, 122 and 123, wherein the at least one B7H3 VHH domain, or each of the first and second B7H3 VHH domain, independently comprises the sequence of amino acids set forth in any one of SEQ ID NOs: 515-518 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 515-518 and binds B7H3.

125. The multispecific polypeptide construct of any of embodiments 60-102 and 122-124, wherein the at least one B7H3 VHH domain, or each of the first and second B7H3 VHH domain, independently comprises the sequence of amino acids set forth in any one of SEQ ID NOS: 515-518.

126. The multispecific polypeptide construct of any of embodiments 60-102, wherein the at least one B7H3 VHH domain, or each of the first and second B7H3 VHH domain, independently comprises the sequence set forth in (i) SEQ ID NO:92, 93, 94, 95, 96, 97, 98, 99, 100, 101, 102, 103 or 104 (ii) a humanized variant of SEQ ID NO: 92, 93, 94, 95, 96, 97, 98, 99, 100, 101, 102, 103 or 104, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO: 92, 93, 94, 95, 96, 97, 98, 99, 100, 101, 102, 103 or 104, and binds B7H3.

127. The multispecific polypeptide construct of any of embodiments 60-102 and 126, wherein the at least one B7H3 VHH domain, or each of the first and second B7H3 VHH domain, independently comprises a CDR1, CDR2 and CDR3 set forth in SEQ ID NOS:134, 155 and 184, respectively; 135, 156 and 168, respectively; 136, 157 and 185, respectively; 137, 158 and 186, respectively; 138, 159 and 187, respectively; 138, 160 and 188, respectively; 139, 161 and 189, respectively; 140, 162 and 483, respectively; 141, 163 and 484, respectively; 139, 161 and 189, respectively; 142, 164 and 485, respectively; 143, 165 and 486, respectively; 144, 166 and 487, respectively.

128. The multispecific polypeptide construct of any of embodiments 60-102, 126 and 127, wherein the at least one B7H3 VHH domain, or each of the first and second B7H3 VHH domain, independently is set forth in SEQ ID NO: 92, 93, 94, 95, 96, 97, 98, 99, 100, 101, 102, 103 or 104.

129. The multispecific polypeptide construct of any of embodiments 60-128, wherein one or both of the first and second components comprises at least one co-stimulatory receptor binding region (CRBR) that binds a co-stimulatory receptor.

130. The multispecific polypeptide construct of embodiment 129, wherein the at least one co-stimulatory receptor binding region (CRBR) is positioned amino-terminally relative to the Fc region and/or carboxy-terminally relative to the CD3 binding region of the multispecific polypeptide construct.

131. The multispecific polypeptide construct of embodiment 129 or embodiment 130, wherein the multispecific polypeptide construct comprises only one co-stimulatory receptor binding region (CRBR).

132. The multispecific polypeptide construct of any of embodiments 129-131, wherein:

the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second component comprises the CRBR and comprises in order of N-terminus to C-terminus the second Fc polypeptide of the heterodimeric Fc region, the linker, optionally the same linker as present in the first component, the other of the VH or VL domain of the anti-CD3 antibody or antigen binding fragment, wherein the CRBR is positioned amino-terminally relative to the Fc region or carboxy-terminally relative to the anti-CD3 antibody or antigen binding fragment of the second component.

133. The multispecific polypeptide construct of any of embodiments 129-132, wherein the at least one co-stimulatory receptor binding region (CRBR) is or comprises the extracellular domain or binding fragment thereof of the native cognate binding partner of the co-stimulatory receptor, or a variant thereof that exhibits binding activity to the co-stimulatory receptor.

134. The multispecific polypeptide construct of any of embodiments 129-132, wherein the at least one co-stimulatory receptor binding region (CRBR) is an antibody or antigen-binding fragment thereof selected from the group consisting of a Fab fragment, a F(ab′)2 fragment, an Fv fragment, a scFv, a scAb, a dAb, a single domain heavy chain antibody, and a single domain light chain antibody.

135. The multispecific polypeptide construct of embodiment 134, wherein the antibody or antigen-binding fragment thereof is a Fv, a scFv, a Fab, a single domain antibody (sdAb), a VNAR, or a VHH.

136. The multispecific polypeptide construct of embodiment 134 or embodiment 135, wherein the antibody or antigen-binding fragment is an sdAb.

137. The multispecific polypeptide construct of embodiment 136, wherein the sdAb is a human or humanized sdAb.

138. The multispecific polypeptide construct of any of embodiments 129-137, wherein the at least one co-stimulatory receptor binding region (CRBR) binds a co-stimulatory receptor selected from among 41BB (CD137), OX40 (CD134), CD27, glucocorticoid-induced TNFR-related protein (GITR), CD28, ICOS, CD40, B-cell activating factor receptor (BAFF-R), B-cell maturation antigen (BCMA), Transmembrane activator and CAML interactor (TACI), and NKG2D.

139. The multispecific polypeptide construct of any of embodiments 129-138, wherein the at least one co-stimulatory receptor binding region (CRBR) binds a co-stimulatory receptor selected from among 41BB (CD137), OX40 (CD134), and glucocorticoid-induced TNFR-related protein (GITR).

140. The multispecific polypeptide construct of any of embodiments 129-139, wherein the at least one co-stimulatory receptor binding region (CRBR) comprises the sequence of amino acids set forth in SEQ ID NO:400 or a sequence that has at least 85%, 86%, 87%, 88%, 89%, 90%, 91%, 92%, 93%, 94%, 95%, 96%, 97%, 98%, or 99% sequence identity to the sequence set forth in SEQ ID NO:400 and binds 4-1BB.

141. The multispecific polypeptide construct of any of embodiments 60-140, wherein one or both of the first and second components comprises at least one inhibitory receptor binding region (IRBR) that binds an inhibitory receptor.

142. The multispecific polypeptide construct of embodiment 141, wherein the at least one inhibitory receptor binding region (IRBR) is positioned amino-terminally relative to the Fc region and/or carboxy-terminally relative to the CD3 binding region of the multispecific polypeptide construct.

143. The multispecific polypeptide construct of embodiment 141 or embodiment 142, wherein the multispecific polypeptide construct comprises only one inhibitory receptor binding region (IRBR).

144. The multispecific polypeptide construct of any of embodiments 141-143, wherein: the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second component comprises the IRBR and comprises in order of N-terminus to C-terminus the second Fc polypeptide of the heterodimeric Fc region, the linker, optionally the same linker as present in the first component, the other of the VH or VL domain of the anti-CD3 antibody or antigen binding fragment, wherein the IRBR is positioned amino-terminally relative to the Fc region or carboxy-terminally relative to the anti-CD3 antibody or antigen-binding fragment of the second component.

145. The multispecific polypeptide construct of any of embodiments 141-144, wherein the at least one IRBR is or comprises the extracellular domain or binding fragment thereof of the native cognate binding partner of the inhibitory receptor, or a variant thereof that exhibits binding activity to the inhibitory receptor.

146. The multispecific polypeptide construct of any of embodiments 141-144, wherein the at least one IRBR is an antibody or antigen-binding fragment thereof selected from the group consisting of a Fab fragment, a F(ab′)2 fragment, an Fv fragment, a scFv, a scAb, a dAb, a single domain heavy chain antibody, and a single domain light chain antibody.

147. The multispecific polypeptide construct of embodiment 146, wherein the antibody or antigen-binding fragment thereof is a Fv, a scFv, a Fab, a single domain antibody (sdAb), a VNAR, or a VHH.

148. The multispecific polypeptide construct of embodiment 146 or embodiment 147, wherein the antibody or antigen-binding fragment is an sdAb.

149. The multispecific polypeptide construct of any of embodiments 146-148, wherein the sdAb is a human or humanized sdAb.

150. The multispecific polypeptide construct of any of embodiments 141-149, wherein the at least one IRBR binds a inhibitory receptor selected from among PD-1, CTLA-4, TIGIT, VISTA and TIM3.

151. The multispecific polypeptide construct of any of embodiments 141-149, wherein the at least one IRBR binds PD-1.

152. The multispecific polypeptide construct of any of embodiments 141-151, wherein: the first component comprises in order of N-terminus to C-terminus a first B7H3 VHH domain that binds B7H3, the first Fc polypeptide of the heterodimeric Fc region, the linker, the VH or VL domain of the anti-CD3 antibody or antigen binding fragment and a second B7H3 VHH domain that binds B7H3; and the second component comprises in order of N-terminus to C-terminus one of the IRBR or the CRBR, the second Fc polypeptide of the heterodimeric Fc region, the linker, optionally the same linker as present in the first component, the other of the VH or VL domain of the anti-CD3 antibody or antigen binding fragment, and the other of the CRBR or IRBR.

153. The multispecific polypeptide construct of any of embodiments 60-152, wherein the linker is a peptide or polypeptide linker, optionally wherein the linker is 3, 4, 5, 6, 7, 8, 9, 10, 11, 12, 13, 14, 15, 16, 17, 18, 19 or 20 amino acids in length.

154. The multispecific polypeptide construct of any of embodiments 60-153, wherein the linker is a non-cleavable linker 155. The multispecific polypeptide construct of embodiment 153, wherein the non-cleavable linker comprises GS, GGS, GGGGS (SEQ ID NO:315), GGGGGS (SEQ ID NO:316) and combinations thereof.

156. The multispecific polypeptide construct of any of embodiments 60-155, wherein the linker is or comprises the sequence GGGGGSGGGGGSGGGGGS (SEQ ID NO:317).

157. The multispecific polypeptide construct of any of embodiments 60-153, wherein the linker is a cleavable linker.

158. The multispecific polypeptide construct of embodiment 157, wherein the cleavable linker is a polypeptide that functions as a substrate for a protease.

159. The multispecific polypeptide construct of embodiment 158, wherein the protease is produced by an immune effector cell, by a tumor, or by cells present in the tumor microenvironment.

160. The multispecific polypeptide construct of embodiment 158 or embodiment 159, wherein the protease is produced by an immune effector cell and the immune effector cell is an activated T cell, a natural killer (NK) cell, or an NK T cell.

161. The multispecific polypeptide construct of any of embodiments 158-160, wherein the protease is selected from among matriptase, a matrix metalloprotease (MMP), granzyme B, and combinations thereof.

162. The multispecific polypeptide construct of embodiment 161, wherein the protease is granzyme B.

163. The multispecific polypeptide construct of any of embodiments 158-162, wherein the cleavable linker comprises the amino acid sequence GGSGGGG [symbol] IGGSGGS (SEQ ID NO:361).

164. An isolated single domain antibody that binds B7H3, comprising a complementarity determining region 1 (CDR1) comprising an amino acid sequence selected from the group consisting of SEQ ID NO: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and 145; a complementarity determining region 2 (CDR2) comprising an amino acid sequence selected from the group consisting of SEQ ID NO: 146, 147, 148, 149, 150, 151, 152, 153, 154, 155, 156, 157, 158, 159, 160, 161, 162, 163, 164, 165, 166 and 167; and a complementarity determining region 3 (CDR3) comprising an amino acid sequence selected from the group consisting of SEQ ID NO: 168, 169, 170, 171, 172, 173, 174, 175, 176, 177, 178, 179, 180, 181, 182, 183, 184, 185, 186, 187, 188, 189, and 483-488.

165. The isolated single domain antibody of embodiment 164, comprising the amino acid sequence set forth in any of SEQ ID NOS: 1, 8-35, 40, 41, 44, 56-110, 466, 467, 489, 490, or 492-518, or a sequence of amino acids that exhibits at least 85%, 86%, 87%, 88%, 89%, 90%, 91%, 92%, 93%, 94%, 95%, 96%, 97%, 98%, or 99% sequence identity to any of SEQ ID NOS: 1, 8-35, 40, 41, 44, 56-110, 466, 467, 489, 490, or 492-518 , and binds B7H3.

166. The isolated single domain antibody of embodiment 164 or embodiment 165, wherein the single domain antibody comprises the sequence set forth in (i) SEQ ID NO:1, (ii) a humanized variant of SEQ ID NO:1, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:1, and binds B7H3.

167. The isolated single domain antibody of any of embodiments 164-166, wherein the sdAb comprises a CDR1 comprising an amino acid sequence selected from the group consisting of SEQ ID NO: 115, 116, 117, 118, 119, 120 and 121; a CDR2 comprising an amino acid sequence selected from the group consisting of SEQ ID NO: 146, 147, 148, 149, 150 and 151; and a CDR3 comprising an amino acid sequence selected from the group consisting of SEQ ID NO: 168 and 169.

168. The isolated single domain antibody of any of embodiments 164-167, wherein the sdAb comprises a CDR1, CDR2 and CDR3 set forth in SEQ ID NOS: 115, 146 and 168, respectively; SEQ ID NOS: 115, 147 and 168, respectively; SEQ ID NOS: 115, 148 and 168, respectively; SEQ ID NOS: 115, 149 and 168, respectively; SEQ ID NOS: 115, 150 and 168, respectively; SEQ ID NOS: 116, 146 and 168, respectively; SEQ ID NOS: 117, 146 and 168, respectively; SEQ ID NOS: 118, 146 and 168, respectively; SEQ ID NOS: 115, 146 and 169, respectively; SEQ ID NOS: 119, 146 and 168, respectively; SEQ ID NOS: 120, 146 and 168, respectively; SEQ ID NOS: 115, 151 and 168, respectively; SEQ ID NOS: 116, 147 and 168, respectively; SEQ ID NOS: 118, 147 and 168, respectively; SEQ ID NOS: 119, 147 and 168, respectively; SEQ ID NOS: 116, 151 and 168, respectively; SEQ ID NOS: 115, 146 and 168, respectively; SEQ ID NOS: 121, 147 and 168, respectively; SEQ ID NOS: 115, 146 and 168, respectively; SEQ ID NOs: 119, 149 and 168, respectively; or SEQ ID NOS: 122, 151 and 168, respectively.

169. The isolated single domain antibody of any of embodiments 164-168, wherein the sdAb comprises the sequence of amino acids set forth in any one of SEQ ID NOs: 8-34, 467, 489-490, and 492-497 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 8-34, 467, 489-490, and 492-497, and binds B7H3.

160. The isolated single domain antibody of any of embodiments 164-169, wherein the sdAb comprises the sequence of amino acids set forth in any one of SEQ ID NOS: 8-34, 467, 489-490, and 492-497.

171. The isolated single domain antibody of embodiment 164 or embodiment 165, wherein the sdAb comprises the sequence set forth in (i) SEQ ID NO:35, (ii) a humanized variant of SEQ ID NO:35, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:35, and binds B7H3.

172. The isolated single domain antibody of any of embodiments 164, 165 and 171, wherein the sdAb comprising a CDR1 comprising an amino acid sequence set forth in SEQ ID NO: 123; a CDR2 comprising an amino acid sequence selected from the group consisting of SEQ ID NO: 152 and 153; and a CDR3 comprising an amino acid sequence selected from the group consisting of SEQ ID NO: 170 and 171.

173. The isolated single domain antibody of any of embodiments 164, 165, 171 and 172, wherein the sdAb comprises a CDR1, CDR2 and CDR3 set forth in SEQ ID NOS: 123, 152 and 170, respectively; SEQ ID NOS: 123, 152 and 171, respectively; SEQ ID NOS: 123, 153 and 170, respectively; or SEQ ID NOS: 123, 153 and 171, respectively.

174. The isolated single domain antibody of any of embodiments 164, 165 and 171-173, wherein the sdAb comprises the sequence of amino acids set forth in any one of SEQ ID NOs: 40, 41, or 498-503 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 40, 41, or 498-503, and binds B7H3.

175. The isolated single domain antibody of any of embodiments 164, 165 and 171-174, wherein the sdAb comprises the sequence of amino acids set forth in any one of SEQ ID NOS: 40, 41, or 498-503.

176. The isolated single domain antibody of embodiment 164 or embodiment 165, wherein the sdAb comprises the sequence set forth in (i) SEQ ID NO:44 (ii) a humanized variant of SEQ ID NO:44, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:44, and binds B7H3.

177. The isolated single domain antibody of embodiment 164, embodiment 165 or embodiment 176, wherein the sdAb comprises a CDR1 comprising an amino acid sequence selected from the group consisting of SEQ ID NO: 124, 125, 126, 127, 128, 129, 130 131, 132, or 133; a CDR2 comprising an amino acid sequence set forth in SEQ ID NO: 154; and a CDR3 comprising an amino acid sequence selected from the group consisting of SEQ ID NO: 172, 173, 174, 175, 176, 177, 178, 179, 180, 181, 182 and 183.

178. The isolated single domain antibody of any of embodiments 164, 165, 176 and 177, wherein the sdAb comprises a CDR1, CDR2 and CDR3 set forth in SEQ ID NOS: 124, 154 and 172, respectively; SEQ ID NOS: 124, 154 and 173, respectively; SEQ ID NOS: 124, 154 and 174, respectively; SEQ ID NOS: 124, 154 and 175, respectively; SEQ ID NOS: 125, 154 and 173, respectively; SEQ ID NOS: 126, 154 and 173, respectively; SEQ ID NOS: 127, 154 and 173, respectively; SEQ ID NOS: 128, 154 and 173, respectively; SEQ ID NOS: 129, 154 and 173, respectively; SEQ ID NOS: 130, 154 and 173, respectively; SEQ ID NOS: 131, 154 and 173, respectively; SEQ ID NOS: 124, 154 and 176, respectively; SEQ ID NOS: 124, 154 and 177, respectively; SEQ ID NOS: 124, 154 and 178, respectively; SEQ ID NOS: 124, 154 and 179, respectively; SEQ ID NOS: 124, 154 and 180, respectively; SEQ ID NOS: 124, 154 and 181, respectively; SEQ ID NOS: 124, 154 and 182, respectively; SEQ ID NOS: 124, 154 and 183, respectively; SEQ ID NOS: 126, 154 and 176, respectively; SEQ ID NOS: 124, 154 and 179, respectively; SEQ ID NOS: 124, 154 and 182, respectively; SEQ ID NOS: 132, 154 and 176, respectively; or SEQ ID NOS: 133, 154 and 173, respectively.

179. The isolated single domain antibody of any of embodiments 164, 165 and 176-178, wherein the sdAb comprises the sequence of amino acids set forth in any one of SEQ ID NOs: 56-91, 466, and 504-514 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 56-91, 466, and 504-514, and binds B7H3.

180. The isolated single domain antibody of any of embodiments 164, 165 and 176-179, wherein the at least one B7H3 VHH domain comprises the sequence of amino acids set forth in any one of SEQ ID NOS: 56-91, 466, and 504-514.

181. The isolated single domain antibody of embodiment 164 or embodiment 165, wherein the sdAb comprises the sequence set forth in (i) SEQ ID NO:105 (ii) a humanized variant of SEQ ID NO:105, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:105, and binds B7H3.

182. The isolated single domain antibody of embodiment 164, 165 or 181, wherein the sdAb comprising a CDR1 comprising an amino acid sequence set forth in SEQ ID NO: 145; a CDR2 comprising an amino acid sequence set forth in SEQ ID NO: 167; and a CDR3 comprising an amino acid sequence set forth in SEQ ID NO: 488.

183. The isolated single domain antibody of any of embodiments 164, 165, 181 and 182, wherein the sdAb comprises the sequence of amino acids set forth in any one of SEQ ID NOs:106-109 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:106-109, and binds B7H3.

184. The isolated single domain antibody of any of embodiments 164, 165 and 181-183, wherein the sdAb comprises the sequence of amino acids set forth in any one of SEQ ID NOS: 106-109.

185. The isolated single domain antibody of embodiment 164 or embodiment 165, wherein the sdAb comprises the sequence set forth in (i) SEQ ID NO:110 (ii) a humanized variant of SEQ ID NO:110, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:110, and binds B7H3.

186. The isolated single domain antibody of any of embodiments 164, 165 and 185, wherein the sdAb comprising a CDR1 comprising an amino acid sequence set forth in SEQ ID NO: 139; a CDR2 comprising an amino acid sequence set forth in SEQ ID NO: 161; and a CDR3 comprising an amino acid sequence set forth in SEQ ID NO: 189.

187. The isolated single domain antibody of any of embodiments 164, 165, 185 and 186, wherein the sdAb comprises the sequence of amino acids set forth in any one of SEQ ID NOs: 515-518 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 515-518, and binds B7H3.

188. The isolated single domain antibody of any of embodiments 164, 165 and 185-187, wherein the sdAb comprises the sequence of amino acids set forth in any one of SEQ ID NOS: 515-518.

189. The isolated single domain antibody of embodiment 164 or embodiment 165, wherein the sdAb comprises the sequence set forth in (i) SEQ ID NO:92, 93, 94, 95, 96, 97, 98, 99, 100, 101, 102, 103 or 104 (ii) a humanized variant of SEQ ID NO: 92, 93, 94, 95, 96, 97, 98, 99, 100, 101, 102, 103 or 104, or (iii) a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 92, 93, 94, 95, 96, 97, 98, 99, 100, 101, 102, 103 or 104, and binds B7H3.

190. The isolated single domain antibody of any of embodiments 164, 165 and 189, wherein the at least one B7H3 VHH domain comprises a CDR1, CDR2 and CDR3 set forth in SEQ ID NOS:134, 155 and 184, respectively; 135, 156 and 168, respectively; 136, 157 and 185, respectively; 137, 158 and 186, respectively; 138, 159 and 187, respectively; 138, 160 and 188, respectively; 139, 161 and 189, respectively; 140, 162 and 483, respectively; 141, 163 and 484, respectively; 139, 161 and 189, respectively; 142, 164 and 485, respectively; 143, 165 and 486, respectively; 144, 166 and 487, respectively.

191. A polynucleotide(s) encoding the B7H3-binding polypeptide of any of embodiments 1-59.

192. A polynucleotide(s) encoding the multispecific polypeptide construct of any of embodiments 60-163.

193. A polynucleotide, comprising a first nucleic acid sequence encoding a first polypeptide of a multispecific construct of any of embodiments 60-163 and a second nucleic acid sequence encoding a second polypeptide of the multispecific construct, wherein the first and second nucleic acid sequence are separated by an internal ribosome entry site (IRES), or a nucleic acid encoding a self-cleaving peptide or a peptide that causes ribosome skipping.

194. The polynucleotide of embodiment 193, wherein the first nucleic acid sequence and second nucleic acid sequence are operably linked to the same promoter.

195. The polynucleotide of embodiment 194, wherein the nucleic acid encoding a self-cleaving peptide or a peptide that causes ribosome skipping is selected from a T2A, a P2A, a E2A or a F2A.

196. A polynucleotide encoding the single domain antibody of any of embodiments 164-190.

197. A vector, comprising the polynucleotide of any of embodiments 191-196.

198. The vector of embodiment 197 that is an expression vector.

199. The vector of embodiment 197 or embodiment 198 that is a viral vector or a eukaryotic vector, optionally wherein the eukaryotic vector is a mammalian vector.

200. A cell, comprising polynucleotide or polynucleotides of-any of embodiments 191-196, or a vector or vectors of any of embodiments 197-199.

201. The cell of embodiment 200, wherein the cell is recombinant or isolated.

202. The cell of embodiment 201, wherein the cell is a mammalian cell.

203. A method of producing a polypeptide, the method comprising introducing into a cell a polynucleotide or polynucleotides of any of embodiments 191-196 or a vector or vectors of any of embodiments 197-199 and culturing the cell under conditions to produce the multispecific polypeptide construct.

204. The method of embodiment 203, further comprising isolating or purifying the polypeptide from the cell.

205. A polypeptide produced by the method of embodiment 203 or embodiment 204.

206. An engineered immune cell, comprising a chimeric antigen receptor comprising: an extracellular domain comprising the single domain antibody of any of embodiments 164-190; a transmembrane domain; and an intracellular signaling domain.

207. The engineered immune cell of embodiment 206, wherein the cell is a lymphocyte.

208. The engineered immune cell of embodiment 206 or embodiment 207, wherein the cell is a T cell or a natural killer (NK) cell.

209. The engineered immune cell of any of embodiments 206-208, wherein the intracellular signaling domain comprises an immunoreceptor tyrosine-based activation motif (ITAM) signaling domain.

210. The engineered immune cell of any of embodiments 206-209, wherein the intracellular signaling domain is or comprises a CD3zeta signaling domain, optionally a human CD3zeta signaling domain.

211. The engineered immune cell of embodiment 209 or embodiment 210, wherein the intracellular signaling domain further comprises a signaling domain of a costimulatory molecule.

212. The engineered immune cell of embodiment 211, wherein the costimulatory molecule is CD28, ICOS, 41BB or OX40, optionally a human CD28, a human ICOS, a human 41BB or a human OX40.

213. A pharmaceutical composition comprising the B7H3-binding polypeptide of any of embodiments 1-59, the multispecific polypeptide construct of any of embodiments 60-163, the single domain antibody of any of embodiments 164-190 or the engineered immune cell of any of embodiments 206-212.

214. The pharmaceutical composition of embodiment 213, comprising a pharmaceutically acceptable carrier.

215. The pharmaceutical composition of embodiment 213 or embodiment 214 that is sterile.

216. A method of stimulating or inducing an immune response in a subject, the method comprising administering, to a subject in need thereof, the B7H3-binding polypeptide of any of embodiments 1-59, the multispecific polypeptide construct of any of embodiments 60-163, the single domain antibody of any of embodiments 164-190 or the engineered immune cell of any of embodiments 206-212 or a pharmaceutical composition of embodiment 213-215.

217. The method of embodiment 216, wherein the immune response is increased against a tumor or cancer, optionally a tumor or a cancer that expresses B7H3.

218. The method of embodiment 216 or embodiment 217, wherein the method treats a disease or condition in the subject.

219. A method of treating a disease or condition in a subject, the method comprising administering, to a subject in need thereof, a therapeutically effective amount of the B7H3-binding polypeptide of any of embodiments 1-59, the multispecific polypeptide construct of any of embodiments 60-163, the single domain antibody of any of embodiments 164-190 or the engineered immune cell of any of embodiments 206-212 or a pharmaceutical composition of embodiment 213-215.

220. The method of embodiment 218 or embodiment 219, wherein the disease or condition is a tumor or a cancer.

221. The method of any of embodiments 216-220, wherein said subject is a human.

### VIII. EXAMPLES

The following examples are included for illustrative purposes only and are not intended to limit the scope of the invention.

### Example 1: Generation of B7H3 sdAb

Single domain antibodies targeting human B7H3 were generated via immunization of llamas and alpaca. Llamas and alpaca were immunized with a recombinant version of the human B7H3 extracellular domain (ECD: amino acids 29-466 of human B7H3 set forth in SEQ ID NO:190, e.g. UniProt No. Q5ZPR3) set forth as follows:

LEVQVPEDPVVALVGTDATLCCSFSPEPGFS
LAQLNLIWQLTDTKQLVHSFAEGQDQGSAYA
NRTALFPDLLAQGNASLRLQRVRVADEGSFT
CFVSIRDFGSAAVSLQVAAPYSKPSMTLEPN
KDLRPGDTVTITCSSYQGYPEAEVFWQDGQG
VPLTGNVTTSQMANEQGLFDVHSILRVVLGA
NGTYSCLVRNPVLQQDAHSSVTITPQRSPTG
AVEVQVPEDPVVALVGTDATLRCSFSPEPGF
SLAQLNLIWQLTDTKQLVHSFTEGRDQGSAY
ANRTALFPDLLAQGNASLRLQRVRVADEGSF
TCFVSIRDFGSAAVSLQVAAPYSKPSMTLEP
NKDLRPGDTVTITCSSYRGYPEAEVFWQDGQ
GVPLTGNVTTSQMANEQGLFDVHSVLRVVLG
ANGTYSCLVRNPVLQQDAHGSVTITGQPMTF
PPEA

Following the development of specific anti-B7H3 antibody titers, llama/alpaca peripheral blood mononuclear cells (PBMCs) were isolated from 500mL of blood from the immunized animal and total mRNA was isolated using the Qiagen RNeasy Maxi Kit and subsequently converted to first strand cDNA using Thermo Superscript IV Reverse Transcriptase and oligo-dT priming. Single domain antibody (sdAb; also called VHH) sequences were specifically amplified via PCR using the cDNA as template and cloned into a yeast surface display vector as sdAb-Fc-AGA2 fusion proteins. The Fc was a human IgG1 Fc (set forth in SEQ ID NO:198) or, in some cases, a variant thereof with reduced effector function (Fc xELL; SEQ ID NO:199).

Yeast libraries displaying these sdAbs were enriched using recombinant forms of the B7H3 ECD via magnetic bead isolation followed by fluorescence activated cell sorting (FACS). Sorted yeast were plated out and isolated colonies were picked into 96-well blocks and grown in media that switched the expression from surface displayed sdAb-Fc to secretion into the media. Supernatants from the 96-well yeast secretion cultures were applied to A375 cells (B7H3 positive) or CCRF-CEM cells (B7H3 negative), washed, treated with fluorophore labelled anti-human Fc secondary antibody, and analyzed by 96-well flow cytometry.

Nucleic acid sequences encoding sdAbs that bound to B7H3 positive cells and not to B7H3 negative cells were cloned in-frame with a human Fc encoding region into mammalian expression vectors and expressed by transient transfection in HEK293 freestyle cells (293F cells) or CHO cells using polyethylenimine. Supernatant was collected after 3-7 days, secreted recombinant protein was purified by protein A chromatography, and concentration was calculated from the absorbance at 280 nm and extinction coefficient.

Exemplary identified sdAbs are set forth in Table E1. In some cases, the sdAbs can include a flexible linker (e.g. GG) for linkage to another polypeptide, such as an Fc or another sdAb.

TABLE El
B7H3 sdAbs

| | | | | | | | VHH |
|---|---|---|---|---|---|---|---|
| | | SEQ | | SEQ | | SEQ | SEQ |
| Clone | | ID | | ID | | ID | ID |
| name | CDR1 | NO | CDR2 | NO | CDR3 | NO | NO |
| L1A5 | GFSFGSNVMM | 115 | TIYSSGTGTF | 146 | SGPVRGWGP | 168 | 1 |
| L57B04 | ERTFSTYTMG | 123 | VVNWSGGSKY | 152 | GGAYSGPYYDTRQYTY | 170 | 35 |
| L58E05 | GSTFSMYHMS | 124 | TSHHGGTTN | 154 | DHGYNGRGY | 172 | 44 |
| 1A10 | GLTFDEHHMG | 134 | AITWHTGTTW | 155 | GRRPFFIREVGVEPDY | 184 | 92 |
| 1E4 | GSSFGSNVMM | 135 | TINSSGTGTF | 156 | SGPVRGWGP | 168 | 93 |
| D9 | GFTFASTGMS | 136 | SINSGSDSTM | 157 | WALSCSGYGCDDLPQD | 185 | 94 |
| A3 | GFTFASYGMS | 137 | SINSGSDTSM | 158 | WALSCSQYGCDDLPRP | 186 | 95 |
| E9 | GRTFSSYAMS | 138 | TITSSGSTTY | 159 | YTSRTVRDY | 187 | 96 |
| B4 | GRTFSSYAMS | 138 | TITTGGGTTY | 160 | YTSRFPRDY | 188 | 97 |
| 57B06 | GGTFSSYAMG | 139 | AISSEGGSTY | 161 | KGVGWPQEQASYDY | 189 | 98 |
| 57B10 | GSIPSIDHMG | 140 | SIDLNGRTN | 162 | RWGSPDYYHDDVDY | 190 | 99 |
| | | | | | | 483 | |
| 58B06 | GRSFSTYAMG | 141 | AVGWRGTNTY | 163 | GEPIRVGEKSGYDY | 191 | 100 |
| | | | | | | 484 | |
| 57A12 | GGTFSSYAMG | 139 | AISSEGGSTY | 161 | KGVGWPQEQASYDY | 189 | 101 |
| 57B08 | GLTFSSYAMG | 142 | AISWSGGNTL | 164 | GPRDYFSDLEVDFGS | 192 | 102 |
| | | | | | | 485 | |
| 58A08 | GRTFSSLAVG | 143 | AISWSGGNTY | 165 | GLPIRVGVPGGYDY | 193 | 103 |
| | | | | | | 486 | |
| 57D1 | GHTFSTYAMG | 144 | GITRSGDSTH | 166 | ASFAYLSTYTHHYDY | 194 | 104 |
| | | | | | | 487 | |
| 1H5 | GRSFGDYTVG | 145 | GLSWLGGTIY | 167 | SRSAISRKATDFGS | 195 | 105 |
| | | | | | | 488 | |
| L57B06 | GGTFSSYAMG | 139 | AISSEGGSTY | 161 | KGVGWPQEQASYDY | 189 | 110 |

### Example 2: Binding of sdAb to B7H3 Expressing Cells by Flow Cytometry

Specificity and relative affinity were assessed for purified sdAb-Fcs on B7H3-expressing cells. Binding of B7H3-sdAb-Fc fusion proteins was assessed by flow cytometry using B7H3-expressing cells, lung carcinoma cell line (NCIH460), melanoma cell line (A375) or a 293 cell line transfected with B7H3 (B7H3+293). A titration series of the fusion protein was incubated with the B7H3-expressing cell lines (approx. 2.5-5×10⁴ cells/well) for 30 minutes at 4° C. in FACS Buffer (PBS 1% BSA, 0.1% NaN₃ pH 7.4) in 96 well plates. Following 3 washes with FACS buffer, an APC-conjugated anti-human Fcγ specific secondary antibody (Jackson ImmunoResearch) was added and cells were incubated for 30 minutes at 4° C. Following three additional washes in FACS buffer bound antibody was detected via flow cytometry (iQue Intellicyte).

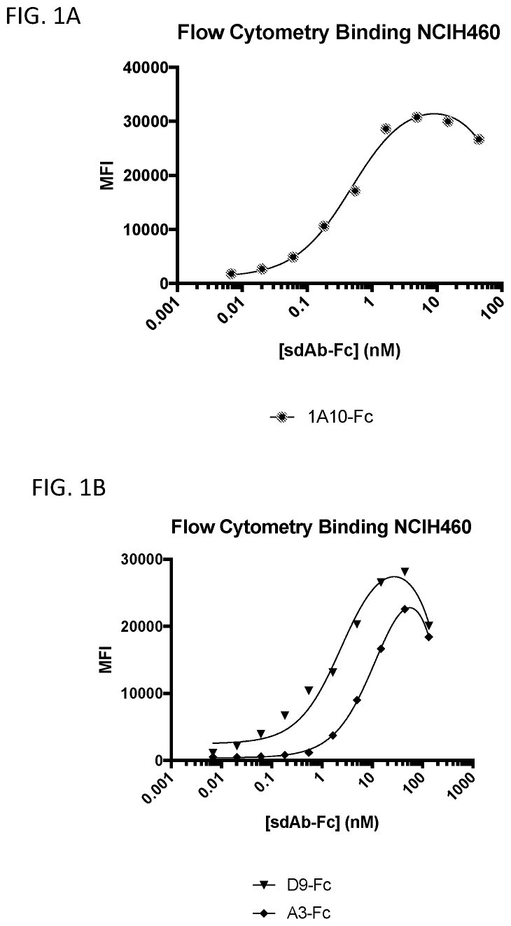

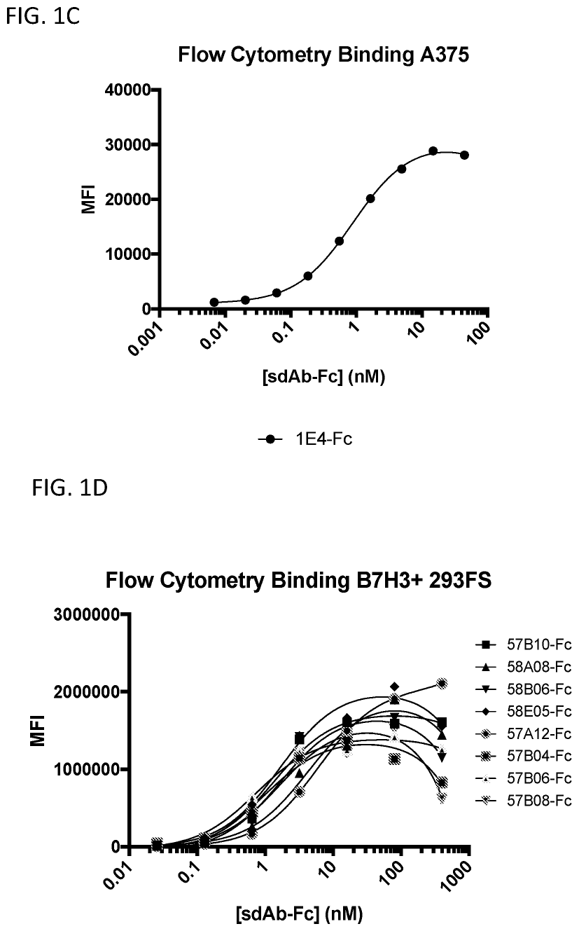

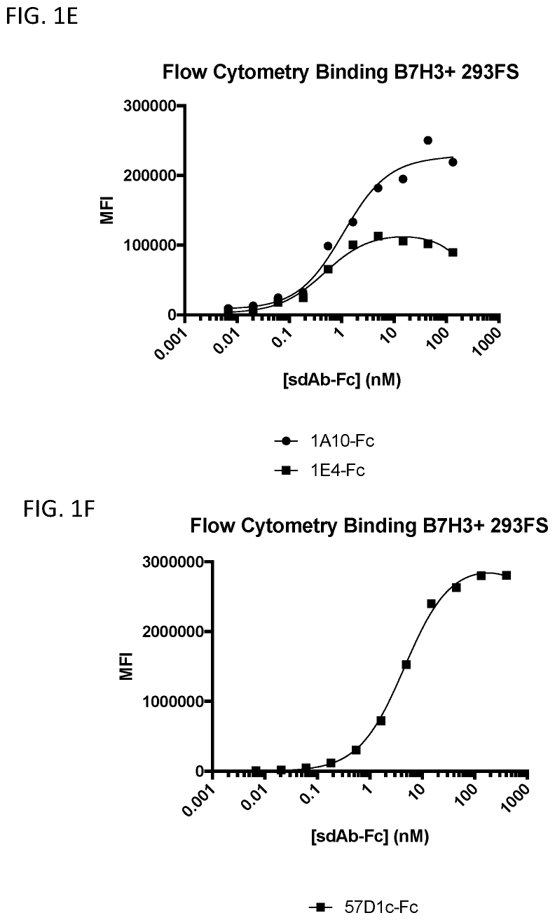

FIGS. 1A-1F set forth results for exemplary B7H3 sdAbs, namely 1A10 (SEQ ID NO: 92), 1E4 (SEQ ID NO: 93), D9 (SEQ ID NO: 94), A3 (SEQ ID NO: 95), 57D1c (SEQ ID NO: 104), 57B10 (SEQ ID NO: 99), 58A08 (SEQ ID NO: 103), 58B06 (SEQ ID NO: 100), 58E05 (SEQ ID NO: 44), 57Al2 (SEQ ID NO: 101), 57B04 (SEQ ID NO: 35), 57B06 (SEQ ID NO: 98), and 57B08 (SEQ ID NO: 102).

### Example 3: Humanization of Camelid Derived B7H3 sdAb

Exemplary camelid derived B7H3 sdAb, L1A5(SEQ ID NO:1), L57B04 (SEQ ID NO:35), L58E05 (SEQ ID NO:44), 1H5 (SEQ ID NO:105) and L57B06 (SEQ ID NO:110), were humanized using the human VH3-23 germline as scaffold. Camelid residues that contribute to solubility, specificity, stability and/or affinity remained unmodified. In addition all humanized variants contained the modification of Leu11Glu (L11E) and the carboxy-terminal modifications of Ser112Lys (S112K) and Ser113Pro (S113P) as these are known prevent or reduce the recognition of pre-existing ADA directed toward sdAbs (as described in US20160207981).

Table E2 sets forth exemplary B7H3 sdAbs humanized variants. In some cases, the sdAbs can include a flexible linker (e.g. GG) for linkage to another polypeptide, such as an Fc or another sdAb.

TABLE E2
B7H3 sdAbs Humaized Variants

| | | | | | | | VHH |
|---|---|---|---|---|---|---|---|
| | | SEQ | | SEQ | | SEQ | SEQ |
| Clone | | ID | | ID | | ID | ID |
| name | CDR1 | NO | CDR2 | NO | CDR3 | NO | NO |
| L1A5 Humanized Variants | | | | | | | |
| hz1A5v1 | GFSFGSNVMM | 115 | TIYSSGTGTF | 146 | SGPVRGWGP | 168 | 2 |
| | | | | | | | 492 |
| hz1A5v2 | GFSFGSNVMM | 115 | TIYSSGTGTF | 146 | SGPVRGWGP | 168 | 3 |
| | | | | | | | 493 |
| hz1A5v3 | GFSFGSNVMM | 115 | TIYSSGTGTF | 146 | SGPVRGWGP | 168 | 4 |
| | | | | | | | 494 |
| hz1A5v7 | GFSFGSNVMM | 115 | TIYSSGTGTF | 146 | SGPVRGWGP | 168 | 5 |
| | | | | | | | 495 |
| hz1A5v8 | GFSFGSNVMM | 115 | TIYSSGTGTF | 146 | SGPVRGWGP | 168 | 6 |
| | | | | | | | 496 |
| hz1A5v9 | GFSFGSNVMM | 115 | TIYSRGTGTF | 147 | SGPVRGWGP | 168 | 7 |
| | | | | | | | 497 |
| hz1A5v12 | GFSFGSNVMM | 115 | TIYSSGTGTF | 146 | SGPVRGWGP | 168 | 8 |
| hz1A5v17 | GFSFGSNVMM | 115 | TIYSRGTGTF | 147 | SGPVRGWGP | 168 | 9 |
| hz1A5v24 | GFSFGSNVMM | 115 | TIYSSGTGTF | 146 | SGPVRGWGP | 168 | 10 |
| hz1A5v25 | GFSFGSNVMM | 115 | TIYSSGTGTF | 146 | SGPVRGWGP | 168 | 11 |
| hz1A5v28 | GFSFGSNVMM | 115 | TIYSSGTGTF | 146 | SGPVRGWGP | 168 | 12 |
| hz1A5v29 | GFSFGSNVMM | 115 | TIYSSGTGTF | 146 | SGPVRGWGP | 168 | 13 |
| hz1A5v30 | GFSFGSNVMM | 115 | TIYSRGGSTF | 148 | SGPVRGWGP | 168 | 14 |
| hz1A5v31 | GFSFGSNVMM | 115 | TIYSSGTGTY | 149 | SGPVRGWGP | 168 | 15 |
| hz1A5v32 | GFSFGSNVMM | 115 | TIYSRGGSTY | 150 | SGPVRGWGP | 168 | 16 |
| hz1A5v33 | GFSFGSNVMM | 115 | TIYSRGTGTF | 147 | SGPVRGWGP | 168 | 17 |
| hz1A5v34 | GFSFGSNVMM | 115 | TIYSSGTGTF | 146 | SGPVRGWGP | 168 | 18 |
| hz1A5v35 | GFSFGSYVMM | 116 | TIYSSGTGTF | 146 | SGPVRGWGP | 168 | 19 |
| hz1A5v36 | GFSFGSFVMM | 117 | TIYSSGTGTF | 146 | SGPVRGWGP | 168 | 20 |
| hz1A5v37 | GFTFGSNVMM | 118 | TIYSSGTGTF | 146 | SGPVRGWGP | 168 | 21 |
| hz1A5v38 | GFSFGSNVMM | 115 | TIYSSGTGTF | 146 | SGPVRGWGY | 169 | 22 |
| hz1A5v39 | GFSFSSNVMM | 119 | TIYSSGTGTF | 146 | SGPVRGWGP | 168 | 23 |
| hz1A5v40 | GFSFTSNVMM | 120 | TIYSSGTGTF | 146 | SGPVRGWGP | 168 | 24 |
| hz1A5v41 | GFSFGSNVMM | 115 | TIYSRGTGTF | 147 | SGPVRGWGP | 168 | 25 |
| hz1A5v42 | GFSFGSNVMM | 115 | TIYSRGTGTF | 147 | SGPVRGWGP | 168 | 26 |
| hz1A5v43 | GFSFGSNVMM | 115 | TIYSRGTGTF | 147 | SGPVRGWGP | 168 | 27 |
| hz1A5v44 | GFSFGSNVMM | 115 | TIYSRGTGTY | 151 | SGPVRGWGP | 168 | 28 |
| hz1A5v45 | GFSFGSYVMM | 116 | TIYSRGTGTF | 147 | SGPVRGWGP | 168 | 29 |
| hz1A5v46 | GFTFGSNVMM | 118 | TIYSRGTGTF | 147 | SGPVRGWGP | 168 | 30 |
| hz1A5v47 | GFSFSSNVMM | 119 | TIYSRGTGTF | 147 | SGPVRGWGP | 168 | 31 |
| hz1A5v48 | GFSFGSYVMM | 116 | TIYSRGTGTY | 151 | SGPVRGWGP | 168 | 32 |
| hz1A5v49 | GFTFSSNVMM | 121 | TIYSRGTGTF | 147 | SGPVRGWGP | 168 | 33 |
| hz1A5v50 | GFTFSSYVMM | 122 | TIYSRGTGTY | 151 | SGPVRGWGP | 168 | 34 |
| hz1A5v51 | GFSFSSNVMM | 119 | TIYSSGTGTF | 146 | SGPVRGWGP | 168 | 467 |
| hz1A5v52 | GFSFSSNVMM | 119 | TIYSSGTGTY | 149 | SGPVRGWGP | 168 | 489 |
| hz1A5v53 | GFSFGSNVMM | 115 | TIYSSGTGTF | 146 | SGPVRGWGP | 168 | 490 |
| L57B04 Humanized Variants | | | | | | | |
| hz57B04v3 | ERTFSTYTMG | 123 | VVNWSGGSKY | 152 | GGAYSGPYYDTRQYTY | 170 | 36 |
| | | | | | | | 498 |
| hz57B04v4 | ERTFSTYTMG | 123 | VVNWSGGSKY | 152 | GGAYSGPYYDTRQYTY | 170 | 37 |
| | | | | | | | 499 |
| hz57B04v8 | ERTFSTYTMG | 123 | VVNWSGGSKY | 152 | GGAYSGPYYDTRQYTY | 170 | 38 |
| | | | | | | | 500 |
| hz57B04v9 | ERTFSTYTMG | 123 | VVNWSGGSKY | 152 | GGAYSGPYYDTRQYTY | 170 | 39 |
| | | | | | | | 501 |
| hz57B04v15 | ERTFSTYTMG | 123 | VVNWGGGSKY | 153 | GGAYSGPYYDTRQYTY | 170 | 40 |
| hz57B04v20 | ERTFSTYTMG | 123 | VVNWGGGSKY | 153 | GGAYSTPYYDTRQYTY | 171 | 41 |
| hz57B04v23 | ERTFSTYTMG | 123 | VVNWSGGSKY | 152 | GGAYSGPYYDTRQYTY | 170 | 42 |
| | | | | | | | 502 |
| hz57B04v24 | ERTFSTYTMG | 123 | VVNWGGGSKY | 153 | GGAYSGPYYDTRQYTY | 170 | 43 |
| | | | | | | | 503 |
| L58E05 Humanized Variants | | | | | | | |
| hz58E05v1 | GSTFSMYHMS | 124 | TSHHGGTTN | 154 | DHGYNGRGY | 172 | 45 |
| | | | | | | | 504 |
| hz58E05v2 | GSTFSMYHMS | 124 | TSHHGGTTN | 154 | DHGYNGRGY | 172 | 46 |
| | | | | | | | 505 |
| hz58E05v3 | GSTFSMYHMS | 124 | TSHHGGTTN | 154 | DHGYNGRGY | 172 | 47 |
| | | | | | | | 506 |
| hz58E05v4 | GSTFSMYHMS | 124 | TSHHGGTTN | 154 | DHGYNGRGY | 172 | 48 |
| | | | | | | | 507 |
| hz58E05v5 | GSTFSMYHMS | 124 | TSHHGGTTN | 154 | DHGYQGRGY | 173 | 49 |
| | | | | | | | 508 |
| hz58E05v6 | GSTFSMYHMS | 124 | TSHHGGTTN | 154 | DHGYNARGY | 174 | 50 |
| | | | | | | | 509 |
| hz58E05v7 | GSTFSMYHMS | 124 | TSHHGGTTN | 154 | DHGYNTRGY | 175 | 51 |
| | | | | | | | 510 |
| hz58E05v8 | GSTFSMYHMS | 124 | TSHHGGTTN | 154 | DHGYNGRGY | 172 | 52 |
| | | | | | | | 511 |
| hz58E05v9 | GSTFSMYHMS | 124 | TSHHGGTTN | 154 | DHGYQGRGY | 173 | 53 |
| | | | | | | | 512 |
| hz58E05v10 | GSTFSMYHMS | 124 | TSHHGGTTN | 154 | DHGYNARGY | 174 | 54 |
| | | | | | | | 513 |
| hz58E05v11 | GSTFSMYHMS | 124 | TSHHGGTTN | 154 | DHGYNTRGY | 175 | 55 |
| | | | | | | | 514 |
| hz58E05v13 | GSTFSMYHMS | 124 | TSHHGGTTN | 154 | DHGYQGRGY | 173 | 56 |
| hz58E05v16 | GSTFSLYHMS | 125 | TSHHGGTTN | 154 | DHGYQGRGY | 173 | 57 |
| hz58E05v18 | GSTFSSYHMS | 126 | TSHHGGTTN | 154 | DHGYQGRGY | 173 | 58 |
| hz58E05v19 | GSTFSMYHMS | 124 | TSHHGGTTN | 154 | DHGYQGRGY | 173 | 59 |
| hz58E05v20 | GSTFSTYHMS | 127 | TSHHGGTTN | 154 | DHGYQGRGY | 173 | 60 |
| hz58E05v21 | GSTFSKYHMS | 128 | TSHHGGTTN | 154 | DHGYQGRGY | 173 | 61 |
| hz58E05v22 | GSTFSFYHMS | 129 | TSHHGGTTN | 154 | DHGYQGRGY | 173 | 62 |
| hz58E05v23 | GSTFSDYHMS | 130 | TSHHGGTTN | 154 | DHGYQGRGY | 173 | 63 |
| hz58E05v24 | GSTFSRYHMS | 131 | TSHHGGTTN | 154 | DHGYQGRGY | 173 | 64 |
| hz58E05v25 | GSTFSMYHMS | 124 | TSHHGGTTN | 154 | DHGYQGRGY | 173 | 65 |
| hz58E05v26 | GSTFSMYHMS | 124 | TSHHGGTTN | 154 | DHGYQGRGY | 173 | 66 |
| hz58E05v27 | GSTFSSYHMS | 126 | TSHHGGTTN | 154 | DHGYQGRGY | 173 | 67 |
| hz58E05v28 | GSTFSMYHMS | 124 | TSHHGGTTN | 154 | DHGYNGRGY | 172 | 68 |
| hz58E05v29 | GSTFSMYHMS | 124 | TSHHGGTTN | 154 | DHGYNGRGY | 172 | 69 |
| hz58E05v30 | GSTFSMYHMS | 124 | TSHHGGTTN | 154 | DHGYNGRGY | 172 | 70 |
| hz58E05v31 | GSTFSMYHMS | 124 | TSHHGGTTN | 154 | DHGYQGRGY | 173 | 71 |
| hz58E05v32 | GSTFSSYHMS | 126 | TSHHGGTTN | 154 | DHGYQGRGY | 173 | 72 |
| hz58E05v36 | GSTFSMYHMS | 124 | TSHHGGTTN | 154 | DHGYGGRGY | 176 | 73 |
| hz58E05v37 | GSTFSMYHMS | 124 | TSHHGGTTN | 154 | DHGYSGRGY | 177 | 74 |
| hz58E05v38 | GSTFSMYHMS | 124 | TSHHGGTTN | 154 | DHGYVGRGY | 178 | 75 |
| hz58E05v39 | GSTFSMYHMS | 124 | TSHHGGTTN | 154 | DHGYEGRGY | 179 | 76 |
| hz58E05v40 | GSTFSMYHMS | 124 | TSHHGGTTN | 154 | DHGYNPRGY | 180 | 77 |
| hz58E05v41 | GSTFSMYHMS | 124 | TSHHGGTTN | 154 | DHGYGNRGY | 181 | 78 |
| hz58E05v42 | GSTFSMYHMS | 124 | TSHHGGTTN | 154 | DHGNYGRGY | 182 | 79 |
| hz58E05v43 | GSTFSMYHMS | 124 | TSHHGGTTN | 154 | DHGYNRGGY | 183 | 80 |
| hz58E05v44 | GSTFSSYHMS | 126 | TSHHGGTTN | 154 | DHGYGGRGY | 176 | 81 |
| hz58E05v45 | GSTFSSYHMS | 126 | TSHHGGTTN | 154 | DHGYEGRGY | 179 | 82 |
| hz58E05v46 | GSTFSSYHMS | 126 | TSHHGGTTN | 154 | DHGNYGRGY | 182 | 83 |
| hz58E05v47 | GSTFSIYHMS | 132 | TSHHGGTTN | 154 | DHGYGGRGY | 176 | 84 |
| hz58E05v48 | GFTFSSYHMS | 133 | TSHHGGTTN | 154 | DHGYQGRGY | 173 | 85 |
| hz58E05v49 | GSTFSSYHMS | 126 | TSHHGGTTN | 154 | DHGYQGRGY | 173 | 86 |
| hz58E05v50 | GSTFSSYHMS | 126 | TSHHGGTTN | 154 | DHGYQGRGY | 173 | 87 |
| hz58E05v51 | GSTFSSYHMS | 126 | TSHHGGTTN | 154 | DHGYQGRGY | 173 | 88 |
| hz58E05v52 | GSTFSSYHMS | 126 | TSHHGGTTN | 154 | DHGYQGRGY | 173 | 89 |
| hz58E05v53 | GSTFSSYHMS | 126 | TSHHGGTTN | 154 | DHGYQGRGY | 173 | 90 |
| hz58E05v54 | GSTFSSYHMS | 126 | TSHHGGTTN | 154 | DHGYQGRGY | 173 | 91 |
| hz58E05v55 | GFTFSSYHMS | 126 | TSHHGGTTN | 154 | DHGYQGRGY | 173 | 466 |
| IH5 Humanized Variants | | | | | | | |
| hz1H5v1 | GRSFGDYTVG | 145 | GLSWLGGTIY | 167 | SRSAISRKATDFGS | 488 | 106 |
| hz1H5v2 | GRSFGDYTVG | 145 | GLSWLGGTIY | 167 | SRSAISRKATDFGS | 488 | 107 |
| hz1H5v3 | GRSFGDYTVG | 145 | GLSWLGGTIY | 167 | SRSAISRKATDFGS | 488 | 108 |
| hz1H5v4 | GRSFGDYTVG | 145 | GLSWLGGTIY | 167 | SRSAISRKATDFGS | 488 | 109 |
| L57B06 Humanized Variants | | | | | | | |
| h57B06v5 | GGTFSSYAMG | 139 | AISSEGGSTY | 161 | KGVGWPQEQASYDY | 189 | 111 |
| | | | | | | | 515 |
| h57B06v6 | GGTFSSYAMG | 139 | AISSEGGSTY | 161 | KGVGWPQEQASYDY | 189 | 112 |
| | | | | | | | 516 |
| h57B06v7 | GGTFSSYAMG | 139 | AISSEGGSTY | 161 | KGVGWPQEQASYDY | 189 | 113 |
| | | | | | | | 517 |
| h57B06v8 | GGTFSSYAMG | 139 | AISSEGGSTY | 161 | KGVGWPQEQASYDY | 189 | 114 |
| | | | | | | | 518 |

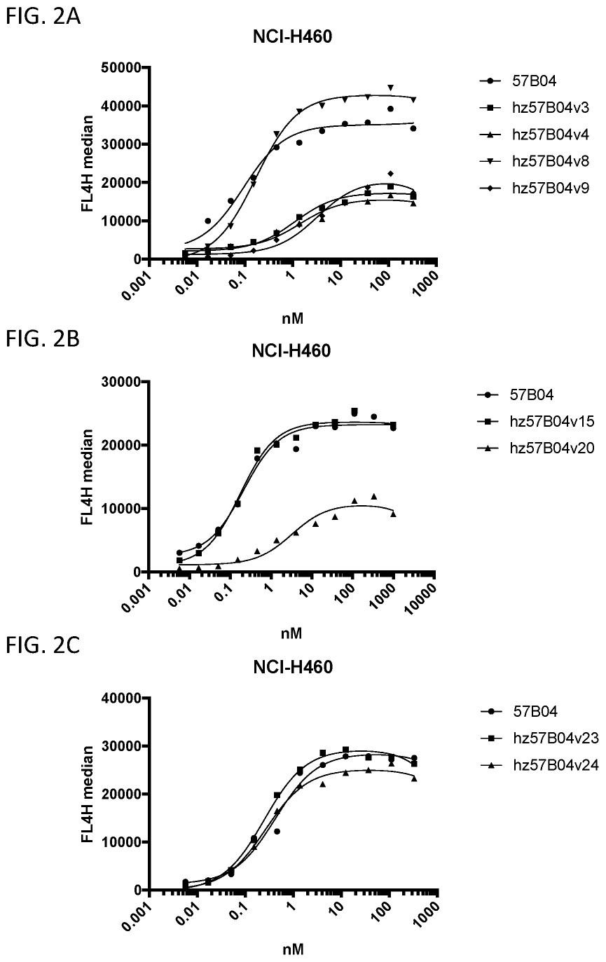

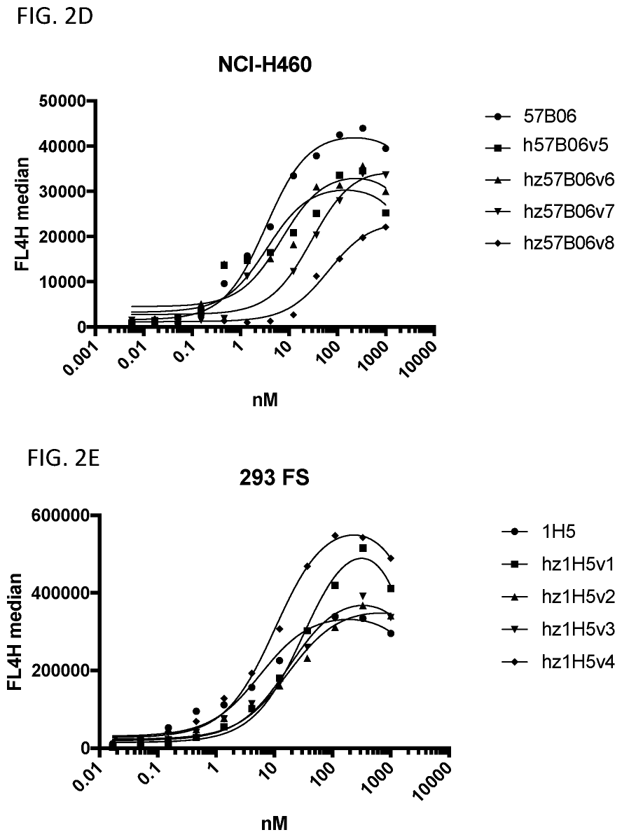

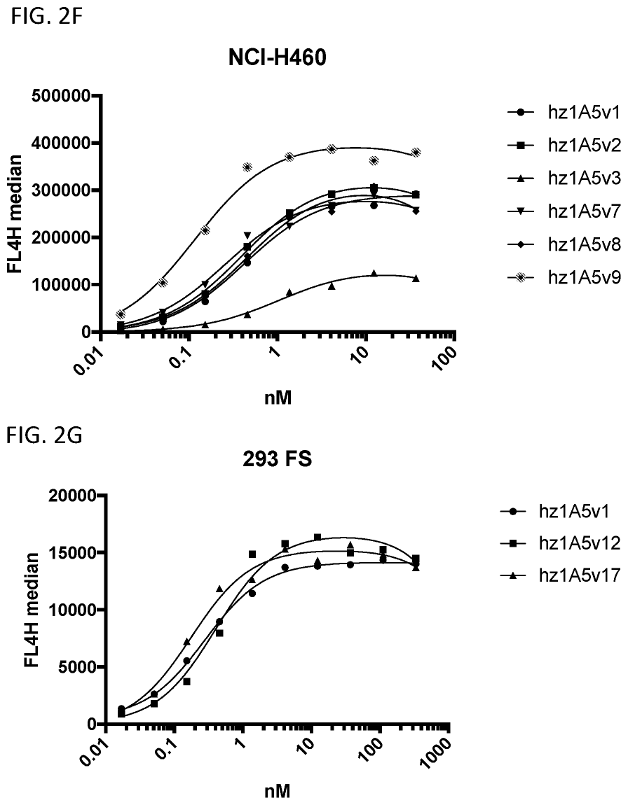

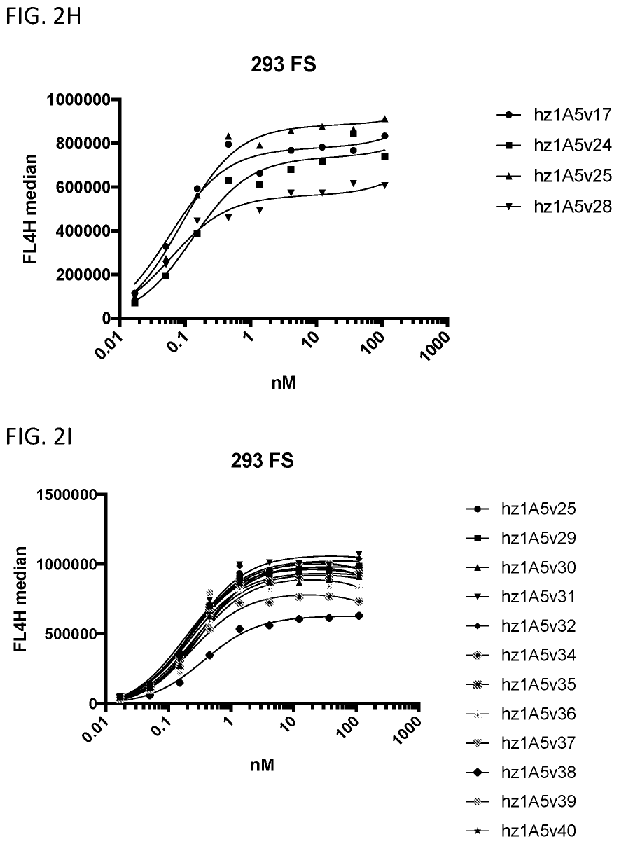

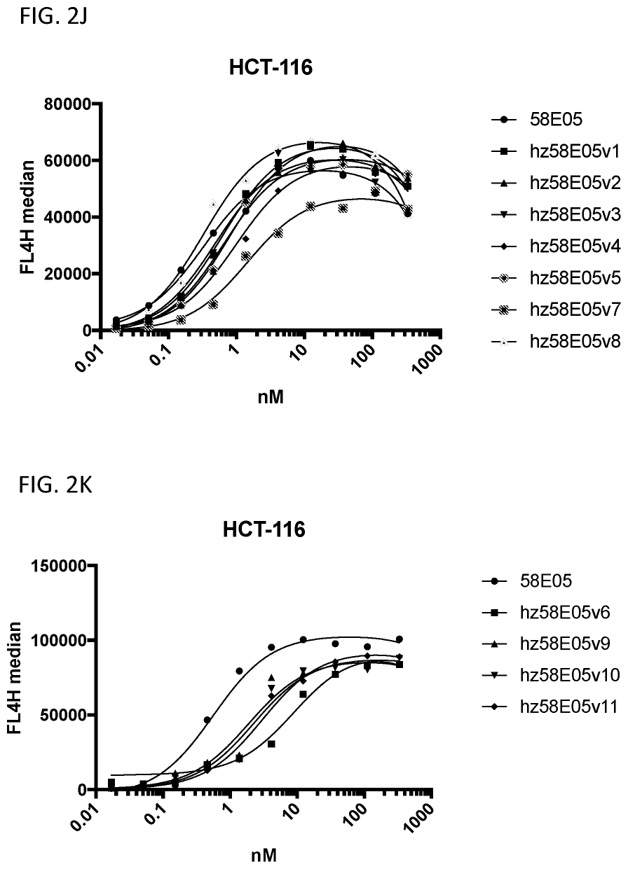

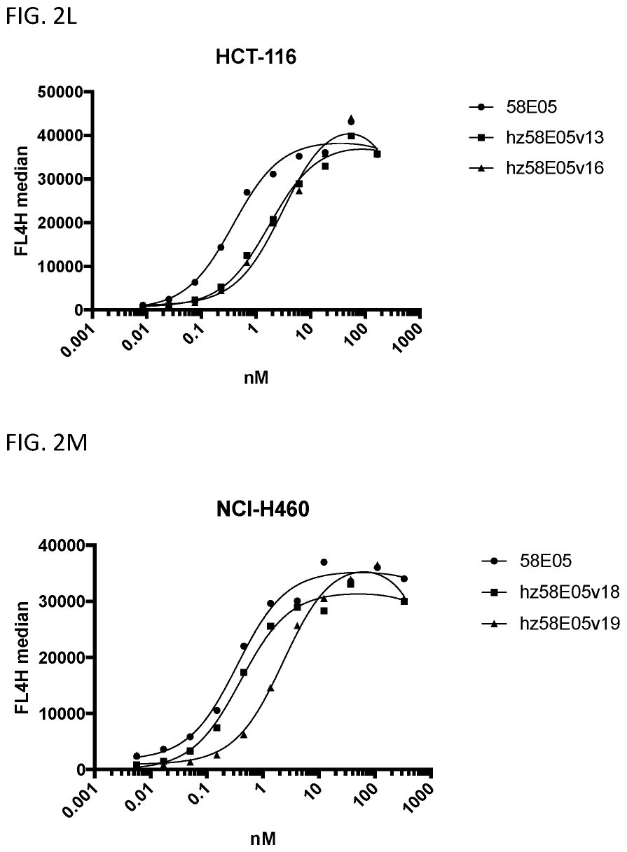

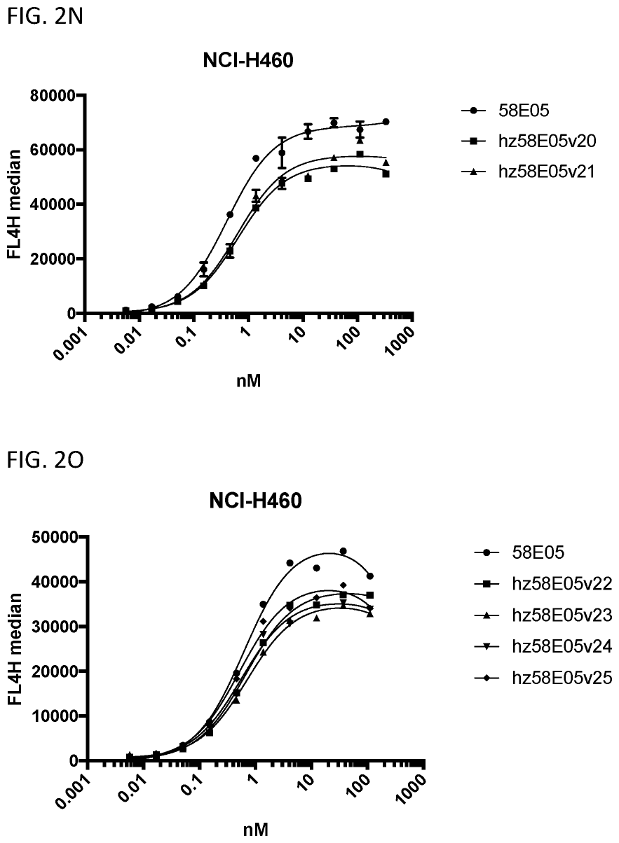

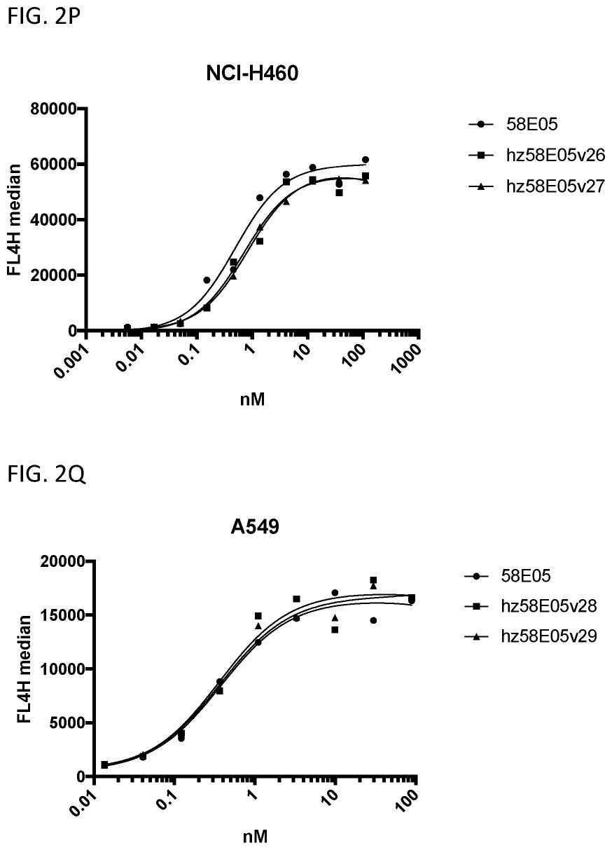

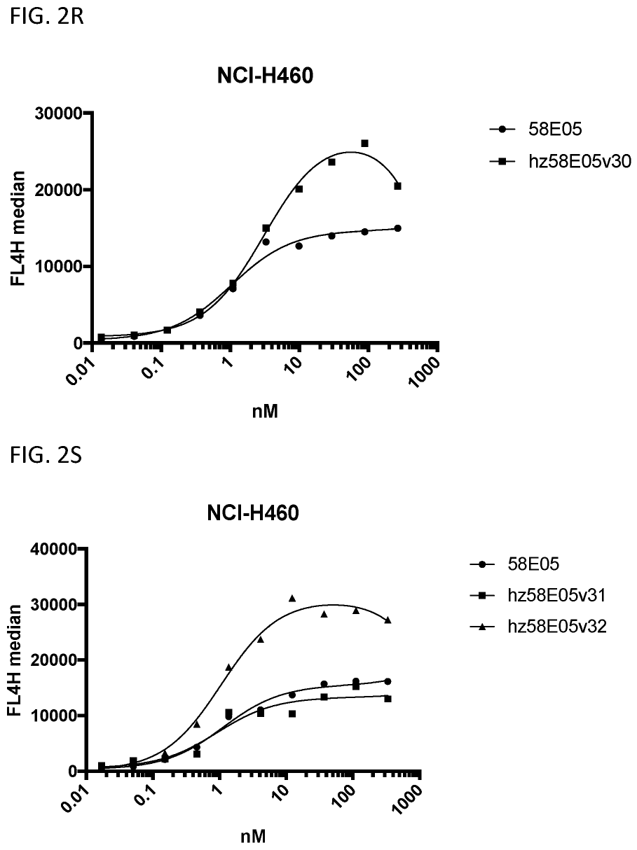

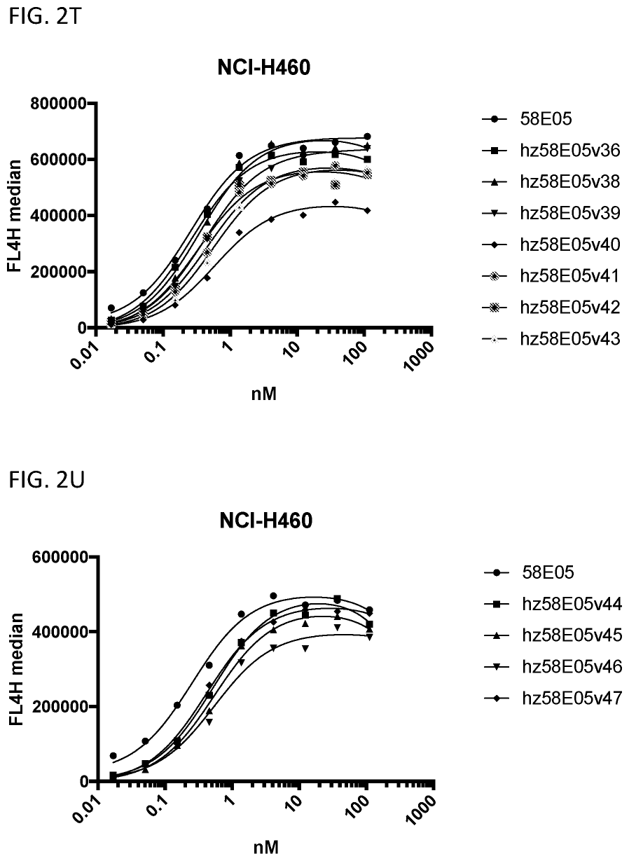

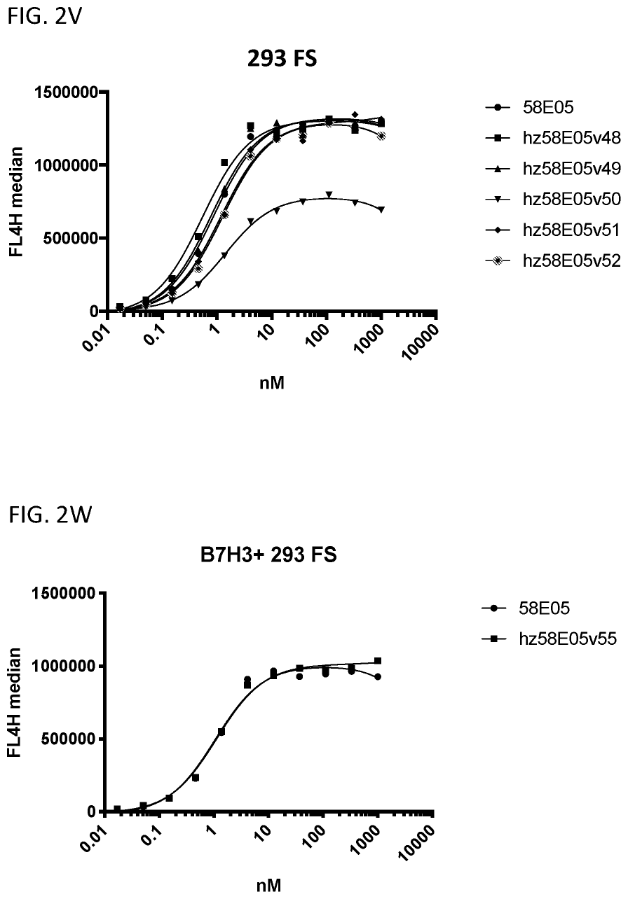

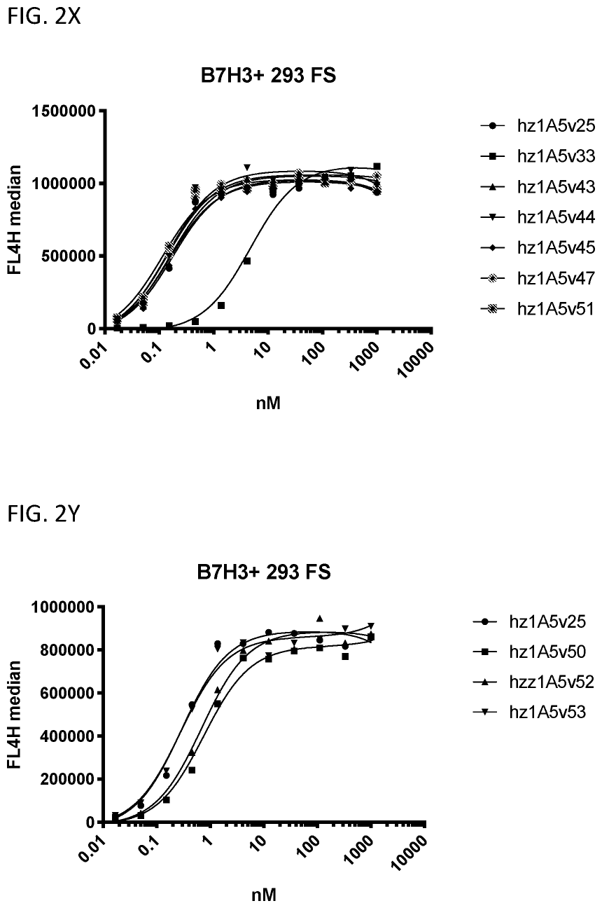

Humanized variants of the B7H3 sdAbs were tested for their ability to bind B7H3 expressing cells substantially as described in Example 2, and binding was compared to the parental sdAb in most cases. In some cases, HCT-116 or A549 cell lines, which both endogenously express B7H3, were used in these studies. Results are shown in FIGS. 2A-2Y, which confirm binding of the humanized variants.

### Example 4: Method of Producing B7H3-Targeted Constrained CD3 Binding Proteins

Multispecific polypeptide constructs were generated containing a disulfide stabilized anti-CD3 Fv binding region that exhibits constrained CD3 binding, a heterodimeric Fc domain, and one or more B7H3 sdAb described above positioned amino-terminally relative to the Fc region and/or carboxy-terminally relative to the CD3 binding region. The multispecific constructs were generated in various configurations, as shown in FIG. 3A-3E. In some cases, the multispecific polypeptide constructs contained at least one co-stimulatory receptor binding region (CRBR) positioned amino-terminally relative to the Fc region and/or carboxy-terminally relative to the CD3 binding region. An exemplary CRBR is a sdAb targeting a 4-1BB co-stimulatory receptor (e.g. containing a CDR1, a CDR2 and a CDR3 set forth in SEQ ID Nos: 468, 469 and 470, respectively; e.g. set forth in SEQ ID NO:400).

In the exemplary constructs, polynucleotides encoding at least a first polypeptide chain and a second polypeptide chain of the heterodimeric multispecific polypeptide construct were generated and cloned into a plasmid for expression. The first polypeptide chain generally included in order, from the N-terminus to C-terminus, an Fc hole polypeptide (e.g. set forth in SEQ ID NO:302, or in some cases SEQ ID NO:304); a cleavable or a non-cleavable linker, such as one containing one or more substrate recognition sites for a protease; and a variable light (VL) domain of a dsFv anti-CD3 antibody (e.g. set forth in SEQ ID NO:417). The second polypeptide chain generally included in order, from the N-terminus to C-terminus, an Fc knob polypeptide (e.g. set forth in SEQ ID NO: 295, or in some cases SEQ ID NO:299); the same cleavable linker or the same non-cleavable linker as the first polypeptide chain; and a variable heavy domain of a dsFv anti-CD3 antibody (e.g. set forth in SEQ ID NO:237). The constructs were generated with the exemplary non-cleavable linker, GGGGGSGGGGGSGGGGGS (SEQ ID NO:317), or the exemplary cleavable linker, GGSGGGG [symbol] IGGSGGS (SEQ ID NO:361) containing a substrate recognition site for granzyme B. One or both of the polypeptide chains additionally encoded one or more B7H3 sdAb amino terminal to the Fc domain and/or carboxy terminal to the CD3 binding region, and/or a co-stimulatory receptor binding domain amino terminal to the Fc domain and/or carboxy terminal to the CD3 binding region, in various configurations.

Separate plasmids encoding each chain of a heterodimeric constrained CD3 binding protein were transiently transfected at an equimolar ratio into mammalian cells (either HEK293 or CHO) using polyethylenimine. Supernatant was collected after 3-7 days, and secreted recombinant protein was purified by protein A chromatography, followed by either preparative size exclusion chromatography (SEC) or flow-through hydrophobic interaction chromatography (HIC). Heterodimeric protein was selectively purified owing to a mutation designed into one chain of the heterodimeric Fc at position I253R or H435R (usually the hole-Fc) such that it did not bind protein A. The second chromatography step on SEC (AKTA with Superdex-200 resin) or FT-HIC (AKTA with butyl/phenyl sepharose) was used to remove undesired cross-paired species containing two heterodimeric Fcs that were more hydrophobic and twice the expected molecular weight.

The method favored production of heterodimeric multispecific polypeptide constructs, containing properly paired species of heterodimeric Fc and the disulfide stabilized anti-CD3 Fv as described (e.g. anti-CD3 VH with the mutation G44C as set forth in SEQ ID NO: 237 and VL with the mutation G100C as set forth in SEQ ID NO: 417). Purified heterodimeric constrained CD3 binding protein was stable and did not accumulate cross-paired species upon prolonged incubation at 4° C. or increased protein concentration.

Table E3 sets forth exemplary generated constrained multispecific constructs:

TABLE E3
B7H3 VHH Constrained Multispecific Constructs

| | | N-term sdAb | | | | C-term sdAb |
|---|---|---|---|---|---|---|
| Construct | | (B7H3) | | | CD3 Binding | (B7H3) |
| # | Chain | (SEQ ID NO) | Fc | Linker | Domain | (SEQ ID NO) |
| cx2846 | 1 | 58E05v27 | hFc-Knob | IEPDI | Con1 VH | 57B04v24 |
| | | (67) | | | | (503, e.g. 43) |
| | 2 | — | hFc-Hole | IEPDI | Con1 VL | — |
| cx3072 | 1 | 58E05v27 | hFc-Knob | (G5S)3 | Con1 VH | 57B04v24 |
| | | (67) | | | | (503) |
| | 2 | — | hFc-Hole | (G5S)3 | Con1 VL | — |
| cx3090 | 1 | 58E05v27 | hFc-Knob | IEPDI | Con1 VH | 57B04v24 |
| | | (67) | | | | (503, e.g. 43) |
| | 2 | — | hFc-Hole | IEPDI | Con1 VL | RH3v5-1 (41BB) |
| | | | | | | (400) |
| cx3243 | 1 | 58E05v27 | hFc-Knob | (G5S)3 | Con1 VH | 57B04v24 |
| | | (67) | | | | (503, e.g. 43) |
| | 2 | — | hFc-Hole | (G5S)3 | Con1 VL | RH3v5-1 (41BB) |
| | | | | | | (400) |
| cx3834 | 1 | 58E05v27 | hFc-Knob | IEPDI | Con1 VH | 58E05v27 |
| | | (67) | | | | (67) |
| | 2 | — | hFc-Hole | IEPDI | Con1 VL | — |
| cx3855 | 1 | 58E05v27 | xELL-Knob | IEPDI | Con1 VH | 58E05v27 |
| | | (67) | | | | (67) |
| | 2 | — | xELL-Hole | IEPDI | Con1 VL | RH3v5-1 (41BB) |
| | | | | | | (400) |
| cx3960 | 1 | 58E05v27 | hFc-Knob | IEPDI | Con1 VH | 1A5 |
| | | (67) | | | | (1) |
| | 2 | — | hFc-Hole | IEPDI | Con1 VL | — |
| cx4136 | 1 | 58E05v27 | xELL-Knob | (G5S)3 | Con1 VH | 58E05v27 |
| | | (67) | | | | (67) |
| | 2 | — | xELL-Hole | (G5S)3 | Con1 VL | — |
| cx4137 | 1 | 58E05v27 | xELL-Knob | (G5S)3 | Con1 VH | 58E05v27 |
| | | (67) | | | | (67) |
| | 2 | — | xELL-Hole | (G5S)3 | Con1 VL | RH3v5-1 (41BB) |
| | | | | | | (400) |
| cx4641 | 1 | 58E05v27 | xELL-Knob | IEPDI | Con1 VH | 1A5 |
| | | (67) | | | | (1) |
| | 2 | — | xELL-Hole | IEPDI | Con1 VL | — |
| cx4645 | 1 | 58E05v27 | xELL-Knob | IEPDI | Con1 VH | 1A5 |
| | | (67) | | | | (1) |
| | 2 | — | xELL-Hole | IEPDI | Con1 VL | — |
| cx4736 | 1 | 58E05v27 | xELL-Knob | IEPDI | Con1 VH | 58E05v27 |
| | | (67) | | | | (67) |
| | 2 | — | xELL-Hole | IEPDI | Con2 VL | — |
| cx4904 | 1 | 58E05v27 | xELL-Knob | IEPDI | Con1 VH | 58E05v27 |
| | | (67) | | | | (67) |
| | 2 | — | xELL-Hole | IEPDI | Con1 VL | RH3v5-1 (41BB) |
| | | | | | | (400) |
| cx4908 | 1 | 58E05v27 | xELL-Knob | IEPDI | Con1 VH | 1A5v12 |
| | | (67) | | | | (8) |
| | 2 | — | xELL-Hole | IEPDI | Con1 VL | RH3v5-1 (41BB) |
| | | | | | | (400) |

### Example 5: Comparison of Binding to Isolated Primary T-cells vs. B7H3-Expressing Cancer Cells

Binding of exemplary B7H3-targeting constrained CD3 engaging constructs set forth in Table E3 to CD3 on the surface of primary T cells or B7H3-expressing cells (A375) was assessed by flow cytometry. The T cells were primary T-cells that were negatively enriched from PBMCs isolated from healthy human donor peripheral blood leukopaks. Bound construct was detected with fluorophore-conjugated secondary antibodies specific for the human Fc (anti-human IgG APC secondary antibody) and binding was measured by flow cytometry. Cells incubated with secondary antibody only served as negative controls. Binding was compared to Dual-affinity Re-targeting Antibody (DART)-monomeric Fc format targeting B7H3 and CD3 (DART-Fc B7H3xCD3; see e.g. WO2017030926A1).

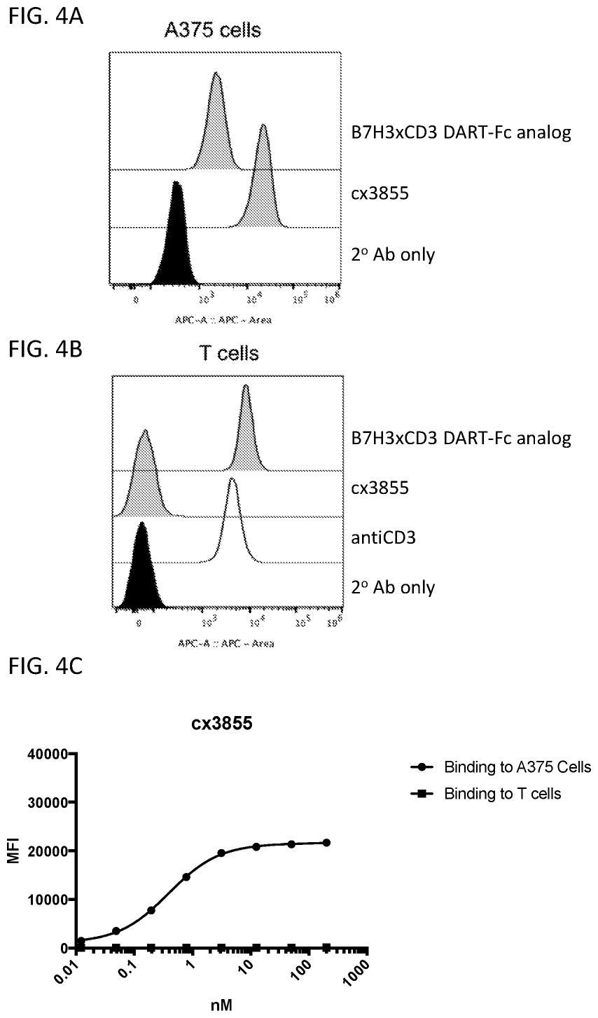

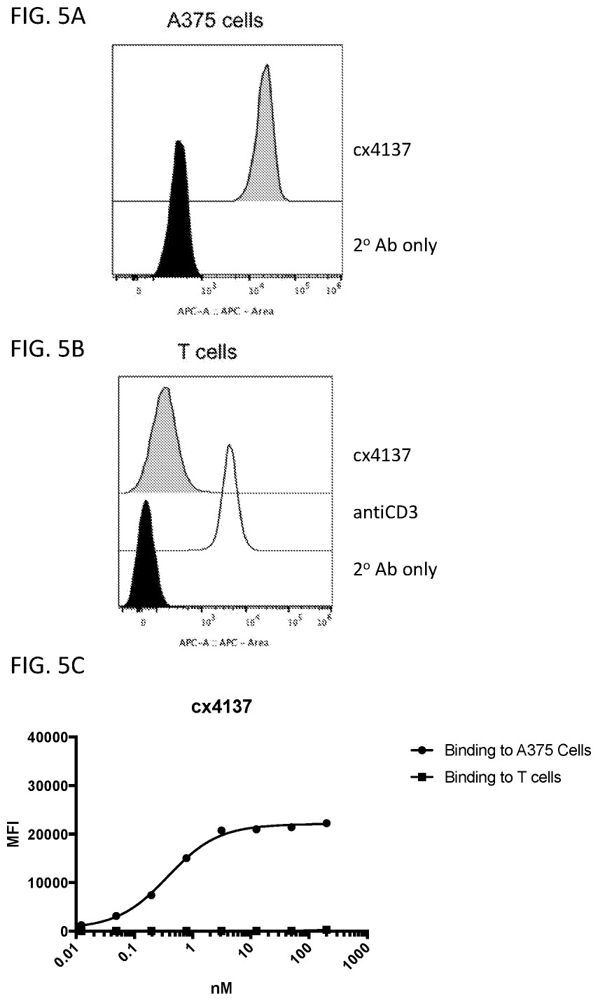

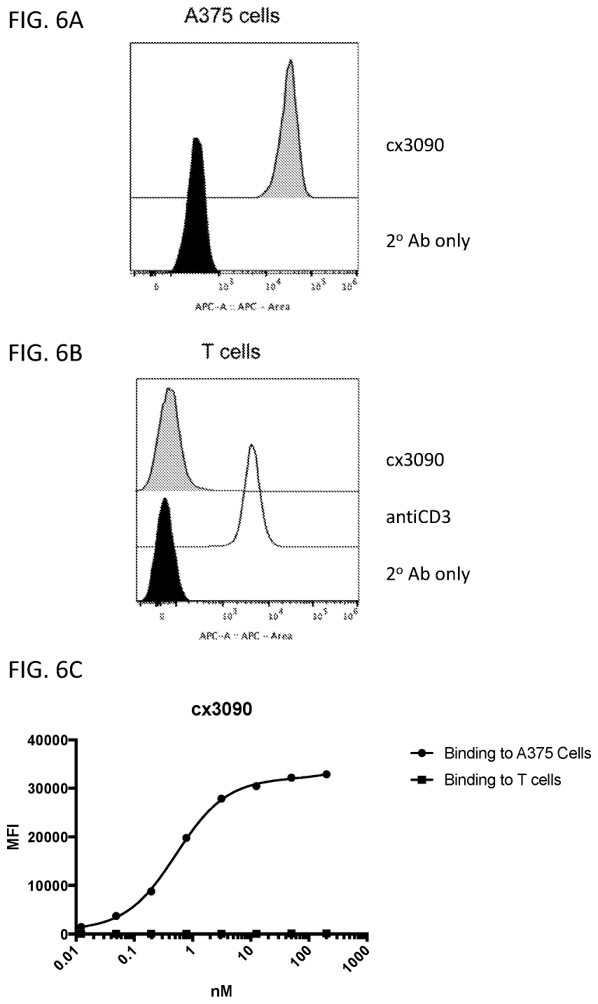

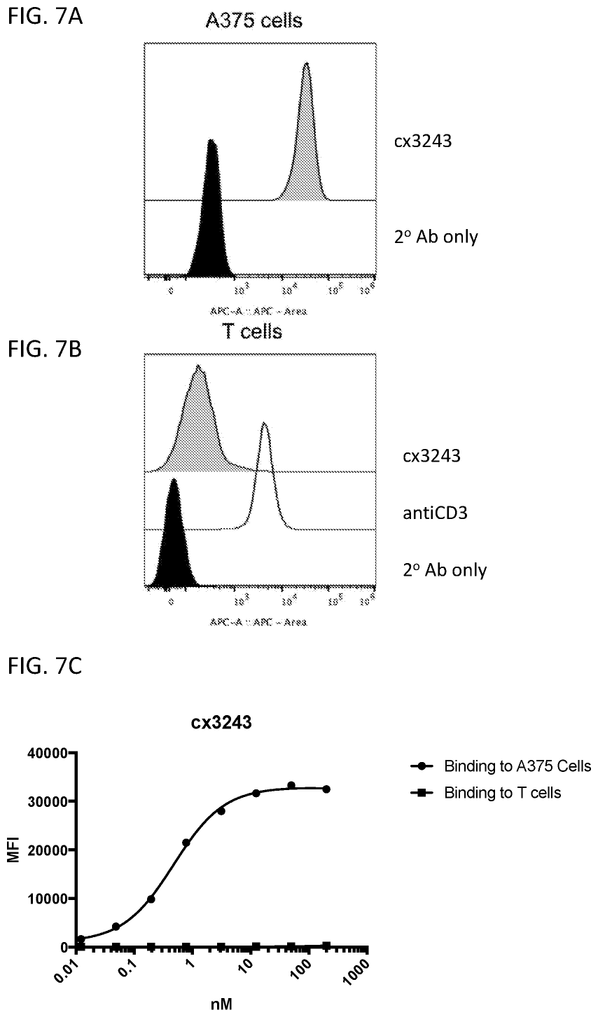

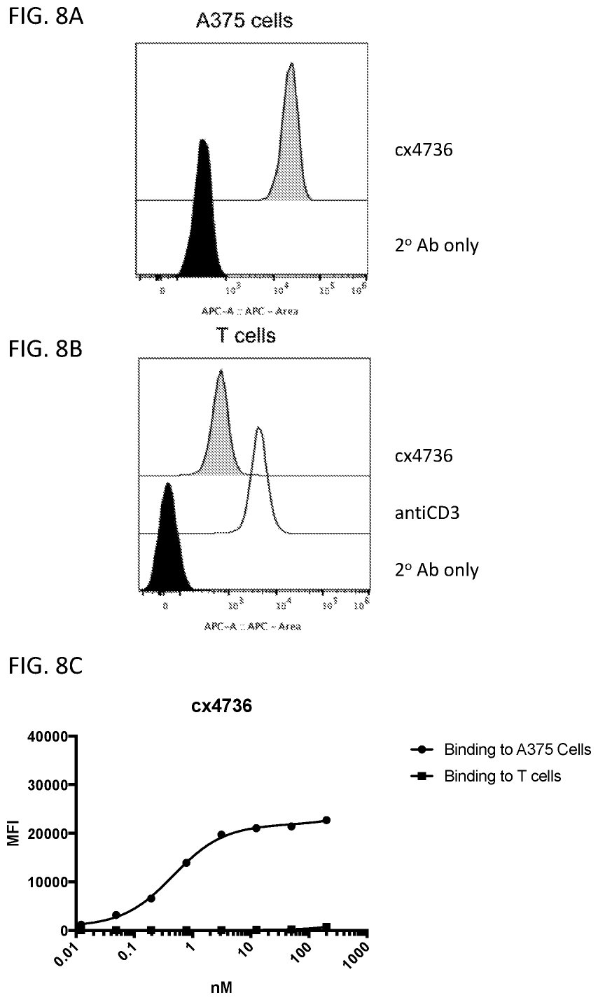

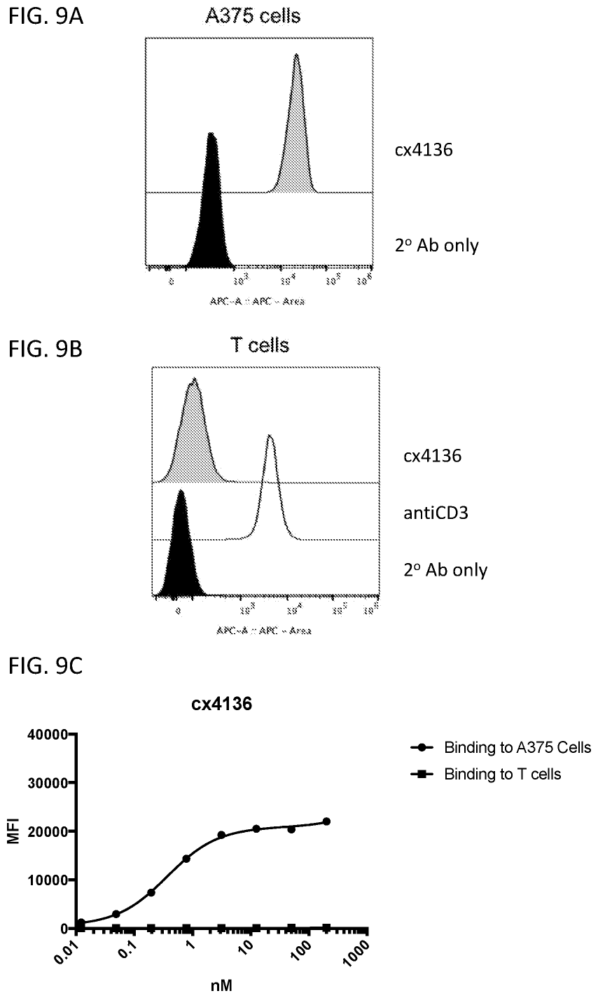

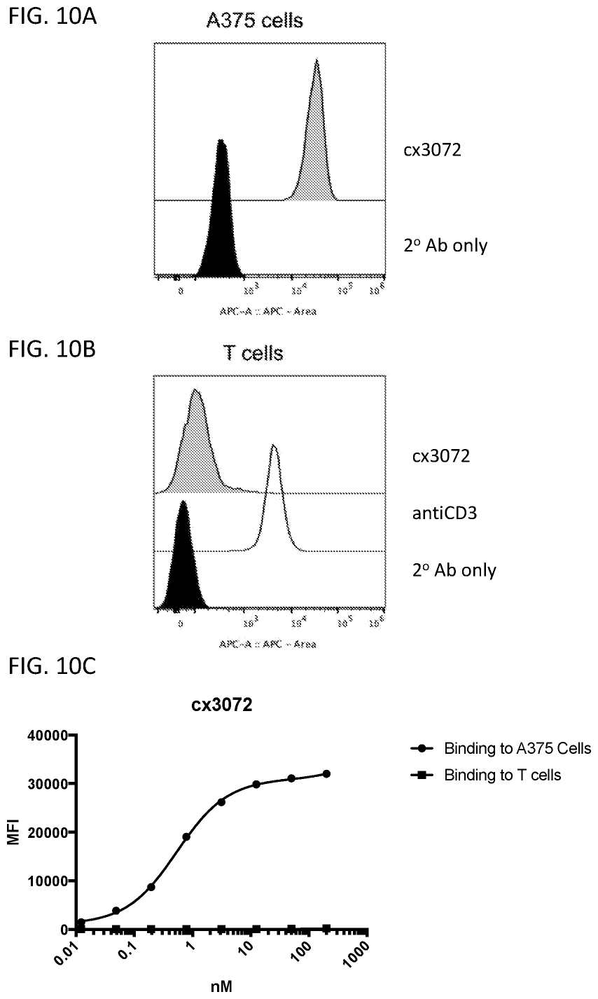

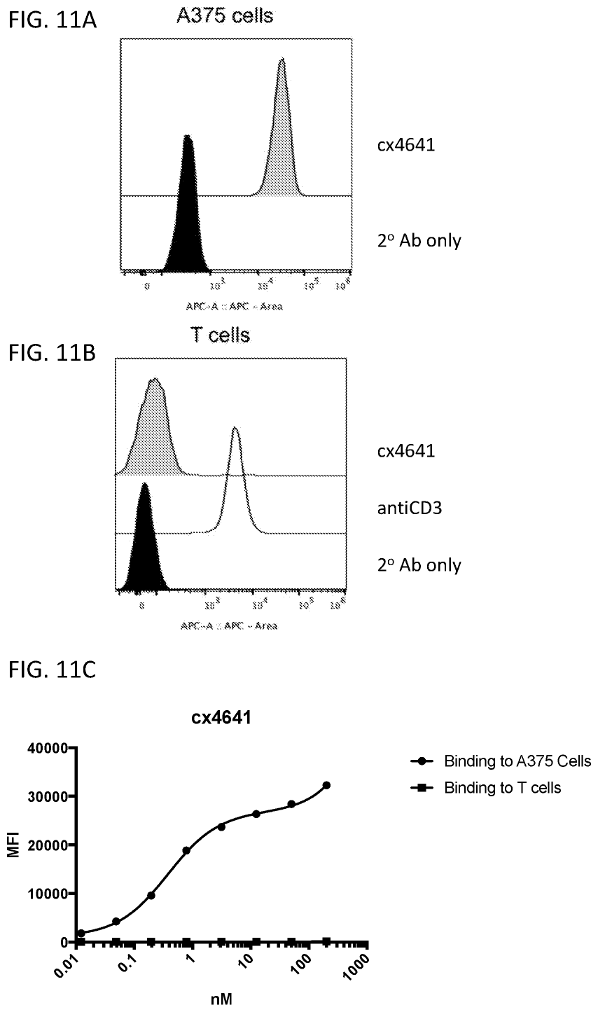

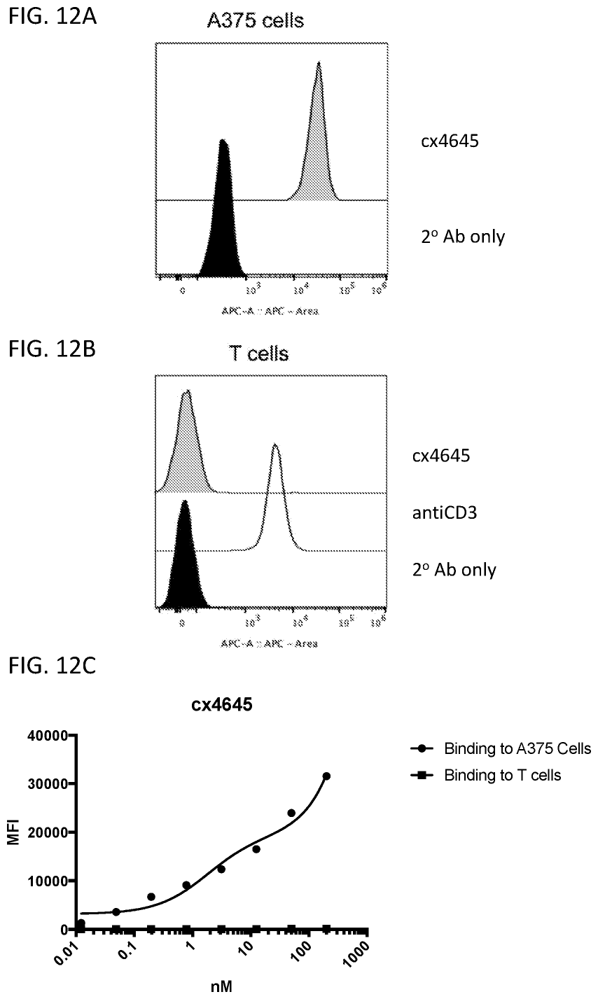

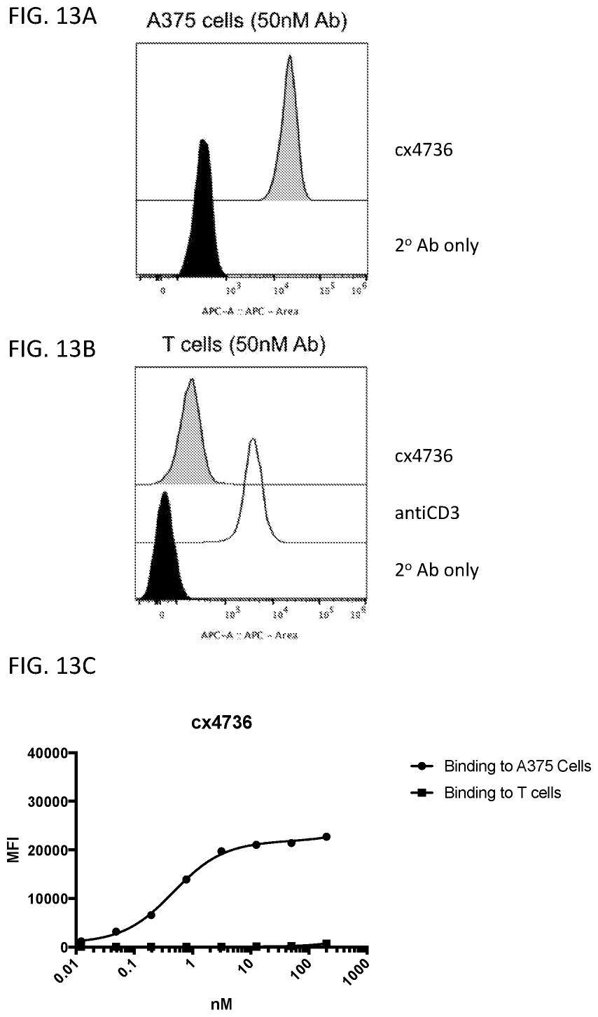

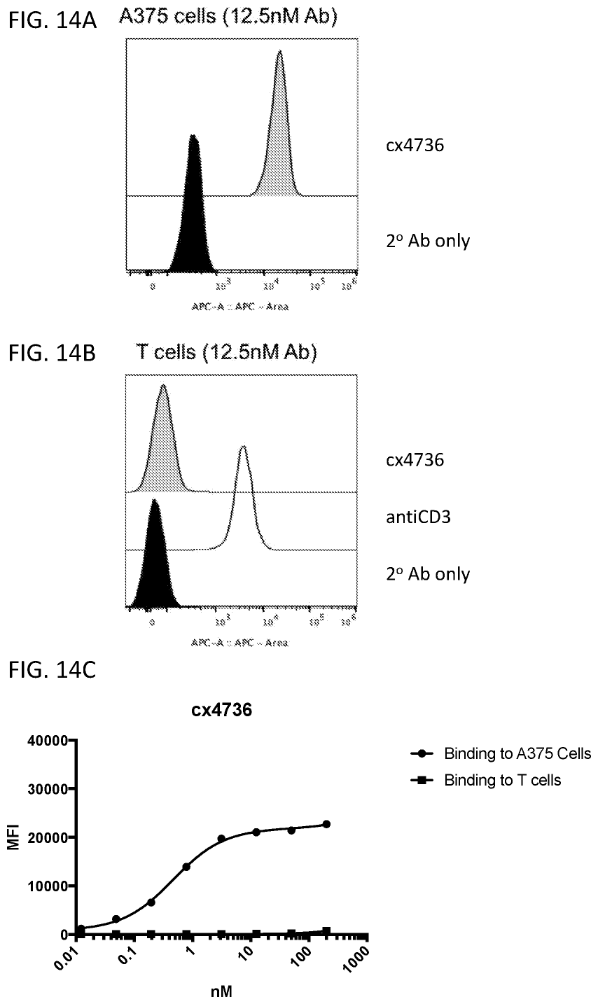

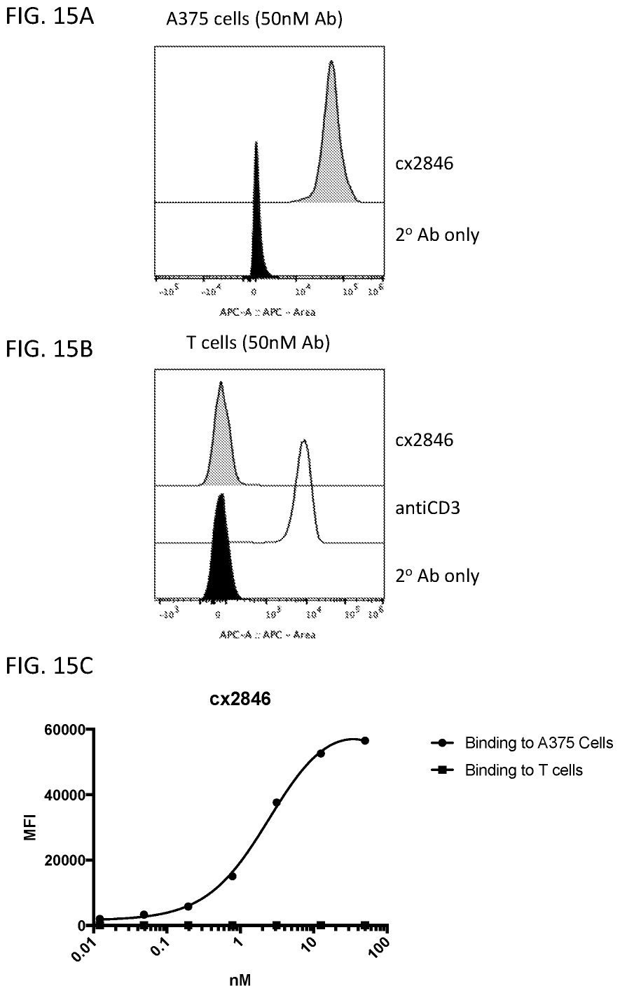

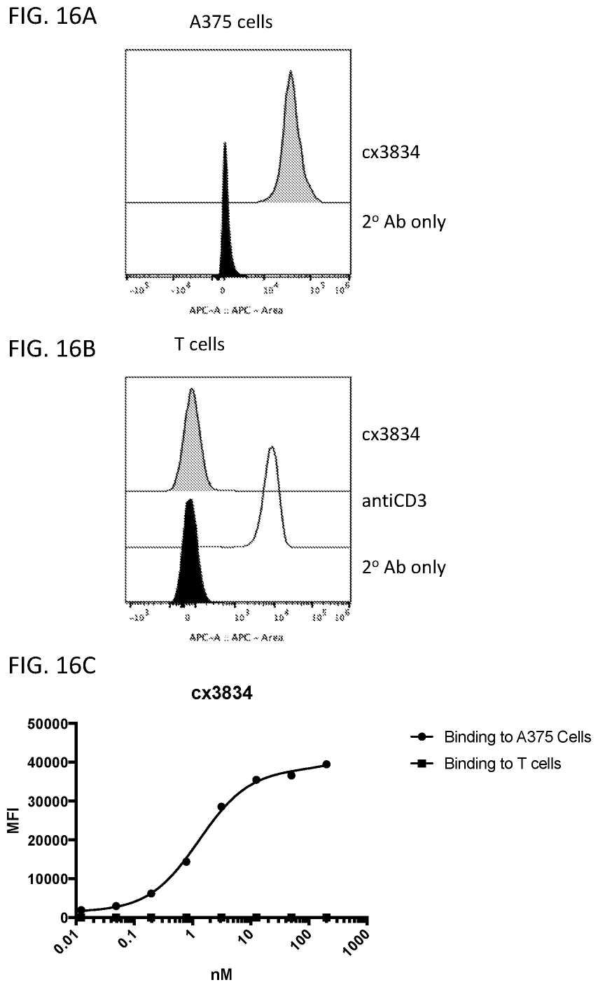

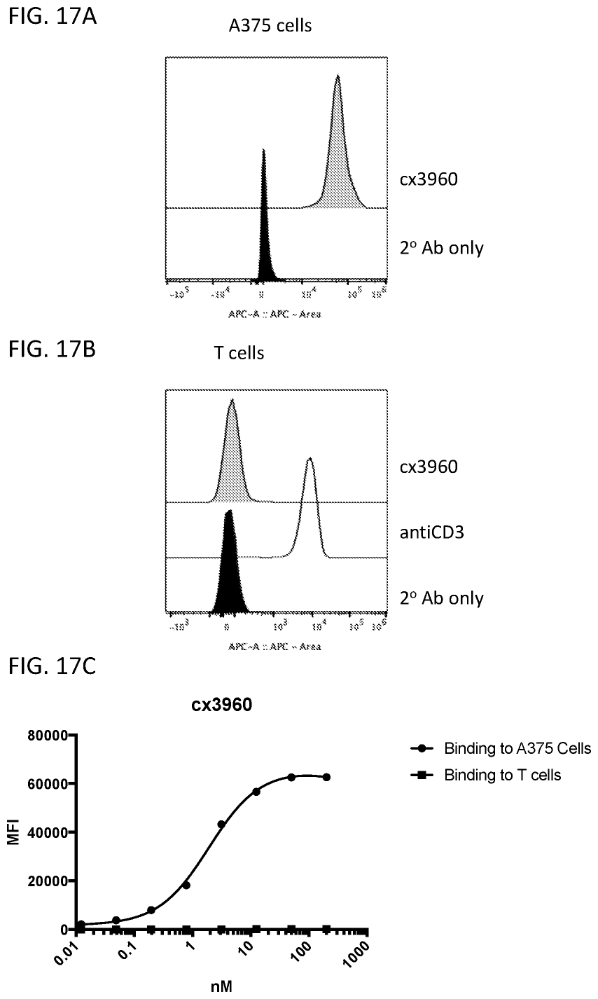

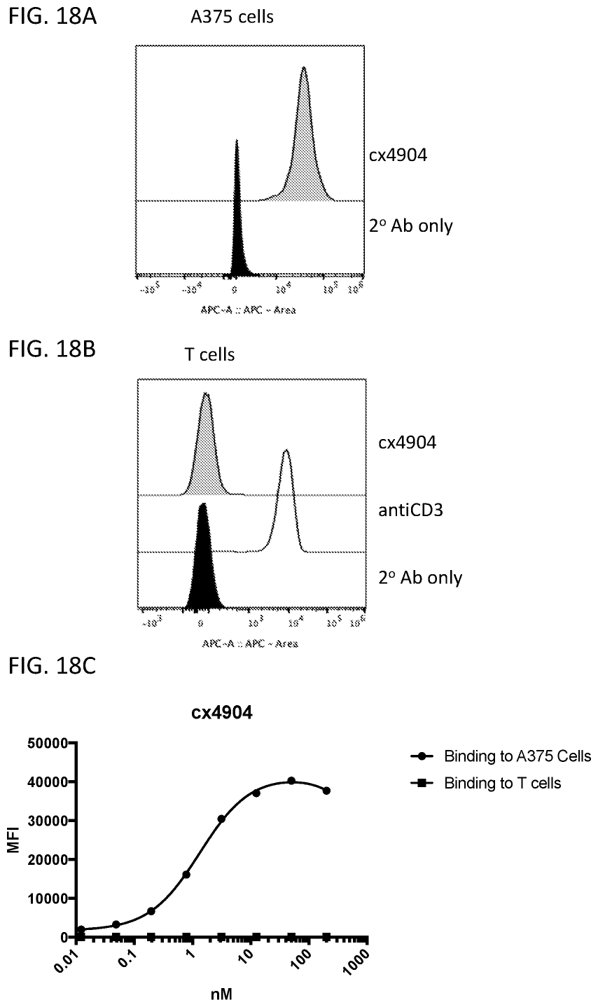

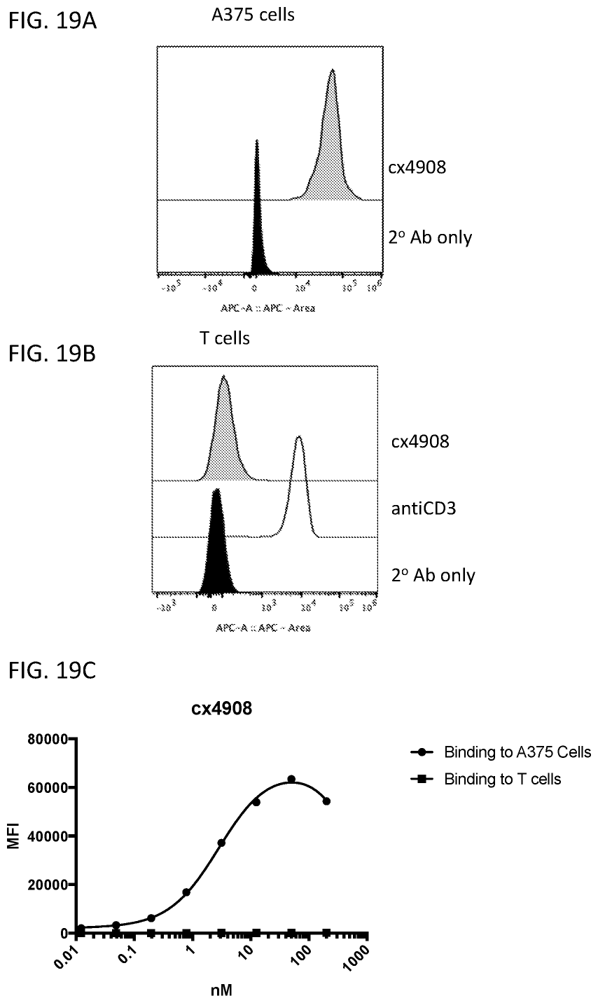

The results, from flow cytometry histograms displaying normalized cell counts versus fluorescence at 200 nM of each construct, are shown in FIGS. 4A-C (cx3855), FIGS. 5A-C (cx4137), FIGS. 6A-C (cx3090), FIGS. 7A-C (cx3243), FIGS. 8A-C (cx4736), FIGS. 9A-C (cx4136), FIGS. 10A-C (cx3072), FIGS. 11A-C (cx4641), FIGS. 12A-C (cx4645), FIGS. 13A-C (cx4736, 50 nM), FIGS. 14A-C (cx4736, 12.5 nM), FIGS. 15A-C (cx2846), FIGS. 16A-C (cx3834), FIGS. 17A-C (cx3960), FIGS. 18A-C (cx4904) and FIGS. 19A-C (cx4908). As shown, the representative B7H3-targeting constrained CD3 engagers were found to bind A375 cells expressing B7H3 (part “A” of each of the figures). However, as shown in parts “B” and “C” of each of the figures, the same constructs were not able to bind to T cells in isolation. In these studies, the binding of the representative B7H3-targeting constrained CD3 engager cx3855 was compared to an analog of a Dual-affinity Re-targeting Antibody (DART)-monomeric Fc format targeting B7H3 and CD3 (DART-Fc B7H3xCD3; see e.g. WO2017030926A1). The DART-Fc B7H3xCD3 contained a B7H3 sequence as set forth in SEQ ID NO: 418, 419, 420. Notably only the DART-Fc format was observed to allow for T-cell binding in the absence of B7H3 engagement (FIG. 4B), whereas, both constructs displayed binding to B7H3-expressing cells (FIG. 4A).

Table E4 summarizes the affinity of exemplary molecules for B7H3 or CD3, as determined from flow cytometry, in these studies.

TABLE E4
Construct Binding Affinity

| | Affinity | Affinity |
|---|---|---|
| Construct # | B7H3 | CD3 |
| cx3072 | 0.539 nM | >200 nM |
| cx3090 | 0.523 nM | >200 nM |
| cx3243 | 0.477 nM | >200 nM |
| cx3855 | 0.395 nM | >200 nM |
| cx4136 | 0.368 nM | >200 nM |
| cx4137 | 0.375 nM | >200 nM |
| cx4641 | 0.379 nM | >200 nM |
| cx4645 | 1.776 nM | >200 nM |
| cx4736 | 0.468 nM | >200 nM |

### Example 6: Assessment of B7H3-Dependent CD3 Reporter T Cell Activation using a Reporter Assay

This example describes assessment of the ability of various representative B7H3-targeting constrained CD3 engaging constructs to activate a CD3 NFAT reporter Jurkat cell line in co-culture with B7H3-expressing cells. In the reporter assay, engagement of CD3 in the Jurkat cells results in NFAT signaling and production of green fluorescence. These assays were used to demonstrate that while T-cell binding via the CD3-binding domain is restricted or inhibited on isolated T-cells (as shown in Example 5), once the B7H3-targeted constrained CD3 engaging constructs provided herein are bound to a cognate antigen they are capable of engaging T-cells and mediating T-cell activation.

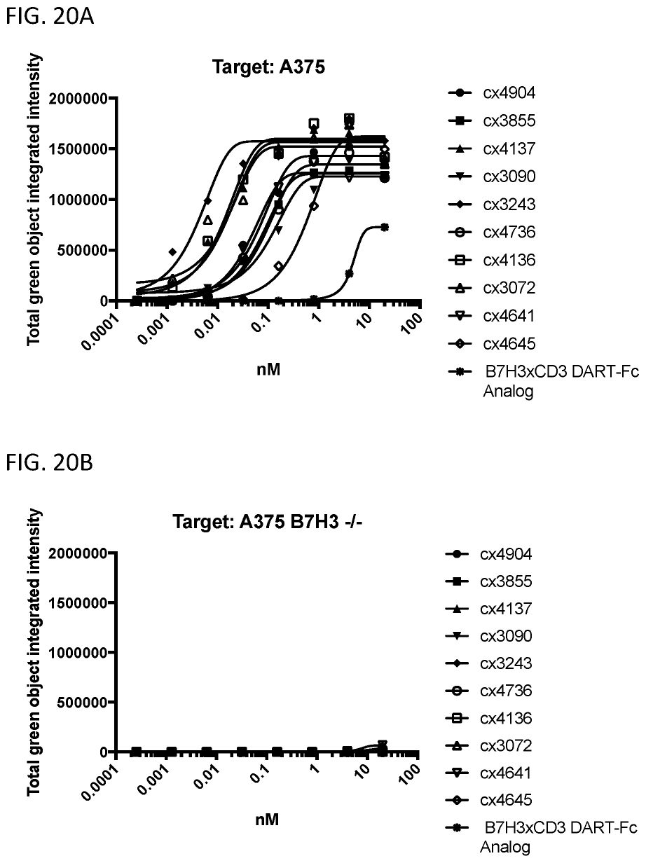

Antigen targeting constrained CD3 engaging constructs were titrated onto co-cultures of B7H3-expressing target cells, A375, and engineered Jurkat cells that expressed NFAT-driven green fluorescence protein (GFP). For reporter assays utilizing adherent target cells, 2.5×10⁴ target cells were seeded, allowed to settle at room temperature for uniform distribution, and incubated for several hours at 37° C. to permit adherence prior to addition of 5.0×10⁴ reporter cells per well and antigen targeting constrained CD3 engaging constructs. Assay plates were serially imaged using an IncuCyte ZOOM system and CD3 reporter cell activation was determined by measuring total green object integrated intensity. As shown in FIG. 20A and 20B, assessed B7H3-targeted constrained CD3 engaging constructs induced reporter activity in cultures containing B7H3 positive cells (A375) (FIG. 20A), but no measurable reporter activity was observed when T cells were cultured with B7H3 negative cells (A375 B7H3−/−) (FIG. 20B).

### Example 7: Assessment of Functional Activity

This example describes the assessment and characterization of exemplary generated B7H3-target constrained CD3 engaging constructs in human primary T cell in vitro assays. Functional activity was compared to the B7H3xCD3 DART-Fc described above.

### 1. T Cell-Mediated Cytotoxicity

Target cells were fluorescently labeled with CytolD red. For cytotoxicity assays utilizing adherent target cells (e.g. A375, A355del B7H3 (B7H3−/−), A549), 1.0×10⁴ target cells were seeded per well, allowed to settle at room temperature for uniform distribution, and incubated for several hours at 37° C. to permit adherence prior to addition of other assay components. Primary T cells were negatively enriched from PBMCs isolated from healthy human donor leukopaks and added at a 10:1 T cell-to-target cell ratio. Green caspase-3/7 reagent was added, which fluorescently labels nuclear DNA of cells undergoing apoptosis was added (Essen BioScience). Antibodies were titrated onto the co-culture and assay plates were serially imaged using an IncuCyte ZOOM system. Target cell death was determined by measuring total red/green overlap object area.

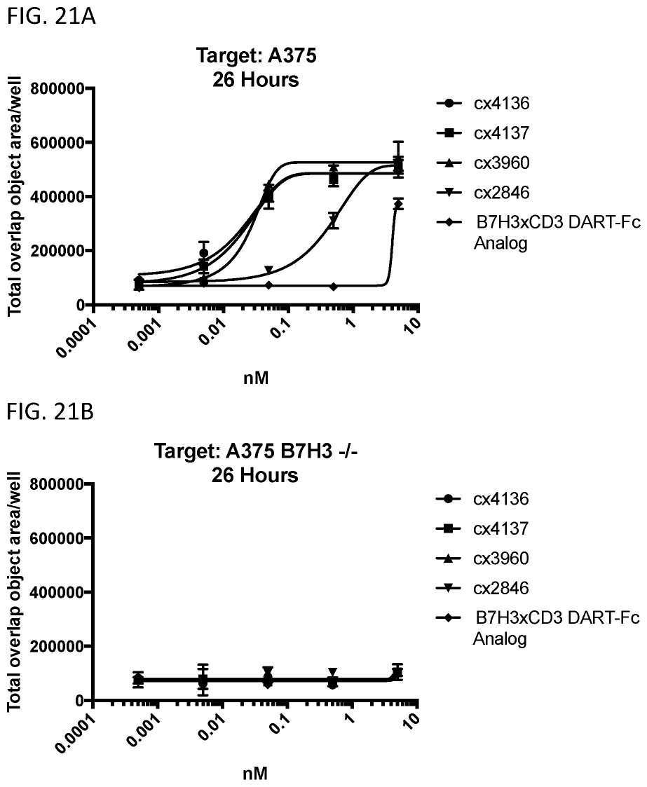

As shown in FIG. 21A, assessed B7H3-targeted constrained CD3 engaging constructs induced potent T-cell-mediated cytotoxicity of B7H3 positive cells (A375). As shown in FIG. 21B, no measurable T cell cytotoxicity was observed against B7H3 negative cells (A375 B7H3−/−), consistent with the capacity to potently induce antigen-dependent T-cell activation. These constructs displayed similar potencies to an alternative format, DART-Fc B7H3xCD3. These observations support that the antigen-targeted constrained CD3 format provided herein compared to other CD3 engaging formats, lack or exhibit reduced T-cell binding in isolation while maintaining potent B7H3-dependent T-cell cytotoxicity inducing capacities.

### 2. T Cell Activation

To assess T cell activation, suspension cells from T cell-mediated cytotoxicity assays were collected and stained with a live/dead stain and fluorophore-conjugated anti-CD4, anti-CD8, anti-CD25, anti-CD69, and/or anti-CD71 antibodies. Cells were analyzed using a SONY SA3800 spectral analyzer and CD4+ or CD8+ T cell activation was determined by measuring expression levels of CD25, CD69 or CD71 or percent CD25-, CD69- or CD71-positive.

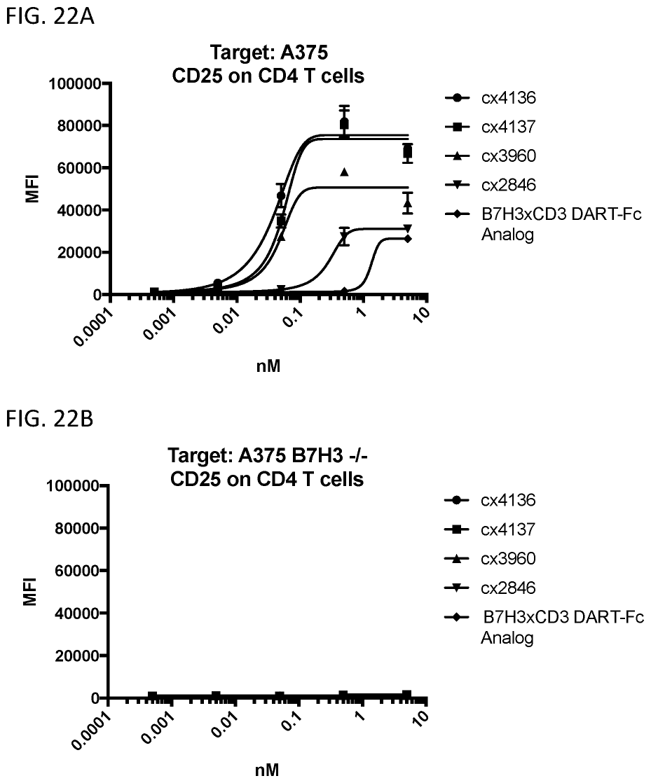

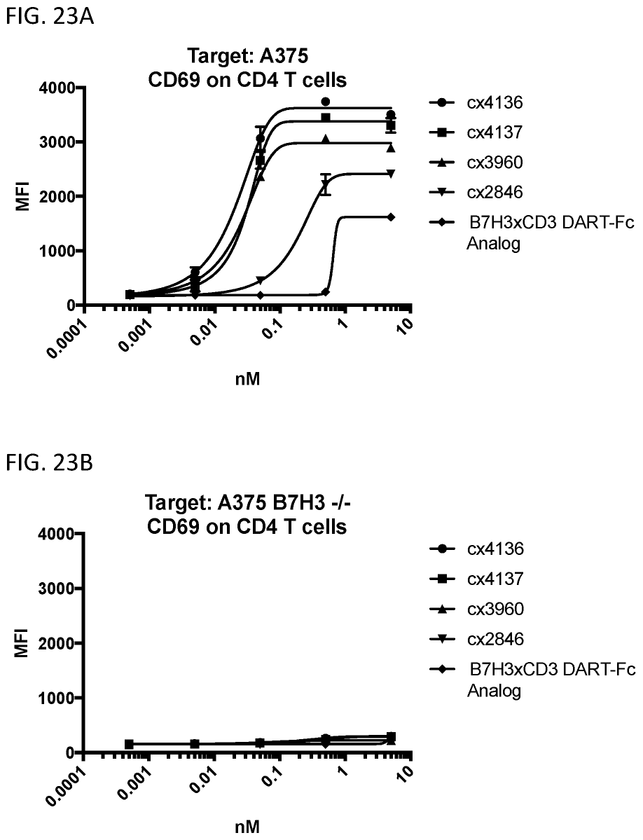

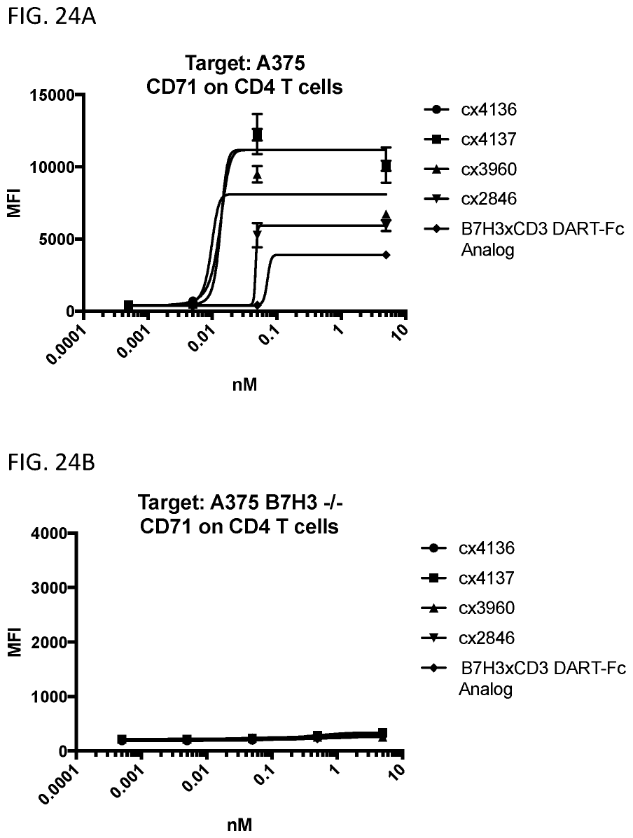

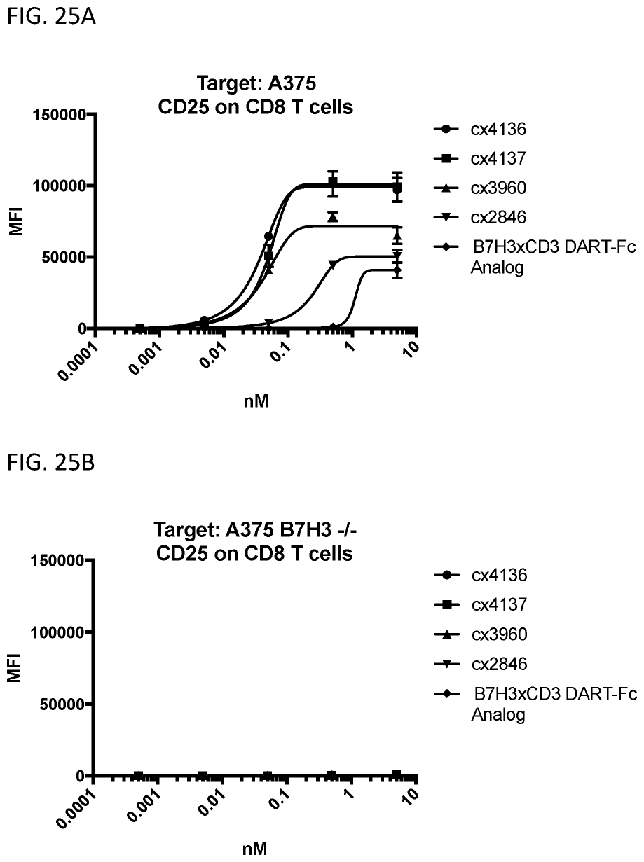

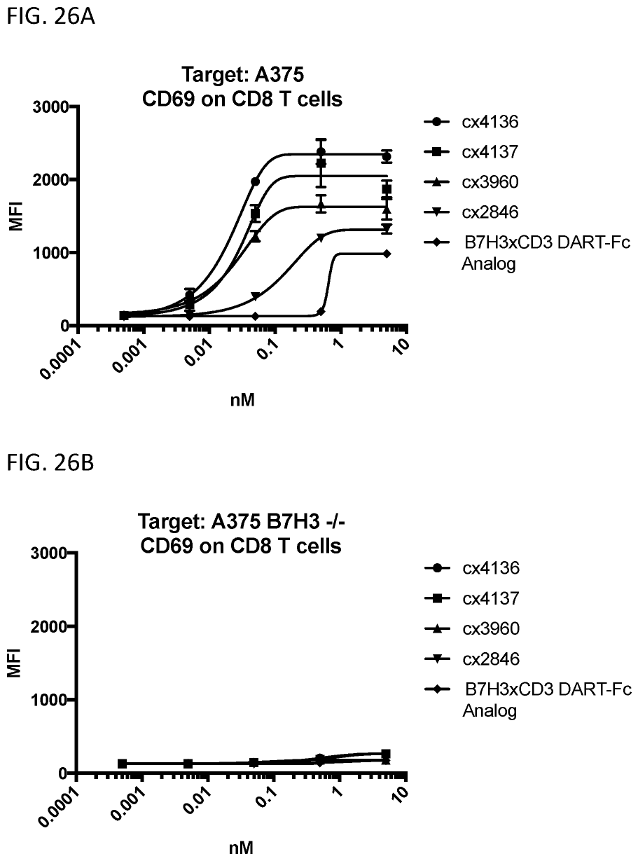

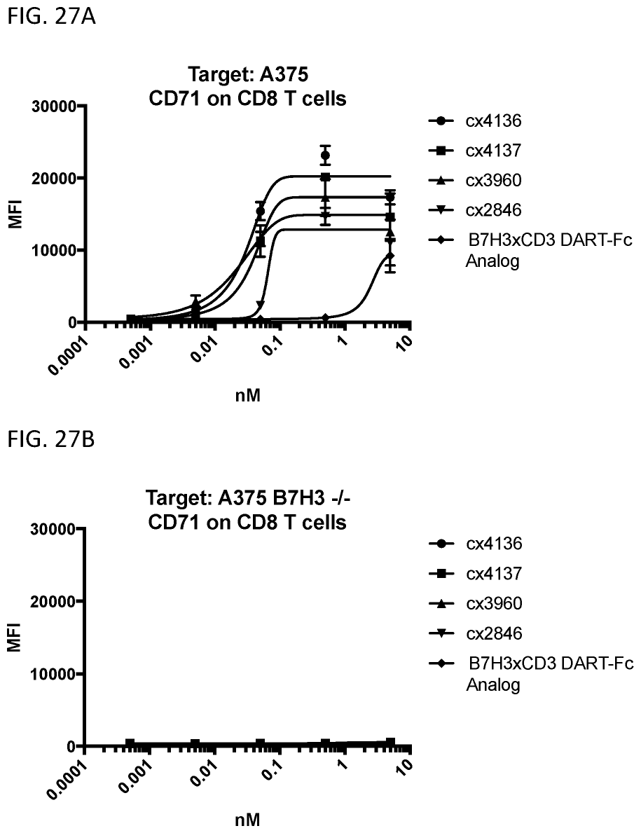

FIGS. 22A-22B and FIGS. 25A-25B depict results for CD25 expression on CD4 T cells and CD8 T cells, respectively, upon culture of T cells with B7H3 positive cells (A375) or B7H3 negative cells (A375 B7H3−/−) in the presence of exemplary constructs. FIGS. 23A-23B and FIGS. 26A-26B depict results for CD69 expression on CD4 cells and CD8 T cells, respectively, upon culture of T cells with B7H3 positive cells (A375) or B7H3 negative cells (A375 B7H3−/−) in the presence of exemplary constructs. FIGS. 24A-24B and FIGS. 27A-27B depict results for CD71 expression on CD4 T cells and CD8 T cells, respectively, upon culture of T cells with B7H3 positive cells (A375) or B7H3 negative cells (A375 B7H3−/−) in the presence of exemplary constructs. The results showed that exemplary assessed B7H3-targeting constrained CD3 engaging constructs mediated a dose-dependent B7H3-dependent T-cell activation via CD3 binding, as evidenced by increased expression of CD25, CD69 and CD71 in CD4+ and CD8+ T cells Similar potencies of T-cell activation by the constrained CD3 engaging constructs and the DART-Fc format were observed despite the significant differences in T-cell binding by these two formats.

Thus, the results demonstrated that the B7H3-targeting constrained CD3 engaging constructs of the present invention induced potent antigen-dependent activation of both CD4 and CD8 T-cells.

### 3. T Cell Cytokine Production (ELISA)

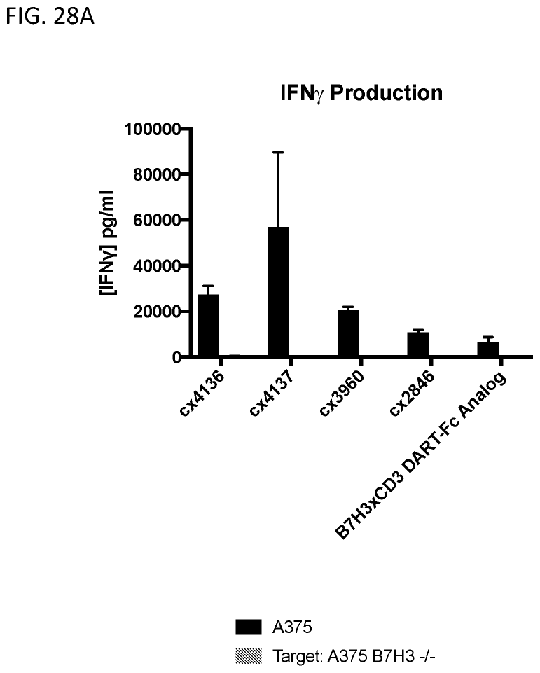

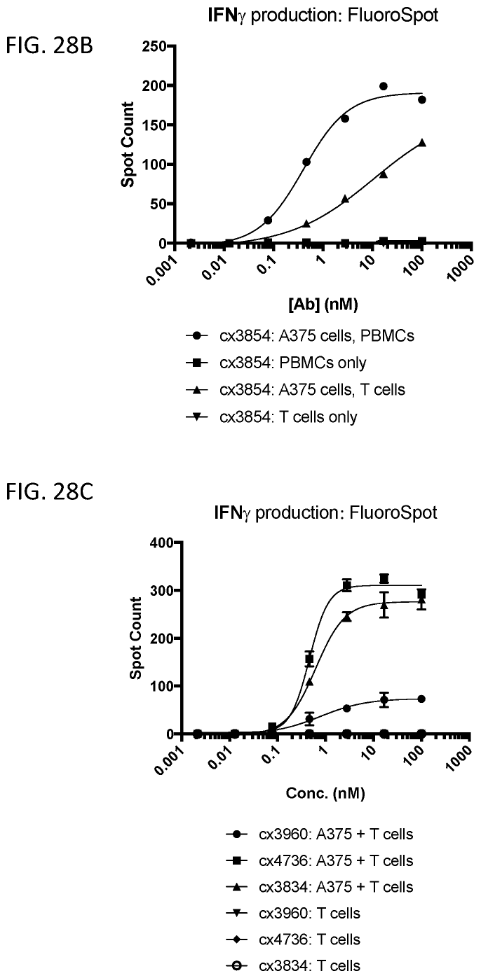

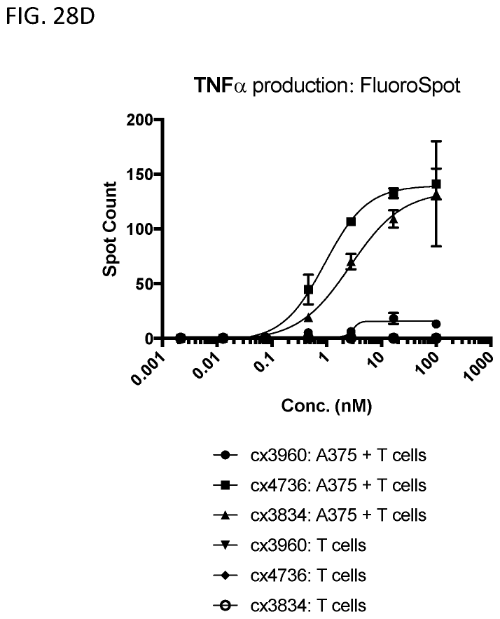

Supernatants from T cell-mediated cytotoxicity assays were analyzed for IFNγ content by sandwich ELISA (BioLegend, USA). The manufacturer's instructions were followed, and a standard curve was generated from which cytokine concentration values of supernatant samples were interpolated. Samples that had absorbance values below the lower limit of detection were assigned a cytokine concentration equal to half that of the lowest standard concentration. FIG. 28A shows that representative B7H3-targeted constrained CD3 engaging constructs cx4136, cx4137, cx3960, and cx2846 were observed to elicit IFNγ production by T-cells in an antigen-dependent manner.

### 4. T Cell Cytokine Production (FluoroSpot)

FluoroSpot membranes were coated with IFNγ and TNFα capture antibodies overnight at 4° C. Membranes were washed with PBS and antibody titrations, target cells, and PBMCs or purified T cells negatively enriched from PBMCs were added. Cells were seeded at a 1:10 ratio of target cell to effector cell. Assay plates were incubated for ˜24 h at 37° C. and membranes were prepared according to the manufacturer's (C.T.L.) instructions. Membranes were imaged using a CTL-ImmunoSpot S6 Universal Analyzer. Cytokine spot count was measured using uniform exposure time and intensity settings among assay wells. FIG. 28B and 28C (IFNγ) and FIG. 28D (TNFα) depict the ability of exemplary B7H3-targeted constrained CD3 engaging constructs to elicit cytokine production from PBMCs or T cells in B7H3-dependent manner.

### Example 8: ADCC Reporter Activation

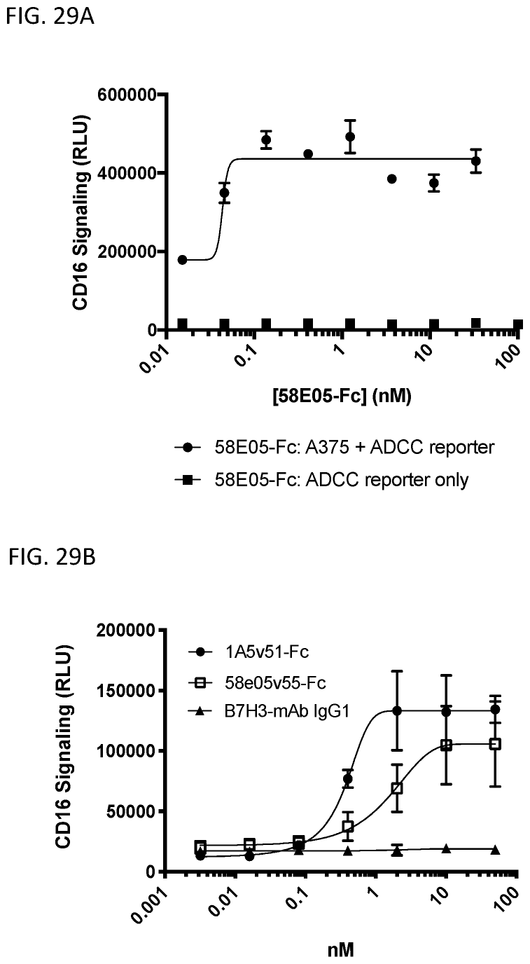

To assess the Fc effector function of exemplary B7H3 targeting sdAbs, a Jurkat reporter cell line engineered to stably express CD16a with an NFAT-driven luciferase reporter gene was used. Jurkat reporter cells were seeded (approximately 9×10⁴ cells/well) in the absence or presence of high B7H3 expressing A375 cells (3×10⁴ cells/well). 58E05-Fc, hz58E05v55-Fc, hz1A5v53-Fc or anti-B7H3 IgG1 antibody (derived from sequence published in U.S. Pat. No. 9,896,508) were titrated onto cells and assay plates were incubated at 37 degrees Celsius for 5.5 hours, with a final assay volume of 125 microliters. Assay plates were equilibrated to room temperature, 100 microliters Bio-Glo was added to sample wells, and assay plates were incubated at room temperature for an additional 10 minutes. 100 microliter aliquots were transferred to white 96-well plates and luminescence was measured using a SpectraMax L. FIG. 29A depicts the ability of 58E05-Fc to activate CD16a reporter cells in an antigen-dependent manner. FIG. 29B depicts the ability of hz58E05v55-Fc and hz1A5v53Fc to activate the Jurkat reporter cells, whereas the comparator B7H3-mAb IgG1 antibody did not display this ability.

### Example 9: Generation of Additional Constructs with Constrained CD3 Binding

Example 9 describes the generation and expression of multispecific polypeptide constructs containing a CD3 binding region that exhibits constrained CD3 binding. The multispecific constructs were generated in various configurationsto contain a heterodimeric Fc region of an immunoglobulin coupled by a linker (e.g. a non-cleavable linker) to the CD3 binding region, and one or more antigen binding domains that binds a tumor associated antigen (TAA) positioned amino-terminally relative to the Fc region and/or carboxy-terminally relative to the CD3 binding region of the multispecific polypeptide construct.

### A. Design and Generation of Constructs

Polynucleotides encoding at least a first polypeptide chain and a second polypeptide chain of the heterodimeric multispecific polypeptide construct were generated and cloned into a plasmid for expression. The first polypeptide chain generally included in order, from the N-terminus to C-terminus, a first Fc polypeptide (e.g. an Fc hole polypeptide); a non-cleavable linker; and a variable light (VL) domain of an anti-CD3 antibody. The second polypeptide chain generally included in order, from the N-terminus to C-terminus, a second Fc polypeptide (e.g. an Fc knob polypeptide); the same non-cleavable linker as the first polypeptide chain; and a variable heavy (VH) domain of an anti-CD3 antibody. The anti-CD3 antibody included either a disulfide-stabilized (dsFv) antibody (anti-CD3 VH with the mutation G44C and VL with the mutation G100C) or contained a non-disulfide stabilized Fv antibody, as set forth in Table ES. Various exemplary Fc polypeptide pairs to facilitate heterodimerization of the polypeptide chains were used as set forth in Table ES. One or both of the polypeptide chains additionally encoded one or more TAA antigen binding domain amino-terminal to the Fc domain and/or carboxy-terminal to the CD3 binding region, in various configurations. Similar constructs can be generated using other heterodimeric Fc configurations, including other knob-into-hole configurations, such as any as described; other CD3-binding regions, including other anti-CD3 antibodies, including dsFv or other monovalent fragments; or other TAA antigen-binding fragments, such as scFv, sdAb or Fab formats can also be used.

Among generated constructs, the non-cleavable linker included linkers ranging from 3-18 amino acids in size. Examples of non-cleavable linkers used in exemplary generated molecules were GGS, GGSGGS (SEQ ID NO:191), GGSGGSGGS (SEQ ID NO:192), GGSGGSGGSGGS (SEQ ID NO:193), GGSGGSGGSGGSGGS (SEQ ID NO:194), and GGGGGSGGGGGSGGGGGS (SEQ ID NO:317, contained in exemplary construct cx5823 and cx5952) or GGSGGGGSGGGGSGGGGS (SEQ ID NO:319).

Any antigen binding domain that binds to a TAA can be employed in the provided multispecific polypeptide constructs. Exemplary generated proteins contained an antigen binding domain that binds B7H3 (CD276). The antigen-binding domain can include single chain fragments (e.g. sdAb or scFv) or two chain antigen-binding fragments (Fabs). When the TAA was provided as a single chain fragment, e.g. sdAb or scFv, the TAA antigen binding domain was linked at the N-terminus to one or both polypeptide chains of the Fc heterodimer (e.g. hole and/or knob) by a peptide linker, e.g. PGGGG (SEQ ID NO:444) and/or was linked at the C-terminus to one or both domains (e.g. VH and/or VL) of the CD3 binding region by a peptide linker, e.g. GGGG (SEQ ID NO:195). Other similar peptide linkers can be employed. When the TAA was provided as a Fab antigen-binding fragment the construct was composed of a VH and CH1 linked directly to one or both Fc polypeptides without a linker, as well as a light chain composed of a VL and CL. These TAA binding Fabs can be located on the amino- or carboxy-terminus of the heterodimeric Fc.

Multispecific polypeptide constructs were generated containing 1, 2, 3 or 4 TAA antigen binding domain, such as to provide for monovalent, bivalent, trivalent, or tetravalent binding, respectively. In some cases, the TAA antigen binding domains were the same (mono-epitopic). In some cases, the TAA antigen binding domains were different, such that the generated multispecific polypeptide constructs exhibited specificity for at least two different TAAs, to different epitopes of the same TAA (bi-epitopic) or the same epitopes of the same TAA (mono-epitopic).

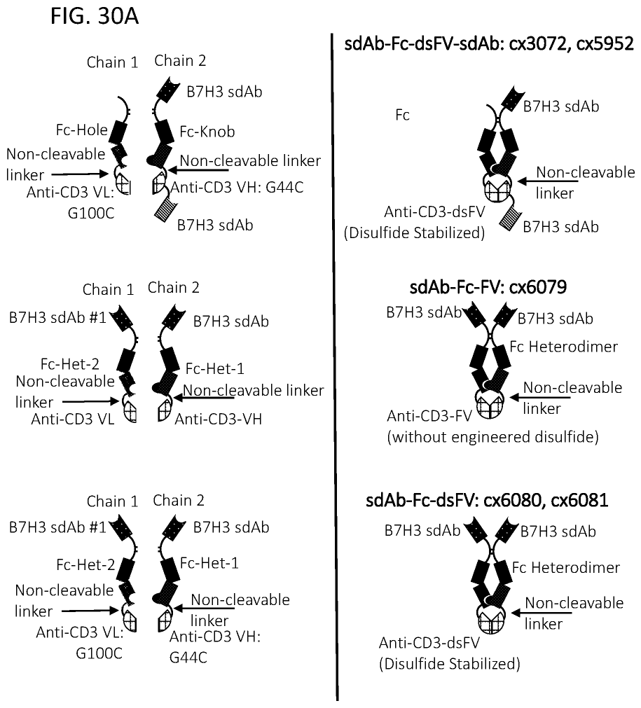

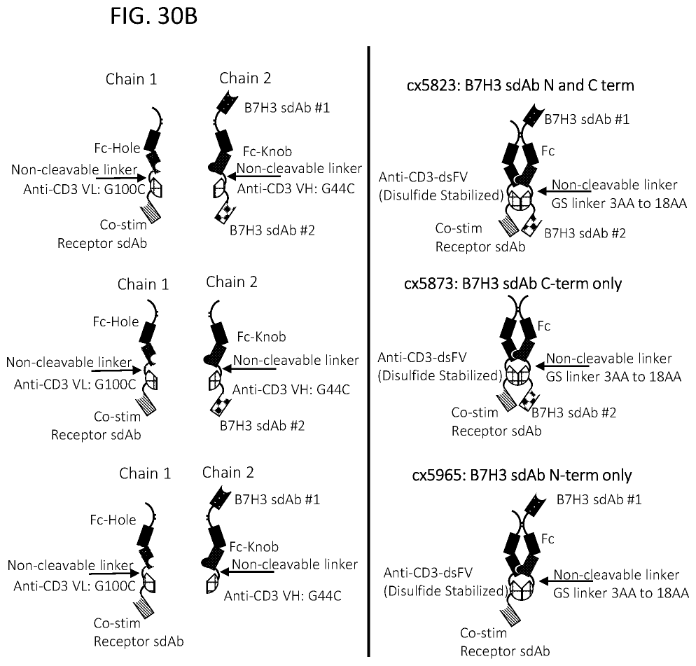

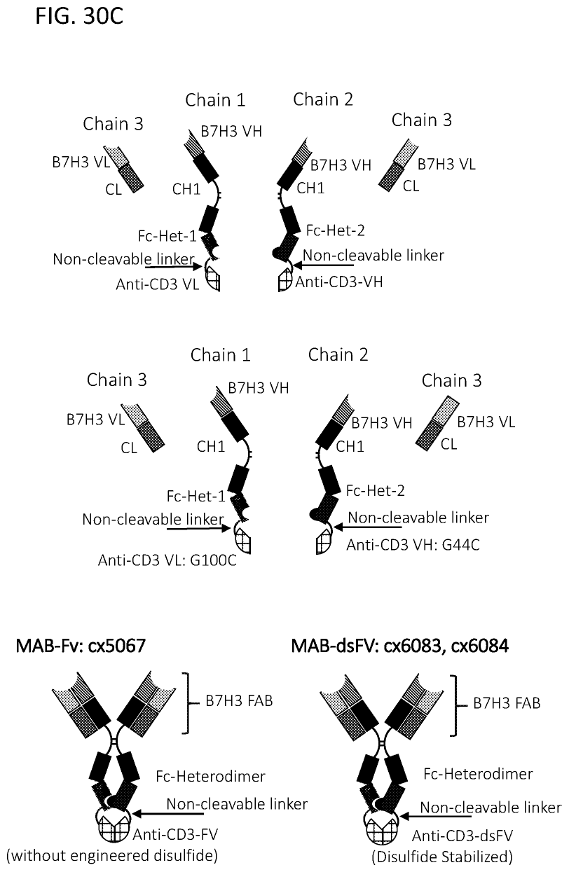

Among the generated proteins were constructs in which the TAA antigen binding domains were composed as single domain antibodies (sdAbs) of antigen-binding fragments (Fabs). Polynucleotides were generated to encode polypeptide chains of exemplary multispecific polypeptide constructs containing non-cleavable linkers. These included sdAb-containing constructs designated cx3072, cx5952, cx6079, cx6080, cx6081, cx5823, cx5873, and cx5965, targeting B7H3 as depicted in FIGS. 30A and 30B; and Fab-containing constructs designated cx5067, 6083, and 6084, targeting B7H3 as depicted in FIG. 30C. Some constructs were generated wherein the VH domain of the dsFv anti-CD3 antibody and the sdAb were both linked to the same side (e.g. hole or knob side) of the Fc heterodimer (e.g. cx3072 and cx5952, shown in FIG. 30A). Constructs were engineered without a disulfide stabilized Fv or were engineered with a disulfide linkage stabilizing the VH and VL domains of the anti-CD3 antibody. Notably, some of the exemplary constructs generated additionally contained a sdAb (containing a CDR1, a CDR2 and a CDR3 set forth in SEQ ID Nos: 468, 469 and 470, respectively; e.g. SEQ ID NO:400) targeting a 4-1BB co-stimulatory receptor (e.g. cx5823, cx5873, cx5965). A list of exemplary constrained CD3 binding constructs having sdAb and Fab TAA domains is given below in Table E5.

TABLE E5
Exemplary Constrained CD3 engaging constructs

| | | N-term | | | CD3 | C-term | |
|---|---|---|---|---|---|---|---|
| Construct | | sdAb | | | Binding | sdAb | Disulfide |
| ID | Chain | (Target) | Fc | Linker | Domain | (Target) | Stabilized |
| cx5823 | 1 | B7H3 | xELL- | GGGGGSGGGG | VH13 (SEQ | B7H3 sdAb | yes |
| | | sdAb 4 | Knob | GSGGGGGS | ID NO: 237) | 5 | |
| | | hz1A5v5 | (SEQ ID | (SEQ ID NO: 317) | | hz58E05v55 | |
| | | 1 (SEQ | NO: 299) | | | (SEQ ID | |
| | | ID | | | | NO: 466) | |
| | | NO: 467) | | | | | |
| | 2 | None | xELL- | GGGGGSGGGG | VL10 (SEQ | Co-stim | |
| | | | Hole | GSGGGGGS | ID NO: 265) | Recept or | |
| | | | (SEQ ID | (SEQ ID NO: 317) | | sdAb | |
| | | | NO: 304) | | | | |
| cx5952 | 1 | B7H3 | xELL- | GGGGGSGGGG | VH13 (SEQ | B7H3 sdAb | yes |
| | | sdAb 4 | Knob | GSGGGGGS | ID NO: 237) | 5 | |
| | | hz1A5v5 | (SEQ ID | (SEQ ID NO: 317) | | hz58E05v55 | |
| | | 1 (SEQ | NO: 299) | | | (SEQ ID | |
| | | ID | | | | NO: 466) | |
| | | NO: 467) | | | | | |
| | 2 | None | xELL- | GGGGGSGGGG | VL10 (SEQ | None | |
| | | | Hole | GSGGGGGS | ID NO: 265) | | |
| | | | (SEQ ID | (SEQ ID NO: 317) | | | |
| | | | NO: 304) | | | | |
| cx6079 | 1 | B7H3 | Fc-Het-1 | GGGGSGGGGS | VH32 (SEQ | None | no |
| | | sdAb 4 | (SEQ ID | GGGGS (SEQ ID | ID NO: 460) | | |
| | | hz1A5v5 | NO: 457) | NO: 318) | | | |
| | | 1 (SEQ | | | | | |
| | | ID | | | | | |
| | | NO: 467) | | | | | |
| | 2 | B7H3 | Fc-Het-2 | GGGGSGGGGS | VL20 (SEQ | None | |
| | | sdAb 4 | (SEQ ID | GGGGS (SEQ ID | ID NO: 461) | | |
| | | hz1A5v5 | NO: 458) | NO: 318) | | | |
| | | 1 (SEQ | | | | | |
| | | ID | | | | | |
| | | NO: 467) | | | | | |
| cx6080 | 1 | B7H3 | Fc-Het-1 | GGGGSGGGGS | VH33 (SEQ | None | yes |
| | | sdAb 4 | (SEQ ID | GGGGS (SEQ ID | ID NO: 480) | | |
| | | hz1A5v5 | NO: 457) | NO: 318) | | | |
| | | 1 (SEQ | | | | | |
| | | ID | | | | | |
| | | NO: 467) | | | | | |
| | 2 | B7H3 | Fc-Het-2 | GGGGSGGGGS | VL21 (SEQ | None | |
| | | sdAb 4 | (SEQ ID | GGGGS (SEQ ID | ID NO: 459) | | |
| | | hz1A5v5 | NO: 458) | NO: 318) | | | |
| | | 1 (SEQ | | | | | |
| | | ID | | | | | |
| | | NO: 467) | | | | | |
| cx6081 | 1 | B7H3 | Fc-Het-1 | GGGGSGGGGS | VH13 (SEQ | None | yes |
| | | sdAb 4 | (SEQ ID | GGGGS (SEQ ID | ID NO: 237) | | |
| | | hz1A5v5 | NO: 457) | NO: 318) | | | |
| | | 1 (SEQ | | | | | |
| | | ID | | | | | |
| | | NO: 467) | | | | | |
| | 2 | B7H3 | Fc-Het-2 | GGGGSGGGGS | VL10 (SEQ | None | |
| | | sdAb 4 | (SEQ ID | GGGGS (SEQ ID | ID NO: 265) | | |
| | | hz1A5v5 | NO: 458) | NO: 318) | | | |
| | | 1 (SEQ | | | | | |
| | | ID | | | | | |
| | | NO: 467) | | | | | |
| cx3072 | 1 | B7H3 | IgG1- | GGGGGSGGGG | VH13 (SEQ | B7H3 sdAb | yes |
| | | sdAb 2 | Knob | GSGGGGGS | ID NO: 237) | 1 | |
| | | hz58E05 | (SEQ ID | (SEQ ID NO: 317) | | hz57B04v24 | |
| | | v27 | NO: 293, | | | (SEQ ID | |
| | | (SEQ ID | 297 or | | | NO: 503, | |
| | | NO: 67) | 445) | | | e.g. 43) | |
| | 2 | None | IgG1- | GGGGGSGGGG | VL10 (SEQ | None | |
| | | | Hole | GSGGGGGS | ID NO: 265) | | |
| | | | (SEQ ID | (SEQ ID NO: 317) | | | |
| | | | NO: 294, | | | | |
| | | | 298 or | | | | |
| | | | 446) | | | | |
| cx5873 | 1 | None | xELL- | GGGGGSGGGG | VH13 (SEQ | B7H3 sdAb | yes |
| | | | Knob | GSGGGGGS | ID NO: 237) | 3 | |
| | | | (SEQ ID | (SEQ ID NO: 317) | | hz58E05v48 | |
| | | | NO: 295, | | | (SEQ ID | |
| | | | 299) | | | NO: 85) | |
| | 2 | None | xELL- | GGGGGSGGGG | VL10 (SEQ | Co-stim | |
| | | | Hole | GSGGGGGS | ID NO: 265) | Recept or | |
| | | | (SEQ ID | (SEQ ID NO: 317) | | sdAb | |
| | | | NO: 302, | | | | |
| | | | 304) | | | | |
| cx5965 | 1 | B7H3 | xELL- | GGGGGSGGGG | VH13 (SEQ | none | yes |
| | | sdAb 4 | Knob | GSGGGGGS | ID NO: 237) | | |
| | | hz1A5v5 | (SEQ ID | (SEQ ID NO: 317) | | | |
| | | 1 (SEQ | NO: 295, | | | | |
| | | ID | 299) | | | | |
| | | NO: 467) | | | | | |
| | 2 | None | xELL- | GGGGGSGGGG | VL10 (SEQ | Co-stim | |
| | | | Hole | GSGGGGGS | ID NO: 265) | Recept or | |
| | | | (SEQ ID | (SEQ ID NO: 317) | | sdAb | |
| | | | NO: 302, | | | | |
| | | | 304) | | | | |
| cx5187 | 1 | B7H3 | xELL- | GGGGGSGGGG | VH13 (SEQ | B7H3 sdAb | yes |
| | | sdAb 4 | Knob | GSGGGGGS | ID NO: 237) | 3 | |
| | | hz1A5v5 | (SEQ ID | (SEQ ID NO: 317) | | hz58E05v48 | |
| | | 1 (SEQ | NO: 295, | | | (SEQ ID | |
| | | ID | 299) | | | NO: 85) | |
| | | NO: 467) | | | | | |
| | 2 | None | xELL- | GGGGGSGGGG | VL10 (SEQ | Co-stim | |
| | | | Hole | GSGGGGGS | ID NO: 265) | Recept or | |
| | | | (SEQ ID | (SEQ ID NO: 317) | | sdAb | |
| | | | NO: 302, | | | | |
| | | | 304) | | | | |
| cx5067 | 1 | B7H3- | Fc-Het-1 | GGGGSGGGGS | VH32 (SEQ | None | no |
| | | Fab | (SEQ ID | GGGGS (SEQ ID | ID NO: 460) | | |
| | | (SEQ ID | NO: 457) | NO: 318) | | | |
| | | NOs: | | | | | |
| | | 455, 456) | | | | | |
| | 2 | B7H3- | Fc-Het-2 | GGGGSGGGGS | VL20 (SEQ | None | |
| | | Fab | (SEQ ID | GGGGS (SEQ ID | ID NO: 461) | | |
| | | (SEQ ID | NO: 458) | NO: 318) | | | |
| | | NOs: | | | | | |
| | | 455, 456) | | | | | |
| cx6083 | 1 | B7H3- | Fc-Het-1 | GGGGSGGGGS | VH33 (SEQ | None | yes |
| | | Fab | (SEQ ID | GGGGS (SEQ ID | ID NO: 480) | | |
| | | (SEQ ID | NO: 457) | NO: 318) | | | |
| | | NOs: | | | | | |
| | | 455, 456) | | | | | |
| | 2 | B7H3- | Fc-Het-2 | GGGGSGGGGS | VL21 (SEQ | None | |
| | | Fab | (SEQ ID | GGGGS (SEQ ID | ID NO: 459) | | |
| | | (SEQ ID | NO: 458) | NO: 318) | | | |
| | | NOs: | | | | | |
| | | 455, 456) | | | | | |
| cx6084 | 1 | B7H3- | Fc-Het-1 | GGGGSGGGGS | VH13 (SEQ | None | yes |
| | | Fab | (SEQ ID | GGGGS (SEQ ID | ID NO: 237) | | |
| | | (SEQ ID | NO: 457) | NO: 318) | | | |
| | | NOs: | | | | | |
| | | 455, 456) | | | | | |
| | 2 | B7H3- | Fc-Het-2 | GGGGSGGGGS | VL10 (SEQ | None | |
| | | Fab | (SEQ ID | GGGGS (SEQ ID | ID NO: 265) | | |
| | | (SEQ ID | NO: 458) | NO: 318) | | | |
| | | NOs: | | | | | |
| | | 455, 456) | | | | | |

### B. Expression and Purification of Generated Constructs

Separate plasmids encoding each chain of the heterodimeric constrained CD3 binding protein were transiently transfected at an equimolar ratio into mammalian cells (either HEK293 or CHO) using polyethylenimine Recombinant protein secreted into the supernatant was collected after 3-14 days, and purified by protein A chromatography, followed by either preparative size exclusion chromatography (SEC) or flow-through hydrophobic interaction chromatography (HIC). In some cases, heterodimeric protein was enriched for during purification due to a mutation designed into one chain of the heterodimeric Fc at position I253R or H435R (e.g. in the hole-Fc) such that it did not bind protein A, and thus homodimers of I253R or H435R were not purified. The second chromatography step by SEC (AKTA with Superdex-200 resin) or FT-HIC (AKTA with butyl/phenyl sepharose) was used to remove undesired cross-paired species containing two heterodimeric Fcs that were more hydrophobic and twice the expected molecular weight.

The method favored production of heterodimeric multispecific polypeptide constructs, containing properly paired species of heterodimeric Fc and the anti-CD3 Fv (e.g. disulfide stabilized anti-CD3 Fv). Purified heterodimeric constrained CD3 binding protein was stable and did not accumulate cross-paired species upon prolonged incubation at 4° C. or increased protein concentration.

### Example 10: Assessment of Binding of Constrained CD3 Binding Constructs to Cancer Cells ad Primary T cells by Flow Cytometry

This Example describes studies assessing binding of exemplary constructs to T cells or to cancer cells. These studies were carried out in single cultures containing either only the T cells or only the cancer cells in isolation from each other.

Binding of exemplary multispecific constructs containing an antigen-binding domain directed against B7H3 were assessed for binding to B7H3 positive A375 tumor cells or primary T-cells. The constructs were generated containing antigen-binding domain(s) that were either sdAbs or a FABs, and that were positioned either only N-terminal to the Fc or both N-terminal to the Fc and C-terminal to the anti-CD3 binding domain (see FIGS. 30A and 30C and Table E5). Among the various formats of constructs tested included: sdAb-Fc-dsFV-sdAb (cx3072, cx5952) sdAb-Fc-FV (cx6079), sdAb-Fc-dsFV (cs6080, cx6081), MAB-FV (cx5067) and MAB-dsFV (cx6083, cx6084), where the FV represents the anti-CD3 binding domain composed of VH and VL domain pairs and “ds” notes disulfide stabilized via an engineered interdomain disulfide bond.

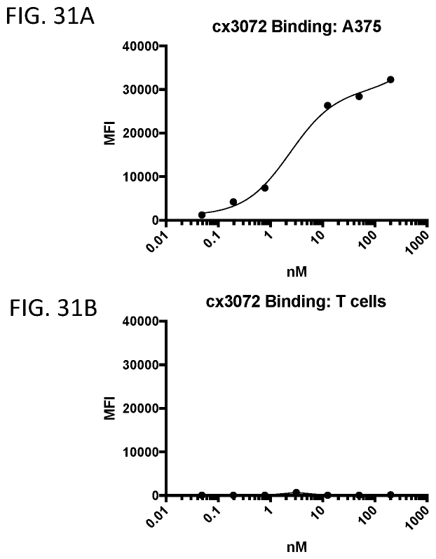

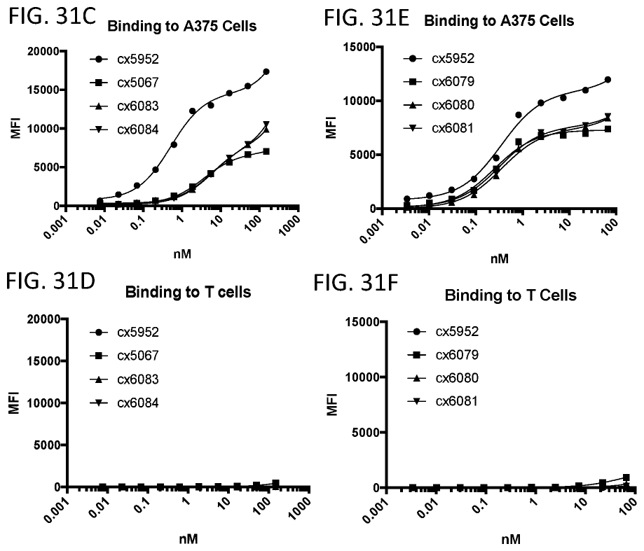

FIGS. 31A-F demonstrate that these constructs were capable of binding to B7H3 but not T-cells in isolation. Binding was assessed as described above via flow cytometry using a fluorophore-conjugated anti-human Fc secondary antibody. cx3072 bound to A375 cells with high affinity (FIG. 31A) but not to isolated T-cells (FIG. 31B). The tested sdAb-Fc-dsFV-sdAb (cx5952) displayed higher binding affinity compared to a FAB containing MAB-FV (cx5067) and MAB-dsFV constructs (cx6083, cx6084) (FIG. 31C). The B7H3 -targeting sdAb containing constructs (cx5952, cx6079, cx6080, and cx6081) bound to B7H3 positive cells with similar affinities (depicted in FIG. 31E), with cx5952 displaying higher maximal binding. cx6079, cx6080 and cx6081 contain two identical B7H3-targeting sdAbs, whereas cx5952, and cx3072 contain two distinct B7H3-targeting sdAb that bind different epitopes. The MAB-FV, cx5067, contains two identical B7H3-targeting FAB domains. Notably none of the exemplary B7H3-targeted constrained CD3 engaging constructs bound primary human T-cells, as depicted in FIGS. 31B, 31D, and 31F. These results further support that binding to CD3 on T cells in isolation is constrained in the provided formats.

### Example 11: Assessment of T Cell Activating Activity of B7H3-Targeted Constrained CD3 Binding Constructs

Constructs containing either a B7H3-targeted sdAb or a Fab as the tumor-associated antigen-binding domain were assessed for T-cell activating activity in a T cell reporter assay and in a T cell cytotoxicity assay. Activity of B7H3-targeted constrained CD3 engaging constructs that were formatted with an anti-B7H3 sdAb (e.g. cx5823, cx6079, cx6080, cx6081, cx3072 and cx5952) or anti-B7H3 MAB constructs formatted with a Fab (e.g. cx5067, cx6083 or cx6084) as the antigen-binding domain(s) were assessed (see FIGS. 30A-30C and Table E5). All tested constructs, except cx5067 and cx6079, contained a disulfide-stabilized anti-CD3 Fv (dsFv) containing an interchain disulfide bond created by the modification of anti-CD3 VH G44C paired with VL G100C. The anti-CD3 Fv of cx5067, designated MAB-Fv, and the anti-CD3 Fv of cx6079, designated sdAb-Fc-Fv, were not disulfide-stabilized.

### A. T Cell Reporter Activity

The NFAT-GFP CD3 Jurkat reporter was used to compare the CD3 agonistic properties of B7H3-targeted constrained CD3 engaging constructs when co-cultured in the presence of B7H3-positive cells (A375) or non-target CCRF-CEM cells that naturally lack B7H3 expression. The Jurkat cells express NFAT-driven green fluorescence protein (GFP). Agonism of CD3 results in NFAT signaling and production of green fluorescence.

Antigen targeting constrained CD3 engaging constructs were titrated onto co-cultures of target cells and engineered Jurkat cells that express NFAT-driven green fluorescence protein (GFP). In this assay, target cell lines included either A375 or CCRF-CEM. For reporter assays utilizing adherent antigen expressing target cells, target cells were seeded, allowed to settle at room temperature for uniform distribution, and incubated for several hours at 37° C. to permit adherence prior to addition of reporter cells and antigen targeting constrained CD3 engaging constructs. Assay plates were serially imaged using an IncuCyte ZOOM system and CD3 reporter cell activation was determined by measuring total green object integrated.

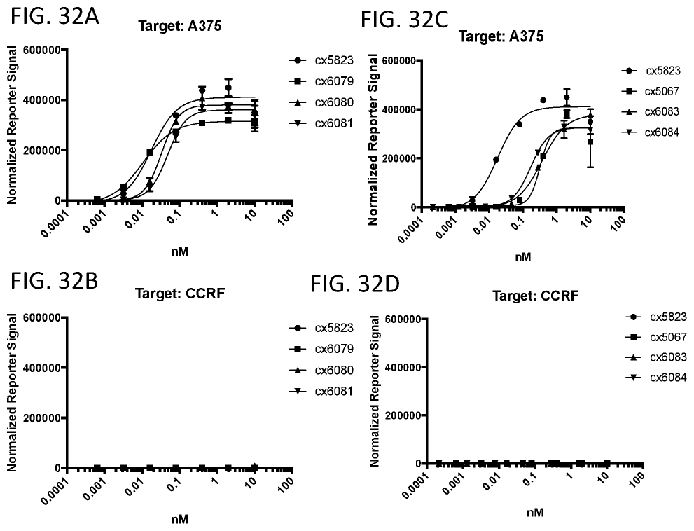

In this assay, anti-B7H3 sdAb constructs, cx5823, cx6079, cx6080 and cx6081, or anti-B7H3 Fabs constructs, cx5067, cx6083 and cx6084, were used as the B7H3-targeting domains. As shown in FIG. 32A, the constructs containing B7H3-targeted sdAb displayed similar potencies of antigen-dependent CD3 activation. As shown in FIG. 32C the exemplary cx5823 construct containing B7H3-targeted sdAbs was found to be superior at mediating antigen-dependent CD3 activation compared to the constructs containing B7H3-targeted Fabs. Although cx5823 is formatted with a binding domain for a costimulatory receptor, it is unlikely that this component contributed to the difference in results, since Jurkat T cells do not express the costimulatory receptor. None of the constructs demonstrated activity against the B7H3-negative CCRF-CEM cells (FIGS. 32B and 32D).

### B. Cytotoxicity

To further assess activity of the molecules, exemplary B7H3-targeted constructs cx3072 and cx5952 (each formatted as sdAb-dsFv), cx6083 and cx6084 (MAB-dsFv), cx5067 (MAB-Fv), cx6079 (sdAb-Fv), and cx6080 and cx 6081 (sdAb-dsFv) were tested in a T-cell-mediated cytotoxicity assay. Target cells included the B7H3 positive cell line, A375, and either modified A375 cells, wherein B7H3 gene was disrupted by CRISPR (A375:B7H3 KD), or CCRF-CEM cells that naturally lacked B7H3 expression. Target cells were seeded at 1.0×10⁴ cells per well, allowed to settle at room temperature for uniform distribution, and incubated for several hours at 37° C. Primary T cells were negatively enriched from PBMCs isolated from healthy human donor leukopaks and added at a 10:1 T cell-to-target cell ratio. Green caspase-3/7 reagent was added, which fluorescently labels nuclear DNA of cells undergoing apoptosis was added. Multispecific constructs with constrained CD3 engaging activity were titrated onto the co-culture and assay plates were serially imaged using an IncuCyte ZOOM system. Target cell death was determined by measuring total red/green overlap object area.

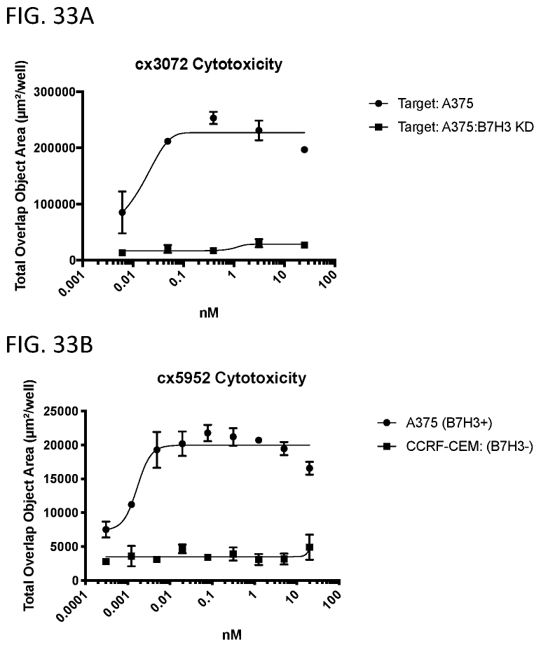

As shown in FIGS. 33A and 33B, exemplary constructs cx3072 and cx5952 containing sdAb B7H3-targeted antigen-binding domains induced potent T-cell-mediated cytotoxicity of B7H3 positive cells (A375) but not B7H3 negative cells.

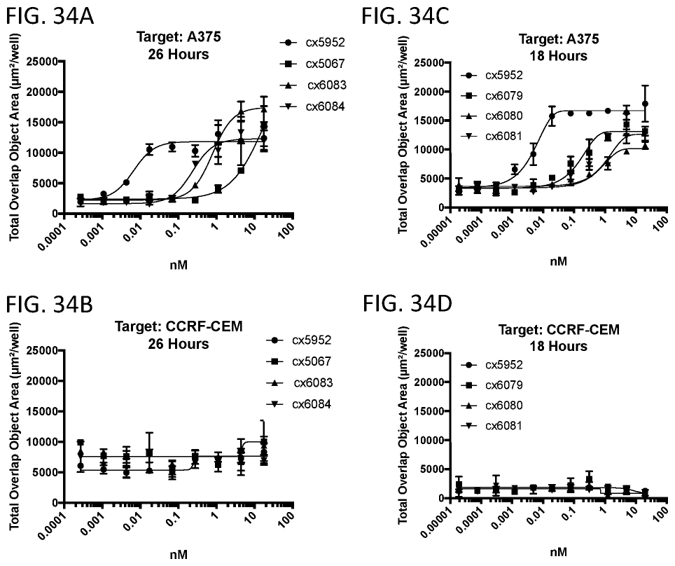

When compared to exemplary B7H3-targeting constrained CD3 engagers with Fab B7H3-targeting domains (cx5067, cx6083 and cx6084), the exemplary cx5952 sdAb B7H3-targeting constrained CD3 engager mediated enhanced target-dependent T-cell cytotoxicity (FIG. 34A). No measurable T cell cytotoxicity was observed against the B7H3 negative cell line CCRF-CEM for any of the tested constructs, consistent with the capacity to potently induce antigen-dependent T-cell activation (FIG. 34B). Of the constructs tested, representative MAB-dsFV constructs cx6084 and cx6083 contained the engineered disulfide, whereas the representative MAB-FV construct cx5067 lacked this stabilizing modification. Notably cx6083 and cx5067 are identical with the exception of the presence (cx6083) or absence (cx5067) of the engineered disulfide within the anti-CD3 FV domain (depicted in FIG. 30C). The engineered disulfide was created by the modification of G44C within VH and G100C within VL. As shown in FIG. 34A, cx6083 displayed superior potency in mediating target-dependent T-cell cytotoxicity compared to cx5067, suggesting that the incorporation of the inter-domain disulfide is beneficial in T-cell mediated cytotoxicity, likely by enhancing proper association of the VH and VL domains that comprise the anti-CD3 FV.

When compared to other exemplary B7H3-targeting constrained CD3 engagers with sdAb B7H3-targeting domains (cx6079, cx6080 and cx6081), the exemplary cx5952 sdAb B7H3-targeting constrained CD3 engager mediated enhanced target-dependent T-cell cytotoxicity (FIG. 34C). No measurable T cell cytotoxicity was observed against the B7H3 negative cell line CCRF-CEM for any of the tested constructs, consistent with the capacity to potently induce antigen-dependent T-cell activation (FIG. 34D). Of the constructs tested, sdAb-dsFV constructs cx5952, cx6080 and cx6081 contained the engineered disulfide linkage, whereas the sdAb-FV construct cx6079 lacked this stabilizing modification. The engineered disulfide was created by the modification of G44C within VH and G100C within VL. Notably, cx5952 was engineered to have two distinct B7H3 targeting domains, one located at the amino terminal and one located at the carboxy terminal. cx6079, cx6080, and cx6081 were engineered to have two identical B7H3 targeting domains, both located at the amino terminal (see FIG. 30A).

### C. T Cell Modulation

To further assess T cell modulation, exemplary multispecific CD3 constrained binding constructs were assessed by monitoring the ability of the constructs to modulate T cell activation markers. To assess T cell activation, suspension cells from the T cell cytotoxicity assays described above, involving culture of T cells with B7H3 positive cells (A375) or B7H3 negative cells (CCRF-CEM) in the presence of an exemplary B7H3-targeted constrained CD3 engaging construct, cx5952, were collected. Cells were stained with a live/dead stain and fluorophore-conjugated anti-CD4, anti-CD8, anti-CD25, anti-CD69, and/or anti-CD71 antibodies. Cells were analyzed using a SONY SA3800 spectral analyzer and CD4+ or CD8+ T cell activation was determined by measuring expression levels of CD25, CD69 or CD71 or percent CD25-, CD69- or CD71-positive cells.

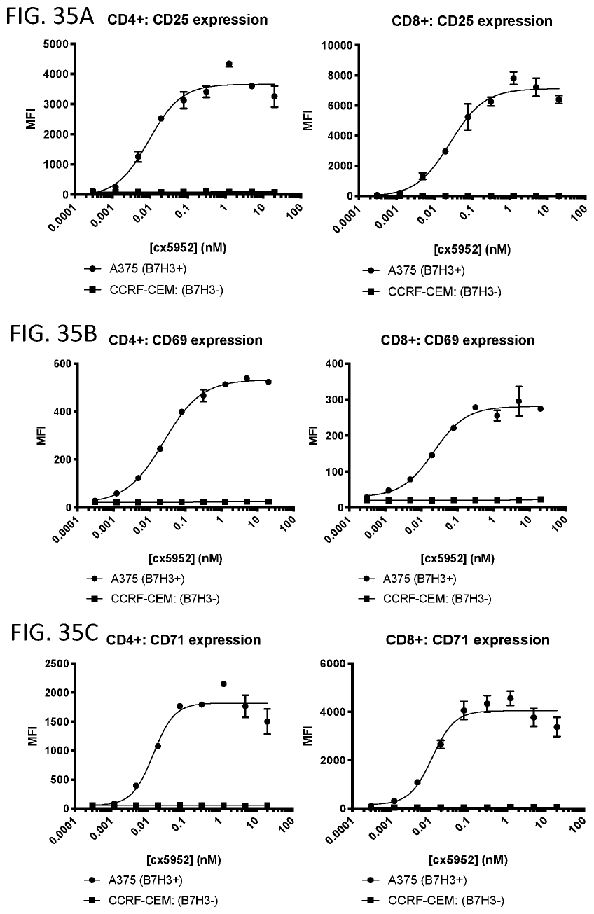

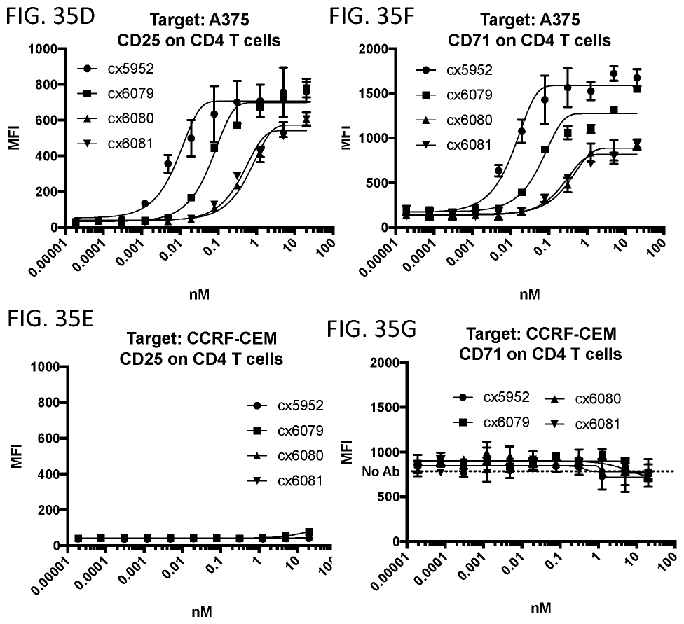

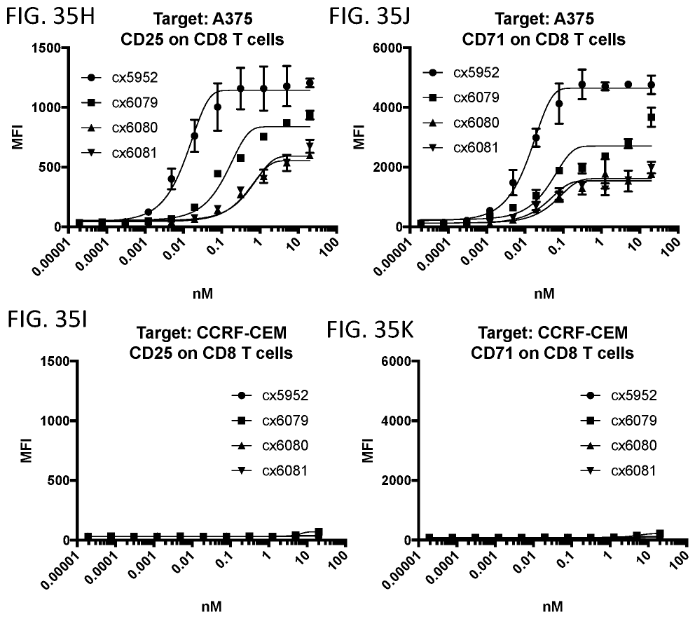

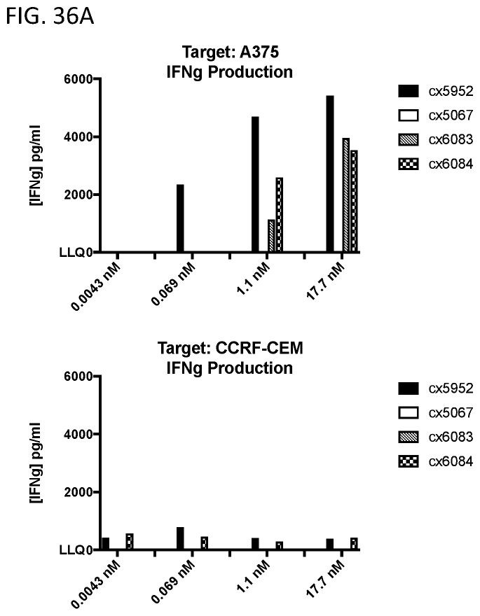

Results are shown for CD25 expression (FIG. 35A), CD69 expression (FIG. 35B) and CD71 expression (FIG. 35C) on CD4+ and CD8+ T cells following the co-culture with B7H3 positive cells (A375) or B7H3 negative cells (CCRF-CEM) in the presence of cx5952. The results showed that cx5952 mediated a dose-dependent B7H3-dependent T-cell activation via CD3 binding, as evidenced by increased expression of CD25, CD69 and CD71 in CD4+ and CD8+ T cells.

When compared to the other exemplary B7H3-targeting constrained CD3 engagers with sdAb B7H3-targeting domains (cx6079, cx6080 and cx6081), the exemplary cx5952 sdAb B7H3-targeting constrained CD3 engager mediated increased T cell activation as evidenced by increased expression of CD25 in CD4+ T cells (FIG. 35D) and in CD8+ T cells (FIG. 35H) and increased expression of CD71 in CD4+ T cells (FIG. 35F) and in CD8+ T cells (FIG. 35J). Increased expression of the surface markers on T cells was not observed in the presence of the B7H3-targeting constrained CD3 engager constructs in cultures with B7H3 negative cell lines (FIGS. 35E and 35G for CD4+ T cells and FIGS. 351 and 35K for CD8+ T cells).

### D. T Cell Cytokine Production

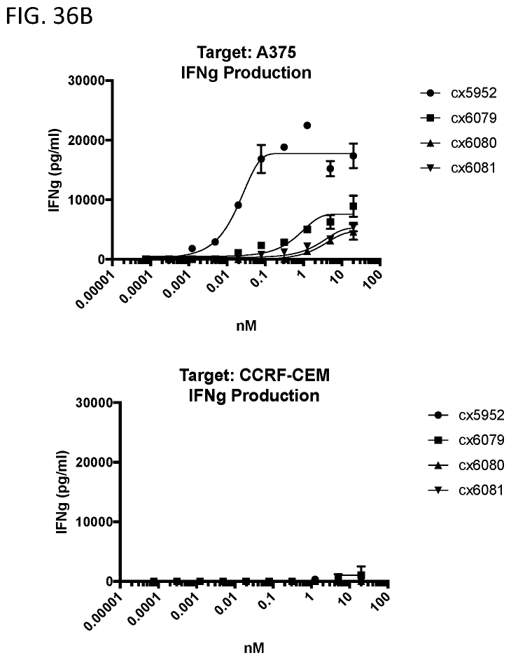

Supernatants from T cell cytotoxicity tumor cell co-culture assays, involving co-culture of T cells with B7H3 positive A375 cells or B7H3 negative CCRF-CEM cells in the presence of cx5952, cx6083, cx6084 or cx5067, were analyzed for IFNγ content by sandwich ELISA. A standard curve was generated from which cytokine concentration values of supernatant samples were interpolated. Samples that had absorbance values below the lower limit of detection were assigned a cytokine concentration equal to half that of the lowest standard concentration. As shown in FIG. 36A, the representative sdAb-Fc-dsFV-sdAb construct, cx5952, was superior to the tested B7H3-targeted FAB containing constructs, cx6083, cx6084 and cx5067 at eliciting target-dependent cytokine release from activated T-cells. Importantly, the MAB-dsFV constructs, cx6083 and cx6084 were superior to the MAB-FV construct, cx5067, demonstrating the importance of interdomain disulfide stabilizing modification for enhancing T-cell function.

When compared to the other exemplary B7H3-targeting constrained CD3 engagers with sdAb B7H3-targeting domains (cx6079, cx6080 and cx6081), the exemplary cx5952 sdAb B7H3-targeting constrained CD3 engager mediated substantially increased production of IFNγ in the presence of B7H3-target cells T cells but not in cultures with B7H3 negative cell lines (FIG. 36B).

### E. Summary

These observations further support that the antigen-targeted constrained CD3 format provided herein lack or exhibit reduced T-cell binding in isolation while maintaining potent B7H3-dependent T-cell cytotoxicity inducing capacities. Without wishing to be bound by theory, together these results show that utilization of antigen targeted sdAbs instead of a Fabs may reduce the immune synapse distance between the TAA expressing tumor cell and the CD3 expressing T-cells and enhance T cell activity and cytotoxicity. Notably, it was found that the inclusion of an interchain disulfide bond created by the modification of anti-CD3 VH G44C paired with VL G100C greatly enhanced the activity of constrained CD3 engaging constructs. Further, the more potent B7H3-dependent T cell activity by cx5952 compared to other sdAb B7H3-targeting domain constructs suggests that the positioning of the B7H3-targeting sdAb C-terminal to the anti-CD3 binding domain or the fact that cx5952 binds two distinct epitopes on B7H3 whereas the other constructs tested bind to a single epitope in a bivalent manner, contributed to this enhanced activity.

### Example 12: Assessment of CD3 Constrained Multispecific Constructs Containing Single or Multiple B7H3-Binding Targeting Domains

Activity of constructs containing a monovalent sdAb antigen-binding domain (positioned at either the N or C-terminus) was compared to activity of dual binding (bivalent) constructs that contained antigen-targeting sdAbs positioned at both the N and C-termini. Binding was assessed substantially as described in Example 10 and T cell activity was assessed in the Jurkat reporter assay and T cell cytotoxicity assays substantially as described in Example 11.

### A. Binding

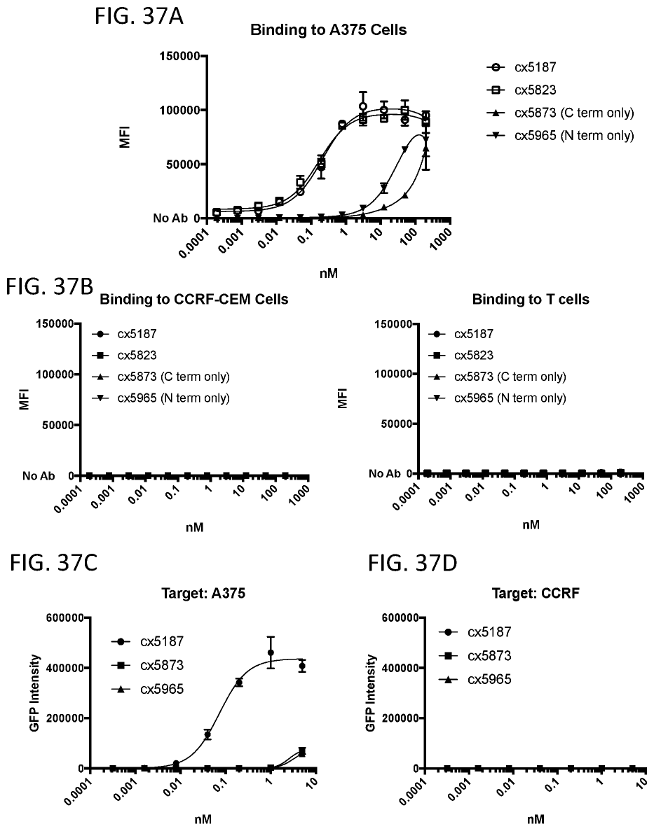

As shown in FIG. 37A the bivalent B7H3-targeting constrained CD3 engaging constructs, cx5187 and cx5823, displayed higher affinity binding to B7H3 positive A375 cells, compared to the monovalent versions, cx5873 and cx5965. None of these constructs displayed any detectable binding to B7H3 negative CCRF-CEM cells or isolated T-cells (FIG. 37B).

### B. T Cell Reporter Activity

B7H3 Antigen-dependent CD3 agonistic capacities of antigen-targeted constrained CD3 engaging constructs that engage the antigen in a monovalent or bivalent manner were assessed using CD3-NFAT Jurkat reporter cells, in an assay substantially as described above. As shown in FIG. 37C, substantially increased fluorescence reporter activity was observed in the presence of the exemplary bivalent B7H3-targeted construct cx5187 compared to reporter activity for the exemplary monovalent constructs cx5873 and cx5965. No reporter activity was observed when constructs were incubated with Jurkat reporter cells co-cultured with B7H3-negiatve CCRF target cells (FIG. 37D).

### C. Cytotoxic Activity

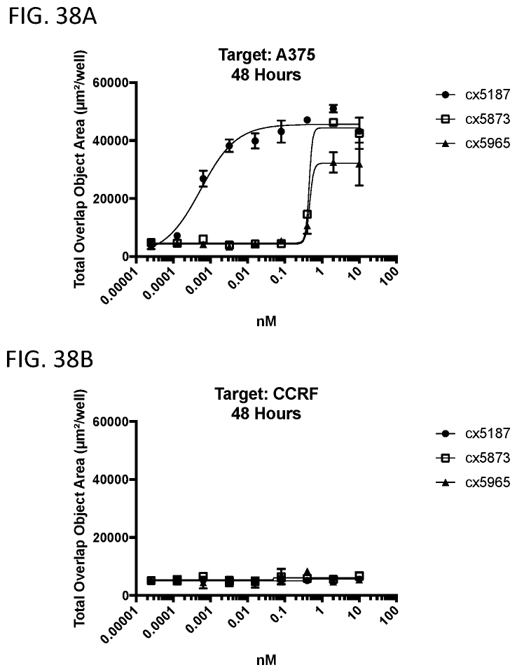

Cytotoxicity of B7H3-targeted CD3 constrained binding constructs was assessed against a melanoma cell line, A375, and a T-cell acute lymphoblastic leukemia cell line, CCRF-CEM, which were used as B7H3 positive and negative cell lines, respectively. Cytotoxicity was assessed substantially as described in Example 11. As shown in FIG. 38A an exemplary bivalent B7H3-targeted constrained CD3 engaging construct, cx5187, displayed enhanced target-dependent T-cell mediated cytotoxicity compared to the monovalent versions of the constructs, cx5873 and cx5965. In these assays, no cytotoxicity was observed in the absence of B7H3 expression of the target cells, as shown in FIG. 38B wherein the CCRF-CEM cells were used as target cells. D. T Cell Modulation

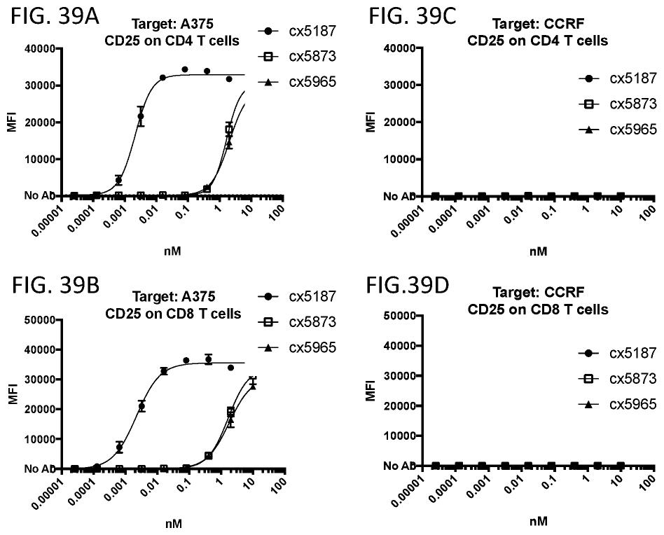

T cell modulation was assessed by monitoring expression of CD25, substantially as described in Example 11, in suspension cells from T cell cytotoxicity assays above, involving culture of T cells with B7H3 positive cells (A375) or B7H3 negative cells (CCRF-CEM) in the presence of cx5187, cx5873 or cx5965. As shown in FIG. 39A and 39B, an exemplary bivalent B7H3-targeted constrained CD3 engaging construct, cx5187, displayed enhanced target-dependent T-cell mediated activation compared to the monovalent versions of the constructs, cx5873 and cx5965, as evidenced by enhanced potency of CD25 upregulation on CD4 and CD8 T-cells. In these assays, no T-cell activation was observed in the absence of B7H3 expression of the target cells, as shown in FIG. 39C and 39D, wherein the CCRF-CEM cells were used as target cells. These results demonstrated that the B7H3-targeting constrained CD3 engaging constructs induced potent antigen-dependent activation of both CD4 and CD8 T-cells.

### D. Summary

Together, these results demonstrate that bivalent antigen-targeted constrained CD3 engaging constructs displayed superior antigen-dependent CD3 binding and activity than the monovalent antigen-targeted constrained CD3 engaging constructs. These results are consistent with a finding that constructs containing dual antigen-binding domains positioned at both the N and C-termini have superior binding and T cell activity than monovalent constructs containing only a single monovalent antigen-binding domain. Furthermore, without wishing to be bound by theory, positioning one of the sdAbs C-terminal to the CD3 binding domain may form a more optimal immune synapse compared to constructs wherein the sdAbs are only positioned N-terminal to the Fc as the latter may increase the immune synapse distance.

### Example 13: Assessment of CD3 Constrained MultispecificConstructs Containing B7H3-Targeting sdAb and Fab Domains

Constructs containing either B7H3-targeted sdAb(s) or a Fab as the tumor-associated antigen-binding domain were assessed for T-cell activating activity. Activity of B7H3-targeted constrained CD3 engaging constructs that were formatted with anti-B7H3 sdAbs (e.g. cx5952 and cx6079) or anti-B7H3 MAB constructs formatted with a Fab (e.g. cx5067, cx6083 or cx6084) as the antigen-binding domain(s) were assessed (see FIGS. 30A and 30C and Table E5). All tested constructs, except cx6079 and cx5067, contained a disulfide-stabilized anti-CD3 Fv (dsFv) containing an interchain disulfide bond created by the modification of anti-CD3 VH G44C paired with VL G100C. The anti-CD3 Fv of cx5067, designated MAB-Fv, and the anti-CD3 Fv of cx6079, designated sdAb-Fc-Fv, were not disulfide-stabilized. Additionally, cx5952 was engineered to contain two distinct B7H3-targeting sdAb domains, with one located N-terminal to the Fc domain and one located C-terminal to the CD3-binding domain. By contrast, cx6079 was engineered to contain two identical B7H3-targeting sdAb domains, both located N-terminal to the Fc domain. The Fvs of all three Fab constructs were engineered to be N-terminal to the Fc domain.

### A. Cytotoxicity

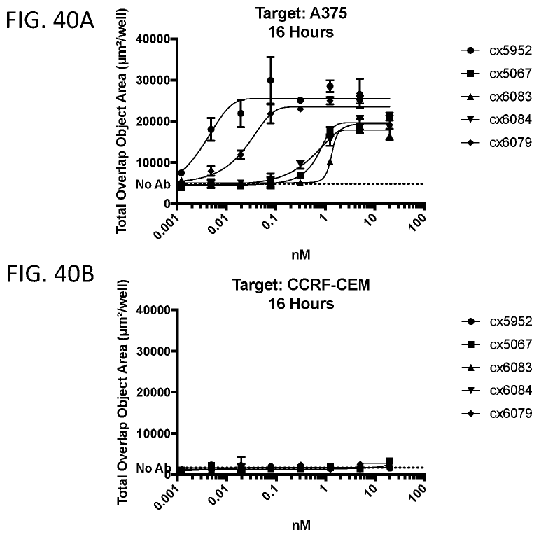

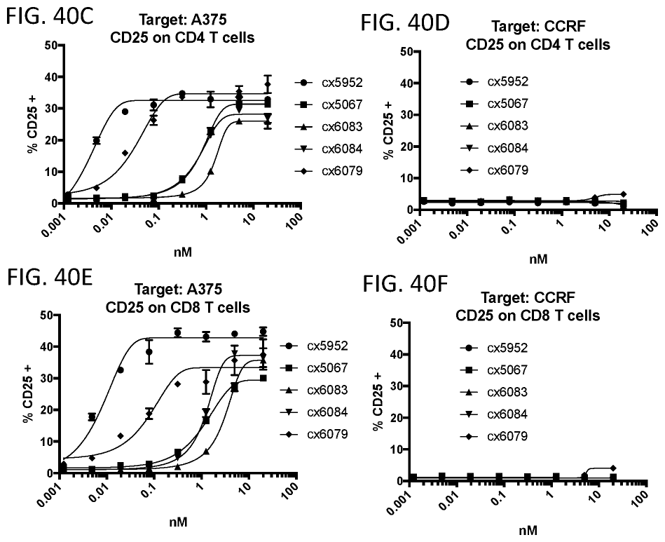

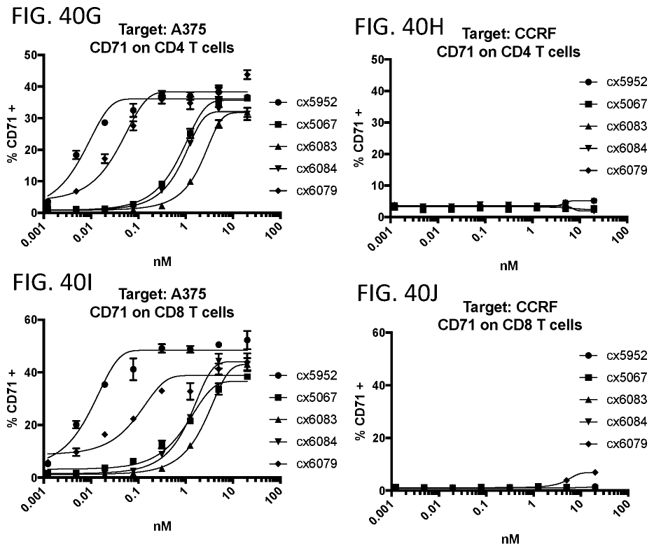

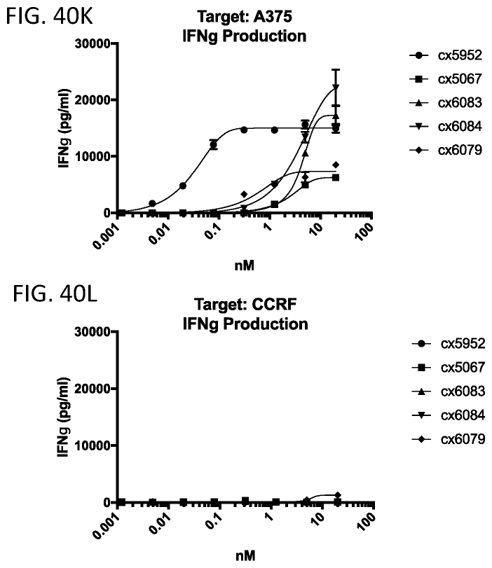

Cytotoxicity of B7H3-targeted CD3 constrained binding constructs was assessed substantially as described in Example 11. Cytotoxicity was assessed against a melanoma cell line, A375, and a T-cell acute lymphoblastic leukemia cell line, CCRF-CEM, which were used as B7H3 positive and negative cell lines, respectively. As shown in FIG. 40A the exemplary constrained CD3 engaging constructs formatted with B7H3-targeting sdAbs, cx5952 and cx6079, were superior at eliciting antigen-dependent T-cell cytotoxicity compared to to the anti-B7H3 MAB constructs formatted with a Fab, cx5067, cx6083, and cx6084. Notably, cx5952 was more potent than cx6079, suggesting the positioning of the B7H3-targeting sdAb C-terminal to the anti-CD3 binding domain and/or the stabilization of the anti-CD3 FV via engineered disulfide contributed to this enhanced activity. In these assays, no cytotoxicity was observed in the presence of B7H3-negative CRF-CEM cells target cells, as shown in FIG. 40B.

### B. T Cell Modulation

To further assess T cell modulation, exemplary multispecific CD3 constrained binding constructs were assessed by monitoring the ability of the constructs to modulate T cell activation markers, substantially as described in Example 11. To assess T cell activation, suspension cells from T cell cytotoxicity assays above, involving culture of T cells with B7H3 positive cells (A375) or B7H3 negative cells (CCRF-CEM) in the presence of an exemplary B7H3-targeted constrained CD3 engaging constructs, were collected. Tested constructs included anti-B7H3 constructs formatted with sdAbs (e.g. cx5952 and cx6079) and anti-B7H3 constructs formatted with a Fab (e.g. cx5067, cx6083 and cx6084)

Cells were stained with a live/dead stain and fluorophore-conjugated anti-CD4, anti-CD8, anti-CD25 and/or anti-CD71 antibodies. Cells were analyzed using a SONY SA3800 spectral analyzer and CD4+ or CD8+ T cell activation was determined by measuring expression levels of CD25 or CD71 or percent CD25- or CD71-positive cells.

Results are shown for CD25 expression (FIG. 40C-F) and CD71 expression (FIG. 40G-J) on CD4+ and CD8+ T cells following the co-culture with B7H3 positive cells (A375) or B7H3 negative cells (CCRF-CEM) in the presence of the described constructs. The results showed that cx5952 mediated a dose-dependent B7H3-dependent T-cell activation via CD3 binding, as evidenced by increased expression of CD25 and CD71 in CD4+ and CD8+ T cells. cx5952 was the most potent over other B7H3-targeted constrained CD3 engaging constructs at inducing T-dependent T-cell activation.

### C. T Cell Cytokine Production

Supernatants from T cell cytotoxicity tumor cell co-culture assays, involving co-culture of T cells with B7H3 positive, A375 or negative, CCRF-CEM cells in the presence of cx5952, cx6079, cx6083, cx6084 or cx5067, were analyzed for IFNγ content by sandwich ELISA. A standard curve was generated from which cytokine concentration values of supernatant samples were interpolated. Samples that had absorbance values below the lower limit of detection were assigned a cytokine concentration equal to half that of the lowest standard concentration. As shown in FIG. 40K, the representative sdAb-Fc-dsFV-sdAb construct, cx5952, was superior to the tested B7H3-targeted FAB containing constructs, cx6083, cx6084 and cx5067 at eliciting target-dependent cytokine release from activated T-cells. The increase in cytokine production was target dependent as it was not observed in the presence of B7H3 negative CCRF-CEM cells (FIG. 40L). This is consistent with the findings from the antigen dependent cytotoxicity and activation assays. Importantly, the MAB-dsFV constructs, cx6083 and cx6084 were superior to the MAB-FV construct, cx5067, demonstrating the importance of interdomain disulfide stabilizing modification for enhancing T-cell function.

### D. Summary

Together, these results demonstrate that constrained anti-CD3 constructs formatted with anti-B7H3 sdAb binding domains were superior at eliciting antigen-dependent T-cell cytotoxicity compared to the anti-B7H3 MAB constructs formatted with a Fab B7H3 binding domain. Further, that cx5952 was more potent than cx6079 suggested that the positioning of the B7H3-targeting sdAb C-terminal to the CD3 binding domain, the stabilization of the anti-CD3 FV via engineered disulfide, or both, contribute to enhanced activity. Without wishing to be bound by theory, positioning one of the sdAbs C-terminal to the CD3 binding domain may form a more optimal immune synapse compared to constructs wherein the sdAbs are only positioned N-terminal to the Fc as the latter may increase the immune synapse distance.

### Example 14: Comparison of Orientation of CD3 Binding Region in CD3-constrained Multispecific Constructs Containing B7H3-Targeting Domains

Additional B7H3-targetig multispecific polypeptide constructs were generated containing an Fv as a CD3 binding region in which the VH or VL of the anti-CD3 Fv in the constructs was positioned C-terminally to either the Fc-knob or Fc-hole of the heterodimeric Fc region. The generated CD3-constrained multispecific polypeptide constructs were assessed for the ability to activate T cells via CD3 engagement in a T cell reporter assay.

### A. Design and Generation of Constructs

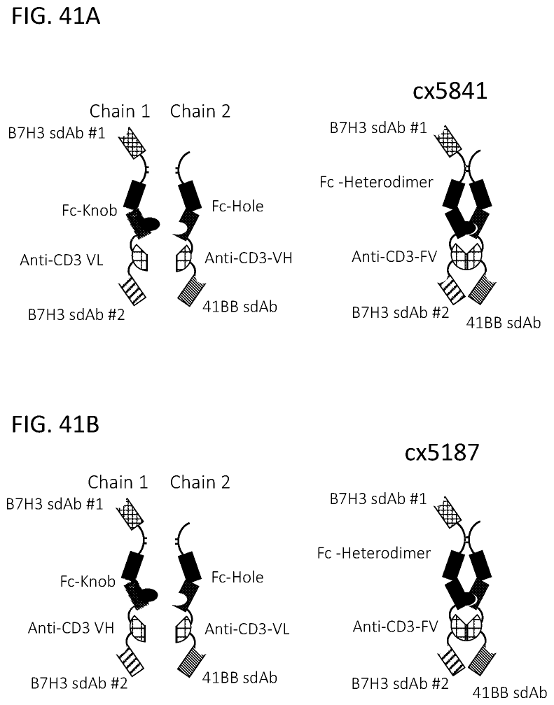

The multispecific constructs were generated as shown in FIGS. 41A-B, to contain a heterodimeric Fc region of an immunoglobulin coupled by a linker (e.g. a non-cleavable linker) to the CD3 binding region, a 4-1BB antigen binding domain (e.g. sdAb, containing a CDR1, a CDR2 and a CDR3 set forth in SEQ ID Nos: 468, 469 and 470, respectively; e.g. SEQ ID NO:400) as a CRBR positioned carboxy-terminally relative to the CD3 binding region, and dual antigen binding domains that bind the B7H3 tumor associated antigen (TAA) positioned amino-terminally relative to the Fc region and carboxy-terminally relative to the CD3 binding region of the multispecific polypeptide construct.

Polynucleotides encoding at least a first polypeptide chain and a second polypeptide chain of the heterodimeric multispecific polypeptide construct were generated and cloned into a plasmid for expression. The first polypeptide chain generally included in order, from the N-terminus to C-terminus, a first Fc polypeptide (e.g. an Fc hole polypeptide); a non-cleavable linker; a variable light (VL; e.g. cx5187) or variable heavy (VH; e.g. cx5841) domain of an anti-CD3 antibody; and a 4-1BB binding domain (e.g. sdAb) as a CRBR. The second polypeptide chain generally included in order, from the N-terminus to C-terminus, a first B7H3 antigen binding domain (e.g. B7H3 sdAb #1), a second Fc polypeptide (e.g. an Fc knob polypeptide); the same linker as the first polypeptide chain; the other of the variable heavy (VH) or variable light (VL) domain of an anti-CD3 antibody; and a second B7H3 antigen binding domain (e.g. B7H3 sdAb #2). The anti-CD3 antibody included a disulfide-stabilized (dsFv) antibody (anti-CD3 VH with the mutation G44C and VL with the mutation G100C).

Notably, as shown in FIGS. 41A-B, the orientation of the anti-CD3 VH and anti-CD3 VL of the CD3 Fv were positioned differently relative to the Fc knob or Fc hole of the heterodimeric Fc region. As shown in FIG. 41A, cx5841 was generated in which the first polypeptide of the heterodimeric construct had the VL of CD3 Fv positioned C-terminal to the Fc knob and B7H3 binding domain on the extreme N and C-termini and the second polypepide of the heterodimeric construct had the VH of CD3 Fv positioned C-terminal to the Fc Hole and a 41BB binding on the extreme C-termini. In constrast, FIG. 41B depicts the exemplary construct cx5187 (described in Example 9) in which the first polypeptide of the heterodimeric construct had the VH of CD3 Fv positioned C-terminal to the Fc knob and B7H3 binding domain on the extreme N and C-termini and the second polypepide of the heterodimeric construct had the VL of CD3 Fv positioned C-terminal to the Fc Hole and a 41BB binding on the extreme C-termini

Components of the exemplary generated constrained CD3 binding constructs are shown in Table E6. The constructs were expressed and purified substantially as described in Example 9.

TABLE E6
Exemplary constrained CD3 engaging constructs containing B7H3-targeting domain

| | | N-term | | | CD3 | C-term | |
|---|---|---|---|---|---|---|---|
| Construct | | sdAb | | | Binding | sdAb | Disulfide |
| ID | Chain | (Target) | Fc | Linker | Domain | (Target) | Stabilized |
| cx5841 | 1 | B7H3 | xELL- | GGGGGSGGGGG | VL10 | B7H3 sdAb | yes |
| | | sdAb 4 | Knob | SGGGGGS (SEQ | (SEQ ID | 3 | |
| | | hz1A5v5 | (SEQ ID | ID NO: 317) | NO: 265) | hz58E05v48 | |
| | | 1 (SEQ | NO: 295, | | | (SEQ ID | |
| | | ID | 299) | | | NO: 85) | |
| | | NO: 467) | | | | | |
| | 2 | None | xELL- | GGGGGSGGGGG | VH13 | Co-stim | |
| | | | Hole | SGGGGGS (SEQ | (SEQ ID | Recept or | |
| | | | (SEQ ID | ID NO: 317) | NO: 237) | sdAb (e.g. | |
| | | | NO: 302, | | | SEQ ID | |
| | | | 304) | | | NO: 400) | |
| cx5187 | 1 | B7H3 | xELL- | GGGGGSGGGGG | VH13 | B7H3 sdAb | yes |
| | | sdAb 4 | Knob | SGGGGGS (SEQ | (SEQ ID | 3 | |
| | | hz1A5v5 | (SEQ ID | ID NO: 317) | NO: 237) | hz58E05v48 | |
| | | 1 (SEQ | NO: 295, | | | (SEQ ID | |
| | | ID | 299) | | | NO: 85) | |
| | | NO: 467) | | | | | |
| | 2 | None | xELL- | GGGGGSGGGGG | VL10 | Co-stim | |
| | | | Hole | SGGGGGS (SEQ | (SEQ ID | Recept or | |
| | | | (SEQ ID | ID NO: 317) | NO: 265) | sdAb (e.g. | |
| | | | NO: 302, | | | SEQ ID | |
| | | | 304) | | | NO: 400) | |

### B. T Cell Reporter Activity

To compare CD3 engagement, the exemplary constructs were tested in an antigen-dependent CD3 reporter assay by assessing their ability to activate a CD3 NFAT reporter Jurkat cell line in a co-culture with target antigen-expressing cells. Activation was assessed by monitoring either green fluorescent or luciferase reporter signal in Jurkat reporter cells.

The B7H3-targeting constrained CD3 engaging constructs were titrated onto co-cultures of either A375 cells expressing B7H3 or control CCRF-CEM cells not expressing B7H3, and engineered Jurkat cells that express NFAT-driven green fluorescence protein (GFP). Engagement of CD3 results in NFAT signaling and production of green fluorescence. For reporter assays utilizing adherent target cells, target cells were seeded, allowed to settle at room temperature for uniform distribution, and incubated for several hours at 37° C. to permit adherence prior to addition of reporter cells and antigen targeting constrained CD3 engaging constructs. Assay plates were serially imaged using an IncuCyte ZOOM system and CD3 reporter cell activation was determined by measuring total green object integrated intensity.

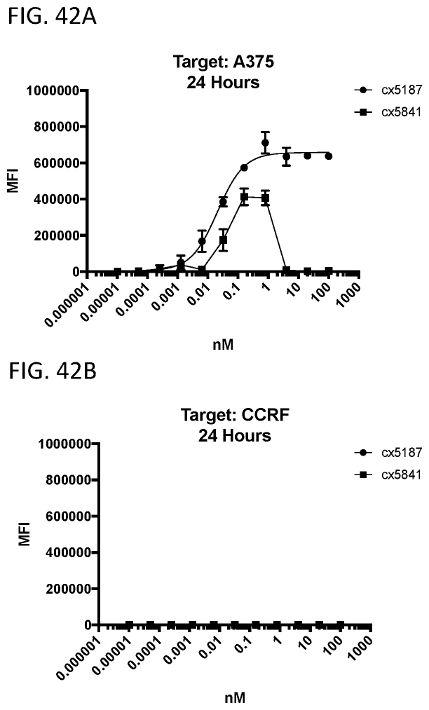

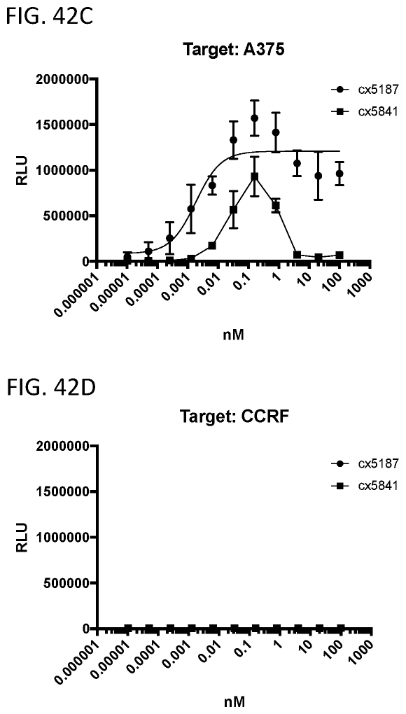

As shown in FIG. 42A, the exemplary B7H3-targeted constrained CD3 engaging constructs exhibited capacity to mediate target antigen specific T-cell activation when incubated in reporter T cell co-cultures in the presence of B7H3-expressing target cells. Reporter activity, however, was not observed in co-cultures with cells not expressing B7H3 (FIG. 42B). Notably, cx5187 with the Knob-VH; Hole-VL format displayed enhanced T cell activation compared to cx5841 with the Knob-VL; Hole-VH format.

In a similar assay, the same B7H3-targeting constrained CD3 engaging constructs were titrated onto co-cultures of either A375 cells expressing B7H3 or control CCRF-CEM cells not expressing B7H3, and engineered Jurkat cells that express NFAT-driven luciferase. As shown in FIG. 42C, the exemplary B7H3-targeted constrained CD3 engaging constructs exhibited capacity to mediate target antigen specific T-cell activation when incubated in reporter T cell co-cultures in the presence of B7H3-expressing target cells. Again, reporter activity, was not observed in co-cultures with cells not expressing B7H3 (FIG. 42D). As in the GFP reporter assay, the construct with the Knob-VH; Hole-VL format (cx5187) displayed enhanced T cell activation compared to the construct with the Knob-VL; Hole-VH format (cx5841).

These results are consistent with an observation that enhanced CD3 engagement and activity is observed when the components of the CD3 Fv are oriented so that the VH and VL are positioned C-terminally to the Fc Knob and Fc Hole regions, respectively.

### Example 15: Epitope Binning of B7H3 sdAbs

The ability of various B7H3 sdAbs to bind distinct eptitopes was assessed by Bio-Layer Interferometry (BLI) using a ForteBio Octet system with streptavidin coated sensor and biotinylated recombinant B7H3 extracellular domain. Exemplary B7H3 sdAbs were pairwise for blocking of another's binding to the epitope of an antigen. Results are set forth below in Table E7.

| Bin 1 | Bin 2 | Bin 3 | Bin 4 |
|---|---|---|---|
| 58E05 | 57B04 | 57B06 | 57B10 |
| | 1A5 | 57H12 | |
| | 1H5 | 58B06 | |
| | | 57A12 | |
| | | 57D01 | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Sequences

| # | SEQUENCE | ANNOTATION |
|---|---|---|
| 1 | QLQLQESGGGLVQAGDSLRLSCAASGFSFGSNVMMWVRQAP | L1A5 |
| | GKGPEWVSTIYSSGTGTFYADSVKGRFTISRNNAKNTLYLQMN | |
| | SLKPDDTAVYYCATSGPVRGWGPRSQGTLVTVKP | |
| 2 | EVQLVESGGGEVQPGGSLRLSCAASGFSFGSNVMMWVRQAPG | hz1A5v1 |
| | KGPEWVSTIYSSGTGTFYAESVKGRFTISRDNAKNTLYLQMSSL | |
| | RAEDTAVYYCATSGPVRGWGPRSQGTLVTVKPGG | |
| 3 | EVQLVESGGGEVQPGGSLRLSCAASGFSFGSNVMMWVRQAPG | hz1A5v2 |
| | KGPEWVSTIYSSGTGTFYAESVKGRFTISRNNAKNTLYLQMSSL | |
| | RAEDTAVYYCATSGPVRGWGPRSQGTLVTVKPGG | |
| 4 | EVQLVESGGGEVQPGGSLRLSCAASGFSFGSNVMMWVRQAPG | hz1A5v3 |
| | KGPEWVSTIYSSGTGTFYAESVKGRFTISRNNAKNTLYLQMSSL | |
| | RAEDTAVYYCATSGPVRGWGPRGQGTLVTVKPGG | |
| 5 | EVQLVESGGGEVQPGGSLRLSCAASGFSFGSNVMMWVRQAPG | hz1A5v7 |
| | KGPEWVSTIYSSGTGTFYAESVKGRFTISRNNAKNTLYLQMSSL | |
| | KPDDTAVYYCATSGPVRGWGPRSQGTLVTVKPGG | |
| 6 | EVQLVESGGGLVQAGDSLRLSCAASGFSFGSNVMMWVRQAP | hz1A5v8 |
| | GKGPEWVSTIYSSGTGTFYAESVKGRFTISRNNAKNTLYLQMS | |
| | SLRAEDTAVYYCATSGPVRGWGPRSQGTLVTVKPGG | |
| 7 | EVQLVESGGGEVQPGGSLRLSCAASGFSFGSNVMMWVRQAPG | hz1A5v9 |
| | KGPEWVSTIYSRGTGTFYAESVKGRFTISRNNAKNTLYLQMSS | |
| | LRAEDTAVYYCATSGPVRGWGPRSQGTLVTVKPGG | |
| 8 | EVQLVESGGGEVQPGGSLRLSCAASGFSFGSNVMMWVRQAPG | hz1A5v12 |
| | KGPEWVSTIYSSGTGTFYAESVKGRFTISRDNAKNTLYLQMSSL | |
| | RAEDTAVYYCATSGPVRGWGPWSQGTLVTVKP | |
| 9 | EVQLVESGGGEVQPGGSLRLSCAASGFSFGSNVMMWVRQAPG | hz1A5v17 |
| | KGPEWVSTIYSRGTGTFYAESVKGRFTISRDNAKNTLYLQMSS | |
| | LRAEDTAVYYCATSGPVRGWGPRSQGTLVTVKP | |
| 10 | EVQLVESGGGEVQPGGSLRLSCAASGFSFGSNVMMWVRQAPG | hz1A5v24 |
| | KGPEWVSTIYSSGTGTFYAESVKGRFTISRDNAKNTLYLQMSSL | |
| | RAEDTAVYYCATSGPVRGWGPRTQGTLVTVKP | |
| 11 | EVQLVESGGGEVQPGGSLRLSCAASGFSFGSNVMMWVRQAPG | hz1A5v25 |
| | KGLEWVSTIYSSGTGTFYAESVKGRFTISRDNAKNTLYLQMSS | |
| | LRAEDTAVYYCATSGPVRGWGPRSQGTLVTVKP | |
| 12 | EVQLVESGGGEVQAGGSLRLSCAASGFSFGSNVMMWVRQAP | hz1A5v28 |
| | GKGPEWVSTIYSSGTGTFYAESVKGRFTISRDNAKNTLYLQMS | |
| | SLRAEDTAVYYCATSGPVRGWGPRSQGTLVTVKP | |
| 13 | EVQLVESGGGEVQPGGSLRLSCAASGFSFGSNVMMWVRQAPG | hz1A5v29 |
| | KGLEWVSTIYSSGTGTFYAESVKGRFTISRDNAKNTLYLQMSS | |
| | LRAEDTAVYYCATSGPVRGWGPRTQGTLVTVKP | |
| 14 | EVQLVESGGGEVQPGGSLRLSCAASGFSFGSNVMMWVRQAPG | hz1A5v30 |
| | KGLEWVSTIYSRGGSTFYAESVKGRFTISRDNAKNTLYLQMSS | |
| | LRAEDTAVYYCATSGPVRGWGPRSQGTLVTVKP | |
| 15 | EVQLVESGGGEVQPGGSLRLSCAASGFSFGSNVMMWVRQAPG | hz1A5v31 |
| | KGLEWVSTIYSSGTGTYYAESVKGRFTISRDNAKNTLYLQMSS | |
| | LRAEDTAVYYCATSGPVRGWGPRSQGTLVTVKP | |
| 16 | EVQLVESGGGEVQPGGSLRLSCAASGFSFGSNVMMWVRQAPG | hz1A5v32 |
| | KGLEWVSTIYSRGGSTYYAESVKGRFTISRDNAKNTLYLQMSS | |
| | LRAEDTAVYYCATSGPVRGWGPRSQGTLVTVKP | |
| 17 | EVQLVESGGGEVQPGGSLRLSCAASGFSFGSNVMMWVRQAPG | hz1A5v33 |
| | KGLEWVSTIYSRGTGTFYAESVKGRFTISRDNAKNTLYLQMSS | |
| | LRAEDTAVYYCATSGPVRGWGPRSQGTLVTVKP | |
| 18 | EVQLVESGGGEVQPGGSLRLSCAASGFSFGSNVMMWVRQAPG | hz1A5v34 |
| | KGREWVSTIYSSGTGTFYAESVKGRFTISRDNAKNTLYLQMSS | |
| | LRAEDTAVYYCATSGPVRGWGPRSQGTLVTVKP | |
| 19 | EVQLVESGGGEVQPGGSLRLSCAASGFSFGSYVMMWVRQAPG | hz1A5v35 |
| | KGLEWVSTIYSSGTGTFYAESVKGRFTISRDNAKNTLYLQMSS | |
| | LRAEDTAVYYCATSGPVRGWGPRSQGTLVTVKP | |
| 20 | EVQLVESGGGEVQPGGSLRLSCAASGFSFGSFVMMWVRQAPG | hz1A5v36 |
| | KGLEWVSTIYSSGTGTFYAESVKGRFTISRDNAKNTLYLQMSS | |
| | LRAEDTAVYYCATSGPVRGWGPRSQGTLVTVKP | |
| 21 | EVQLVESGGGEVQPGGSLRLSCAASGFTFGSNVMMWVRQAPG | hz1A5v37 |
| | KGIEWVSTIYSSGTGTFYAESVKGRFTISRDNAKNTLYLQMSSL | |
| | RAEDTAVYYCATSGPVRGWGPRSQGTLVTVKP | |
| 22 | EVQLVESGGGEVQPGGSLRLSCAASGFSFGSNVMMWVRQAPG | hz1A5v38 |
| | KGLEWVSTIYSSGTGTFYAESVKGRFTISRDNAKNTLYLQMSS | |
| | LRAEDTAVYYCATSGPVRGWGYRSQGTLVTVKP | |
| 23 | EVQLVESGGGEVQPGGSLRLSCAASGFSFSSNVMMWVRQAPG | hz1A5v39 |
| | KGLEWVSTIYSSGTGTFYAESVKGRFTISRDNAKNTLYLQMSS | |
| | LRAEDTAVYYCATSGPVRGWGPRSQGTLVTVKP | |
| 24 | EVQLVESGGGEVQPGGSLRLSCAASGFSFTSNVMMWVRQAPG | hz1A5v40 |
| | KGLEWVSTIYSSGTGTFYAESVKGRFTISRDNAKNTLYLQMSS | |
| | LRAEDTAVYYCATSGPVRGWGPRSQGTLVTVKP | |
| 25 | EVQLVESGGGEVQPGGSLRLSCAPSGFSFGSNVMMWVRQAPG | hz1A5v41 |
| | KGLEWVSTIYSRGTGTFYAESVKGRFTISRDNAKNTLYLQMSS | |
| | LRAEDTAVYYCATSGPVRGWGPRSQGTLVTVKP | |
| 26 | EVQLVESGGGEVQPGGSLRLSCGPSGFSFGSNVMMWVRQAPG | hz1A5v42 |
| | KGLEWVSTIYSRGTGTFYAESVKGRFTISRDNAKNTLYLQMSS | |
| | LRAEDTAVYYCATSGPVRGWGPRSQGTLVTVKP | |
| 27 | EVQLVESGGGEVQPGGSLRLSCAASGFSFGSNVMMWVRQAPG | hz1A5v43 |
| | KGLEWVSTIYSRGTGTFYAESVKGRFTISRDNAKNTLYLQMSS | |
| | LRPEDTAVYYCATSGPVRGWGPRSQGTLVTVKP | |
| 28 | EVQLVESGGGEVQPGGSLRLSCAASGFSFGSNVMMWVRQAPG | hz1A5v44 |
| | KGLEWVSTIYSRGTGTYYAESVKGRFTISRDNAKNTLYLQMSS | |
| | LRAEDTAVYYCATSGPVRGWGPRSQGTLVTVKP | |
| 29 | EVQLVESGGGEVQPGGSLRLSCAASGFSFGSYVMMWVRQAPG | hz1A5v45 |
| | KGLEWVSTIYSRGTGTFYAESVKGRFTISRDNAKNTLYLQMSS | |
| | LRAEDTAVYYCATSGPVRGWGPRSQGTLVTVKP | |
| 30 | EVQLVESGGGEVQPGGSLRLSCAASGFTFGSNVMMWVRQAPG | hz1A5v46 |
| | KG1EWVSTIYSRGTGTFYAESVKGRFTISRDNAKNTLYLQMSSL | |
| | RAEDTAVYYCATSGPVRGWGPRSQGTLVTVKP | |
| 31 | EVQLVESGGGEVQPGGSLRLSCAASGFSFSSNVMMWVRQAPG | hz1A5v47 |
| | KGLEWVSTIYSRGTGTFYAESVKGRFTISRDNAKNTLYLQMSS | |
| | LRAEDTAVYYCATSGPVRGWGPRSQGTLVTVKP | |
| 32 | EVQLVESGGGEVQPGGSLRLSCAASGFSFGSYVMMWVRQAPG | hz1A5v48 |
| | KGLEWVSTIYSRGTGTYYAESVKGRFTISRDNAKNTLYLQMSS | |
| | LRAEDTAVYYCATSGPVRGWGPRSQGTLVTVKP | |
| 33 | EVQLVESGGGEVQPGGSLRLSCAASGFTFSSNVMMWVRQAPG | hz1A5v49 |
| | KGLEWVSTIYSRGTGTFYAESVKGRFTISRDNAKNTLYLQMSS | |
| | LRAEDTAVYYCATSGPVRGWGPRSQGTLVTVKP | |
| 34 | EVQLVESGGGEVQPGGSLRLSCAASGFTFSSYVMMWVRQAPG | hz1A5v50 |
| | KGLEWVSTIYSRGTGTYYAESVKGRFTISRDNAKNTLYLQMSS | |
| | LRAEDTAVYYCATSGPVRGWGPRSQGTLVTVKP | |
| 35 | EVQLVQSGGGLVQAGDSLRLSCAPSERTFSTYTMGWFRQAPG | L57B04 |
| | KEREFVAVVNWSGGSKYYADSVKGRFTISRDNAKNAVYLHM | |
| | NSLKPEDTGVYYCAAGGAYSGPYYDTRQYTYWGQGTQVTVK | |
| | P | |
| 36 | EVQLVESGGGEVQPGGSLRLSCAASERTFSTYTMGWFRQAPG | hz57B04v3 |
| | KEREFVSVVNWSGGSKYYAESVKGRFTISRDNAKNTVYLQMN | |
| | SLRAEDTAVYYCAAGGAYSGPYYDTRQYTYWGQGTLVTVKP | |
| | GG | |
| 37 | EVQLVESGGGEVQPGGSLRLSCAASERTFSTYTMGWFRQAPG | hz57B04v4 |
| | KEREFVAVVNWSGGSKYYAESVKGRFTISRDNAKNTVYLQMN | |
| | SLRAEDTAVYYCAAGGAYSGPYYDTRQYTYWGQGTLVTVKP | |
| | GG | |
| 38 | EVQLVESGGGEVQPGGSLRLSCAPSERTFSTYTMGWFRQAPGK | hz57B04v8 |
| | EREFVAVVNWSGGSKYYAESVKGRFriSRDNAKNTVYLQMNS | |
| | LRAEDTAVYYCAAGGAYSGPYYDTRQYTYWGQGTLVTVKPG | |
| | G | |
| 39 | EVQLVESGGGEVQPGGSLRLSCAASERTFSTYTMGWFRQAPG | hz57B04v9 |
| | KEREFVAVVNWSGGSKYYAESVKGRFTISRDNAKNTVYLHMN | |
| | SLRAEDTAVYYCAAGGAYSGPYYDTRQYTYWGQGTLVTVKP | |
| | GG | |
| 40 | EVQLVESGGGEVQPGGSLRLSCAPSERTFSTYTMGWFRQAPGK | hz57B04v15 |
| | EREFVAVVNWGGGSKYYAESVKGRFTISRDNAKNTVYLQMNS | |
| | LRAEDTAVYYCAAGGAYSGPYYDTRQYTYWGQGTLVTVKP | |
| 41 | EVQLVESGGGEVQPGGSLRLSCAPSERTFSTYTMGWFRQAPGK | hz57B04v20 |
| | EREFVAVVNWGGGSKYYAESVKGRFTISRDNAKNTVYLQMNS | |
| | LRAEDTAVYYCAAGGAYSTPYYDTRQYTYWGQGTLVTVKP | |
| 42 | EVQLVESGGGEVQPGGSLRLSCAPSERTFSTYTMGWFRQAPGK | hz57B04v23 |
| | EREFVAVVNWSGGSKYYAESVKGRFTISRDNAKNTVYLQMSS | |
| | LRAEDTAVYYCAAGGAYSGPYYDTRQYTYWGQGTLVTVKPG | |
| | G | |
| 43 | EVQLVESGGGEVQPGGSLRLSCAPSERTFSTYTMGWFRQAPGK | hz57B04v24 |
| | EREFVAVVNWGGGSKYYAESVKGRFTISRDNAKNTVYLQMSS | |
| | LRAEDTAVYYCAAGGAYSGPYYDTRQYTYWGQGTLVTVKPG | |
| | G | |
| 44 | EVQLVQSGGGLVQAGGSLRLSCAASGSTFSMYHMSWFRQAPG | L58E05 |
| | KQREPVATSHHGGTTNYAGSVKGRFTIFRDNAKNTVYLQMNS | |
| | LKPEDTGVYYCKADHGYNGRGYWGQGTQVTVKP | |
| 45 | EVQLVESGGGEVQPGGSLRLSCAASGSTFSMYHMSWFRQAPG | hz58E05v1 |
| | KGREPVSTSHHGGTTNYAESVKGRFTISRDNAKNTLYLQMNSL | |
| | RAEDTAVYYCKADHGYNGRGYWGQGTLVTVKPGG | |
| 46 | EVQLVESGGGEVQPGGSLRLSCAASGSTFSMYHMSWFRQAPG | hz58E05v2 |
| | KQREPVSTSHHGGTTNYAESVKGRFTISRDNAKNTLYLQMNSL | |
| | RAEDTAVYYCKADHGYNGRGYWGQGTLVTVKPGG | |
| 47 | EVQLVESGGGEVQPGGSLRLSCAASGSTFSMYHMSWFRQAPG | hz58E05v3 |
| | KQREPVSTSHHGGTTNYAESVKGRFTISRDNAKNTVYLQMNSL | |
| | RAEDTAVYYCKADHGYNGRGYWGQGTLVTVKPGG | |
| 48 | EVQLVESGGGEVQPGGSLRLSCAASGSTFSMYHMSWFRQAPG | hz58E05v4 |
| | KQREPVATSHHGGTTNYAESVKGRFTISRDNAKNTVYLQMNS | |
| | LRAEDTAVYYCKADHGYNGRGYWGQGTLVTVKPGG | |
| 49 | EVQLVESGGGEVQPGGSLRLSCAASGSTFSMYHMSWFRQAPG | hz58E05v5 |
| | KQREPVATSHHGGTTNYAESVKGRFTISRDNAKNTVYLQMNS | |
| | LRAEDTAVYYCKADHGYQGRGYWGQGTLVTVKPGG | |
| 50 | EVQLVESGGGEVQPGGSLRLSCAASGSTFSMYHMSWFRQAPG | hz58E05v6 |
| | KQREPVATSHHGGTTNYAESVKGRFTISRDNAKNTVYLQMNS | |
| | LRAEDTAVYYCKADHGYNARGYWGQGTLVTVKPGG | |
| 51 | EVQLVESGGGEVQPGGSLRLSCAASGSTFSMYHMSWFRQAPG | hz58E05v7 |
| | KQREPVATSHHGGTTNYAESVKGRFTISRDNAKNTVYLQMNS | |
| | LRAEDTAVYYCKADHGYNTRGYWGQGTLVTVKPGG | |
| 52 | EVQLVESGGGEVQPGGSLRLSCAASGSTFSMYHMSWFRQAPG | hz58E05v8 |
| | KQREPVATSHHGGTTNYAGSVKGRFTISRDNAKNTVYLQMNS | |
| | LRAEDTAVYYCKADHGYNGRGYWGQGTLVTVKPGG | |
| 53 | EVQLVESGGGEVQPGGSLRLSCAASGSTFSMYHMSWFRQAPG | hz58E05v9 |
| | KQREPVATSHHGGTTNYAESVKGRFTISRDNAKNTLYLQMNS | |
| | LRAEDTAVYYCKADHGYQGRGYWGQGTLVTVKPGG | |
| 54 | EVQLVESGGGEVQPGGSLRLSCAASGSTFSMYHMSWFRQAPG | hz58E05v10 |
| | KQREPVATSHHGGTTNYAESVKGRFTISRDNAKNTLYLQMNS | |
| | LRAEDTAVYYCKADHGYNARGYWGQGTLVTVKPG | |
| 55 | EVQLVESGGGEVQPGGSLRLSCAASGSTFSMYHMSWFRQAPG | hz58E05v11 |
| | KQREPVATSHHGGTTNYAESVKGRFTISRDNAKNTLYLQMNS | |
| | LRAEDTAVYYCKADHGYNTRGYWGQGTLVTVKPG | |
| 56 | EVQLVESGGGEVQPGGSLRLSCAASGSTFSMYHMSWFRQAPG | hz58E05v13 |
| | KQREPVSTSHHGGTTNYAESVKGRFTISRDNAKNTLYLQMNSL | |
| | RAEDTAVYYCKADHGYQGRGYWGQGTLVTVKP | |
| 57 | EVQLVESGGGEVQPGGSLRLSCAASGSTFSLYHMSWFRQAPG | hz58E05v16 |
| | KQREPVATSHHGGTTNYAGSVKGRFTISRDNAKNTVYLQMNS | |
| | LRAEDTAVYYCKADHGYQGRGYWGQGTLVTVKP | |
| 58 | EVQLVESGGGEVQPGGSLRLSCAASGSTFSSYHMSWFRQAPGK | hz58E05v18 |
| | QREPVATSHHGGTTNYAGSVKGRFTISRDNAKNTVYLQMNSL | |
| | RAEDTAVYYCKADHGYQGRGYWGQGTLVTVKP | |
| 59 | EVQLVESGGGEVQPGGSLRLSCAASGSTFSMYHMSWFRQAPG | hz58E05v19 |
| | KEREPVATSHHGGTTNYAGSVKGRFTISRDNAKNTVYLQMNS | |
| | LRAEDTAVYYCKADHGYQGRGYWGQGTLVTVKP | |
| 60 | EVQLVESGGGEVQPGGSLRLSCAASGSTFSTYHMSWFRQAPG | hz58E05v20 |
| | KQREPVATSHHGGTTNYAGSVKGRFTISRDNAKNTVYLQMNS | |
| | LRAEDTAVYYCKADHGYQGRGYWGQGTLVTVKP | |
| 61 | EVQLVESGGGEVQPGGSLRLSCAASGSTFSKYHMSWFRQAPG | hz58E05v21 |
| | KQREPVATSHHGGTTNYAGSVKGRFTISRDNAKNTVYLQMNS | |
| | LRAEDTAVYYCKADHGYQGRGYWGQGTLVTVKP | |
| 62 | EVQLVESGGGEVQPGGSLRLSCAASGSTFSFYHMSWFRQAPGK | hz58E05v22 |
| | QREPVATSHHGGTTNYAGSVKGRFTISRDNAKNTVYLQMNSL | |
| | RAEDTAVYYCKADHGYQGRGYWGQGTLVTVKP | |
| 63 | EVQLVESGGGEVQPGGSLRLSCAASGSTFSDYHMSWFRQAPG | hz58E05v23 |
| | KQREPVATSHHGGTTNYAGSVKGRFTISRDNAKNTVYLQMNS | |
| | LRAEDTAVYYCKADHGYQGRGYWGQGTLVTVKP | |
| 64 | EVQLVESGGGEVQPGGSLRLSCAASGSTFSRYHMSWFRQAPG | hz58E05v24 |
| | KQREPVATSHHGGTTNYAGSVKGRFTISRDNAKNTVYLQMNS | |
| | LRAEDTAVYYCKADHGYQGRGYWGQGTLVTVKP | |
| 65 | EVQLVESGGGEVQPGGSLRLSCAASGSTFSMYHMSWFRQAPG | hz58E05v25 |
| | KQREPVATSHHGGTTNYAGSVKGRFTISRDNAKNTVYLQMSS | |
| | LRAEDTAVYYCKADHGYQGRGYWGQGTLVTVKP | |
| 66 | EVQLVESGGGEVQPGGSLRLSCAASGSTFSMYHMSWFRQAPG | hz58E05v26 |
| | KQREPVATSHHGGTTNYAGSVKGRFTISRDNAKNTVYLQMNT | |
| | LRAEDTAVYYCKADHGYQGRGYWGQGTLVTVKP | |
| 67 | EVQLVESGGGEVQPGGSLRLSCAASGSTFSSYHMSWFRQAPGK | hz58E05v27 |
| | QREPVATSHHGGTTNYAGSVKGRFTISRDNAKNTVYLQMNTL | |
| | RAEDTAVYYCKADHGYQGRGYWGQGTLVTVKP | |
| 68 | EVQLVESGGGEVQPGGSLRLSCAASGSTFSMYHMSWFRQAPG | hz58E05v28 |
| | KQREPVATSHHGGTTNYAGSVKGRFTIFRDNAKNTVYLQMNS | |
| | LRAEDTAVYYCKADHGYNGRGYWGQGTLVTVKP | |
| 69 | EVQLVESGGGEVQPGGSLRLSCAASGSTFSMYHMSWFRQAPG | hz58E05v29 |
| | KQREPVATSHHGGTTNYAGSVKGRFTISRDNAKNTVYLQMNS | |
| | LKPEDTGVYYCKADHGYNGRGYWGQGTLVTVKP | |
| 70 | EVQLVESGGGEVQPGGSLRLSCAASGSTFSMYHMSWFRQAPG | hz58E05v30 |
| | KQREPVATSHHGGTTNYAGSVKGRFTISRDNAKNTVYLQMNS | |
| | LRPEDTAVYYCKADHGYNGRGYWGQGTLVTVKP | |
| 71 | EVQLVESGGGEVQPGGSLRLSCAASGSTFSMYHMSWFRQAPG | hz58E05v31 |
| | KQREPVATSHHGGTTNYAGSVKGRFTISRDNAKNTVYLQMNS | |
| | LRPEDTAVYYCKADHGYQGRGYWGQGTLVTVKP | |
| 72 | EVQLVESGGGEVQPGGSLRLSCAASGSTFSSYHMSWFRQAPGK | hz58E05v32 |
| | QREPVATSHHGGTTNYAGSVKGRFTISRDNAKNTVYLQMNSL | |
| | RPEDTAVYYCKADHGYQGRGYWGQGTLVTVKP | |
| 73 | EVQLVESGGGEVQPGGSLRLSCAASGSTFSMYHMSWFRQAPG | hz58E05v36 |
| | KQREPVATSHHGGTTNYAGSVKGRFTISRDNAKNTVYLQMNS | |
| | LRPEDTAVYYCKADHGYGGRGYWGQGTLVTVKP | |
| 74 | EVQLVESGGGEVQPGGSLRLSCAASGSTFSMYHMSWFRQAPG | hz58E05v37 |
| | KQREPVATSHHGGTTNYAGSVKGRFTISRDNAKNTVYLQMNS | |
| | LRPEDTAVYYCKADHGYSGRGYWGQGTLVTVKP | |
| 75 | EVQLVESGGGEVQPGGSLRLSCAASGSTFSMYHMSWFRQAPG | hz58E05v38 |
| | KQREPVATSHHGGTTNYAGSVKGRFTISRDNAKNTVYLQMNS | |
| | LRPEDTAVYYCKADHGYVGRGYWGQGTLVTVKP | |
| 76 | EVQLVESGGGEVQPGGSLRLSCAASGSTFSMYHMSWFRQAPG | hz58E05v39 |
| | KQREPVATSHHGGTTNYAGSVKGRFTISRDNAKNTVYLQMNS | |
| | LRPEDTAVYYCKADHGYEGRGYWGQGTLVTVKP | |
| 77 | EVQLVESGGGEVQPGGSLRLSCAASGSTFSMYHMSWFRQAPG | hz58E05v40 |
| | KQREPVATSHHGGTTNYAGSVKGRFTISRDNAKNTVYLQMNS | |
| | LRPEDTAVYYCKADHGYNPRGYWGQGTLVTVKP | |
| 78 | EVQLVESGGGEVQPGGSLRLSCAASGSTFSMYHMSWFRQAPG | hz58E05v41 |
| | KQREPVATSHHGGTTNYAGSVKGRFTISRDNAKNTVYLQMNS | |
| | LRPEDTAVYYCKADHGYGNRGYWGQGTLVTVKP | |
| 79 | EVQLVESGGGEVQPGGSLRLSCAASGSTFSMYHMSWFRQAPG | hz58E05v42 |
| | KQREPVATSHHGGTTNYAGSVKGRFTISRDNAKNTVYLQMNS | |
| | LRPEDTAVYYCKADHGNYGRGYWGQGTLVTVKP | |
| 80 | EVQLVESGGGEVQPGGSLRLSCAASGSTFSMYHMSWFRQAPG | hz58E05v43 |
| | KQREPVATSHHGGTTNYAGSVKGRFTISRDNAKNTVYLQMNS | |
| | LRPEDTAVYYCKADHGYNRGGYWGQGTLVTVKP | |
| 81 | EVQLVESGGGEVQPGGSLRLSCAASGSTFSSYHMSWFRQAPGK | hz58E05v44 |
| | QREPVATSHHGGTTNYAGSVKGRFTISRDNAKNTVYLQMNSL | |
| | RPEDTAVYYCKADHGYGGRGYWGQGTLVTVKP | |
| 82 | EVQLVESGGGEVQPGGSLRLSCAASGSTFSSYHMSWFRQAPGK | hz58E05v45 |
| | QREPVATSHHGGTTNYAGSVKGRFTISRDNAKNTVYLQMNSL | |
| | RPEDTAVYYCKADHGYEGRGYWGQGTLVTVKP | |
| 83 | EVQLVESGGGEVQPGGSLRLSCAASGSTFSSYHMSWFRQAPGK | hz58E05v46 |
| | QREPVATSHHGGTTNYAGSVKGRFTISRDNAKNTVYLQMNSL | |
| | RPEDTAVYYCKADHGNYGRGYWGQGTLVTVKP | |
| 84 | EVQLVESGGGEVQPGGSLRLSCAASGSTFSIYHMSWFRQAPGK | hz58E05v47 |
| | QREPVATSHHGGTTNYAGSVKGRFTISRDNAKNTVYLQMNSL | |
| | RPEDTAVYYCKADHGYGGRGYWGQGTLVTVKP | |
| 85 | EVQLVESGGGEVQPGGSLRLSCAASGFTFSSYHMSWFRQAPGK | hz58E05v48 |
| | QREPVATSHHGGTTNYAGSVKGRFTISRDNAKNTVYLQMNTL | |
| | RAEDTAVYYCKADHGYQGRGYWGQGTLVTVKP | |
| 86 | EVQLVESGGGEVQPGGSLRLSCAASGSTFSSYHMSWFRQAPGK | hz58E05v49 |
| | QRELVATSHHGGTTNYAGSVKGRFTISRDNAKNTVYLQMNTL | |
| | RAEDTAVYYCKADHGYQGRGYWGQGTLVTVKP | |
| 87 | EVQLVESGGGEVQPGGSLRLSCAASGSTFSSYHMSWFRQAPGK | hz58E05v50 |
| | QREWVATSHHGGTTNYAGSVKGRFTISRDNAKNTVYLQMNTL | |
| | RAEDTAVYYCKADHGYQGRGYWGQGTLVTVKP | |
| 88 | EVQLVESGGGEVQPGGSLRLSCAASGSTFSSYHMSWFRQAPGK | hz58E05v51 |
| | ERELVATSHHGGTTNYAGSVKGRFTISRDNAKNTVYLQMNTL | |
| | RAEDTAVYYCKADHGYQGRGYWGQGTLVTVKP | |
| 89 | EVQLVESGGGEVQPGGSLRLSCAASGSTFSSYHMSWFRQAPGK | hz58E05v52 |
| | EREFVATSHHGGTTNYAGSVKGRFTISRDNAKNTVYLQMNTL | |
| | RAEDTAVYYCKADHGYQGRGYWGQGTLVTVKP | |
| 90 | EVQLVESGGGEVQPGGSLRLSCAPSGSTFSSYHMSWFRQAPGK | hz58E05v53 |
| | QREPVATSHHGGTTNYAGSVKGRFTISRDNAKNTVYLQMNTL | |
| | RAEDTAVYYCKADHGYQGRGYWGQGTLVTVKP | |
| 91 | EVQLVESGGGEVQPGGSLRLSCGPSGSTFSSYHMSWFRQAPGK | hz58E05v54 |
| | QREPVATSHHGGTTNYAGSVKGRFTISRDNAKNTVYLQMNTL | |
| | RAEDTAVYYCKADHGYQGRGYWGQGTLVTVKP | |
| 92 | QVQLVQSGGGLVQAGGSLRLSCAASGLTFDEHHMGWFRQAP | 1A10 |
| | GKEREFVAAITWHTGTTWYADSVKGRFTISRDDAKNTVALQM | |
| | NALKTDDTALYYCVDGRRPFFIREVGVEPDYWGQGTQVTVKP | |
| 93 | QVQLQESGGGWVQPGGSLRLSCAASGSSFGSNVMMWVRQAP | 1E4 |
| | GKGPEWVSTINSSGTGTFYADSVKGRFTISRNNAKNTLYLQMN | |
| | SLKPDDTAVYYCATSGPVRGWGPRSQGTQVTVKP | |
| 94 | QVQLVQSGGGLVQPGGSLRLSCAASGFTFASTGMSWVRQAPG | D9 |
| | KGPEWVSSINSGSDSTMYADSVKGRFTISRDNAMNMLFLQMN | |
| | SLKPEDTAVYYCAKWALSCSGYGCDDLPQDAGQGTQVTVKP | |
| 95 | QVQLVQSGGGLVQPGGSLRLSCAASGFTFASYGMSWVRQAPG | A3 |
| | KGPEWVSSINSGSDTSMYADSVKGRFTISRDSAKNMLFLQMNS | |
| | LKPEDTGVYYCAKWALSCSQYGCDDLPRPAGQGTQVTVKP | |
| 96 | QVQLQQSGGGEAQPGGSLRLSCAASGRTFSSYAMSWYRQAPG | E9 |
| | KGREWVATITSSGSTTYYAESVKGRFTISRDNAKNTVYLQMSS | |
| | LRAEDTAVYYCNKYTSRTVRDYWGQGTQVTVKP | |
| 97 | EVQLVQSGGGEVQPGGSLRLSCAASGRTFSSYAMSWYRQAPG | B4 |
| | KGREWVATITTGGGTTYYAESVKGRFTISRDNAKNTVYLQMS | |
| | SLRAEDTAVYYCNKYTSRFPRDYWGQGTQVTVKP | |
| 98 | QVQLVQDGDSLRLSCKASGGTFSSYAMGWFRQAPGQEREFVA | 57B06 |
| | AISSEGGSTYYADNMEGRFTTSRDNAKNTVYLQMNSLKPEDT | |
| | AVYYCAVKGVGWPQEQASYDYWGQGTQVTVKP | |
| 99 | QVQLQQSGGGLVQPGGSMRLSCAASGSIPSIDHMGWYRQAPG | 57B10 |
| | KERELVASIDLNGRTNYAGPVKGRFAISRDSAKNTMYLQMNSL | |
| | LPEDTAVYYCNHRWGSPDYYHDDVDYWGQGTQVTVKP | |
| 100 | QVQLVQSGGGLVQAGGSLRLSCAASGRSFSTYAMGWFRQAPG | 58B06 |
| | KELEFVAAVGWRGTNTYYQDSVKGRFTISRDNAKNTVYLQMS | |
| | SLKPEDTAVYYCAMGEPIRVGEKSGYDYWGQGTQVTVKP | |
| 101 | QVQLQQSGGGLVQAGDSLRLSCEASGGTFSSYAMGWFRQAPR | 57A12 |
| | QEREFVAAISSEGGSTYYADNLEGRFTTSRDNAKNTVYLQMNS | |
| | LKPEDTAVYYCAVKGVGWPQEQASYDYWGQGTQVTVKP | |
| 102 | QVQLVQSGGGLVQAGGSLRLSCAASGLTFSSYAMGWFRHPPG | 57B08 |
| | KEREFVAAISWSGGNTLYADSVKGRFTISRDNSKNTVYLQMNS | |
| | LKPEDTAVYSCAVGPRDYFSDLEVDFGSWGQGTQVTVKP | |
| 103 | QVQLVQSGGGLVQAGDSLRLSCAASGRTFSSLAVGWFRQAPW | 58A08 |
| | KEREFVGAISWSGGNTYYVDAVEGRITISRDNAENTVYLQMN | |
| | DLKPEDTAVYYCAAGLPIRVGVPGGYDYWGQGTQVTVKP | |
| 104 | QVQLQQSGGGLVQAGGSLRLSCAASGHTFSTYAMGWFRQAP | 57D1 |
| | GKEREFVAGITRSGDSTHYEDSVKGRFTISRDNTKNTVYLRMN | |
| | SLKPEDTAVYYCAAASFAYLSTYTHHYDYWGQGTQVTVKP | |
| 105 | QVQLQQSGGGLVQAGGSLRLSCAASGRSFGDYTVGWFRQAPG | 1H5 |
| | KERDFVAGLSWLGGTIYYADSVKGRFTISRDNAKNTVYLQMT | |
| | TLKPEDTAVYYCAASRSAISRKATDFGSWGQGTQVTVKP | |
| 106 | EVQLVESGGGEVQPGGSLRLSCAASGRSFGDYTVGWFRQAPG | hz1H5v1 |
| | KERDFVAGLSWLGGTIYYAESVKGRFTISRDNAKNTLYLQMSS | |
| | LRAEDTAVYYCAASRSAISRKATDFGSWGQGTLVTVKP | |
| 107 | EVQLVESGGGEVQPGGSLRLSCAASGRSFGDYTVGWFRQAPG | hz1H5v2 |
| | KERDFVAGLSWLGGTIYYAESVKGRFTISRDNAKNTVYLQMSS | |
| | LRAEDTAVYYCAASRSAISRKATDFGSWGQGTLVTVKP | |
| 108 | EVQLVESGGGEVQPGGSLRLSCAASGRSFGDYTVGWFRQAPG | hz1H5v3 |
| | KERDFVAGLSWLGGTIYYAESVKGRFTISRDNAKNTVYLQMT | |
| | TLKPEDTAVYYCAASRSAISRKATDFGSWGQGTLVTVKP | |
| 109 | EVQLVESGGGEVQPGGSLRLSCAASGRSFGDYTVGWFRQAPG | hz1H5v4 |
| | KERDFVAGLSWLGGTIYYAESVKGRFTISRDNAKNTVYLQMSS | |
| | LRAEDTAVYYCAASRSAISRKATDFGSWGQGTQVTVKP | |
| 110 | QVQLVQSGGGLVQDGDSLRLSCKASGGTFSSYAMGWFRQAPG | L57B06 |
| | QEREFVAAISSEGGSTYYADNMEGRFTTSRDNAKNTVYLQMN | |
| | SLKPEDTAVYYCAVKGVGWPQEQASYDYWGQGTQVTVKP | |
| ill | EVQLVESGGGEVQPGGSLRLSCAASGGTFSSYAMGWFRQAPG | h57B06v5 |
| | KEREFVAAISSEGGSTYYADNMEGRFTISRDNAKNTVYLQMSS | |
| | LRAEDTAVYYCAVKGVGWPQEQASYDYWGQGTLVTVKPG | |
| 112 | EVQLVESGGGEVQPGGSLRLSCAASGGTFSSYAMGWFRQAPG | h57B06v6 |
| | KEREFVAAISSEGGSTYYAESMEGRFTISRDNAKNTVYLQMSS | |
| | LRAEDTAVYYCAVKGVGWPQEQASYDYWGQGTLVTVKPG | |
| 113 | QVQLVQDGDSLRLSCAASGGTFSSYAMGWFRQAPGKEREFVA | h57B06v7 |
| | AISSEGGSTYYADNMEGRFTISRDNAKNTVYLQMSSLRAEDTA | |
| | VYYCAVKGVGWPQEQASYDYWGQGTLVTVKPG | |
| 114 | EVQLVESGGGEVQPGGSLRLSCAASGGTFSSYAMGWFRQAPG | h57B06v8 |
| | QEREFVAAISSEGGSTYYAESVKGRFTISRDNAKNTVYLQMSSL | |
| | RAEDTAVYYCAVKGVGWPQEQASYDYWGQGTLVTVKPG | |
| 115 | GFSFGSNVMM | CDR-H1 (L1A5, |
| | | hz1A5v1, 2, 3, 7, 8, 9, |
| | | 12, 17, 24, 25, 28, 29, |
| | | 30, 31, 32, 33, 34, 38, |
| | | 41, 42, 43, 44) |
| 116 | GFSFGSYVMM | CDR-H1 |
| | | (hz1A5v35, 45, 48) |
| 117 | GFSFGSFVMM | CDR-H1 |
| | | (hz1A5v36) |
| 118 | GFTFGSNVMM | CDR-H1 |
| | | (hz1A5v37, 46) |
| 119 | GFSFSSNVMM | CDR-H1 |
| | | (hz1A5v39, 47) |
| 120 | GFSFTSNVMM | CDR-H1 |
| | | (hz1A5v40) |
| 121 | GFTFSSNVMM | CDR-H1 |
| | | (hz1A5v49) |
| 122 | GFTFSSYVMM | CDR-H1 |
| | | (hz1A5v50) |
| 123 | ERTFSTYTMG | CDR-H1 (L57B04, |
| | | hz57B04v3, 4, 8, 9, 1 |
| | | 5, 20, 23, 24) |
| 124 | GSTFSMYHMS | CDR-H1 (L58E05, |
| | | hz58E05v1, 2, 3, 4, 5 |
| | | , 6, 7, 8, 9, 10, 11, 13, 1 |
| | | 9, 25, 26, 28, 29, 30, 3 |
| | | 1, 36, 37, 38, 39, 40, 4 |
| | | 1, 42, 43) |
| 125 | GSTFSLYHMS | CDR-H1 |
| | | (hz58E05v16) |
| 126 | GSTFSSYHMS | CDR-H1 |
| | | (hz58E05v18, 27, 3 |
| | | 2, 44, 45, 46, 49, 50, 5 |
| | | 1, 52, 53, 54) |
| 127 | GSTFSTYHMS | CDR-H1 |
| | | (hz58E05v20) |
| 128 | GSTFSKYHMS | CDR-H1 |
| | | (hz58E05v21) |
| 129 | GSTFSFYHMS | CDR-H1 |
| | | (hz58E05v22) |
| 130 | GSTFSDYHMS | CDR-H1 |
| | | (hz58E05v23) |
| 131 | GSTFSRYHMS | CDR-H1 |
| | | (hz58E05v24) |
| 132 | GSTFSIYHMS | CDR-H1 |
| | | (hz58E05v47) |
| 133 | GFTFSSYHMS | CDR-H1 |
| | | (hz58E05v48) |
| 134 | GLTFDEHHMG | CDR-H1 (cx3969 |
| | | pP12-B7H3-1359- |
| | | 1A10 xELL) |
| 135 | GSSFGSNVMM | CDR-H1 (cx3982 |
| | | pP12-B7H3-1675- |
| | | 1E4 xELL) |
| 136 | GFTFASTGMS | CDR-H1 (cx3218 |
| | | pP12-B7H3 avi- |
| | | D9-xELL) |
| 137 | GFTFASYGMS | CDR-H1 (cx3219 |
| | | pP12-B7H3 avi- |
| | | A3-xELL) |
| 138 | GRTFSSYAMS | CDR-H1 |
| | | (synthetic E9, |
| | | synthetic B4) |
| 139 | GGTFSSYAMG | CDR-H1 (pPL2- |
| | | B7H3 VHH-9 |
| | | (57B06) IgG1, |
| | | pPL2-B7H3 VHH- |
| | | 7 (57A12) IgG1, |
| | | L57B06, |
| | | h57B06v5, 6, 7, 8) |
| 140 | GSIPSIDHMG | CDR-H1 (pPL2- |
| | | B7H3 VHH-12 |
| | | (57B10)IgG1) |
| 141 | GRSFSTYAMG | CDR-H1 (pPL2- |
| | | B7H3 VHH-54 |
| | | (58B06) IgG1) |
| 142 | GLTFSSYAMG | CDR-H1 (pPL2- |
| | | B7H3 VHH-10 |
| | | (57B08) IgG1) |
| 143 | GRTFSSLAVG | CDR-H1 (pPL2- |
| | | B7H3 VHH-53 |
| | | (58A08) IgG1) |
| 144 | GHTFSTYAMG | CDR-H1 (pP12- |
| | | B7H3 VHH |
| | | 57D1c-IgG1) |
| 145 | GRSFGDYTVG | CDR-H1 (1H5, |
| | | hz1H5v1, 2, 3, 4) |
| 146 | TIYSSGTGTF | CDR-H2 |
| | | (hz1A5v1, 2, 3, 7, 8, 12, |
| | | 24, 25, 28, 29, 34, 35, |
| | | 36, 37, 38, 39, 40) |
| 147 | TIYSRGTGTF | CDR-H2 |
| | | (hz1A5v9, 17, 33, 41, |
| | | 42, 43, 45, 46, 47, |
| | | 49) |
| 148 | TIYSRGGSTF | CDR-H2 |
| | | (hz1A5v30) |
| 149 | TIYSSGTGTY | CDR-H2 |
| | | (hz1A5v31) |
| 150 | TIYSRGGSTY | CDR-H2 |
| | | (hz1A5v32) |
| 151 | TIYSRGTGTY | CDR-H2 |
| | | (hz1A5v44, 48, 50) |
| 152 | VVNWSGGSKY | CDR-H2 (L57B04, |
| | | hz57B04v3, 4, 8, 9, 2 |
| | | 3) |
| 153 | VVNWGGGSKY | CDR-H2 |
| | | (hz57B04v15, 20, 2 |
| | | 4) |
| 154 | TSHHGGTTN | CDR-H2 (L58E05, |
| | | hz58E05v1, 2, 3, 4, 5, |
| | | 6, 7, 8, 9, 10, 11, 13, |
| | | 16, 18, 19, 20, 21, 22, |
| | | 23, 2, 2, 26, 27, 28, 29, |
| | | 30, 31, 32, 36, 37, 38, |
| | | 39, 40, 41, 42, 43, 44, |
| | | 45, 46, 47, 48, 49, 50, |
| | | 51, 52, 53, 54) |
| 155 | AITWHTGTTW | CDR-H2 (cx3969 |
| | | pP12-B7H3-1359- |
| | | 1A10 xELL) |
| 156 | TINSSGTGTF | CDR-H2 (cx3982 |
| | | pP12-B7H3-1675- |
| | | 1E4 xELL) |
| 157 | SINSGSDSTM | CDR-H2 (cx3218 |
| | | pP12-B7H3 avi- |
| | | D9-xELL) |
| 158 | SINSGSDTSM | CDR-H2 |
| | | (GFTFASYGMS) |
| 159 | TITSSGSTTY | CDR-H2 |
| | | (synthetic E9) |
| 160 | TITTGGGTTY | CDR-H2 |
| | | (synthetic B4) |
| 161 | AISSEGGSTY | CDR-H2 (pPL2- |
| | | B7H3 VHH-9 |
| | | (57B06) IgG1, |
| | | pPL2-B7H3 VHH- |
| | | 7 (57A12) IgG1, |
| | | L57B06, |
| | | h57B06v5, 6, 7, 8) |
| 162 | SIDLNGRTN | CDR-H2 (pPL2- |
| | | B7H3 VHH-12 |
| | | (57B10)IgG1) |
| 163 | AVGWRGTNTY | CDR-H2 (pPL2- |
| | | B7H3 VHH-54 |
| | | (58B06) IgG1) |
| 164 | AISWSGGNTL | CDR-H2 (pPL2- |
| | | B7H3 VHH-10 |
| | | (57B08) IgG1) |
| 165 | AISWSGGNTY | CDR-H2 (pPL2- |
| | | B7H3 VHH-53 |
| | | (58A08) IgG1) |
| 166 | GITRSGDSTH | CDR-H2 (pP12- |
| | | B7H3 VHH |
| | | 57D1c-IgG1) |
| 167 | GLSWLGGTIY | CDR-H2 (1H5, |
| | | hz1H5v1, 2, 3, 4) |
| 168 | SGPVRGWGP | CDR-H3 (L1A5, |
| | | hz1A5v1, 2, 3, 7, 8, 9, |
| | | 12, 17, 24, 25, 28, 29, |
| | | 30, 31, 32, 33, 34, 35, |
| | | 36, 37, 39, 40, 41, 42, |
| | | 43, 44, 45, 46, 47, 48, |
| | | 49, 50, cx3982 |
| | | pP12-B7H3-1675- |
| | | 1E4 xELL) |
| 169 | SGPVRGWGY | CDR-H3 |
| | | (hz1A5v38) |
| 170 | GGAYSGPYYDTRQYTY | CDR-H3 (L57B04, |
| | | hz57B04v3, 4, 8, 9, |
| | | 15, 23, 24) |
| 171 | GGAYSTPYYDTRQYTY | CDR-H3 |
| | | (hz57B04v20) |
| 172 | DHGYNGRGY | CDR-H3 (L58E05, |
| | | hz58E05v1, 2, 3, 4, |
| | | 8, 28, 29, 30) |
| 173 | DHGYQGRGY | CDR-H3 |
| | | (hz58E05v5, 9, 13, 16, |
| | | 18, 19, 20, 21, 22, |
| | | 23, 24, 25, 26, 27, 31, |
| | | 32, 48, 49, 50, 51, 52, |
| | | 53, 54) |
| 174 | DHGYNARGY | CDR-H3 |
| | | (hz58E05v6) |
| 175 | DHGYNTRGY | CDR-H3 |
| | | (hz58E05v7, 11) |
| 176 | DHGYGGRGY | CDR-H3 |
| | | (hz58E05v36, 44, |
| | | 47) |
| 177 | DHGYSGRGY | CDR-H3 |
| | | (hz58E05v37) |
| 178 | DHGYVGRGY | CDR-H3 |
| | | (hz58E05v38) |
| 179 | DHGYEGRGY | CDR-H3 |
| | | (hz58E05v39, 45) |
| 180 | DHGYNPRGY | CDR-H3 |
| | | (hz58E05v40) |
| 181 | DHGYGNRGY | CDR-H3 |
| | | (hz58E05v41) |
| 182 | DHGNYGRGY | CDR-H3 |
| | | (hz58E05v42, 46) |
| 183 | DHGYNRGGY | CDR-H3 |
| | | (hz58E05v43) |
| 184 | GRRPFFIREVGVEPDY | CDR-H3 (cx3969 |
| | | pP12-B7H3-1359- |
| | | 1A10 xELL) |
| 185 | WALSCSGYGCDDLPQD | CDR-H3 (cx3218 |
| | | pP12-B7H3 avi- |
| | | D9-xELL) |
| 186 | WALSCSQYGCDDLPRP | CDR-H3 (cx3219 |
| | | pP12-B7H3 avi- |
| | | A3-xELL) |
| 187 | YTSRTVRDY | CDR-H3 |
| | | (synthetic E9) |
| 188 | YTSRFPRDY | CDR-H3 |
| | | (synthetic B4) |
| 189 | KGVGWPQEQASYDY | CDR-H3 (pPL2- |
| | | B7H3 VHH-7 |
| | | (57A12) IgG1) |
| 190 | MLRRRGSPGMGVHVGAALGALWFCLTGALEVQVPEDPVVAL | B7H3 |
| | VGTDATLCCSFSPEPGFSLAQLNLIWQLTDTKQLVHSFAEGQD | |
| | QGSAYANRTALFPDLLAQGNASLRLQRVRVADEGSFTCFVSIR | |
| | DFGSAAVSLQVAAPYSKPSMTLEPNKDLRPGDTVTITCSSYQG | |
| | YPEAEVFWQDGQGVPLTGNVTTSQMANEQGLFDVHSILRVVL | |
| | GANGTYSCLVRNPVLQQDAHSSVTITPQRSPTGAVEVQVPEDP | |
| | VVALVGTDATLRCSFSPEPGFSLAQLNLIWQLTDTKQLVHSFTE | |
| | GRDQGSAYANRTALFPDLLAQGNASLRLQRVRVADEGSFTCF | |
| | VSIRDFGSAAVSLQVAAPYSKPSMTLEPNKDLRPGDTVTITCSS | |
| | YRGYPEAEVFWQDGQGVPLTGNVTTSQMANEQGLFDVHSVL | |
| | RVVLGANGTYSCLVRNPVLQQDAHGSVTITGQPMTFPPEALW | |
| | VTVGLSVCLIALLVALAFVCWRKIKQSCEEENAGAEDQDGEGE | |
| | GSKTALQPLKHSDSKEDDGQEIA | |
| 191 | GGSGGS | (GGS)2 linker |
| 192 | GGSGGSGGS | (GGS)3 linker |
| 193 | GGSGGSGGSGGS | (GGS)4 linker |
| 194 | GGSGGSGGSGGSGGS | (GGS)5 linker |
| 195 | GGGG | glycine linker |
| 196 | GGGGG | glycine linker |
| 197 | GGGGGG | glycine linker |
| 198 | PAPELLGGPS VFLFPPKPKD TLMISRTPEV TCVVVDVSHE | human IgG1 Fc |
| | DPEVKFNWYV DGVEVHNAKT KPREEQYNST YRVVSVLTVL | |
| | HQDWLNGKEY KCKVSNKALP APIEKTISKA KGQPREPQVY | |
| | TLPPSRDELT KNQVSLTCLV KGFYPSDIAV EWESNGQPEN | |
| | NYKTTPPVLD SDGSFFLYSK LTVDKSRWQQ GNVFSCSVMH | |
| | EALHNHYTQK SLSLSPGK | |
| 199 | PAPGGPSVFL FPPKPKDTLM ISRTPEVTCV VVDVSHEDPE | Fc xELL |
| | VKFNWYVDGV EVHNAKTKPR EEQYNSTYRV VSVLTVLHQD | |
| | WLNGKEYKCK VSNKALPAPI EKTISKAKGQ PREPQVYTLP | |
| | PSRDELTKNQ VSLTCLVKGF YPSDIAVEWE SNGQPENNYK | |
| | TTPPVLDSDG SFFLYSKLTV DKSRWQQGNV FSCSVMHEAL | |
| | HNHYTQKSLS LSPGK | |
| 200 | PAPPVAGPSV FLFPPKPKDT LMISRTPEVT CVVVDVSHED | human IgG2 Fc |
| | PEVQFNWYVD GVEVHNAKTK PREEQFNSTF RVVSVLTVVH | |
| | QDWLNGKEYK CKVSNKGLPA PIEKTISKTK GQPREPQVYT | |
| | LPPSREEMTK NQVSLTCLVK GFYPSDISVE WESNGQPENN | |
| | YKTTPPMLDS DGSFFLYSKL TVDKSRWQQG NVFSCSVMHE | |
| | ALHNHYTQKS LSLSPGK | |
| 201 | PAPELLGGPS VFLFPPKPKD TLMISRTPEV TCVVVDVSHE | human IgG3 Fc |
| | DPEVQFKWYV DGVEVHNAKT KPREEQYNST FRVVSVLTVL | |
| | HQDWLNGKEY KCKVSNKALP APIEKTISKT KGQPREPQVY | |
| | TLPPSREEMT KNQVSLTCLV KGFYPSDIAV EWESSGQPEN | |
| | NYNTTPPMLD SDGSFFLYSK LTVDKSRWQQ GNIFSCSVMH | |
| | EALHNRFTQK SLSLSPGK | |
| 202 | PAPEFLGGPS VFLFPPKPKD TLMISRTPEV TCVVVDVSQE | human IgG4 Fc |
| | DPEVQFNWYV DGVEVHNAKT KPREEQFNST YRVVSVLTVL | |
| | HQDWLNGKEY KCKVSNKGLP SSIEKTISKA KGQPREPQVY | |
| | TLPPSQEEMT KNQVSLTCLV KGFYPSDIAV EWESNGQPEN | |
| | NYKTTPPVLD SDGSFFLYSR LTVDKSRWQE GNVFSCSVMH | |
| | EALHNHYTQK SLSLSLGK | |
| 203 | PAPELLGGPS VFLFPPKPKD TLMISRTPEV TCVVVDVSQE | human IgG4 Fc |
| | DPEVQFNWYV DGVEVHNAKT KPREEQFNST YRVVSVLTVL | |
| | HQDWLNGKEY KCKVSNKGLP SSIEKTISKA KGQPREPQVY | |
| | TLPPSQEEMT KNQVSLTCLV KGFYPSDIAV EWESNGQPEN | |
| | NYKTTPPVLD SDGSFFLYSR LTVDKSRWQE GNVFSCSVMH | |
| | EALHNHYTQK SLSLSLGK | |
| 204 | EPKSSDKTHTCPPC | modified IgG1 |
| | | hinge |
| 205 | DKTHTCPPC | truncated IgG1 |
| | | hinge |
| 206 | ESKYGPPCPPC | modified IgG4 |
| | | hinge |
| 207 | GQGTLVTVKPGG | carboxy-terminal |
| | | sequence |
| 208 | GQGTLVTVEPGG | carboxy-terminal |
| | | sequence |
| 209 | QVQLVQSGGGVVQPGRSLRLSCKASGYTFTRYTMHWVRQAP | OKT3 VH |
| | GKGLEWIGYINPSRGYTNYNQKVKDRFTISRDNSKNTAFLQMD | |
| | SLRPEDTGVYFCARYYDDHYCLDYWGQGTPVTVSS | |
| 210 | DIQMTQSPSSLSASVGDRVTITCSASSSVSYMNWYQQTPGKAP | OKT3 VL |
| | KRWIYDTSKLASGVPSRFSGSGSGTDYTFTISSLQPEDIATYYCQ | |
| | QWSSNPFTFGQGTKLQIT | |
| 211 | QVQLVQSGGGVVQPGRSLRLSCKASGYTFTRYTMHWVRQAP | OKT3 humanized |
| | GKGLEWIGYINPSRGYTNYNQKVKDRFTISRDNSKNTAFLQMD | VH |
| | SLRPEDTGVYFCARYYDDHYSLDYWGQGTPVTVSS | |
| 212 | DVQLVQSGAEVKKPGASVKVSCKASGYTFTRYTMHWVRQAP | OKT3 humanized |
| | GQGLEWIGYINPSRGYTNYADSVKGRFTITTDKSTSTAYMELSS | VH |
| | LRSEDTATYYCARYYDDHYCLDYWGQGTTVTVSS | |
| 213 | QVQLVQSGAELKKPGASVKVSCKASGYTFTRYTMHWVRQAP | OKT3 humanized |
| | GQCLEWMGYINPSRGYTNYNQKFKDKATLTADKSTSTAYMEL | VH |
| | RSLRSDDTAVYYCARYYDDHYSLDYWG QGTLVTVSS | |
| 214 | QIVLTQSPAIMSASPGEKVTMTCSASSSVSYMNWYQQKSGTSP | OKT3 humanized |
| | KRWIYDTSKLASGVPAHFRGSGSGTSYSLTISGMEAEDAATYY | VL |
| | CQQWSSNPFTFGSGTKLEIN | |
| 215 | DIQMTQSPSSLSASVGDRVTITCRASQSVSYMNWYQQKPGKAP | OKT3 humanized |
| | KRWIYDTSKVASGVPARFSGSGSGTDYSLTINSLEAEDAATYY | VL |
| | CQQWSSNPLTFGGGTKVEIK | |
| 216 | DIQLTQSPSILSASVGDRVTITCRASSSVSYMNWYQQKPGKAPK | OKT3 humanized |
| | RWIYDT | VL |
| | SKVASGVPYRFSGSGSGTEYTLTISSMQPEDFATYYCQQWSSN | |
| | PLTFGCGTKVEIKRT | |
| 217 | EVQLVESGGGLVQPKGSLKLSCAASGFTFNTYAMNWVRQAPG | anti-CD3 Hv |
| | KGLEWVARIRSKYNNYATYYADSVKDRFTISRDDSQSILYLQM | |
| | NNLKTEDTAMYYCVRHGNFGNSYVSWFAYWGQGTLVTVSA | |
| 218 | QAVVTQESALTTSPGETVTLTCRSSTGAVTTSNYANWVQEKPD | anti-CD3 Lv |
| | HLFTGLIGGTNKRAPGVPARFSGSLIGDKAALTITGAQTEDEAI | |
| | YFCALWYSNLWVFGGGTKLTVL | |
| 219 | TYAMN | anti-CD3 VH |
| | | CDR1 |
| 220 | RIRSKYNNYATYYADSVKD | anti-CD3 VH |
| | | CDR2 |
| 221 | HGNFGNSYVSWFAY | anti-CD3 VH |
| | | CDR3 |
| 222 | RSSTGAVTTSNYAN | anti-CD3 VL |
| | | CDR1 |
| 223 | GTNKRAP | anti-CD3 VL |
| | | CDR2 |
| 224 | ALWYSNLWV | anti-CD3 VL |
| | | CDR3 |
| 225 | EVQLVESGGGLVQPGGSLRLSCAASGFTFSTYAMNWVRQAPG | anti-CD3 VH1 |
| | KGLEWVGRIRSKYNNYATYYADSVKDRFTISRDDSKNSLYLQ | |
| | MNSLKTEDTAVYYCVRHGNFGNSYVSWFAYWGQGTLVTVSS | |
| 226 | EVKLVESGGGLVQPGGSLRLSCAASGFTFNTYAMNWVRQAPG | anti-CD3 VH2 |
| | KGLEWVARIRSKYNNYATYYADSVKDRFTISRDDSKSSLYLQ | |
| | MNNLKTEDTAMYYCVRHGNFGNSYVSWFAYWGQGTLVTVSS | |
| 227 | EVKLVESGGGLVKPGRSLRLSCAASGFTFNTYAMNWVRQAPG | anti-CD3 VH3 |
| | KGLEWVARIRSKYNNYATYYADSVKDRFTISRDDSKSILYLQM | |
| | NNLKTEDTAMYYCVRHGNFGNSYVSWFAYWGQGTLVTVSS | |
| 228 | EVKLVESGGGLVKPGRSLRLSCAASGFTFNTYAMNWVRQAPG | anti-CD3 VH4 |
| | KGLEWVARIRSKYNNYATYYADSVKDRFTISRDDSKSILYLQM | |
| | NSLKTEDTAMYYCVRHGNFGNSYVSWFAYWGQGTLVTVSS | |
| 229 | EVKLVESGGGLVKPGRSLRLSCAASGFTFNTYAMNWVRQAPG | anti-CD3 VH5 |
| | KGLEWVARIRSKYNNYATYYADSVKDRFTISRDDSKSILYLQM | |
| | NSLKTEDTAMYYCVRHGNFGNSYVSWFAYWGQGTLVTVSS | |
| 230 | EVQLLESGGGLVQPGGSLRLSCAASGFTFSTYAMNWVRQAPG | anti-CD3 VH6 |
| | KGLEWVSRIRSKYNNYATYYADSVKGRFTISRDDSKNTLYLQ | |
| | MNSLRAEDTAVYYCVRHGNFGNSYVSWFAYWGQGTLVTVSS | |
| 231 | EVQLVESGGGLVQPGGSLRLSCAASGFTFSTYAMSWVRQAPG | anti-CD3 VH7 |
| | KGLEWVGRIRSKYNNYATYYADSVKGRFTISRDDSKNTLYLQ | |
| | MNSLRAEDTAVYYCVRHGNFGDSYVSWFAYWGQGTLVTVSS | |
| 232 | EVQLVESGGGLVQPGGSLKLSCAASGFTFNKYAMNWVRQAPG | anti-CD3 VH8 |
| | KGLEWVARIRSKYNNYATYYADSVKDRFTISRDDSKNTAYLQ | |
| | MNNLKTEDTAVYYCVRHGNFGNSYISYWAYWGQGTLVTVS | |
| 233 | EVQLVESGGGLVQPGGSLRLSCAASGFTFNTYAMNWVRQAPG | anti-CD3 VH9 |
| | KGLEWVARIRSKYNNYATYYADSVKGRFTISRDDSKNTLYLQ | |
| | MNSLRAEDTAVYYCVRHGNFGNSYVSWFAYWGQGTTVTVSS | |
| 234 | EVQLVESGGGLVQPGGSLRLSCAASGFTFNTYAMNWVRQAPG | anti-CD3 VH10 |
| | KGLEWVARIRSKYNNYATYYADSVKGRFTISRDDSKNTLYLQ | |
| | MNSLRAEDTAVYYCVRHGNFGNSYVSYFAYWGQGTTVTVSS | |
| 235 | EVQLVESGGGLVQPKGSLKLSCAASGFTFNTYAMNWVRQAPG | anti-CD3 VH11 |
| | KGLEWVARIRSKYNNYATYYADSVKDRFTISRDDSQSILYLQM | |
| | NNLKTEDTAMYYCVRHGNFGNSYVSWFAYWGQGTLVTVSS | |
| 236 | EVQLVESGGGLVQPGGSLRLSCAASGFTFNTYAMNWVRQAPG | anti-CD3 VH12 |
| | KGLEWVARIRSKYNNYATYYADSVKGRFTISRDDAKNTLYLQ | |
| | MSSLRAEDTAVYYCVRHGNFGNSYVSWFAYWGQGTLVTVKP | |
| 237 | EVQLVESGGGLVQPGGSLRLSCAASGFTFNTYAMNWVRQAPG | anti-CD3 VH13 |
| | KCLEWVARIRSKYNNYATYYADSVKGRFTISRDDAKNTLYLQ | |
| | MSSLRAEDTAVYYCVRHGNFGNSYVSWFAYWGQGTLVTVKP | |
| 238 | EVQLVESGGGLVQPGGSLRLSCAASGFTFNTYAMNWVRQAPG | anti-CD3 VH14 |
| | KGLEWVARIRSKYNNYATYYADSVKGRFTISRDDAKNTLYLQ | |
| | MSSLRAEDTAVYYCVRHGNFGNSYVSWFAYWGCGTLVTVKP | |
| 239 | EVQLVESGGGLVQPGGSLRLSCAASGFTFSTYAMNWVRQAPG | anti-CD3 VH15 |
| | KGLEWVARIRSKYNNYATYYADSVKGRFTISRDDAKNTLYLQ | |
| | MSSLRAEDTAVYYCVRHGNFGNSYVSWFAYWGQGTLVTVSS | |
| 240 | EVQLVESGGGLVQPGGSLRLSCAASGFTFNTYAMNWVRQAPG | anti-CD3 VH16 |
| | KGLEWVSRIRSKYNNYATYYADSVKGRFTISRDDAKNTLYLQ | |
| | MSSLRAEDTAVYYCVRHGNFGNSYVSWFAYWGQGTLVTVSS | |
| 241 | EVQLVESGGGLVQPGGSLRLSCAASGFTFSTYAMNWVRQAPG | anti-CD3 VH17 |
| | KGLEWVSRIRSKYNNYATYYADSVKGRFTISRDDAKNTLYLQ | |
| | MSSLRAEDTAVYYCVRHGNFGNSYVSWFAYWGQGTLVTVSS | |
| 242 | EVQLVESGGGLVQPGGSLRLSCAASGFTFSTYAMNWVRQAPG | anti-CD3 VH18 |
| | KCLEWVARIRSKYNNYATYYADSVKGRFTISRDDAKNTLYLQ | |
| | MSSLRAEDTAVYYCVRHGNFGNSYVSWFAYWGQGTLVTVSS | |
| 243 | EVQLVESGGGLVQPGGSLRLSCAASGFTFNTYAMNWVRQAPG | anti-CD3 VH19 |
| | KCLEWVSRIRSKYNNYATYYADSVKGRFTISRDDAKNTLYLQ | |
| | MSSLRAEDTAVYYCVRHGNFGNSYVSWFAYWGQGTLVTVSS | |
| 244 | EVQLVESGGGLVQPGGSLRLSCAASGFTFSTYAMNWVRQAPG | anti-CD3 VH20 |
| | KCLEWVSRIRSKYNNYATYYADSVKGRFTISRDDAKNTLYLQ | |
| | MSSLRAEDTAVYYCVRHGNFGNSYVSWFAYWGQGTLVTVSS | |
| 245 | EVQLVESGGGLVQPGGSLRLSCAASGFTFSTYAMNWVRQAPG | anti-CD3 VH21 |
| | KCLEWVGRIRSKYNNYATYYADSVKDRFTISRDDSKNSLYLQ | |
| | MNSLKTEDTAVYYCVRHGNFGNSYVSWFAYWGQGTLVTVSS | |
| 246 | EVKLVESGGGLVQPGGSLRLSCAASGFTFNTYAMNWVRQAPG | anti-CD3 VH22 |
| | KCLEWVARIRSKYNNYATYYADSVKDRFTISRDDSKSSLYLQ | |
| | MNNLKTEDTAMYYCVRHGNFGNSYVSWFAYWGQGTLVTVSS | |
| 247 | EVKLVESGGGLVKPGRSLRLSCAASGFTFNTYAMNWVRQAPG | anti-CD3 VH23 |
| | KCLEWVARIRSKYNNYATYYADSVKDRFTISRDDSKSILYLQM | |
| | NNLKTEDTAMYYCVRHGNFGNSYVSWFAYWGQGTLVTVSS | |
| 248 | EVKLVESGGGLVKPGRSLRLSCAASGFTFNTYAMNWVRQAPG | anti-CD3 VH24 |
| | KCLEWVARIRSKYNNYATYYADSVKDRFTISRDDSKSILYLQM | |
| | NSLKTEDTAMYYCVRHGNFGNSYVSWFAYWGQGTLVTVSS | |
| 249 | EVKLVESGGGLVKPGRSLRLSCAASGFTFNTYAMNWVRQAPG | anti-CD3 VH25 |
| | KCLEWVARIRSKYNNYATYYADSVKDRFTISRDDSKSILYLQM | |
| | NSLKTEDTAMYYCVRHGNFGNSYVSWFAYWGQGTLVTVSS | |
| 250 | EVQLLESGGGLVQPGGSLRLSCAASGFTFSTYAMNWVRQAPG | anti-CD3 VH26 |
| | KCLEWVSRIRSKYNNYATYYADSVKGRFTISRDDSKNTLYLQ | |
| | MNSLRAEDTAVYYCVRHGNFGNSYVSWFAYWGQGTLVTVSS | |
| 251 | EVQLVESGGGLVQPGGSLRLSCAASGFTFSTYAMSWVRQAPG | anti-CD3 VH27 |
| | KCLEWVGRIRSKYNNYATYYADSVKGRFTISRDDSKNTLYLQ | |
| | MNSLRAEDTAVYYCVRHGNFGDSYVSWFAYWGQGTLVTVSS | |
| 252 | EVQLVESGGGLVQPGGSLKLSCAASGFTFNKYAMNWVRQAPG | anti-CD3 VH28 |
| | KCLEWVARIRSKYNNYATYYADSVKDRFTISRDDSKNTAYLQ | |
| | MNNLKTEDTAVYYCVRHGNFGNSYISYWAYWGQGTLVTVS | |
| 253 | EVQLVESGGGLVQPGGSLRLSCAASGFTFNTYAMNWVRQAPG | anti-CD3 VH29 |
| | KCLEWVARIRSKYNNYATYYADSVKGRFTISRDDSKNTLYLQ | |
| | MNSLRAEDTAVYYCVRHGNFGNSYVSWFAYWGQGTTVTVSS | |
| 254 | EVQLVESGGGLVQPGGSLRLSCAASGFTFNTYAMNWVRQAPG | anti-CD3 VH30 |
| | KCLEWVARIRSKYNNYATYYADSVKGRFTISRDDSKNTLYLQ | |
| | MNSLRAEDTAVYYCVRHGNFGNSYVSYFAYWGQGTTVTVSS | |
| 255 | EVQLVESGGGLVQPKGSLKLSCAASGFTFNTYAMNWVRQAPG | anti-CD3 VH31 |
| | KCLEWVARIRSKYNNYATYYADSVKDRFTISRDDSQSILYLQM | |
| | NNLKTEDTAMYYCVRHGNFGNSYVSWFAYWGQGTLVTVSS | |
| 256 | QAVVTQESALTTSPGETVTLTCRSSTGAVTTSNYANWVQEKPD | anti-CD3 VL1 |
| | HLFTGLIGGTNKRAPGVPARFSGSLIGDKAALTITGAQTEDEAI | |
| | YFCALWYSNLWVFGGGTKLTVL | |
| 257 | QAVVTQEPSLTVSPGGTVTLTCGSSTGAVTTSNYANWVQQKP | anti-CD3 VL2 |
| | GKSPRGLIGGTNKRAPGVPARFSGSLLGGKAALTISGAQPEDEA | |
| | DYYCALWYSNHWVFGCGTKLEIK | |
| 258 | QAVVTQEPSLTVSPGGTVTLTCRSSTGAVTTSNYANWVQQKP | anti-CD3 VL3 |
| | GQAPRGLIGGTNKRAPWTPARFSGSLLGGKAALTITGAQAEDE | |
| | ADYYCALWYSNLWVFGGGTKLTVL | |
| 259 | QAVVTQEPSFSVSPGGTVTLTCRSSTGAVTTSNYANWVQQTPG | anti-CD3 VL4 |
| | QAFRGLIGGTNKRAPGVPARFSGSLIGDKAALTITGAQADDESI | |
| | YFCALWYSNLWVFGGGTKLTVL | |
| 260 | QAVVTQEPSFSVSPGGTVTLTCRSSTGAVTTSNYANWVQQTPG | anti-CD3 VL5 |
| | QAFRGLIGGTNKRAPGVPARFSGSILGNKAALTITGAQADDESI | |
| | YFCALWYSNLWVFGGGTKLTVL | |
| 261 | QAVVTQEPSFSVSPGGTVTLTCRSSTGAVTTSNYANWVQQTPG | anti-CD3 VL6 |
| | QAFRGLIGGTNKRAPGVPARFSGSILGNKAALTITGAQADDESD | |
| | YYCALWYSNLWVFGGGTKLTVL | |
| 262 | QAVVTQEPSLTVSPGGTVTLTCGSSTGAVTTSNYANWVQEKP | anti-CD3 VL7 |
| | GQAFRGLIGGTNKRAPGTPARFSGSLLGGKAALTLSGAQPEDE | |
| | AEYYCALWYSNLWVFGGGTKLTVL | |
| 263 | QTVVTQEPSLTVSPGGTVTLTCGSSTGAVTSGNYPNWVQQKPG | anti-CD3 VL8 |
| | QAPRGLIGGTKFLAPGTPARFSGSLLGGKAALTLSGVQPEDEAE | |
| | YYCVLWYSNRWVFGGGTKLTVL | |
| 264 | QAVVTQEPSLTVSPGGTVTLTCGSSTGAVTTSNYANWVQQKP | anti-CD3 VL9 |
| | GQAFRGLIGGTNKRAPGVPARFSGSLLGGKAALTISGAQPEDE | |
| | ADYYCALWYSNHWVFGGGTKLEIK | |
| 265 | QAVVTQEPSLTVSPGGTVTLTCGSSTGAVTTSNYANWVQQKP | anti-CD3 VL10 |
| | GQAFRGLIGGTNKRAPGVPARFSGSLLGGKAALTISGAQPEDE | |
| | ADYYCALWYSNHWVFGGGTKLEIK | |
| 266 | QAVVTQEPSLTVSPGGTVTLTCGSSTGAVTTSNYANWVQQKP | anti-CD3 VL11 |
| | GQCFRGLIGGTNKRAPGVPARFSGSLLGGKAALTISGAQPEDE | |
| | ADYYCALWYSNHWVFGEGTKLEIK | |
| 267 | QAVVTQESALTTSPGETVTLTCRSSTGAVTTSNYANWVQEKPD | anti-CD3 VL12 |
| | HLFTGLIGGTNKRAPGVPARFSGSLIGDKAALTITGAQTEDEAI | |
| | YFCALWYSNLWVFGCGTKLTVL | |
| 268 | QAVVTQEPSLTVSPGGTVTLTCGSSTGAVTTSNYANWVQQKP | anti-CD3 VL13 |
| | GKSPRGLIGGTNKRAPGVPARFSGSLLGGKAALTISGAQPEDEA | |
| | DYYCALWYSNHWVFGGGTKLEIK | |
| 269 | QAVVTQEPSLTVSPGGTVTLTCRSSTGAVTTSNYANWVQQKP | anti-CD3 VL14 |
| | GQAPRGLIGGTNKRAPWTPARFSGSLLGGKAALTITGAQAEDE | |
| | ADYYCALWYSNLWVFGCGTKLTVL | |
| 270 | QAVVTQEPSFSVSPGGTVTLTCRSSTGAVTTSNYANWVQQTPG | anti-CD3 VL15 |
| | QAFRGLIGGTNKRAPGVPARFSGSLIGDKAALTITGAQADDESI | |
| | YFCALWYSNLWVFGGGTKLTVL | |
| 271 | QAVVTQEPSFSVSPGGTVTLTCRSSTGAVTTSNYANWVQQTPG | anti-CD3 VL16 |
| | QAFRGLIGGTNKRAPGVPARFSGSILGNKAALTITGAQADDESI | |
| | YFCALWYSNLWVFGCGTKLTVL | |
| 272 | QAVVTQEPSFSVSPGGTVTLTCRSSTGAVTTSNYANWVQQTPG | anti-CD3 VL17 |
| | QAFRGLIGGTNKRAPGVPARFSGSILGNKAALTITGAQADDESD | |
| | YYCALWYSNLWVFGCGTKLTVL | |
| 273 | QAVVTQEPSLTVSPGGTVTLTCGSSTGAVTTSNYANWVQEKP | anti-CD3 VL18 |
| | GQAFRGLIGGTNKRAPGTPARFSGSLLGGKAALTLSGAQPEDE | |
| | AEYYCALWYSNLWVFGCGTKLTVL | |
| 274 | QTVVTQEPSLTVSPGGTVTLTCGSSTGAVTSGNYPNWVQQKPG | anti-CD3 VL19 |
| | QAPRGLIGGTKFLAPGTPARFSGSLLGGKAALTLSGVQPEDEAE | |
| | YYCVLWYSNRWVFGCGTKLTVL | |
| 275 | QVQLQESGGGLVQAGGSLRLSCAASGRTFSNYHMGWFRQAPG | anti-CD3 VHH |
| | KERELVAAISGSGGSTYYTDSVKGRFTISRNNAKNTMSLQMSN | |
| | LKPEDTGVYYCTTPTEKGSSIDYWGQGTQVTVSSGRYPYDVPD | |
| | Y | |
| 276 | MPEEGSGCSVRRRPYGCVLRAALVPLVAGLVICLVVCIQRFAQ | UniProt No. |
| | AOOOLPLESLGWDVAELOLNHTGPOODPRLYWOGGPALGRSF | P32970, CD70- |
| | LHGPELDKGOLRIHRDGIYMVHIOVTLAICSSTTASRHHPTTLA | ECD residues 39-193 |
| | VGICSPASRSISLLRLSFHOGCTIASORLTPLARGDTLCTNLTGT | |
| | LLPSRNTDETFFGVQWVRP | |
| 277 | QVQLVESGGGVVQPGRSLRLSCAASGFTFSSYDMHWVRQAPG | CD70 VH |
| | KGLEWVAVIWYDGSNKYYADSVKGRFTISRDNSKNTLYLQM | |
| | NSLRAEDTAVYYCARGSGNWGFFDYWGQGTLVTVSS | |
| 278 | DIQMTQSPSSLSASVGDRVTITCRASQGISRWLAWYQQKPEKA | CD70 VL |
| | PKSLIYAASSLQSGVPSRFSGSGSGTDFTLTISSLQPEDFATYYC | |
| | QQYNTYPRTFGQGTKVEIK | |
| 279 | QVQLQQSGGGLVQPGGSLRLSCAASGSIFSINGMGWYRQAPG | ICOS sdAb |
| | KERELVAGLTSGGSVTNYADSVKGRFTISRDNAKNTVYLQMN | |
| | SLKPEDTAVYYCRAEIFTRTGENYYGMDYWGKGTQVTVKP | |
| 280 | EVQLVESGGGEVQPGGSLRLSCAASGRMFSNYAMGWFRQAPG | CD28 sdAb |
| | KEREFVAAINYRRDAADYAESVKGRFTISRDNAKNTVYLQMN | |
| | SLRAEDTAVYYCGFTYAGWASSRRDDYNYWGQGTLVTVKP | |
| 281 | RVKFSRSADAPAYQQGQNQLYNELNLGRREEYDVLDKRRGRD | CD3zeta signaling |
| | PEMGGKPRRKNPQEGLYNELQKDKMAEAYSEIGMKGERRRG | domain |
| | KGHDGLYQGLSTATKDTYDALHMQALPPR | |
| 282 | KRGRKKLLYIFKQPFMRPVQTTQEEDGCSCRFPEEEEGGCEL | 4-1BB-derived |
| | | costimulatory |
| | | domain |
| 283 | SKRSRLLHSDYMNMTPRRPGPTRKHYQPYAPPRDFAAYRS | CD28-derived |
| | | costimulatory |
| | | domain |
| 284 | RSKRSRLLHSDYMNMTPRRPGPTRKHYQPYAPPRDFAAYRS | CD28-derived |
| | | costimulatory |
| | | domain 2 |
| 285 | FWVRSKRSRLLHSDYMNMTPRRPGPTRKHYQPYAPPRDFAAY | CD28-derived |
| | RS | costimulatory |
| | | domain 3 |
| 286 | KPTTTPAPRPPTPAPTIASQPLSLRPEASRPAAGGAVHTRGLDFA | CD8-derived hinge |
| | SDIYIWAPLAGTCGVLLLSLVITLYC | and |
| | | transmembrane |
| | | domain |
| 287 | AKPTTTPAPRPPTPAPTIASQPLSLRPEACRPAAGGAVHTRGLD | CD8-derived hinge |
| | FACDIYIWAPLAGTCGVLLLSLVIT | and |
| | | transmembrane |
| | | domain |
| 288 | KPTTTPAPRPPTPAPTIASQPLSLRPEACRPAAGGAVHTRGLDF | CD8 hinge and |
| | ACDIYIWAPLAGTCGVLLLSLVIT | transmembrane |
| | | domain |
| 289 | QVQLQESGPGLVKPSETLSLTCTVSGGSISSGGYFWSWIRQPPG | GITR VH |
| | KGLEWIGYIYYSGTTYYNPSLKSRVTISIDTSKNQFSLKLSSVTA | |
| | ADTAVYYCARDLFYYDTSGPRGFDPWGQGTLVTVSS | |
| 290 | EIVLTQSPGTLSLSPGERATLSCRASQTVSSNYLAWYQQKPGQ | GITR VL |
| | APRLLIYGSSTRATGIPDRFSGSGSGTDFTLTISRLEPEDFAVYY | |
| | CQQYDSSPWTFGQGTKVEIK | |
| 291 | QVQLVESGGGVVQPGRSLRLSCAASGFTFSSYGMHWVRQAPG | GITR VH |
| | KGLEWVAVIWYPGSNKYYAESVKGRFTISRDNSKNTLYLQMN | |
| | SLRAEDTAVYYCARGGELGRYYYYGMDVWGQGTTVTVSS | |
| 292 | DIQMTQSPSSLSASVGDRVTVTCRASQGIRNDLGWYQQKPGK | GITR VL |
| | APKRLIYAASSLQSGVPSRFSGSGSGTEFTLTISSLQPEDFATYY | |
| | CLQHNNYPWTFGQGTKVDIK | |
| 293 | DKTHTCPPCPAPELLGGPSVFLFPPKPKDTLMISRTPEVTCVVV | Knob Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPCRDELTKNQVSLWCLVKGFYPSDIAVEWESNGQPENNY | |
| | KTTPPVLDSDGSFFLYSKLTVDKSRWQQGNVFSCSVMHEALH | |
| | NHYTQKSLSLSPT | |
| 294 | DKTHTCPPCPAPELLGGPSVFLFPPKPKDTLMRSRTPEVTCVVV | Hole Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | CTLPPSRDELTKNQVSLSCAVKGFYPSDIAVEWESNGQPENNY | |
| | KTTPPVLDSDGSFFLVSKLTVDKSRWQQGNVFSCSVMHEALH | |
| | NHYTQKSLSLSPT | |
| 295 | DKTHTCPPCPAPGGPSVFLFPPKPKDTLMISRTPEVTCVVVDVS | Knob Fc |
| | HEDPEVKFNWYVDGVEVHNAKTKPREEQYNSTYRVVSVLTVL | |
| | HQDWLNGKEYKCKVSNKALPAPIEKTISKAKGQPREPQVYTLP | |
| | PCRDELTKNQVSLWCLVKGFYPSDIAVEWESNGQPENNYKTTP | |
| | PVLDSDGSFFLYSKLTVDKSRWQQGNVFSCSVMHEALHNHYT | |
| | QKSLSLSPT | |
| 296 | DKTHTCPPCPAPGGPSVFLFPPKPKDTLMRSRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVMHEALHNHYT | |
| | QKSLSLSPT | |
| 297 | DKTHTCPPCPAPELLGGPSVFLFPPKPKDTLMISRTPEVTCVVV | Knob Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPCRDELTKNQVSLWCLVKGFYPSDIAVEWESNGQPENNY | |
| | KTTPPVLDSDGSFFLYSKLTVDKSRWQQGNVFSCSVMHEALH | |
| | NHYTQKSLSLSPG | |
| 298 | DKTHTCPPCPAPELLGGPSVFLFPPKPKDTLMRSRTPEVTCVVV | Hole Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | CTLPPSRDELTKNQVSLSCAVKGFYPSDIAVEWESNGQPENNY | |
| | KTTPPVLDSDGSFFLVSKLTVDKSRWQQGNVFSCSVMHEALH | |
| | NHYTQKSLSLSPG | |
| 299 | DKTHTCPPCPAPGGPSVFLFPPKPKDTLMISRTPEVTCVVVDVS | Knob Fc |
| | HEDPEVKFNWYVDGVEVHNAKTKPREEQYNSTYRVVSVLTVL | |
| | HQDWLNGKEYKCKVSNKALPAPIEKTISKAKGQPREPQVYTLP | |
| | PCRDELTKNQVSLWCLVKGFYPSDIAVEWESNGQPENNYKTTP | |
| | PVLDSDGSFFLYSKLTVDKSRWQQGNVFSCSVMHEALHNHYT | |
| | QKSLSLSPG | |
| 300 | DKTHTCPPCPAPGGPSVFLFPPKPKDTLMRSRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVMHEALHNHYT | |
| | QKSLSLSPG | |
| 301 | DKTHTCPPCPAPELLGGPSVFLFPPKPKDTLMISRTPEVTCVVV | Hole Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | CTLPPSRDELTKNQVSLSCAVKGFYPSDIAVEWESNGQPENNY | |
| | KTTPPVLDSDGSFFLVSKLTVDKSRWQQGNVFSCSVMHEALH | |
| | NRYTQKSLSLSPT | |
| 302 | DKTHTCPPCPAPGGPSVFLFPPKPKDTLMISRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVMHEALHNRYT | |
| | QKSLSLSPT | |
| 303 | DKTHTCPPCPAPELLGGPSVFLFPPKPKDTLMISRTPEVTCVVV | Hole Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | CTLPPSRDELTKNQVSLSCAVKGFYPSDIAVEWESNGQPENNY | |
| | KTTPPVLDSDGSFFLVSKLTVDKSRWQQGNVFSCSVMHEALH | |
| | NRYTQKSLSLSPG | |
| 304 | DKTHTCPPCPAPGGPSVFLFPPKPKDTLMISRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVMHEALHNRYT | |
| | QKSLSLSPG | |
| 305 | DKTHTCPPCPAPELLGGPSVFLFPPKPKDTLYISRTPEVTCVVVD | Knob Fc |
| | VSHEDPEVKFNWYVDGVEVHNAKTKPREEQYNSTYRVVSVLT | |
| | VLHQDWLNGKEYKCKVSNKALPAPIEKTISKAKGQPREPQVYT | |
| | LPPCRDELTKNQVSLWCLVKGFYPSDIAVEWESNGQPENNYKT | |
| | TPPVLDSDGSFFLYSKLTVDKSRWQQGNVFSCSVVHEALHNH | |
| | YTQKSLSLSPT | |
| 306 | DKTHTCPPCPAPGGPSVFLFPPKPKDTLYISRTPEVTCVVVDVS | Knob Fc |
| | HEDPEVKFNWYVDGVEVHNAKTKPREEQYNSTYRVVSVLTVL | |
| | HQDWLNGKEYKCKVSNKALPAPIEKTISKAKGQPREPQVYTLP | |
| | PCRDELTKNQVSLWCLVKGFYPSDIAVEWESNGQPENNYKTTP | |
| | PVLDSDGSFFLYSKLTVDKSRWQQGNVFSCSVVHEALHNHYT | |
| | QKSLSLSPT | |
| 307 | DKTHTCPPCPAPELLGGPSVFLFPPKPKDTLYISRTPEVTCVVVD | Knob Fc |
| | VSHEDPEVKFNWYVDGVEVHNAKTKPREEQYNSTYRVVSVLT | |
| | VLHQDWLNGKEYKCKVSNKALPAPIEKTISKAKGQPREPQVYT | |
| | LPPCRDELTKNQVSLWCLVKGFYPSDIAVEWESNGQPENNYKT | |
| | TPPVLDSDGSFFLYSKLTVDKSRWQQGNVFSCSVVHEALHNH | |
| | YTQKSLSLSPG | |
| 308 | DKTHTCPPCPAPGGPSVFLFPPKPKDTLYISRTPEVTCVVVDVS | Knob Fc |
| | HEDPEVKFNWYVDGVEVHNAKTKPREEQYNSTYRVVSVLTVL | |
| | HQDWLNGKEYKCKVSNKALPAPIEKTISKAKGQPREPQVYTLP | |
| | PCRDELTKNQVSLWCLVKGFYPSDIAVEWESNGQPENNYKTTP | |
| | PVLDSDGSFFLYSKLTVDKSRWQQGNVFSCSVVHEALHNHYT | |
| | QKSLSLSPG | |
| 309 | DKTHTCPPCPAPELLGGPSVFLFPPKPKDTLYISRTPEVTCVVVD | Hole Fc |
| | VSHEDPEVKFNWYVDGVEVHNAKTKPREEQYNSTYRVVSVLT | |
| | VLHQDWLNGKEYKCKVSNKALPAPIEKTISKAKGQPREPQVCT | |
| | LPPSRDELTKNQVSLSCAVKGFYPSDIAVEWESNGQPENNYKT | |
| | TPPVLDSDGSFFLVSKLTVDKSRWQQGNVFSCSVVHEALHNRY | |
| | TQKSLSLSPT | |
| 310 | DKTHTCPPCPAPGGPSVFLFPPKPKDTLYISRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVVHEALHNRYT | |
| | QKSLSLSPT | |
| 311 | DKTHTCPPCPAPELLGGPSVFLFPPKPKDTLYISRTPEVTCVVVD | Hole Fc |
| | VSHEDPEVKFNWYVDGVEVHNAKTKPREEQYNSTYRVVSVLT | |
| | VLHQDWLNGKEYKCKVSNKALPAPIEKTISKAKGQPREPQVCT | |
| | LPPSRDELTKNQVSLSCAVKGFYPSDIAVEWESNGQPENNYKT | |
| | TPPVLDSDGSFFLVSKLTVDKSRWQQGNVFSCSVVHEALHNRY | |
| | TQKSLSLSPG | |
| 312 | DKTHTCPPCPAPGGPSVFLFPPKPKDTLYISRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVVHEALHNRYT | |
| | QKSLSLSPG | |
| 313 | (GGGGS)n, wherein n is 1 to 5 | Linker |
| 314 | (GGGGGS)n, wherein n is 1 to 4 | linker |
| 315 | GGGGS | Linker |
| 316 | GGGGGS | Linker |
| 317 | GGGGGSGGGGGSGGGGGS | Linker |
| 318 | GGGGSGGGGSGGGGS | Linker |
| 319 | GGSGGGGSGGGGSGGGGS | Linker |
| 320 | GlyxXaa-Glyy-Xaa-Glyz | Linker |
| | Xaa is independently selected from A, | |
| | V, L, I, M, F, W, P, G, S, T, C, | |
| | Y, N, Q, K, R, H, D, or E | |
| | x, y, and z are each integers | |
| | in the range from 1-5 | |
| 321 | Gly-Gly-Gly-Xaa-Gly-Gly-Gly-Xaa-Gly-Gly-Gly | Linker |
| | Xaa is independently selected from A, | |
| | V, L, I, M, F, W, P, G, S, T, C, | |
| | Y, N, Q, K, R, H, D, or E | |
| 322 | (SSSSG)n | Linker |
| | n = l-9 | |
| 323 | GGGGG-C-GGGGG | Linker |
| 324 | (EAAAK)n | Linker |
| | n = 2-20 | |
| 325 | AS-(AP)n-GT | Linker |
| | n = 2-20 | |
| 326 | AS-(EAAAK)n-GT | Linker |
| | n = 2-20 | |
| 327 | (GGGGA)n | Linker |
| | n = 2-20 | |
| 328 | (PGGGS)n | Linker |
| | n = 2-20 | |
| 329 | (AGGGS)n | Linker |
| | n = 2-20 | |
| 330 | GGS-(EGKSSGSGSESKST)n-GGS | Linker |
| | n = 2-20 | |
| 331 | SSSASASSA | Linker |
| 332 | GSPGSPG | Linker |
| 333 | ATTTGSSPGPT | Linker |
| 334 | X1 X2 X3 X4 X5 (P4 P3 P2 P1 ↓ P1′) | Linker consensus |
| | Xl = I, L, Y, M, F, V, or A; | |
| | (P4 = 1, L, Y, M, F, V, or A) | |
| | X2 = A, G, S, V, E, D, Q, N, or Y; | |
| | (P3 = A, G, S, V, E, D, Q, N, or Y) | |
| | X3 = H, P, A, V, G, S, or T; | |
| | (P2 = H, P, A, V, G, S, or T) | |
| | X4 = D or E; (P1 = D or E) | |
| | X5 = 1, L, Y, M, F, V, T, S, G or A | |
| | (P1′ = I, L, Y, M, F, V, T, S, G or | |
| | A) | |
| 335 | X1 E X3 D X5 (P4 P3 P2 P1 ↓ P1′) | Linker consensus |
| | X1 = I or L; (P4 = I or L) | |
| | (P3 = E) | |
| | X3 = P or A; (P2 = P or A) | |
| | X5 = 1, V, T, S, or G | |
| | (P1′ = I, V, T, S, or G) | |
| 336 | LEAD | granzyme B |
| | | substrate |
| 337 | LEPD | Linker |
| 338 | LEAE | Linker |
| 339 | IEPDI | Linker |
| 340 | LEPDG | Linker |
| 341 | LEADT | Linker |
| 342 | IEPDG | Linker |
| 343 | IEPDV | Linker |
| 344 | IEPDS | Linker |
| 345 | IEPDT | Linker |
| 346 | X1QARX5 (P1QAR↓A/V)) | Linker consensus |
| | X1 = any amino acid; | |
| | (Pl is any amino acid) | |
| | X5 = A or V | |
| 347 | RQARX5 (RQAR(A/V)) | Linker |
| | X5 = A or V | |
| 348 | RQAR | matriptase |
| | | substrate |
| 349 | RQARV | linker |
| 350 | X1X2 X3 X4 (P3 P2 P1 ↓ P1′) | Linker consensus |
| | X1 = P, V or A; (P3 = P, V or A) | |
| | X2 = Q or D; (P2 = Q or D) | |
| | X3 = A or N; (P1 = A or N) | |
| | X4 = L, I or M (PF = L, I or M) | |
| 351 | PX2X3X4 (P3 P2 P1 ↓ P1′) | Linker consensus |
| | (P3 = P) | |
| | X2 = Q or D; (P2 = Q or D) | |
| | X3 = A or N; (P1 is A or N) | |
| | X4 = L or I (P1′ is L or I) | |
| 352 | PAGL | MMP substrate |
| 353 | TGLEADGSPAGLGRQARVG | Linker |
| 354 | TGLEADGSRQARVGPAGLG | Linker |
| 355 | TGSPAGLEADGSRQARVGS | Linker |
| 356 | TGPAGLGLEADGSRQARVG | Linker |
| 357 | TGRQARVGLEADGSPAGLG | Linker |
| 358 | TGSRQARVGPAGLEADGS | Linker |
| 359 | TGPAGLGSRQARVGLEADGS | Linker |
| 360 | GPAGLGLEPDGSRQARVG | Linker |
| 361 | GGSGGGGIEPDIGGSGGS | Linker |
| 362 | GGSGGGGLEADTGGSGGS | Linker |
| 363 | GSIEPDIGS | Linker |
| 364 | GSLEADTGS | Linker |
| 365 | GGSGGGGIEPDGGGSGGS | Linker |
| 366 | GGSGGGGIEPDVGGSGGS | Linker |
| 367 | GGSGGGGIEPDSGGSGGS | Linker |
| 368 | GGSGGGGIEPDTGGSGGS | Linker |
| 369 | GGGSLEPDGSGS | Linker |
| 370 | GPAGLGLEADGSRQARVG | Linker |
| 371 | GGEGGGGSGGSGGGS | Linker |
| 372 | GSSAGSEAGGSGQAGVGS | Linker |
| 373 | GGSGGGGLEAEGSGGGGS | Linker |
| 374 | GGSGGGGIEPDPGGSGGS | Linker |
| 375 | TGGSGGGGIEPDIGGSGGS | Linker |
| 376 | ACPWAVSGARASPGSAASPRLREGPELSPDDPAGLLDLRQGMF | 41BBL |
| | AQLVAQNVLLIDGPLSWYSDPGLAGVSLTGGLSYKEDTKELV | |
| | VAKAGVYYVFFQLELRRVVAGEGSGSVSLALHLQPLRSAAGA | |
| | AALALTVDLPPASSEANSAFGFQGRLLHLSAGQRLGVHLHTEA | |
| | RARHAWQLTQGATVLGLFRVTPEIPAGLPSPRSE | |
| 377 | EVQLVQSGAEVKKPGESLRISCKGSGYSFSTYWISWVRQMPGK | 41BB VH |
| | GLEWMGKIYPGDSYTNYSPSFQGQVTISADKSISTAYLQWSSL | |
| | KASDTAMYYCARGYGIFDYWGQGTLVTVSS | |
| 378 | SYELTQPPSVSVSPGQTASITCSGDNIGDQYAHWYQQKPGQSP | 41BB VL |
| | VLVIYQDKNRPSGIPERFSGSNSGNTATLTISGTQAMDEADYYC | |
| | ATYTGFGSLAVFGGGTKLTVL | |
| 379 | QVQLQQWGAGLLKPSETLSLTCAVYGGSFSGYYWSWIRQSPE | 41BB VH |
| | KGLEWIGEINHGGYVTYNPSLESRVTISVDTSKNQFSLKLSSVT | |
| | AADTAVYYCARDYGPGNYDWYFDLWGRGTLVTVSS | |
| 380 | EIVLTQSPATLSLSPGERATLSCRASQSVSSYLAWYQQKPGQAP | 41BB VL |
| | RLLIYDASNRATGIPARFSGSGSGTDFTLTISSLEPEDFAVYYCQ | |
| | QRSNWPPALTFGGGTKVEIK | |
| 381 | QMQLVQSGAEVKKPGASVKVSCKASGYSFSGYYMHWVRQAP | 41BB VH |
| | GQGLEWMGWVNPMSGGTNYAQKFQGRVTITRDTSASTAYME | |
| | LSSLRSEDTAVYYCAREGMAMRLELDKWGQGTLVTVSS | |
| 382 | SYELTQPPSVSVAPGKTARITCGGNNIGSKSVHWYQQKPGQAP | 41BB VL |
| | VLVIYYDSDRPSGIPERFSGSNSGNTATLTISRVEAGDEADYYC | |
| | QVWDSSSVVFGGGTQLTVL | |
| 383 | QDSTSDLIPAPPLSKVPLQQNFQDNQFHGKWYVVGQAGNIKLR | 4IBB Antica1in |
| | EDKDPNKMMATIYELKEDKSYNVTGVTFDDKKCTYAISTFVP | |
| | GSQPGEFTLGKIKSFPGHTSSLVRVVSTNYNQHAMVFFKFVFQ | |
| | NREEFYITLYGRTKELTSELKENFIRFSKSLGLPENHIVFPVP | |
| | IDQCIDG | |
| 384 | QDSTSDLIPAPPLSKVPLQQNFQDNQFHGKWYVVGQAGNIRLR | 4IBB Antica1in |
| | EDKDPIKMMATIYELKEDKSYDVTMVKFDDKKCMYDIWTFVP | |
| | GSQPGEFTLGKIKSFPGHTSSLVRVVSTNYNQHAMVFFKFVFQ | |
| | NREEFYITLYGRTKELTSELKENFIRFSKSLGLPENHIVFPVP | |
| | IDQCIDG | |
| 385 | QDSTSDLIPAPPLSKVPLQQNFQDNQFHGKWYVVGQAGNIRLR | 4IBB Antica1in |
| | EDKDPNKMMATIYELKEDKSYDVTAVAFDDKKCTYDIWTFVP | |
| | GSQPGEFTLGKIKSFPGHTSSLVRVVSTNYNQHAMVFFKFVFQ | |
| | NREEFYITLYGRTKELTSELKENFIRFSKSLGLPENHIVFPVP | |
| | IDQCIDG | |
| 386 | QDSTSDLIPAPPLSKVPLQQNFQDNQFHGKWYVVGQAGNIKLR | 4IBB Antica1in |
| | EDKDPNKMMATIYELKEDKSYDVTAVAFDDKKCTYDIWTFVP | |
| | GSQPGEFTLGKIKSFPGHTSSLVRVVSTNYNQHAMVFFKFVFQ | |
| | NREEFYITLYGRTKELTSELKENFIRFSKSLGLPENHIVFPVP | |
| | IDQCIDG | |
| 387 | QDSTSDLIPA PPLSKVPLQQ NFQDNQFHGK WYVVGQAGNI | 4IBB Antica1in |
| | KLREDSKMMA TIYELKEDKS YDVTGVSFDD KKCTYAIMTF | |
| | VPGSQPGEFT LGKIKSFPGH TSSLVRVVST NYNQHAMVFF | |
| | KFVFQNREEF YITLYGRTKE LTSELKENFI RFSKSLGLPE | |
| | NHIVFPVPID QCIDG | |
| 388 | QDSTSDLIPA PPLSKVPLQQ NFQDNQFHGK WYVVGQAGNI | 4IBB Antica1in |
| | KLREDKDPVK MMATIYELKE DKSYDVTGVT FDDKKCRYDI | |
| | STFVPGSQPG EFTFGKIKSF PGHTSSLVRV VSTNYNQHAM | |
| | VFFKFVFQNR EEFYITLYGR TKELTSELKE NFIRFSKSLG | |
| | LPENHIVFPV PIDQCIDG | |
| 389 | QDSTSDLIPAPPLSKVPLQQNFQDNQFHGKWYVVGQAGNIRLR | 4IBB Antica1in |
| | EDKDPHKMMATIYELKEDKSYDVTGVTFDDKKCTYAISTFVP | |
| | GSQPGEFTLGKIKSFPGHTSSLVRVVSTNYNQHAMVFFKFVFQ | |
| | NREEFYITLYGRTKELTSELKENFIRFSKSLGLPENHIVFPVP | |
| | IDQCIDG | |
| 390 | QDSTSDLIPAPPLSKVPLQQNFQDNQFHGKWYVVGQAGNIKLR | 4IBB Antica1in |
| | EDKDPNKMMATIYELKEDKSYDVTGVTFDDKKCTYAISTLVP | |
| | GSQPGEFTFGKIKSFPGHTSSLVRVVSTNYNQHAMVFFKFVFQ | |
| | NREEFYITLYGRTKELTSELKENFIRFSKSLGLPENHIVFPVP | |
| | IDQCIDG | |
| 391 | QDSTSDLIPAPPLSKVPLQQNFQDNQFHGKWYVVGQAGNIRLR | 4IBB Antica1in |
| | EDKDPSKMMATIYELKEDKSYDVTAVTFDDKKCNYAISTFVPG | |
| | SQPGEFTLGKIKSFPGHTSSLVRVVSTNYNQHAMVFFKFVFQN | |
| | REEFYITLYGRTKELTSELKENFIRFSKSLGLPENHIVFPVPI | |
| | DQCIDG | |
| 392 | REGPELSPDDPAGLLDLRQGMFAQLVAQNVLLIDGPLSWYSDP | 71-254 of human |
| | GLAGVSLTGGLSYKEDTKELVVAKAGVYYVFFQLELRRVVAG | 41BBL |
| | EGSGSVSLALHLQPLRSAAGAAALALTVDLPPASSEARNSAFG | |
| | FQGRLLHLSAGQRLGVHLHTEARARHAWQLTQGATVLGLFRV | |
| | TPEIPAGLPSPRSE | |
| 393 | LDLRQGMFAQLVAQNVLLIDGPLSWYSDPGLAGVSLTGGLSY | 85-254 of human |
| | KEDTKELVVAKAGVYYVFFQLELRRVVAGEGSGSVSLALHLQ | 41BBL |
| | PLRSAAGAAALALTVDLPPASSEARNSAFGFQGRLLHLSAGQR | |
| | LGVHLHTEARARHAWQLTQGATVLGLFRVTPEIPAGLPSPRSE | |
| 394 | DPAGLLDLRQGMFAQLVAQNVLLIDGPLSWYSDPGLAGVSLT | 80-254 of human |
| | GGLSYKEDTKELVVAKAGVYYVFFQLELRRVVAGEGSGSVSL | 41BBL |
| | ALHLQPLRSAAGAAALALTVDLPPASSEARNSAFGFQGRLLHL | |
| | SAGQRLGVHLHTEARARHAWQLTQGATVLGLFRVTPEIPAGLP | |
| | SPRSE | |
| 395 | PWAVSGARASPGSAASPRLREGPELSPDDPAGLLDLRQGMFAQ | 52-254 of human |
| | LVAQNVLLIDGPLSWYSDPGLAGVSLTGGLSYKEDTKELVVA | 4-1BBL |
| | KAGVYYVFFQLELRRVVAGEGSGSVSLALHLQPLRSAAGAAA | |
| | LALTVDLPPASSEARNSAFGFQGRLLHLSAGQRLGVHLHTEAR | |
| | ARHAWQLTQGATVLGLFRVTPEIPAGLPSPRSE | |
| 396 | REGPELSPDDPAGLLDLRQGMFAQLVAQNVLLIDGPLSWYSDP | 71-248 of human |
| | GLAGVSLTGGLSYKEDTKELVVAKAGVYYVFFQLELRRVVAG | 41BBL |
| | EGSGSVSLALHLQPLRSAAGAAALALTVDLPPASSEARNSAFG | |
| | FQGRLLHLSAGQRLGVHLHTEARARHAWQLTQGATVLGLFRV | |
| | TPEIPAGL | |
| 397 | LDLRQGMFAQLVAQNVLLIDGPLSWYSDPGLAGVSLTGGLSY | 85-248 of human |
| | KEDTKELVVAKAGVYYVFFQLELRRVVAGEGSGSVSLALHLQ | 41BBL |
| | PLRSAAGAAALALTVDLPPASSEARNSAFGFQGRLLHLSAGQR | |
| | LGVHLHTEARARHAWQLTQGATVLGLFRVTPEIPAGL | |
| 398 | DPAGLLDLRQGMFAQLVAQNVLLIDGPLSWYSDPGLAGVSLT | 80-248 of human |
| | GGLSYKEDTKELVVAKAGVYYVFFQLELRRVVAGEGSGSVSL | 41BBL |
| | ALHLQPLRSAAGAAALALTVDLPPASSEARNSAFGFQGRLLHL | |
| | SAGQRLGVHLHTEARARHAWQLTQGATVLGLFRVTPEIPAGL | |
| 399 | PWAVSGARASPGSAASPRLREGPELSPDDPAGLLDLRQGMFAQ | 52-248 of human |
| | LVAQNVLLIDGPLSWYSDPGLAGVSLTGGLSYKEDTKELVVA | 41BBL |
| | KAGVYYVFFQLELRRVVAGEGSGSVSLALHLQPLRSAAGAAA | |
| | LALTVDLPPASSEARNSAFGFQGRLLHLSAGQRLGVHLHTEAR | |
| | ARHAWQLTQGATVLGLFRVTPEIPAGL | |
| 400 | EVQLLESGGGEVQPGGSLRLSCAASGFSFSINAMGWYRQAPGK | 4IBB sdAb |
| | RREFVAAIESGRNTVYAESVKGRFTISRDNAKNTVYLQMSSLR | |
| | AEDTAVYYCGLLKGNRVVSPSVAYWGQGTLVTVKP | |
| 401 | QVSHRYPRIQSIKVQFTEYKKEKGFILTSQKEDEIMKVQNNSVII | 0X40 1igand |
| | NCDGFYLISLKGYFSQEVNISLHYQKDEEPLFQLKKVRSVNSL | |
| | MVASLTYKDKVYLNVTTDNTSLDDFHVNGGELILIHQNPGEFC | |
| | VL | |
| 402 | QVSHRYPRFQ SIKVQFTEYK KEKGFILTSQ KEDEIMKVQN | 0X40 1igand |
| | NSVIINCDGF YLISLKGYFS QEVNISLHYQ KDEEPLFQLK | |
| | KVRSVNSLMV ASLTYKDKVY LNVTTDNTSL DDFHVNGGEL | |
| | ILIHQNPGEFCVL | |
| 403 | QVSHRYPRIQSIKVQFTEYKKEKGFILTSQKEDEIMKVQNNSVII | 0X40 1igand |
| | NCDGFYLISLKGYFSQEVNISLHYQKDEEPLFQLKKVRSVNSL | |
| | MVASLTYKDKVYLNVTTDNTSLDDFHVNGGELILIHQNPGEFC | |
| | VL | |
| 404 | QVSHRYPRIQ SIKVQFTEYK KEKGFILTSQ KEDEIMKVQN | 0X40 1igand |
| | NSVIINCDGF YLISLKGYFS QEVNISLHYQ KDEEPLFQLK | |
| | KVRSVNSLMV ASLTYKDKVY LNVTTDNTSL DDFHVNGGEL | |
| | ILIHQNPGEF CVL | |
| 405 | VSHRYPRIQSIKVQFTEYKKEKGFILTSQKEDEIMKVQNNSVIIN | 0X40 1igand |
| | CDGFYLISLKGYFSQEVNISLHYQKDEEPLFQLKKVRSVNSLM | |
| | VASLTYKDKVYLNVTTDNTSLDDFHVNGGELILIHQNPGEFCV | |
| | L | |
| 406 | EVQLVQSGAEVKKPGASVKVSCKASGYTFTDSYMSWVRQAP | 0X40 VH |
| | GQGLEWIGDMYPDNGDSSYNQKFRERVTITRDTSTSTAYLELS | |
| | SLRSEDTAVYYCVLAPRWYFSVWGQGTLVTVSS | |
| 407 | DIQMTQSPSSLSASVGDRVTITCRASQDISNYLNWYQQKPGKA | 0X40 VL |
| | PKLLIYYTSRLRSGVPSRFSGSGSGTDFTLTISSLQPEDFATYYC | |
| | QQGHTLPPTFGQGTKVEIKRT | |
| 408 | EVQLVESGGGLVQPGGSLKLSCAASGFTFSGSAMHWVRQASG | 0X40 VH |
| | KGLEWVGRIRSKANSYATAYAASVKGRFTISRDDSKNTAYLQ | |
| | MNSLKTEDTAVYYCTSGIYDSSGYDYWGQGTLVTVSS | |
| 409 | DIVMTQSPLSLPVTPGEPASISCRSSQSLLHSNGYNYLDWYLQK | 0X40 VL |
| | PGQSPQLLIYLGSNRASGVPDRFSGSGSGTDFTLKISRVEAEDV | |
| | GVYYCMQALQTPLTFGGGTKVEIK | |
| 410 | EVQLLESGGGEVQPGGSLRLSCAASGFTFSDAFMYWVRQAPG | 0X40 sdAb |
| | KGLEWVSSISNRGLKTAYAESVKGRFTISRDNAKNTLYLQMSS | |
| | LRAEDTAVYYCSRDVDGDFRGQGTLVTVKP | |
| 411 | QLETAKEPCMAKFGPLPSKWQMASSEPPCVNKVSDWKLEILQ | GITR 1igand |
| | NGLYLIYGQVAPNANYNDVAPFEVRLYKNKDMIQTLTNKSKI | |
| | QNVGGTYELHVGDTIDLIFNSEHQVLKNNTYWGIILLANPQFIS | |
| 412 | QVQLVESGGGVVQPGRSLRLSCAASGFTFSSYAMSWVRQAPG | GITR VH |
| | KGLEWVASISSGGTTYYPDSVKGRFTISRDNSKNTLYLQMNSL | |
| | RAEDTAVYYCARVGGYYDSMDYWGQGTLVTVSS | |
| 413 | EIVLTQSPGTLSLSPGERATLSCRASESVDNYGVSFMNWYQQK | GITR VL |
| | PGQAPRLLIYAASNQGSGIPDRFSGSGSGTDFTLTISRLEPEDFA | |
| | VYYCQQTKEVTWTFGQGTKVEIK | |
| 414 | QVTLRESGPALVKPTQTLTLTCTFSGFSLSTSGMGVGWIRQPPG | GITR VH |
| | KALEWLAHIWWDDDKYYQPSLKSRLTISKDTSKNQVVLTMTN | |
| | MDPVDTATYYCARTRRYFPFAYWGQGTLVTVSS | |
| 415 | EIVMTQSPATLSVSPGERATLSCKASQNVGTNVAWYQQKPGQ | GITR VL |
| | APRLLIYSASYRYSGIPARFSGSGSGTEFTLTISSLQSEDFAVYYC | |
| | QQYNTDPLTFGGGTKVEIK | |
| 416 | EVQLLESGGGEVQPGGSLRLSCAASGSVFSIDAMGWYRQAPG | GITR sdAb |
| | KQRELVAVLSGISSAKYAASAPGRFTISRDNAKNTVYLQMSSL | |
| | RAEDTAVYYCYADVSTGWGRDAHGYWGQGTLVTV | |
| 417 | QAVVTQEPSLTVSPGGTVTLTCGSSTGAVTTSNYANWVQQKP | anti-CD3 VL |
| | GQAFRGLIGGTNKRAPGVPARFSGSLLGGKAALTISGAQPEDE | (CON) |
| | ADYYCALWYSNHWVFGCGTKLTVL | |
| 418 | DIQLTQSPSF LSASVGDRVT ITCKASQNVD TNVAWYQQKP | First Po1ypeptide |
| | GKAPKALIYSASYRYSGVPS RFSGSGSGTD FTLTISSLQP | Chain ofB7-H3x |
| | EDFATYYCQQ YNNYPFTFGQGTKLEIKGGG SGGGGEVQLV | CD3 Bispecific |
| | ESGGGLVQPG GSLRLSCAAS GFTFSTYAMNWVRQAPGKGL | DART-A Diabody |
| | EWVGRIRSKY NNYATYYADS VKDRFTISRD DSKNSLYLQM | |
| | NSLKTEDTAV YYCVRHGNFG NSYVSWFAYW GQGTLVTVSS | |
| | GGCGGGEVAA LEKEVAALEK EVAALEKEVA ALEKGGGDKT | |
| | HTCPPCPAPE AAGGPSVFLFPPKPKDTLMI SRTPEVTCVV | |
| | VDVSHEDPEV KFNWYVDGVE VHNAKTKPRE EQYNSTYRVV | |
| | SVLTVLHQDW LNGKEYKCKV SNKALPAPIE | |
| | KTISKAKGQPREPQVYTLPP SREEMTKNQV SLWCLVKGFY | |
| | PSDIAVEWES NGQPENNYKTTPPVLDSDGS FFLYSKLTVD | |
| | KSRWQQGNVF SCSVMHEALH NHYTQKSLSLSPGK | |
| 419 | QAVVTQEPSLTVSPGGTVTLTCRSSTGAVTTSNYANWVQQKP | Second |
| | GQAPRGLIGGTNKRAPWTPARFSGSLLGGKAALTITGAQAEDE | Po1ypeptide Chain |
| | ADYYCALWYSNLWVFGGGTKLTVLGGGGSGGGGEVQLVESG | ofB7-H3xCD3 |
| | GGLVQPGGSLRLSCAASGFTFSSFGMHWVRQAPGKGLEWVAY | Bispecific DART- |
| | ISSDSSAIYYADTVKGRFTISRDNAKNSLYLQMNSLRDEDTAVY | A Diabody |
| | YCGRGRENIYYGSRLDYWGQGTTVTVSSGGCGGGKVAALKE | |
| | KVAALKEKVAALKEKVAALKE | |
| 420 | DKTHTCPPCPAPEAAGGPSVFLFPPKPKDTLMISRTPEVTCVVV | Third Po1ypeptide |
| | DVSHEDPEVKFNWYVDGVEVHNAKTKPREEQYNSTYRVVSV | Chain ofB7-H3x |
| | LTVLHQDWLNGKEYKCKVSNKALPAPIEKTISKAKGQPREPQV | CD3 Bispecific |
| | YTLPPSREEMTKNQVSLSCAVKGFYPSDIAVEWESNGQPENNY | DART-A Diabody |
| | KTTPPVLDSDGSFFLVSKLTVDKSRWQQGNVFSCSVMHEALH | |
| | NRYTQKSLSLSPGK | |
| 421 | EVQLVESGGGEVQPGGSLRLSCAASGSMTGANTMGWYRQAP | hz18H10v1 |
| | GKGRDLVSLIGNYVTHYAESVKGRFTISRDNAKNTLYLQMSSL | |
| | RAEDTAVYYCYLYTDNLGTSWGQGTLVTVKPGG | |
| 422 | EVQLVESGGGEVQPGGSLRLSCAASGSMTGANTMGWYRQAP | hz18H10v2 |
| | GKQRDLVSLIGNYVTHYAESVKGRFTISRDNAKNTLYLQMSSL | |
| | RAEDTAVYYCYLYTDNLGTSWGQGTLVTVKPGG | |
| 423 | EVQLVESGGGEVQPGGSLRLSCAASGSMTGANTMGWYRQAP | hz18H10v3 |
| | GKQRDLVSLIGNYVTHYAESVKGRFTISRDNAKNTVYLQMSSL | |
| | RAEDTAVYYCYLYTDNLGTSWGQGTLVTVKPGG | |
| 424 | EVQLVESGGGEVQPGGSLRLSCAASGSMTGANTMGWYRQAP | hz18H10v4 |
| | GKQRDLVALIGNYVTHYAESVKGRFTISRDNAKNTVYLQMSS | |
| | LRAEDTAVYYCYLYTDNLGTSWGQGTLVTVKPGG | |
| 425 | EVQLVESGGGEVQPGGSLRLSCAASGSMTGANTMGWYRQAP | hz18H10v5 |
| | GKQRDLVALIGNYVTHYAESVKGRFTISRENAKNTVYLQMSSL | |
| | RAEDTAVYYCYLYTDNLGTSWGQGTLVTVKPGG | |
| 426 | EVQLVESGGGEVQPGGSLRLSCAASGSVTGANTMGWYRQAPG | hz18H10v6 |
| | KQRDLVALIGNYVTHYAESVKGRFTISRDNAKNTVYLQMSSLR | |
| | AEDTAVYYCYLYTDNLGTSWGQGTLVTVKPGG | |
| 427 | EVQLVESGGGEVQPGGSLRLSCAASGSITGANTMGWYRQAPG | hz18H10v7 |
| | KQRDLVALIGNYVTHYAESVKGRFTISRDNAKNTVYLQMSSLR | |
| | AEDTAVYYCYLYTDNLGTSWGQGTLVTVKPGG | |
| 428 | EVQLVESGGGEVQPGGSLRLSCAASGSVTGANTMGWYRQAPG | hz18H10v8 |
| | KQRDLVSLIGNYVTHYAESVKGRFTISRDNAKNTLYLQMSSLR | |
| | AEDTAVYYCYLYTDNLGTSWGQGTLVTVKPGG | |
| 429 | EVQLVESGGGEVQPGGSLRLSCAASGSVTGANTMGWYRQAPG | hz18H10v9 |
| | KQRDLVSLIGNYVTHYAESVKGRFTISRDNAKNTVYLQMSSLR | |
| | AEDTAVYYCYLYTDNLGTSWGQGTLVTVKPGG | |
| 430 | EVQLVESGGGEVQPGGSLRLSCAASGSMTGANTMGWYRQAP | hz18H10v10 |
| | GKQRDLVSLIGNYVTHYAESVKGRFTISRENAKNTVYLQMSSL | |
| | RAEDTAVYYCYLYTDNLGTSWGQGTLVTVKPGG | |
| 431 | EVQLVESGGGEVQPGGSLRLSCAASGSVTGANTMGWYRQAPG | hz18H10v11 |
| | KQRDLVSLIGNYVTHYAESVKGRFTISRENAKNTVYLQMSSLR | |
| | AEDTAVYYCYLYTDNLGTSWGQGTLVTVKPGG | |
| 432 | EVQLVESGGGEVQPGGSLRLSCAASGSVTGANTMGWYRQAPG | hz18H10v12 |
| | KQRDLVALIGNYVTHYAESVKGRFTISRENAKNTVYLQMSSLR | |
| | AEDTAVYYCYLYTDNLGTSWGQGTLVTVKPGG | |
| 433 | EVQLVESGGGEVQPGGSLRLSCAASGSMTGANTMGWYRQAP | hz18H10v13 |
| | GKQRELVALIGNYVTHYAESVKGRFTISRENAKNTVYLQMSSL | |
| | RAEDTAVYYCYLYTDNLGTSWGQGTLVTVKPGG | |
| 434 | EVQLVESGGGEVQPGGSLRLSCAASGSVTGANTMGWYRQAPG | hz18H10v14 |
| | KQRDLVALIGNYVTHYAESVKGRFTISRDNAKNTLYLQMSSLR | |
| | AEDTAVYYCYLYTDNLGTSWGQGTLVTVKPGG | |
| 435 | EVQLVESGGGEVQPGGSLRLSCAASGSITGANTMGWYRQAPG | hz18H10v15 |
| | KQRDLVALIGNYVTHYAESVKGRFTISRDNAKNTLYLQMSSLR | |
| | AEDTAVYYCYLYTDNLGTSWGQGTLVTVKPGG | |
| 436 | EVQLVESGGGEVQPGGSLRLSCAASGSITGANTMGWYRQAPG | hz18H10v16 |
| | KQRDLVALIGNYVTHYAESVKGRFTISRENAKNTVYLQMSSLR | |
| | AEDTAVYYCYLYTDNLGTSWGQGTLVTVKPGG | |
| 437 | EVQLVESGGGEVQPGGSLRLSCAASGSITGANTMGWYRQAPG | hz18H10v17 |
| | KQRDLVALIGNYVTHYAESVKGRFTISRDNAKNTVYLQMNSL | |
| | RAEDTAVYYCYLYTDNLGTSWGQGTLVTVKPGG | |
| 438 | GSMTGANTMG | CDRH1 |
| 439 | GSVTGANTMG | CDRH1 |
| 440 | GSITGANTMG | CDRH1 |
| 441 | LIGNYVTH | CDRH2 |
| 442 | YTDNLGTS | CDRH3 |
| 443 | QVQLVQSGGGLVQPGGSLRLSCVASGSMTGANTMGWYRQAP | 18H10 |
| | GKQRDLVALIGNYHYADSVKGRFTISRENAKNTVILQMNSLNP | |
| | EDTAVYYCYLYTDNLGTSWGQGTLVTVKPGG | |
| 444 | PGGGG | linker |
| 445 | DKTHTCPPCPAPELLGGPSVFLFPPKPKDTLMISRTPEVTCVVV | Knob Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPCRDELTKNQVSLWCLVKGFYPSDIAVEWESNGQPENNY | |
| | KTTPPVLDSDGSFFLYSKLTVDKSRWQQGNVFSCSVMHEALH | |
| | NHYTQKSLSLSP | |
| 446 | DKTHTCPPCPAPELLGGPSVFLFPPKPKDTLMRSRTPEVTCVVV | Hole Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | CTLPPSRDELTKNQVSLSCAVKGFYPSDIAVEWESNGQPENNY | |
| | KTTPPVLDSDGSFFLVSKLTVDKSRWQQGNVFSCSVMHEALH | |
| | NHYTQKSLSLSP | |
| 447 | DKTHTCPPCPAPGGPSVFLFPPKPKDTLMISRTPEVTCVVVDVS | Knob Fc |
| | HEDPEVKFNWYVDGVEVHNAKTKPREEQYNSTYRVVSVLTVL | |
| | HQDWLNGKEYKCKVSNKALPAPIEKTISKAKGQPREPQVYTLP | |
| | PCRDELTKNQVSLWCLVKGFYPSDIAVEWESNGQPENNYKTTP | |
| | PVLDSDGSFFLYSKLTVDKSRWQQGNVFSCSVMHEALHNHYT | |
| | QKSLSLSP | |
| 448 | DKTHTCPPCPAPGGPSVFLFPPKPKDTLMRSRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVMHEALHNHYT | |
| | QKSLSLSP | |
| 449 | DKTHTCPPCPAPELLGGPSVFLFPPKPKDTLMISRTPEVTCVVV | Hole Fc |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | CTLPPSRDELTKNQVSLSCAVKGFYPSDIAVEWESNGQPENNY | |
| | KTTPPVLDSDGSFFLVSKLTVDKSRWQQGNVFSCSVMHEALH | |
| | NRYTQKSLSLSP | |
| 450 | DKTHTCPPCPAPGGPSVFLFPPKPKDTLMISRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVMHEALHNRYT | |
| | QKSLSLSP | |
| 451 | DKTHTCPPCPAPELLGGPSVFLFPPKPKDTLYISRTPEVTCVVVD | Knob Fc |
| | VSHEDPEVKFNWYVDGVEVHNAKTKPREEQYNSTYRVVSVLT | |
| | VLHQDWLNGKEYKCKVSNKALPAPIEKTISKAKGQPREPQVYT | |
| | LPPCRDELTKNQVSLWCLVKGFYPSDIAVEWESNGQPENNYKT | |
| | TPPVLDSDGSFFLYSKLTVDKSRWQQGNVFSCSVVHEALHNH | |
| | YTQKSLSLSP | |
| 452 | DKTHTCPPCPAPGGPSVFLFPPKPKDTLYISRTPEVTCVVVDVS | Knob Fc |
| | HEDPEVKFNWYVDGVEVHNAKTKPREEQYNSTYRVVSVLTVL | |
| | HQDWLNGKEYKCKVSNKALPAPIEKTISKAKGQPREPQVYTLP | |
| | PCRDELTKNQVSLWCLVKGFYPSDIAVEWESNGQPENNYKTTP | |
| | PVLDSDGSFFLYSKLTVDKSRWQQGNVFSCSVVHEALHNHYT | |
| | QKSLSLSP | |
| 453 | DKTHTCPPCPAPELLGGPSVFLFPPKPKDTLYISRTPEVTCVVVD | Hole Fc |
| | VSHEDPEVKFNWYVDGVEVHNAKTKPREEQYNSTYRVVSVLT | |
| | VLHQDWLNGKEYKCKVSNKALPAPIEKTISKAKGQPREPQVCT | |
| | LPPSRDELTKNQVSLSCAVKGFYPSDIAVEWESNGQPENNYKT | |
| | TPPVLDSDGSFFLVSKLTVDKSRWQQGNVFSCSVVHEALHNRY | |
| | TQKSLSLSP | |
| 454 | DKTHTCPPCPAPGGPSVFLFPPKPKDTLYISRTPEVTCVVVDVS | Hole Fc |
| | HEDPEVKFNWYVDGVEVHNAKTKPREEQYNSTYRVVSVLTVL | |
| | HQDWLNGKEYKCKVSNKALPAPIEKTISKAKGQPREPQVCTLP | |
| | PSRDELTKNQVSLSCAVKGFYPSDIAVEWESNGQPENNYKTTP | |
| | PVLDSDGSFFLVSKLTVDKSRWQQGNVFSCSVVHEALHNRYT | |
| | QKSLSLSP | |
| 455 | EVQLVESGGGLVQPGGSLRLSCAASGFTFSSFGMHWVRQAPG | B7H3-LC |
| | KGLEWVAYISSDSSAIYYADTVKGRFTISRDNAKNSLYLQMNS | |
| | LRDEDTAVYYCGRGRENIYYGSRLDYWGQGTTVTVSSASTKG | |
| | PSVFPLAPSSKSTSGGTAALGCLVKDYFPEPVTVSWNSGALTSG | |
| | VHTFPAVLQSSGLYSLSSVVTVPSSSLGTQTYICNVNHKPSNTK | |
| | VDKKVEPKSC | |
| 456 | DIQLTQSPSFLSASVGDRVTITCKASQNVDTNVAWYQQKPGKA | B7H3-Fd |
| | PKALIYSASYRYSGVPSRFSGSGSGTDFTLTISSLQPEDFATYYC | |
| | QQYNNYPFTFGQGTKLEIKRTVAAPSVFIFPPSDEQLKSGTASV | |
| | VCLLNNFYPREAKVQWKVDNALQSGNSQESVTEQDSKDSTYS | |
| | LSSTLTLSKADYEKHKVYACEVTHQGLSSPVTKSFNRGEC | |
| 457 | DKTHTCPPCPAPPVAGPSVFLFPPKPKDTLMISRTPEVTCVVVD | Fc-Het-1 |
| | VKHEDPEVKFNWYVDGVEVHNAKTKPREEEYNSTYRVVSVLT | |
| | VLHQDWLNGKEYKCKVSNKALPAPIEKTISKAKGQPREPQVYT | |
| | LPPSREEMTKNQVSLTCDVSGFYPSDIAVEWESDGQPENNYKT | |
| | TPPVLDSDGSFFLYSKLTVDKSRWEQGDVFSCSVMHEALHNH | |
| | YTQKSLSLSPGK | |
| 458 | DKTHTCPPCPAPPVAGPSVFLFPPKPKDTLMISRTPEVTCVVVD | Fc-Het-2 |
| | VKHEDPEVKFNWYVDGVEVHNAKTKPREEQYNSTYRVVSVL | |
| | TVLHQDWLNGKEYKCKVSNKALPAPIEKTISKAKGQPREPQVY | |
| | TLPPSREQMTKNQVKLTCLVKGFYPSDIAVEWESNGQPENNYK | |
| | TTPPVLDSDGSFFLYSKLTVDKSRWQQGNVFSCSVMHEALHN | |
| | HYTQKSLSLSPGK | |
| 459 | QAVVTQEPSLTVSPGGTVTLTCGSSTGAVTTSNYANWVQQKP | VL21 |
| | GKSPRGLIGGTNKRAPGVPARFSGSLLGGKAALTISGAQPEDEA | |
| | DYYCALWYSNHWVFGCGTKLTVL | |
| 460 | EVQLVESGGGLVQPGGSLRLSCAASGFTFSTYAMNWVRQAPG | VH32 |
| | KGLEWVGRIRSKYNNYATYYADSVKGRFTISRDDSKNTLYLQ | |
| | MNSLRAEDTAVYYCVRHGNFGDSYVSWFAYWGQGTLVTVSS | |
| 461 | QAVVTQEPSLTVSPGGTVTLTCGSSTGAVTTSNYANWVQQKP | VL20 |
| | GKSPRGLIGGTNKRAPGVPARFSGSLLGGKAALTISGAQPEDEA | |
| | DYYCALWYSNHWVFGGGTKLTVL | |
| 462 | EVQLVESGGGLVQPGGSLRLSCAASGFTFSTYAMNWVRQAPG | VH34 |
| | KCLEWVARIRSKYNNYATYYADTVKGRFTISRDDAKNTLYLQ | |
| | MSSLRAEDTAVYYCVRHGNFGDSYVSWFAYWGQGTLVTV | |
| 463 | DKTHTCPPCPAPELLGGPSVFLFPPKPKDTLMISRTPEVTCVVV | IgG1 Knob |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | YTLPPCRDELTKNQVSLWCLVKGFYPSDIAVEWESNGQPENNY | |
| | KTTPPVLDSDGSFFLYSKLTVDKSRWQQGNVFSCSVMHEALH | |
| | NHYTQKSLSLSP | |
| 464 | DKTHTCPPCPAPELLGGPSVFLFPPKPKDTLMRSRTPEVTCVVV | IgG1 Knob |
| | DVSHEDPEVKFNWYVDGVEVHNAKTKPREEQYNSTYRVVSV | |
| | LTVLHQDWLNGKEYKCKVSNKALPAPIEKTISKAKGQPREPQV | |
| | CTLPPSRDELTKNQVSLSCAVKGFYPSDIAVEWESNGQPENNY | |
| | KTTPPVLDSDGSFFLVSKLTVDKSRWQQGNVFSCSVMHEALH | |
| | NHYTQKSLSLSP | |
| 465 | GGGGSGGGGSGGGGS | linker |
| 466 | EVQLVESGGGEVQPGGSLRLSCAASGFTFSSYHMSWFRQAPGK | hz58E05v55 |
| | QRELVATSHHGGTTNYAGSVKGRFTISRDNAKNTVYLQMNTL | |
| | RAEDTAVYYCKADHGYQGRGYWGQGTLVTVKP | |
| 467 | EVQLVESGGGEVQPGGSLRLSCAASGFSFSSNVMMWVRQAPG | hz1A5v51 |
| | KGLEWVSTIYSSGTGTFYAESVKGRFTISRDNAKNTLYLQMSS | |
| | LRPEDTAVYYCATSGPVRGWGPRSQGTLVTVKP | |
| 468 | GFSFSINAMG | 41BB CDR1 |
| 469 | AIESGRNTV | 4IBB CDR2 |
| 470 | LKGNRVVSPSVAY | 4IBB CDR3 |
| 471 | GFTFNTYAMN | anti-CD3 VH |
| | | CDR1 |
| 472 | RIRSKYNNYATY | anti-CD3 VH |
| | | CDR2 |
| 473 | HGNFGDSYVSWFAY | CD3-VH7, VH33 |
| | | CDR3 |
| 474 | ALWYSNHWV | CD3-VL2, VL21 |
| | | CDR3 |
| 475 | VLWYSNRWV | CD3-VL8 CDR3 |
| 476 | GFTFSTYAMN | CD3 VH33 CDR1 |
| 477 | RIRSKYNNYATY | CD3 VH33 CDR1 |
| 478 | GSSTGAVTTSNYAN | CD3 VL21 CDR1 |
| 479 | GTNKRAP | CD3 VL21 CDR2 |
| 480 | EVQLVESGGGLVQPGGSLRLSCAASGFTFSTYAMNWVRQAPG | CD3-VH33 |
| | KCLEWVGRIRSKYNNYATYYADSVKGRFTISRDDSKNTLYLQ | |
| | MNSLRAEDTAVYYCVRHGNFGDSYVSWFAYWGQGTLVTVSS | |
| 481 | EVQLVESGGGEVQPGGSLRLSCAASGFSFSINAMGWYRQAPG | 4IBB sdAb |
| | KRREFVAAIESGRNTVYAESVKGRFTISRDNAKNTVYLQMSSL | |
| | RAEDTAVYYCGLLKGNRVVSPSVAYWGQGTLVTVKP | |
| 482 | IEPDP | Linker |
| 483 | RWGSPDYYHDDVDY | CDRH3 (57B10) |
| 484 | GEPIRVGEKSGYDY | CDRH3 (58B06) |
| 485 | GPRDYFSDLEVDFGS | CDRH3 (57B08) |
| 486 | GLPIRVGVPGGYDY | CDRH3 (58A08) |
| 487 | ASFAYLSTYTHHYDY | CDRH3 (57D1) |
| 488 | SRSAISRKATDFGS | CDRH3 (1H5, |
| | | hz1H5 v1-v4) |
| 489 | EVQLVESGGGEVQPGGSLRLSCAASGFSFSSNVMMWVRQAPG | hz1A5v52 |
| | KGLEWVSTIYSSGTGTYYAESVKGRFTISRDNAKNTLYLQMSS | |
| | LRPEDTAVYYCATSGPVRGWGPRSQGTLVTVKP | |
| 490 | EVQLVESGGGEVQPGGSLRLSCAASGFSFGSNVMMWVRQAPG | hz1A5v53 |
| | KGLEWVSTIYSSGTGTFYAESVKGRFTISRDNAKNTLYLQMSS | |
| | LRPEDTAVYYCATSGPVRGWGPRSQGTLVTVKP | |
| 491 | GGS(GGS)n, where n = 0 to 10 | linker |
| 492 | EVQLVESGGGEVQPGGSLRLSCAASGFSFGSNVMMWVRQAPG | hz1A5v1 |
| | KGPEWVSTIYSSGTGTFYAESVKGRFTISRDNAKNTLYLQMSSL | |
| | RAEDTAVYYCATSGPVRGWGPRSQGTLVTVKP | |
| 493 | EVQLVESGGGEVQPGGSLRLSCAASGFSFGSNVMMWVRQAPG | hz1A5v2 |
| | KGPEWVSTIYSSGTGTFYAESVKGRFTISRNNAKNTLYLQMSSL | |
| | RAEDTAVYYCATSGPVRGWGPRSQGTLVTVKP | |
| 494 | EVQLVESGGGEVQPGGSLRLSCAASGFSFGSNVMMWVRQAPG | hz1A5v3 |
| | KGPEWVSTIYSSGTGTFYAESVKGRFTISRNNAKNTLYLQMSSL | |
| | RAEDTAVYYCATSGPVRGWGPRGQGTLVTVKP | |
| 495 | EVQLVESGGGEVQPGGSLRLSCAASGFSFGSNVMMWVRQAPG | hz1A5v7 |
| | KGPEWVSTIYSSGTGTFYAESVKGRFTISRNNAKNTLYLQMSSL | |
| | KPDDTAVYYCATSGPVRGWGPRSQGTLVTVKP | |
| 496 | EVQLVESGGGLVQAGDSLRLSCAASGFSFGSNVMMWVRQAP | hz1A5v8 |
| | GKGPEWVSTIYSSGTGTFYAESVKGRFTISRNNAKNTLYLQMS | |
| | SLRAEDTAVYYCATSGPVRGWGPRSQGTLVTVKP | |
| 497 | EVQLVESGGGEVQPGGSLRLSCAASGFSFGSNVMMWVRQAPG | hz1A5v9 |
| | KGPEWVSTIYSRGTGTFYAESVKGRFTISRNNAKNTLYLQMSS | |
| | LRAEDTAVYYCATSGPVRGWGPRSQGTLVTVKP | |
| 498 | EVQLVESGGGEVQPGGSLRLSCAASERTFSTYTMGWFRQAPG | hz57B04v3 |
| | KEREFVSVVNWSGGSKYYAESVKGRFTISRDNAKNTVYLQMN | |
| | SLRAEDTAVYYCAAGGAYSGPYYDTRQYTYWGQGTLVTVKP | |
| 499 | EVQLVESGGGEVQPGGSLRLSCAASERTFSTYTMGWFRQAPG | hz57B04v4 |
| | KEREFVAVVNWSGGSKYYAESVKGRFTISRDNAKNTVYLQMN | |
| | SLRAEDTAVYYCAAGGAYSGPYYDTRQYTYWGQGTLVTVKP | |
| 500 | EVQLVESGGGEVQPGGSLRLSCAPSERTFSTYTMGWFRQAPGK | hz57B04v8 |
| | EREFVAVVNWSGGSKYYAESVKGRFTISRDNAKNTVYLQMNS | |
| | LRAEDTAVYYCAAGGAYSGPYYDTRQYTYWGQGTLVTVKP | |
| 501 | EVQLVESGGGEVQPGGSLRLSCAASERTFSTYTMGWFRQAPG | hz57B04v9 |
| | KEREFVAVVNWSGGSKYYAESVKGRFTISRDNAKNTVYLHMN | |
| | SLRAEDTAVYYCAAGGAYSGPYYDTRQYTYWGQGTLVTVKP | |
| 502 | EVQLVESGGGEVQPGGSLRLSCAPSERTFSTYTMGWFRQAPGK | hz57B04v23 |
| | EREFVAVVNWSGGSKYYAESVKGRFTISRDNAKNTVYLQMSS | |
| | LRAEDTAVYYCAAGGAYSGPYYDTRQYTYWGQGTLVTVKP | |
| 503 | EVQLVESGGGEVQPGGSLRLSCAPSERTFSTYTMGWFRQAPGK | hz57B04v24 |
| | EREFVAVVNWGGGSKYYAESVKGRFTISRDNAKNTVYLQMSS | |
| | LRAEDTAVYYCAAGGAYSGPYYDTRQYTYWGQGTLVTVKP | |
| 504 | EVQLVESGGGEVQPGGSLRLSCAASGSTFSMYHMSWFRQAPG | hz58E05v1 |
| | KGREPVSTSHHGGTTNYAESVKGRFTISRDNAKNTLYLQMNSL | |
| | RAEDTAVYYCKADHGYNGRGYWGQGTLVTVKP | |
| 505 | EVQLVESGGGEVQPGGSLRLSCAASGSTFSMYHMSWFRQAPG | hz58E05v2 |
| | KQREPVSTSHHGGTTNYAESVKGRFTISRDNAKNTLYLQMNSL | |
| | RAEDTAVYYCKADHGYNGRGYWGQGTLVTVKP | |
| 506 | EVQLVESGGGEVQPGGSLRLSCAASGSTFSMYHMSWFRQAPG | hz58E05v3 |
| | KQREPVSTSHHGGTTNYAESVKGRFTISRDNAKNTVYLQMNSL | |
| | RAEDTAVYYCKADHGYNGRGYWGQGTLVTVKP | |
| 507 | EVQLVESGGGEVQPGGSLRLSCAASGSTFSMYHMSWFRQAPG | hz58E05v4 |
| | KQREPVATSHHGGTTNYAESVKGRFTISRDNAKNTVYLQMNS | |
| | LRAEDTAVYYCKADHGYNGRGYWGQGTLVTVKP | |
| 508 | EVQLVESGGGEVQPGGSLRLSCAASGSTFSMYHMSWFRQAPG | hz58E05v5 |
| | KQREPVATSHHGGTTNYAESVKGRFTISRDNAKNTVYLQMNS | |
| | LRAEDTAVYYCKADHGYQGRGYWGQGTLVTVKP | |
| 509 | EVQLVESGGGEVQPGGSLRLSCAASGSTFSMYHMSWFRQAPG | hz58E05v6 |
| | KQREPVATSHHGGTTNYAESVKGRFTISRDNAKNTVYLQMNS | |
| | LRAEDTAVYYCKADHGYNARGYWGQGTLVTVKP | |
| 510 | EVQLVESGGGEVQPGGSLRLSCAASGSTFSMYHMSWFRQAPG | hz58E05v7 |
| | KQREPVATSHHGGTTNYAESVKGRFTISRDNAKNTVYLQMNS | |
| | LRAEDTAVYYCKADHGYNTRGYWGQGTLVTVKP | |
| 511 | EVQLVESGGGEVQPGGSLRLSCAASGSTFSMYHMSWFRQAPG | hz58E05v8 |
| | KQREPVATSHHGGTTNYAGSVKGRFTISRDNAKNTVYLQMNS | |
| | LRAEDTAVYYCKADHGYNGRGYWGQGTLVTVKP | |
| 512 | EVQLVESGGGEVQPGGSLRLSCAASGSTFSMYHMSWFRQAPG | hz58E05v9 |
| | KQREPVATSHHGGTTNYAESVKGRFTISRDNAKNTLYLQMNS | |
| | LRAEDTAVYYCKADHGYQGRGYWGQGTLVTVKP | |
| 513 | EVQLVESGGGEVQPGGSLRLSCAASGSTFSMYHMSWFRQAPG | hz58E05v10 |
| | KQREPVATSHHGGTTNYAESVKGRFTISRDNAKNTLYLQMNS | |
| | LRAEDTAVYYCKADHGYNARGYWGQGTLVTVKP | |
| 514 | EVQLVESGGGEVQPGGSLRLSCAASGSTFSMYHMSWFRQAPG | hz58E05v11 |
| | KQREPVATSHHGGTTNYAESVKGRFTISRDNAKNTLYLQMNS | |
| | LRAEDTAVYYCKADHGYNTRGYWGQGTLVTVKP | |
| 515 | EVQLVESGGGEVQPGGSLRLSCAASGGTFSSYAMGWFRQAPG | h57B06v5 |
| | KEREFVAAISSEGGSTYYADNMEGRFTISRDNAKNTVYLQMSS | |
| | LRAEDTAVYYCAVKGVGWPQEQASYDYWGQGTLVTVKP | |
| 516 | EVQLVESGGGEVQPGGSLRLSCAASGGTFSSYAMGWFRQAPG | h57B06v6 |
| | KEREFVAAISSEGGSTYYAESMEGRFTISRDNAKNTVYLQMSS | |
| | LRAEDTAVYYCAVKGVGWPQEQASYDYWGQGTLVTVKP | |
| 517 | QVQLVQDGDSLRLSCAASGGTFSSYAMGWFRQAPGKEREFVA | h57B06v7 |
| | AISSEGGSTYYADNMEGRFTISRDNAKNTVYLQMSSLRAEDTA | |
| | VYYCAVKGVGWPQEQASYDYWGQGTLVTVKP | |
| 518 | EVQLVESGGGEVQPGGSLRLSCAASGGTFSSYAMGWFRQAPG | h57B06v8 |
| | QEREFVAAISSEGGSTYYAESVKGRFTISRDNAKNTVYLQMSSL | |
| | RAEDTAVYYCAVKGVGWPQEQASYDYWGQGTLVTVKP | |
| 519 | QVQLVQSGGGLVQPGGSLRLSCVASGSMTGANTMGWYRQAP | 18H10 |
| | GKQRDLVALIGNYHYADSVKGRFTISRENAKNTVILQMNSLNP | |
| | EDTAVYYCYLYTDNLGTSWGQGTLVTVKP | |
| 520 | EVQLVESGGGEVQPGGSLRLSCAASGSMTGANTMGWYRQAP | hz18H10v1 |
| | GKGRDLVSLIGNYVTHYAESVKGRFTISRDNAKNTLYLQMSSL | |
| | RAEDTAVYYCYLYTDNLGTSWGQGTLVTVKP | |
| 521 | EVQLVESGGGEVQPGGSLRLSCAASGSMTGANTMGWYRQAP | hz18H10v2 |
| | GKQRDLVSLIGNYVTHYAESVKGRFTISRDNAKNTLYLQMSSL | |
| | RAEDTAVYYCYLYTDNLGTSWGQGTLVTVKP | |
| 522 | EVQLVESGGGEVQPGGSLRLSCAASGSMTGANTMGWYRQAP | hz18H10v3 |
| | GKQRDLVSLIGNYVTHYAESVKGRFTISRDNAKNTVYLQMSSL | |
| | RAEDTAVYYCYLYTDNLGTSWGQGTLVTVKP | |
| 523 | EVQLVESGGGEVQPGGSLRLSCAASGSMTGANTMGWYRQAP | hz18H10v4 |
| | GKQRDLVALIGNYVTHYAESVKGRFTISRDNAKNTVYLQMSS | |
| | LRAEDTAVYYCYLYTDNLGTSWGQGTLVTVKP | |
| 524 | EVQLVESGGGEVQPGGSLRLSCAASGSMTGANTMGWYRQAP | hz18H10v5 |
| | GKQRDLVALIGNYVTHYAESVKGRFTISRENAKNTVYLQMSSL | |
| | RAEDTAVYYCYLYTDNLGTSWGQGTLVTVKP | |
| 525 | EVQLVESGGGEVQPGGSLRLSCAASGSVTGANTMGWYRQAPG | hz18H10v6 |
| | KQRDLVALIGNYVTHYAESVKGRFTISRDNAKNTVYLQMSSLR | |
| | AEDTAVYYCYLYTDNLGTSWGQGTLVTVKP | |
| 526 | EVQLVESGGGEVQPGGSLRLSCAASGSITGANTMGWYRQAPG | hz18H10v7 |
| | KQRDLVALIGNYVTHYAESVKGRFTISRDNAKNTVYLQMSSLR | |
| | AEDTAVYYCYLYTDNLGTSWGQGTLVTVKP | |
| 527 | EVQLVESGGGEVQPGGSLRLSCAASGSVTGANTMGWYRQAPG | hz18H10v8 |
| | KQRDLVSLIGNYVTHYAESVKGRFTISRDNAKNTLYLQMSSLR | |
| | AEDTAVYYCYLYTDNLGTSWGQGTLVTVKP | |
| 528 | EVQLVESGGGEVQPGGSLRLSCAASGSVTGANTMGWYRQAPG | hz18H10v9 |
| | KQRDLVSLIGNYVTHYAESVKGRFTISRDNAKNTVYLQMSSLR | |
| | AEDTAVYYCYLYTDNLGTSWGQGTLVTVKP | |
| 529 | EVQLVESGGGEVQPGGSLRLSCAASGSMTGANTMGWYRQAP | hz18H10v10 |
| | GKQRDLVSLIGNYVTHYAESVKGRFTISRENAKNTVYLQMSSL | |
| | RAEDTAVYYCYLYTDNLGTSWGQGTLVTVKP | |
| 530 | EVQLVESGGGEVQPGGSLRLSCAASGSVTGANTMGWYRQAPG | hz18H10v11 |
| | KQRDLVSLIGNYVTHYAESVKGRFTISRENAKNTVYLQMSSLR | |
| | AEDTAVYYCYLYTDNLGTSWGQGTLVTVKP | |
| 531 | EVQLVESGGGEVQPGGSLRLSCAASGSVTGANTMGWYRQAPG | hz18H10v12 |
| | KQRDLVALIGNYVTHYAESVKGRFTISRENAKNTVYLQMSSLR | |
| | AEDTAVYYCYLYTDNLGTSWGQGTLVTVKP | |
| 532 | EVQLVESGGGEVQPGGSLRLSCAASGSMTGANTMGWYRQAP | hz18H10v13 |
| | GKQRELVALIGNYVTHYAESVKGRFTISRENAKNTVYLQMSSL | |
| | RAEDTAVYYCYLYTDNLGTSWGQGTLVTVKP | |
| 533 | EVQLVESGGGEVQPGGSLRLSCAASGSVTGANTMGWYRQAPG | hz18H10v14 |
| | KQRDLVALIGNYVTHYAESVKGRFTISRDNAKNTLYLQMSSLR | |
| | AEDTAVYYCYLYTDNLGTSWGQGTLVTVKP | |
| 534 | EVQLVESGGGEVQPGGSLRLSCAASGSITGANTMGWYRQAPG | hz18H10v15 |
| | KQRDLVALIGNYVTHYAESVKGRFTISRDNAKNTLYLQMSSLR | |
| | AEDTAVYYCYLYTDNLGTSWGQGTLVTVKP | |
| 535 | EVQLVESGGGEVQPGGSLRLSCAASGSITGANTMGWYRQAPG | hz18H10v16 |
| | KQRDLVALIGNYVTHYAESVKGRFTISRENAKNTVYLQMSSLR | |
| | AEDTAVYYCYLYTDNLGTSWGQGTLVTVKP | |
| 536 | EVQLVESGGGEVQPGGSLRLSCAASGSITGANTMGWYRQAPG | hz18H10v17 |
| | KQRDLVALIGNYVTHYAESVKGRFTISRDNAKNTVYLQMNSL | |
| | RAEDTAVYYCYLYTDNLGTSWGQGTLVTVKP | |
| 537 | EVQLVESGGGLVQPGRSLRLSCAASGFTFDDYAMHWVRQAPG | Anti-CD3 VH |
| | KGLEWVSGISWNSGSIGYADSVKGFTISRDNAKNSLYLQMNSL | 312557 |
| | RAEDTALYYCAKDSRGYGDYRLGGAYWGQGTLVTVSS | |
| 538 | EVQLVESGGGLVQPGRSLRLSCAASGFTFDDYAMHWVRQAPG | Anti-CD3 VH |
| | KCLEWVSGISWNSGSIGYADSVKGFTISRDNAKNSLYLQMNSL | 312557 G44C |
| | RAEDTALYYCAKDSRGYGDYRLGGAYWGQGTLVTVSS | |
| 539 | EIVMTQSPATLSVSPGERATLSCRASQSVSSNLAWYQQKPGQA | Anti-CD3 VL |
| | PRLLIYGASTRATGIPARFSGSGSGTEFTLTISSLQSEDFAVYYC | 312557 |
| | QQYNNWPWTFGQGTKVEIK | |
| 540 | EIVMTQSPATLSVSPGERATLSCRASQSVSSNLAWYQQKPGQA | Anti-CD3 VL |
| | PRLLIYGASTRATGIPARFSGSGSGTEFTLTISSLQSEDFAVYYC | 312557 Q100C |
| | QQYNNWPWTFGCGTKVEIK | |
| 541 | EVQLVESGGGLVQPGRSLRLSCVASGFTFDDYSMHWVRQAPG | CD3-VH-G |
| | KGLEWVSGISWNSGSKDYADSVKGRFTISRDNAKNSLYLQMN | |
| | SLRAEDTALYYCAKYGSGYGKFYHYGLDVWGQGTTVTVSS | |
| 542 | EVQLVESGGGLVQPGRSLRLSCVASGFTFDDYSMHWVRQAPG | CD3-VH-G |
| | KCLEWVSGISWNSGSKDYADSVKGRFTISRDNAKNSLYLQMN | |
| | SLRAEDTALYYCAKYGSGYGKFYHYGLDVWGQGTTVTVSS | |
| 543 | DIQMTQSPSSLSASVGDRVTITCRASQSISSYLNWYQQKPGKAP | V_{K1}-39Jk5 |
| | KLLIYAASSLQSGVPSRFSGSGSGTDFTLTISSLQPEDFATYYCQ | |
| | QSYSTPPITFGQGTRLEIK | |
| 544 | DIQMTQSPSSLSASVGDRVTITCRASQSISSYLNWYQQKPGKAP | V_{K1}-39Jk5 Q100C |
| | KLLIYAASSLQSGVPSRFSGSGSGTDFTLTISSLQPEDFATYYCQ | |
| | QSYSTPPITFGCGTRLEIK | |
| 545 | (ADAAP)n | linker |
| | n = 2-20 | |
| 546 | (ADAAP)n-G | linker |
| | n = 2-20 | |
| 547 | (GEPQG)n | linker |
| | n = 2-20 | |
| 548 | (GEPQG)n-G | linker |
| | n = 2-20 | |
| 549 | (AGGEP)n | linker |
| | n = 2-20 | |
| 550 | (AGGEP)n-G | linker |
| | n = 2-20 | |
| 551 | (AGSEP)n | linker |
| | n = 2-20 | |
| 552 | (AGSEP)n-G | linker |
| | n = 2-20 | |
| 553 | (GGGEQ)n | linker |
| | n = 2-20 | |
| 554 | (GGGEQ)n-G | linker |
| | n = 2-20 | |
| 555 | ADAAPADAAPG | linker |
| 556 | GEPQGGEPQGG | linker |
| 557 | AGGEPAGGEPG | linker |
| 558 | AGSEPAGSEPG | linker |
| 559 | GGGEQGGGEQG | linker |

1. A B7H3-binding polypeptide construct, comprising at least one heavy chain only variable domain (B7H3 VHH domain) that specifically binds B7H3 and one or more additional binding domain that binds to a target other than B7H3.

2. (canceled)

3. A B7H3-binding polypeptide construct, comprising at least one heavy chain only variable domain (B7H3 VHH domain) comprising a complementarity determining region 1 (CDR1) comprising an amino acid sequence selected from the group consisting of SEQ ID NO: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and 145; a complementarity determining region 2 (CDR2) comprising an amino acid sequence selected from the group consisting of SEQ ID NO: 146, 147, 148, 149, 150, 151, 152, 153, 154, 155, 156, 157, 158, 159, 160, 161, 162, 163, 164, 165, 166 and 167; and a complementarity determining region 3 (CDR3) comprising an amino acid sequence selected from the group consisting of SEQ ID NO: 168, 169, 170, 171, 172, 173, 174, 175, 176, 177, 178, 179, 180, 181, 182, 183, 184, 185, 186, 187, 188, 189, and 483-488.

4. The B7H3-binding polypeptide construct of claim 3, comprising one or more additional binding domain(s) that binds to a target other than B7H3.

5. The B7H3-binding polypeptide construct of claim 3, wherein the at least one B7H3 VHH domain is humanized.

6. The B7H3-binding polypeptide construct of claim 4, wherein the one or more additional binding domain(s) binds to an activating receptor on an immune cell.

7. (canceled)

8. The B7H3-binding polypeptide construct of claim 6, wherein the activating receptor is CD3.

9. The B7H3-binding polypeptide construct of claim 3, wherein the at least one B7H3 VHH domain is conjugated to a radioactive ligand.

10-20. (canceled)

21. The B7H3-binding polypeptide construct of claim 3, wherein the polypeptide construct comprises an immunoglobulin Fc region.

22. (canceled)

23. The B7H3-binding polypeptide construct of claim 3 that is a dimer.

24-27. (canceled)

28. The B7H3-binding polypeptide construct of claim 21, wherein the Fc region is a heterodimeric Fc region.

29-33. (canceled)

34. The B7H3-binding polypeptide construct of claim 3, wherein the at least one B7H3 VHH domain comprises (i) the sequence set forth in SEQ ID NO:1, (ii) a humanized variant of SEQ ID NO:1, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:1, and binds B7H3.

35. The B7H3-binding polypeptide construct of claim 3, wherein:
(a) the at least one B7H3 VHH domain comprises a CDR1 comprising an amino acid sequence selected from the group consisting of SEQ ID NO: 115, 116, 117, 118, 119, 120 and 121; a CDR2 comprising an amino acid sequence selected from the group consisting of SEQ ID NO: 146, 147, 148, 149, 150 and 151; and a CDR3 comprising an amino acid sequence selected from the group consisting of SEQ ID NO: 168 and 169; and/or
(b) the at least one B7H3 VHH domain comprises a CDR1, CDR2 and CDR3 set forth in SEQ ID NOS: 115, 146 and 168, respectively; SEQ ID NOS: 115, 147 and 168, respectively; SEQ ID NOS: 115, 148 and 168, respectively; SEQ ID NOS: 115, 149 and 168, respectively; SEQ ID NOS: 115, 150 and 168, respectively; SEQ ID NOS: 116, 146 and 168, respectively; SEQ ID NOS: 117, 146 and 168, respectively; SEQ ID NOS: 118, 146 and 168, respectively; SEQ ID NOS: 115, 146 and 169, respectively; SEQ ID NOS: 119, 146 and 168, respectively; SEQ ID NOS: 120, 146 and 168, respectively; SEQ ID NOS: 115, 151 and 168, respectively; SEQ ID NOS: 116, 147 and 168, respectively; SEQ ID NOS: 118, 147 and 168, respectively; SEQ ID NOS: 119, 147 and 168, respectively; SEQ ID NOS: 116, 151 and 168, respectively; SEQ ID NOS: 115, 146 and 168, respectively; SEQ ID NOS: 121, 147 and 168, respectively; SEQ ID NOS: 115, 146 and 168, respectively; SEQ ID NOs: 119, 149 and 168, respectively; or SEQ ID NOS: 122, 151 and 168, respectively.

36. (canceled)

37. The B7H3-binding polypeptide construct of claim 3, wherein the at least one B7H3 VHH domain comprises the sequence of amino acids set forth in any one of SEQ ID NOs:8-34, 467, 489-490, and 492-497 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 8-34, 467, 489-490, and 492-497, and binds B7H3.

38. (canceled)

39. The B7H3-binding polypeptide construct of claim 3, wherein the at least one B7H3 VHH domain comprises (i) the sequence set forth in SEQ ID NO:35, (ii) a humanized variant of SEQ ID NO:35, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:35, and binds B7H3.

40. The B7H3-binding polypeptide of claim 3, wherein:
(a) the at least one B7H3 VHH domain comprises a CDR1 comprising an amino acid sequence set forth in SEQ ID NO: 123; a CDR2 comprising an amino acid sequence selected from the group consisting of SEQ ID NO: 152 and 153; and a CDR3 comprising an amino acid sequence selected from the group consisting of SEQ ID NO: 170 and 171; and/or
(b) the at least one B7H3 VHH domain comprises a CDR1, CDR2 and CDR3 set forth in SEQ ID NOS: 123, 152 and 170, respectively; SEQ ID NOS: 123, 152 and 171, respectively; SEQ ID NOS: 123, 153 and 170, respectively; or SEQ ID NOS: 123, 153 and 171, respectively.

41. (canceled)

42. The B7H3-binding polypeptide construct of claim 3, wherein the at least one B7H3 VHH domain comprises the sequence of amino acids set forth in any one of SEQ ID NOs:40, 41, or 498-503 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 40, 41, or 498-503, and binds B7H3.

43. (canceled)

44. The B7H3-binding polypeptide construct of claim 3, wherein the at least one B7H3 VHH domain comprises (i) the sequence set forth in SEQ ID NO:44 (ii) a humanized variant of SEQ ID NO:44, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:44, and binds B7H3.

45. The B7H3-binding polypeptide construct of claim 3, wherein:
(a) the at least one B7H3 VHH domain comprises a CDR1 comprising an amino acid sequence selected from the group consisting of SEQ ID NO: 124, 125, 126, 127, 128, 129, 130, 131, 132, or 133; a CDR2 comprising an amino acid sequence set forth in SEQ ID NO: 154; and a CDR3 comprising an amino acid sequence selected from the group consisting of SEQ ID NO: 172, 173, 174, 175, 176, 177, 178, 179, 180, 181, 182 and 183; and/or
(b) the at least one B7H3 VHH domain comprises a CDR1, CDR2 and CDR3 set forth in SEQ ID NOS: 124, 154 and 172, respectively; SEQ ID NOS: 124, 154 and 173, respectively; SEQ ID NOS: 124, 154 and 174, respectively; SEQ ID NOS: 124, 154 and 175, respectively; SEQ ID NOS: 125, 154 and 173, respectively; SEQ ID NOS: 126, 154 and 173, respectively; SEQ ID NOS: 127, 154 and 173, respectively; SEQ ID NOS: 128, 154 and 173, respectively; SEQ ID NOS: 129, 154 and 173, respectively; SEQ ID NOS: 130, 154 and 173, respectively; SEQ ID NOS: 131, 154 and 173, respectively; SEQ ID NOS: 124, 154 and 176, respectively; SEQ ID NOS: 124, 154 and 177, respectively; SEQ ID NOS: 124, 154 and 178, respectively; SEQ ID NOS: 124, 154 and 179, respectively; SEQ ID NOS: 124, 154 and 180, respectively; SEQ ID NOS: 124, 154 and 181, respectively; SEQ ID NOS: 124, 154 and 182, respectively; SEQ ID NOS: 124, 154 and 183, respectively; SEQ ID NOS: 126, 154 and 176, respectively; SEQ ID NOS: 124, 154 and 179, respectively; SEQ ID NOS: 124, 154 and 182, respectively; SEQ ID NOS: 132, 154 and 176, respectively; or SEQ ID NOS: 133, 154 and 173, respectively.

46. (canceled)

47. The B7H3-binding polypeptide construct of claim 3, wherein the at least one B7H3 VHH domain comprises the sequence of amino acids set forth in any one of SEQ ID NOs:56-91, 466, and 504-514 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 56-91, 466, and 504-514, and binds B7H3.

48. (canceled)

49. The B7H3-binding polypeptide construct of claim 3, wherein the at least one B7H3 VHH domain comprises (i) the sequence set forth in SEQ ID NO:105 (ii) a humanized variant of SEQ ID NO:105, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:105, and binds B7H3.

50. The B7H3-binding polypeptide construct of claim 3, wherein the at least one B7H3 VHH domain comprises a CDR1 comprising an amino acid sequence set forth in SEQ ID NO: 145; a CDR2 comprising an amino acid sequence set forth in SEQ ID NO: 167; and a CDR3 comprising an amino acid sequence set forth in SEQ ID NO: 488.

51. The B7H3-binding polypeptide construct of claim 3, wherein the at least one B7H3 VHH domain comprises the sequence of amino acids set forth in any one of SEQ ID NOs:106-109 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO:106-109, and binds B7H3.

52. (canceled)

53. The B7H3-binding polypeptide construct of claim 3, wherein the at least one B7H3 VHH domain comprises (i) the sequence set forth in SEQ ID NO:110 (ii) a humanized variant of SEQ ID NO:110, or (iii) a sequence of amino acids that exhibits at least 85%, 86%, 87%, 88%, 89%, 90%, 91%, 92%, 93%, 94%, 95%, 96%, 97%, 98%, or 99% sequence identity to SEQ ID NO:110, and binds B7H3.

54. The B7H3-binding polypeptide of claim 3, wherein the at least one B7H3 VHH domain comprises a CDR1 comprising an amino acid sequence set forth in SEQ ID NO: 139; a CDR2 comprising an amino acid sequence set forth in SEQ ID NO: 161; and a CDR3 comprising an amino acid sequence set forth in SEQ ID NO: 189.

55. The B7H3-binding polypeptide construct of claim 3, wherein the at least one B7H3 VHH domain comprises the sequence of amino acids set forth in any one of SEQ ID NOs:515-518 or a sequence of amino acids that exhibits at least 85%, 86%, 87%, 88%, 89%, 90%, 91%, 92%, 93%, 94%, 95%, 96%, 97%, 98%, or 99% sequence identity to any one of SEQ ID NO: 515-518, and binds B7H3.

56-59. (canceled)

60. The B7H3-binding polypeptide construct of claim 3, comprising: (a) a first component comprising a heterodimeric Fc region comprising a first Fc polypeptide and a second Fc polypeptide and (b) a second component comprising an anti-CD3 antibody or antigen-binding fragment comprising a variable heavy chain region (VH) and a variable light chain region (VL), wherein:
the VH and VL that comprise the anti-CD3 antibody or antigen binding fragment are linked to opposite polypeptides of the heterodimeric Fc;
the first and second components are coupled by a linker, wherein the heterodimeric Fc region is positioned amino-terminally to the anti-CD3 antibody or antigen-binding fragment; and
one or both of the first and second components comprises the at least one B7H3 VHH domain.

61-63.(canceled)

64. The B7H3-binding polypeptide construct of claim 60, wherein each of the first and second Fc polypeptides of the heterodimeric Fc region comprises a knob-into-hole modification or comprises a charge mutation to increase electrostatic complementarity of the polypeptides.

65-86. (canceled)

87. The B7H3-binding polypeptide construct of claim 60, wherein the anti-CD3 antibody or antigen-binding fragment is an Fv antibody fragment.

88. The B7H3-binding polypeptide construct of claim 87, wherein the Fv antibody fragment comprises a disulfide stabilized anti-CD3 binding Fv fragment (dsFv).

89. The B7H3-binding polypeptide construct of claim 60, wherein:
(a) the anti-CD3 antibody or antigen-binding fragment comprises a VH CDR1 comprising the amino acid sequence TYAMN (SEQ ID NO: 219); a VH CDR2 comprising the amino acid sequence RIRSKYNNYATYYADSVKD (SEQ ID NO: 220); a VH CDR3 comprising the amino acid sequence HGNFGNSYVSWFAY (SEQ ID NO: 221), a VL CDR1 comprising the amino acid sequence RSSTGAVTTSNYAN (SEQ ID NO: 222); a VL CDR2 comprising the amino acid sequence GTNKRAP (SEQ ID NO: 223); and a VL CDR3 comprising the amino acid sequence ALWYSNLWV (SEQ ID NO: 224);
(b) the anti-CD antibody or antigen-binding fragment comprises a VH CDR1 comprising the amino acid sequence GFTFNTYAMN (SEQ ID NO: 471); a VH CDR2 comprising the amino acid sequence RIRSKYNNYATY (SEQ ID NO: 472); a VH CDR3 comprising the amino acid sequence HGNFGNSYVSWFAY (SEQ ID NO: 221), a VL CDR1 comprising the amino acid sequence RSSTGAVTTSNYAN (SEQ ID NO: 19); a VL CDR2 sequence comprising the amino acid sequence GTNKRAP (SEQ ID NO: 20); and a VL CDR3 comprising the amino acid sequence ALWYSNLWV (SEQ ID NO: 21);
(c) the anti-CD antibody or antigen-binding fragment comprises a VH CDR1 comprising the amino acid sequence GFTFNTYAMN (SEQ ID NO: 471); a VH CDR2 comprising the amino acid sequence RIRSKYNNYATY (SEQ ID NO: 472); a VH CDR3 comprising the amino acid sequence HGNFGNSYVSWFAY (SEQ ID NO: 221), a VL CDR1 comprising the amino acid sequence RSSTGAVTTSNYAN (SEQ ID NO: 222); a VL CDR2 comprising the amino acid sequence GTNKRAP (SEQ ID NO: 223); and a VL CDR3 comprising the amino acid sequence ALWYSNLWV (SEQ ID NO: 224);
(d) the anti-CD antibody or antigen-binding fragment comprises a VH CDR1 comprising the amino acid sequence GFTFNTYAMN (SEQ ID NO: 471); a VH CDR2 comprising the amino acid sequence RIRSKYNNYATY (SEQ ID NO: 472); a VH CDR3 comprising the amino acid sequence HGNFGNSYVSWFAY (SEQ ID NO: 221), a VL CDR1 comprising the amino acid sequence GSSTGAVTTSNYAN (SEQ ID NO: 478); a VL CDR2 comprising the amino acid sequence GTNKRAP (SEQ ID NO: 479); and a VL CDR3 comprising the amino acid sequence ALWYSNHWV (SEQ ID NO: 474);
(e) the anti-CD antibody or antigen-binding fragment comprises a VH CDR1 comprising the amino acid sequence GFTFNTYAMN (SEQ ID NO: 471); a VH CDR2 comprising the amino acid sequence RIRSKYNNYATY (SEQ ID NO: 472); a VH CDR3 comprising the amino acid sequence HGNFGNSYVSWFAY (SEQ ID NO: 221), a VL CDR1 comprising the amino acid sequence GSSTGAVTTSNYAN (SEQ ID NO: 478); a VL CDR2 comprising the amino acid sequence GTNKRAP (SEQ ID NO: 223); and a VL CDR3 comprising the amino acid sequence ALWYSNHWV (SEQ ID NO: 474);
(f) the anti-CD antibody or antigen-binding fragment comprises a VH CDR1 comprising the amino acid sequence GFTFSTYAMN (SEQ ID NO: 476); a VH CDR2 comprising the amino acid sequence RIRSKYNNYATY (SEQ ID NO: 477); a VH CDR3 comprising the amino acid sequence HGNFGDSYVSWFAY (SEQ ID NO: 473), a VL CDR1 comprising the amino acid sequence GSSTGAVTTSNYAN (SEQ ID NO: 478); a VL CDR2 comprising the amino acid sequence GTNKRAP (SEQ ID NO: 223); and a VL CDR3 comprising the amino acid sequence ALWYSNHWV (SEQ ID NO: 474); and/or
(g) a VH having the amino acid sequence of any of SEQ ID NOS: 225-255, 480, 460, or 462 or a sequence that exhibits at least 90%, 91%, 92%, 93%, 94%, 95%, 96%, 97%, 98% or 99% sequence identity to any of SEQ ID NOS: 225-255,460, or 462 and binds CD3; and
a VL having the amino acid sequence of any of SEQ ID NOS: 256-274, 417,459, or 461 or a sequence that exhibits at least 90%, 91%, 92%, 93%, 94%, 95%, 96%, 97%, 98% or 99% sequence identity to any of SEQ ID NOS: 256-274, 417,459, or 461 and binds CD3.

90-94. (canceled)

95. The B7H3-binding polypeptide construct of claim 60, wherein the at least one B7H3 single domain antibody is positioned amino-terminally relative to the Fc region and/or carboxy-terminally relative to the anti-CD3 antibody or antigen-binding fragment.

96-128. (canceled)

129. The B7H3-binding polypeptide construct of claim 96, wherein one or both of the first and second components comprises at least one co-stimulatory receptor binding region (CRBR) that binds a co-stimulatory receptor.

130-140. (canceled)

141. The B7H3-binding polypeptide construct of claim 96, wherein one or both of the first and second components comprises at least one inhibitory receptor binding region (IRBR) that binds an inhibitory receptor.

142-153. (canceled)

154. The B7H3-binding polypeptide construct of claim 96, wherein the linker is a non-cleavable linker.

155-163. (canceled)

164. An isolated single domain antibody that binds B7H3, comprising a complementarity determining region 1 (CDR1) comprising an amino acid sequence selected from the group consisting of SEQ ID NO: 115, 116, 117, 118, 119, 120, 121, 122, 123, 124, 125, 126, 127, 128, 129, 130, 131, 132, 133, 134, 135, 136, 137, 138, 139, 140, 141, 142, 143, 144 and 145; a complementarity determining region 2 (CDR2) comprising an amino acid sequence selected from the group consisting of SEQ ID NO: 146, 147, 148, 149, 150, 151, 152, 153, 154, 155, 156, 157, 158, 159, 160, 161, 162, 163, 164, 165, 166 and 167; and a complementarity determining region 3 (CDR3) comprising an amino acid sequence selected from the group consisting of SEQ ID NO: 168, 169, 170, 171, 172, 173, 174, 175, 176, 177, 178, 179, 180, 181, 182, 183, 184, 185, 186, 187, 188, 189, and 483-488.

165-190. (canceled)

191. A polynucleotide(s) encoding the B7H3-binding polypeptide of claim 3.

192-195. (canceled)

196. A polynucleotide encoding the single domain antibody of claim 164.

197. A vector, comprising the polynucleotide or polynucleotides of claim 191.

198-199. (canceled)

200. A cell, comprising the polynucleotide or polynucleotides of claim 191.

201-202. (canceled)

203. A method of producing a polypeptide, the method comprising introducing into a cell a polynucleotide or polynucleotides of claim 191 and culturing the cell under conditions to produce the multispecific polypeptide construct.

204-205. (canceled)

206. An engineered immune cell comprising a chimeric antigen receptor comprising:
an extracellular domain comprising the single domain antibody of claim 164;
a transmembrane domain; and
an intracellular signaling domain.

207-212. (canceled)

213. A pharmaceutical composition comprising the B7H3-binding polypeptide of claim 3.

214. A pharmaceutical composition comprising the engineered immune cell of claim 206.

215. (canceled)

216. A method of stimulating or inducing an immune response in a subject, the method comprising administering, to a subject in need thereof, the pharmaceutical composition of claim 213.

217-218. (canceled)

219. A method of treating a disease or condition in a subject, the method comprising administering, to a subject in need thereof, a therapeutically effective amount of the pharmaceutical composition of claim 213.

220. A method of treating a disease or condition in a subject, the method comprising administering, to a subject in need thereof, the pharmaceutical composition of claim 214.

221. (canceled)
